(19) United States
(12) Patent Application Publication
(10) Pub. No.: US 2021/0009564 A1
(43) Pub. Date: Jan. 14, 2021

(54) CALPAIN MODULATORS AND THERAPEUTIC USES THEREOF

(71) Applicant: BLADE THERAPERTICS, INC., South San Francisco, CA (US)

(72) Inventors: Brad Owen Buckman, Oakland, CA (US); Shendong Yuan, San Ramon, CA (US); Kumaraswamy Emayan, Albany, CA (US); Marc Adler, Orinda, CA (US); Prabha Ibrahim, Mountain View, CA (US)

(21) Appl. No.: 17/041,596

(22) PCT Filed: Mar. 21, 2019
(86) PCT No.: PCT/US2019/023457

§ 371 Date: Sep. 25, 2020

Related U.S. Application Data
(60) Provisional application No. 62/649,451, filed on Mar. 28, 2018.

(51) Int. Cl. C07D 403/04; C07D 285/10; C07D 231/12; C07D 263/32; C07D 417/04; C07D 405/04; C07D 401/04; C07D 413/04; C07D 409/04; A61P 25/28

(52) U.S. Cl. CPC C07D 403/04; C07D 285/10; C07D 231/12; C07D 263/32; C07D 417/04; A61K 9/0019; C07D 401/04; C07D 413/04; C07D 409/04; A61P 25/28; C07D 405/04

(57) ABSTRACT

Small molecule calpain modulator compounds, including their pharmaceutically acceptable salts, can be included in pharmaceutical compositions. The compounds can be useful in inhibiting calpain, or competitive binding with calpastatin, by contacting them with CAPN1, CAPN2, and/or CAPN9 enzymes residing inside a subject. The compounds and composition can also be administered to a subject in order to treat a fibrotic disease or a secondary disease state or condition of a fibrotic disease.

### BACKGROUND

### Field of the Invention

The present invention relates to the fields of chemistry and medicine. More particularly, the present invention relates to non-macrocyclic α-keto amide compounds as small molecule calpain modulators, compositions, their preparation, and their use as therapeutic agents.

### Description of the Related Art

Fibrotic disease accounts for an estimated 45% of deaths in the developed world but the development of therapies for such diseases is still in its infancy. The current treatments for fibrotic diseases, such as for idiopathic lung fibrosis, renal fibrosis, systemic sclerosis, and liver cirrhosis, are few in number and only alleviate some of the symptoms of fibrosis while failing to treat the underlying cause.

Despite the current limited understanding of the diverse etiologies responsible for these conditions, similarities in the phenotype of the affected organs, across fibrotic diseases, strongly support the existence of common pathogenic pathways. At present, it is recognized that a primary driver of fibrotic disease is a high transforming growth factor-beta (TGFβ) signaling pathway which can promote the transformation of normally functioning cells into fibrosis-promoting cells. Termed “myofibroblasts,” these transformed cells can secrete large amounts of extracellular matrix proteins and matrix degrading enzymes, resulting in the formation of scar tissue and eventual organ failure. This cellular process is transformative and termed “myofibroblast differentiation” (which includes Epithelial-to-Mesenchymal Transition (EpMT) and its variations like Endothelial-to-Mesenchymal Transition (EnMT) and Fibroblast-to-Myofibroblast Transition (FMT)). This process is a major target for the treatment of fibrotic diseases. Myofibroblast differentiation has also been shown to occur within cancer cells that have been chronically exposed to high TGFβ, causing stationary epithelial cells to become motile, invasive, and metastasize. Thus, within the context of cancer, the signaling has been documented to associate with the acquisition of drug resistance, immune system evasion, and development of stem cell properties.

Despite the tremendous potential of myofibroblast differentiation-inhibiting drugs, and the numerous attempts to develop a working treatment, the data gathered thus far has yet to translate into practical therapy. This is partly due to the lack of an ideal target protein. Initial strategies to target the myofibroblast differentiation process focused on proximal inhibition of the TGFβ signaling pathway by various methods, including targeting ligand activators (e.g. alpha-v integrins), ligand-receptor interactions (e.g., using neutralizing antibodies) or TGFβ receptor kinase activity (e.g., small molecule chemical compound drugs to block signal transduction). Unfortunately, TGFβ is a pleiotropic cytokine with many physiological functions such that global suppression of TGFβ signaling was also associated with severe side effects. Additionally, current data suggests that such proximal inhibition may be vulnerable to pathologic workaround strategies (i.e., due to redundancy or compensation), that would limit the utility of such drugs. Further complicating matters is that, in cancer, TGFβ signaling early on functions as an anti-tumorigenic growth inhibitor but later becomes tumor promoting and is another reason why selective inhibition of pathogenic elements of signaling is so strongly desired. In light of these inherent limitations, current treatment strategies have refocused on identification and inhibition of critical distal events in TGFβ signaling, which in theory would preferentially target the pathologic, but not physiological functions of TGFβ signaling.

### SUMMARY

A compound having the structure of the formula (I):

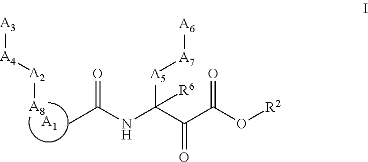

or a pharmaceutically acceptable salt thereof, wherein:

A₁ is selected from the group consisting of optionally substituted 5-10 membered heterocyclyl; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH≡CH—, —C≡C—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁸ is attached;

A₈ is a ring member of A₁ and is selected from the group consisting of C and N;

R is independently selected from —H, halo, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R² is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;

R⁶ is independently selected from —H and optionally substituted C₁₋₄ alkyl; and

each n is independently selected to be an integer from 0 to 3.

Other embodiments disclosed herein include a pharmaceutical composition comprising a therapeutically effective amount of a compound disclosed herein and a pharmaceutically acceptable excipient.

Other embodiments disclosed herein include a method of treating diseases and conditions mediated at least in part by the physiologic effects of CAPN1, CAPN2, or CAP9, or combinations thereof, comprising administering to a subject in need thereof a compound disclosed herein.

In some embodiments, compounds disclosed herein are specific inhibitors of one of: CAPN1, CAPN2 or CAPN9.

In some embodiments, compounds disclosed herein are selective inhibitors of one of: CAPN1, CAPN2 or CAPN9.

In some embodiments, compounds disclosed herein are selective inhibitors of: CAPN1 and CAPN2, or CAPN1 and CAPN9, or CAPN2 and CAPN9.

In some embodiments, compounds disclosed herein are effective inhibitors of CAPN1, CAPN2 and/or CAPN9.

In some embodiments, the non-macrocyclic α-keto amide compounds disclosed herein are broadly effective in treating a host of conditions arising from fibrosis or inflammation, and specifically including those associated with myofibroblast differentiation. Accordingly, compounds disclosed herein are active therapeutics for a diverse set of diseases or disorders that include or that produces a symptom which include, but are not limited to: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases or disorders. In other embodiments, the compounds disclosed herein can be used can be used in metabolic and reaction kinetic studies, detection and imaging techniques and radioactive treatments.

In some embodiments, the compounds disclosed herein are used to treat diseases or conditions or that produces a symptom in a subject which include, but not limited to: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases.

In certain embodiments methods are provided for alleviating or ameliorating a condition or disorder, affected at least in part by the enzymatic activity of calpain 1 (CAPN1), calpain 2 (CAPN2), and/or calpain 9 (CAPN9), or mediated at least in part by the enzymatic activity of CAPN1, CAPN2, and/or CAPN9 wherein the condition includes or produces a symptom which includes: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and/or rheumatoid arthritis.

In some embodiments, the methods, compounds, and/or compositions of the present invention are used for prophylactic therapy.

In some embodiments, the CAPN1, CAPN2, and/or CAPN9 inhibiting compounds demonstrate efficacy in animal models of human disease. Specifically, in-vivo treatment of mice, rabbits, and other mammalian subjects with compounds disclosed herein establish the utility of these compounds as therapeutic agents to modulate CAPN1, CAPN2, and/or CAPN9 activities in humans and thereby ameliorate corresponding medical conditions.

Some embodiments provide compounds, pharmaceutical compositions, and methods of use to inhibit myofibroblast differentiation. Some embodiments provide compounds, pharmaceutical compositions, and methods of use for inhibiting CAPN1, CAPN2, and/or CAPN9 or combinations of these enzyme activities such as CAPN1 and CAPN2, or CAPN1 and CAPN9, or CAPN2 and CAPN9. Some embodiments provide methods for treatment of diseases and disorders by inhibiting CAPN1, CAPN2, and/or CAPN9 or combinations of these enzymatic activities.

### DETAILED DESCRIPTION

In some embodiments, compounds that are non-macrocyclic α-keto amides are provided that act as calpain modulators. Various embodiments of these compounds include compounds having the structures of Formula I as described above or pharmaceutically acceptable salts thereof. The structure of Formula I encompasses all stereoisomers and racemic mixtures, including the following structures and mixtures thereof:

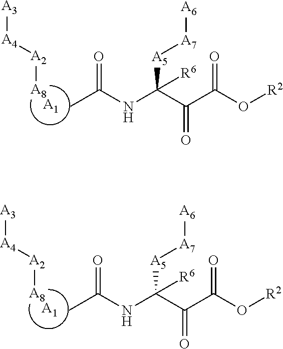

In some embodiments of compounds of Formula (I):

A₁ is selected from the group consisting of optionally substituted 6-10 membered heterocyclyl; optionally substituted 5-, 8-, or 9-membered heteroaryl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted —O—C₁₋₄ alkyl, optionally substituted —O C₂₋₆ alkenyl, and any natural or non-natural amino acid side chain;

R is independently selected from —H, halo, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and

R² is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl.

Some embodiments of compounds of Formula (I) include compounds wherein when A₁ is optionally substituted 5-10 membered heterocyclyl, the 5-10 membered heterocyclyl is not substituted with oxo.

Some embodiments of compounds of Formula (I) include compounds wherein when A₁ is optionally substituted 6-10 membered heterocyclyl, the 6-10-membered heterocyclyl is not substituted with oxo.

Some embodiments of compounds of Formula (I) include compounds having the structure of Formula (I-a):

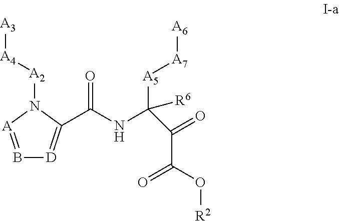

or a pharmaceutically acceptable salt thereof, wherein:

A, B, and D are each independently selected from the group consisting of C(R⁴) and N; and each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy.

In some embodiments of compounds of Formula (I-a) or their pharmaceutically acceptable salts; A, B, and D are independently selected from the group consisting of CH and N. In some embodiments, A is N, B is CH, and D is CH. In some embodiments, A is CH, B is N, and D is CH. In some embodiments, A is N, B is N, and D is N.

Some embodiments of compounds of Formula (I) include compounds having the structure of Formula (I-b):

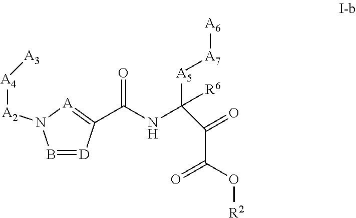

or a pharmaceutically acceptable salt thereof, wherein:

A, B, and D are each independently selected from the group consisting of C(R⁴) and N; and each R⁴ is independently selected from the group consisting of —H, C₁₋₄alkyl, C₁₋₄ haloalkyl, C₃₋₇carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy.

In some embodiments of compounds of Formula (I-b) or their pharmaceutically acceptable salts; A, B, and D are independently selected from the group consisting of CH and N.

Some embodiments of compounds of Formula (I) include compounds having the structure of Formula (I-c):

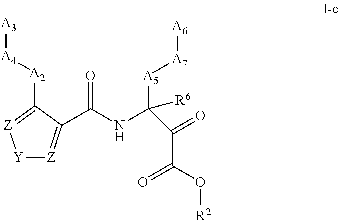

or a pharmaceutically acceptable salt thereof, wherein:

Y is selected from the group consisting of NR⁵, O, S, and SO₂; X and Z are each independently selected from the group consisting of C(R⁴) and N; each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

In some embodiments of compounds of Formula (I-c), Z is N, Y is NR⁵, and X is CH.

In some embodiments of compounds of Formula (I-c), R⁵ is selected from the group consisting of —H, C₁₋₄ alkyl, C₁-C₄ haloalkyl, and cyclopropyl.

In some embodiments of compounds of Formula (I-c), Z is N, Y is O, and X is C(R⁴). In some embodiments of compounds of Formula (I-c), Z is N, Y is S, and X is C(R⁴). In some embodiments of compounds of Formula (I-c), Z is C(R⁴), Y is S, and X is C(R⁴).

In some embodiments of compounds of Formula (I-c), Z is C(R⁴), Y is O, and X is C(R⁴). In some embodiments of compounds of Formula (I-c), Z is C(R⁴), Y is S, and X is N. In some embodiments of compounds of Formula (I-c), Z is C(R⁴), Y is O, and X is N.

In some embodiments of compounds of Formula (I-c), Z is N, Y is S, and X is N. In some embodiments of compounds of Formula (I-c), Z is N, Y is O, and X is N.

Some embodiments of compounds of Formula (I) include compounds having the structure of formula (I-d):

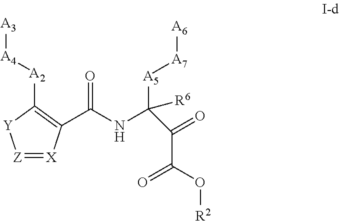

or a pharmaceutically acceptable salt thereof, wherein:

Y is selected from the group consisting of NR⁵, O, S, and SO₂; X and Z are each independently selected from the group consisting of C(R⁴) and N; each R⁴ is independently selected from the group consisting of —H, C₁₋₄ alkyl, C₁₋₄ haloalkyl C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and R⁵ is selected from the group consisting of —H, C₁₋₄ alkyl, C₁₋₄ haloalkyl and C₃₋₇ carbocyclyl (optionally substituted with halo, C₁-C₆ alkyl, C₁-C₆ alkoxy, C₁-C₆ haloalkyl, and C₁-C₆ haloalkoxy).

In some embodiments of compounds of Formula (I-d) or their pharmaceutically acceptable salts; X and Z are independently selected from the group consisting of CH and N. In some embodiments of compounds of Formula (I-d), Y is NR, Z is N, and X is CH. In some embodiments of compounds of Formula (I-d), Z is C(R⁴), Y is O, and X is N. In some embodiments of compounds of Formula (I-d), Z is C(R⁴), Y is S, and X is N.

Some embodiments of compounds of Formula (I) include compounds having the structure of Formula (I-e):

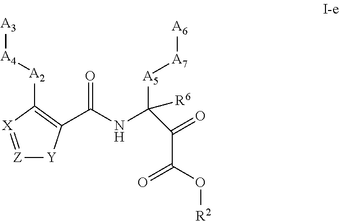

or a pharmaceutically acceptable salt thereof, wherein:

Y is selected from the group consisting of NR⁵, O, S, and SO₂; X and Z are each independently selected from the group consisting of C(R⁴) and N; each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

In some embodiments of compounds of Formula (I-e) or their pharmaceutically acceptable salts; X and Z are independently selected from the group consisting of CH and N. In some embodiments of compounds of Formula (I-e), X is CH, Z is N, and Y is NR.

In some embodiments of compounds of Formula (I-e), X is N, Z is C(R⁴), and Y is O.

In some embodiments of compounds of Formula (I-e), wherein R⁴ is selected from —H and C₁₋₄ alkyl.

In some embodiments of compounds of Formula (I-e), X is N, Z is C(R⁴), and Y is S. In some embodiments of compounds of Formula (I-e), X is N, Z is N, and Y is S.

In some embodiments of compounds of Formula (II),

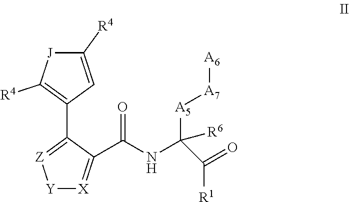

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, and S;

X and Z are each independently selected from the group consisting of C(R⁴) and N;

J is selected from the group consisting of O and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

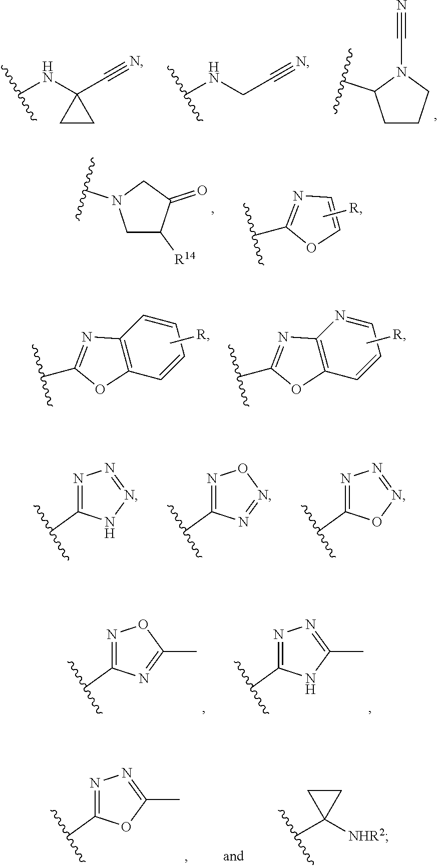

R¹⁴ is halo;

each R, R², and R³ are independently selected from —H, optionally substituted C₁₋₄ alky, optionally substituted C₁₋₈alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄alkyl; and

each n is independently selected to be an integer from 0 to 3; and wherein the compound is not selected from the group consisting of

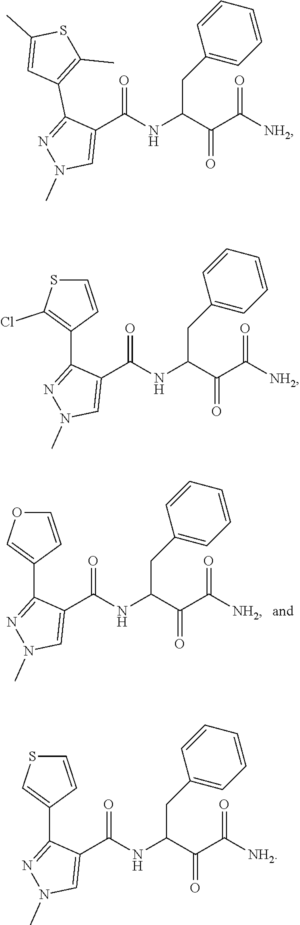 [SMILES: Cc1cc(-c2nn(C)cc2C(=O)NC(Cc2ccccc2)C(=O)C(N)=O)c(C)s1 ; Cn1cc(C(=O)NC(Cc2ccccc2)C(=O)C(N)=O)c(-c2ccsc2Cl)n1 ; Cn1cc(C(=O)NC(Cc2ccccc2)C(=O)C(N)=O)c(-c2ccoc2)n1 ; "and" ; Cn1cc(C(=O)NC(Cc2ccccc2)C(=O)C(N)=O)c(-c2ccsc2)n1]

In some embodiments of compounds of Formula (II) or their pharmaceutically acceptable salts; Z is N, Y is NR, and X is CH. In some embodiments of compounds of Formula (II), R⁵ is selected from the group consisting of —H, C₁₋₄ alkyl, C₁-C₄ haloalkyl, and cyclopropyl. In some embodiments of compounds of Formula (II), Z is N, Y is S, and X is N.

In some embodiments of compounds of Formula (II) or their pharmaceutically acceptable salts; RI is —CONR²R³. In some embodiments of compounds of Formula (II), R¹ is —CONH₂. In some embodiments of compounds of Formula (II), R² is —H and R³ is optionally substituted C₁₋₄ alkyl. In some embodiments of compounds of Formula (II), R² is —H and R³ is selected from the group consisting of —H, C₁-C₄ alkyl optionally substituted with C-amido, and C₃-C₆ cycloalkyl.

In some embodiments of compounds of Formula (II) or their pharmaceutically acceptable salts; R³ is selected from ethyl or cyclopropyl. In some embodiments of compounds of Formula (II), R³ is methyl substituted with C-amido. In some embodiments of compounds of Formula (II), R³ is —H. In some embodiments of compounds of Formula (II), R³ is optionally substituted C₁₋₄ alkyl. In some embodiments of compounds of Formula (II), R³ is benzyl.

In some embodiments of compounds of Formula (II), R¹ is —COOR². In some embodiments of compounds of Formula (I), R² is selected from the group consisting of —H, C₁-C₄ alkyl optionally substituted with C-amido, and C₃-C₆ cycloalkyl.

In some embodiments of compounds of Formula (III),

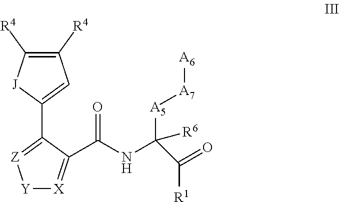

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₈ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached,

Y is selected from the group consisting of NR⁵, and S;

X and Z are each independently selected from the group consisting of C(R⁴) and N;

J is selected from the group consisting of O and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³,

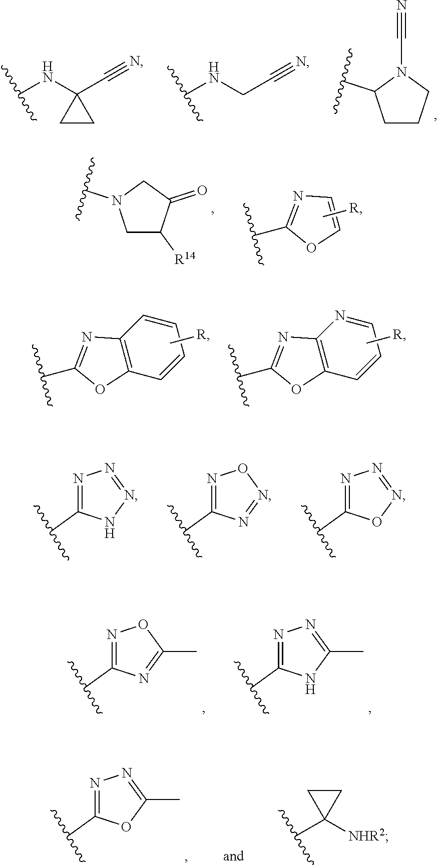

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and

each n is independently selected to be an integer from 0 to 3; and wherein the compound is not selected from the group consisting of

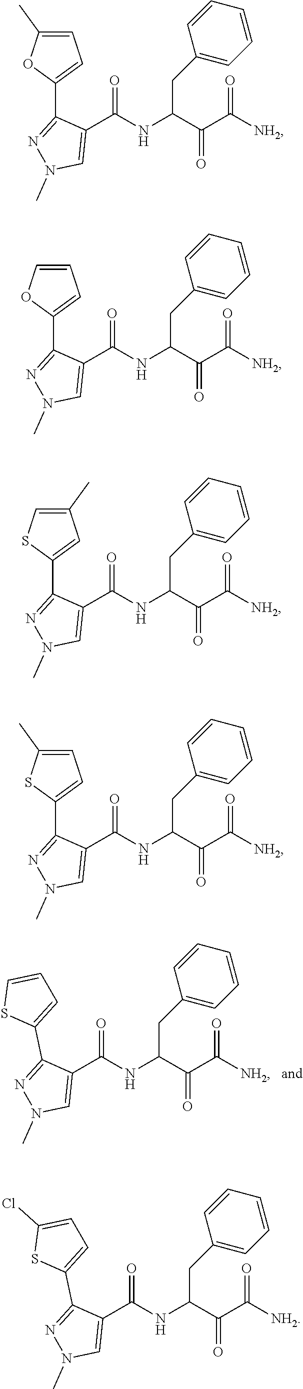

In some embodiments of compounds of Formula (I) or their pharmaceutically acceptable salts; Z is N, Y is NR⁵, and X is CH. In some embodiments of compounds of Formula (I), R⁵ is selected from the group consisting of —H, C₁₋₄ alkyl, C₁-C₄ haloalkyl, and cyclopropyl. In some embodiments of compounds of Formula (I), Z is N, Y is S, and X is N.

In some embodiments of compounds of Formula (III) or their pharmaceutically acceptable salts; R¹ is —CONR²R³. In some embodiments of compounds of Formula (III), R¹ is —CONH₂. In some embodiments of compounds of Formula (III), R² is —H and R³ is optionally substituted C₁₋₄ alkyl. In some embodiments of compounds of Formula (II), R² is —H and R³ is selected from the group consisting of —H, C₁-C₄ alkyl optionally substituted with C-amido, and C₃-C₆ cycloalkyl.

In some embodiments of compounds of Formula (III) or their pharmaceutically acceptable salts; R³ is selected from ethyl or cyclopropyl. In some embodiments of compounds of Formula (I), R³ is methyl substituted with C-amido. In some embodiments of compounds of Formula (III), R³ is —H. In some embodiments of compounds of Formula (III), R³ is optionally substituted C₁₋₄ alkyl. In some embodiments of compounds of Formula (III), R³ is benzyl.

In some embodiments of compounds of Formula (III), R¹ is —COOR². In some embodiments of compounds of Formula (III), R² is selected from the group consisting of —H, C₁-C₄ alkyl optionally substituted with C-amido, and C₃-C₆ cycloalkyl.

In some embodiments of compounds of Formula (IV):

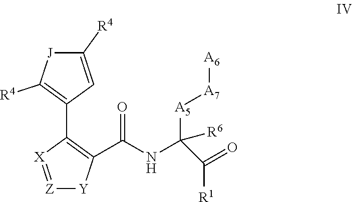

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, O, S, and SO₂;

X and Z are each independently selected from the group consisting of C(R⁴) and N;

J is selected from the group consisting of O and S;

each R⁴ is independently selected from the group consisting of —H, C₁₋₄ alkyl, C₁₋₄ haloalkyl, C₃₋₇carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

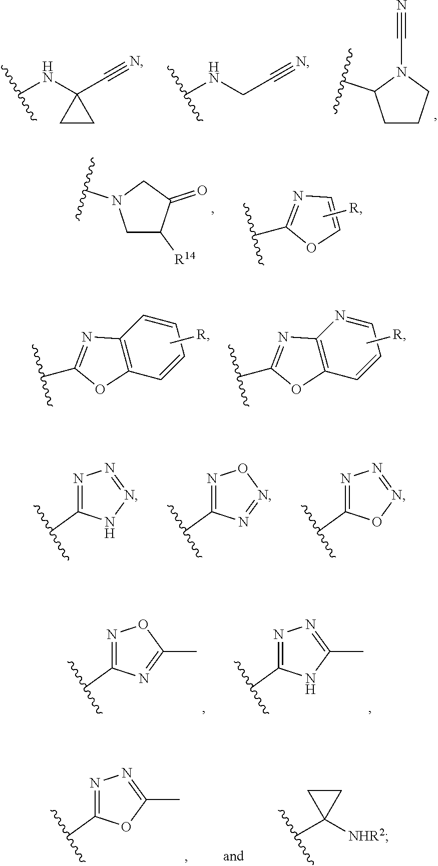

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and

each n is independently selected to be an integer from 0 to 3.

In some embodiments of compounds of Formula (IV) or their pharmaceutically acceptable salts; X and Z are independently selected from the group consisting of C(R⁴) and N. In some embodiments of compounds of Formula (IV), X is N, Z is C(R⁴), and Y is O. In some embodiments of compounds of Formula (IV), R⁴ is selected from —H and C₁₋₄ alkyl.

In some embodiments of compounds of Formula (IV) or their pharmaceutically acceptable salts; R¹ is —CONR²R³. In some embodiments of compounds of Formula (IV), R¹ is —CONH₂. In some embodiments of compounds of Formula (IV), R² is —H and R³ is optionally substituted C₁₋₄ alkyl. In some embodiments of compounds of Formula (IV), R² is —H and R³ is selected from the group consisting of —H, C₁-C₄ alkyl optionally substituted with C-amido, and C₃-C₆ cycloalkyl.

In some embodiments of compounds of Formula (IV) or their pharmaceutically acceptable salts; R³ is selected from ethyl or cyclopropyl. In some embodiments of compounds of Formula (IV), R³ is methyl substituted with C-amido. In some embodiments of compounds of Formula (IV), R³ is —H. In some embodiments of compounds of Formula (IV), R³ is optionally substituted C₁₋₄ alkyl. In some embodiments of compounds of Formula (IV), R³ is benzyl.

In some embodiments of compounds of Formula (IV), R¹ is —COOR². In some embodiments of compounds of Formula (IV), R² is selected from the group consisting of —H, C₁-C₄ alkyl optionally substituted with C-amido, and C₃-C₆ cycloalkyl.

In some embodiments of compounds of Formula (V):

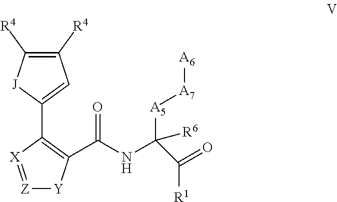

or a pharmaceutically acceptable salt thereof, wherein:

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

Y is selected from the group consisting of NR⁵, O, S, and SO₂;

X and Z are each independently selected from the group consisting of C(R⁴) and N;

J is selected from the group consisting of O and S;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);

R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃,

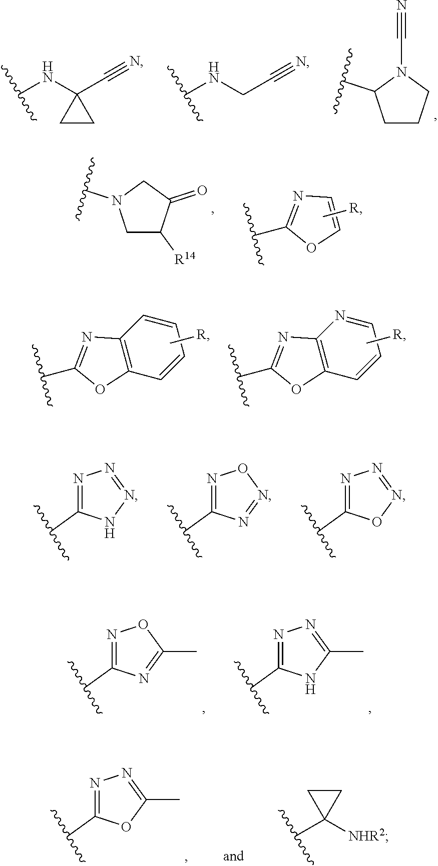

R¹⁴ is halo;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;

R⁶ is independently selected from —H and optionally substituted C₁₋₄ alkyl; and

each n is independently selected to be an integer from 0 to 3.

In some embodiments of compounds of Formula (V) or their pharmaceutically acceptable salts; X and Z are independently selected from the group consisting of C(R⁴) and N. In some embodiments of compounds of Formula (V), X is N, Z is C(R⁴), and Y is O. In some embodiments of compounds of Formula (V), R⁴ is selected from —H and C₁₋₄ alkyl.

In some embodiments of compounds of Formula (V) or their pharmaceutically acceptable salts; R¹ is —CONR²R³. In some embodiments of compounds of Formula (V), R¹ is —CONH₂. In some embodiments of compounds of Formula (V), R² is —H and R³ is optionally substituted C₁₋₄ alkyl. In some embodiments of compounds of Formula (V), R² is —H and R³ is selected from the group consisting of —H, C₁-C₄ alkyl optionally substituted with C-amido, and C₃-C₆ cycloalkyl.

In some embodiments of compounds of Formula (V) or their pharmaceutically acceptable salts; R³ is selected from ethyl or cyclopropyl. In some embodiments of compounds of Formula (V), R³ is methyl substituted with C-amido. In some embodiments of compounds of Formula (V), R³ is —H. In some embodiments of compounds of Formula (V), R³ is optionally substituted C₁₋₄ alkyl. In some embodiments of compounds of Formula (V), R³ is benzyl.

In some embodiments of compounds of Formula (V), R¹ is —COOR². In some embodiments of compounds of Formula (V), R² is selected from the group consisting of —H, C₁-C₄ alkyl optionally substituted with C-amido, and C₃-C₆ cycloalkyl.

In some embodiments of compounds of Formula (VI):

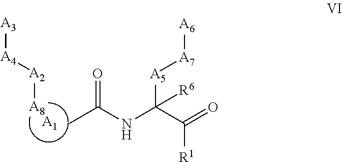

or a pharmaceutically acceptable salt thereof, wherein:

A₁ is selected from the group consisting of optionally substituted 5-10 membered heteroaryl; optionally substituted 5-10 membered heterocyclyl; and optionally substituted C₃₋₁₀ carbocyclyl;

A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;

A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;

when A₂ and A₄ are single bond, A₃ is directly attached to A₈;

A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;

A₈ is a ring member of A₁ and is selected from the group consisting of C and N;

A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₄ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;

A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;

when A₅ and A₇ are single bond, A₆ is directly attached to the carbon to which R⁶ is attached;

R¹ is selected from the group consisting of —C(═O)N(R²)O(R³), —C(═O)N(R²)NR²R³, and —CR₂OR³;

each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and

R⁶ is independently selected from —H and optionally substituted C₁₋₄ alkyl; and each n is independently selected to be an integer from 0 to 3.

Some embodiments of compounds of Formula (VI) include compounds having the structure of Formula (VI-a):

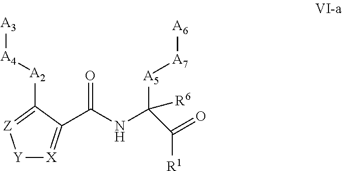

or a pharmaceutically acceptable salt thereof, wherein:

Y is selected from the group consisting of NR⁵, O, S, and SO₂;

X and Z are each independently selected from the group consisting of C(R⁴) and N;

each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and

R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

In some embodiments of compounds of Formula (VI-a) or their pharmaceutically acceptable salts; Z is N, Y is NR⁵, and X is CH. In some embodiments of compounds of Formula (VI-a), R⁵ is selected from the group consisting of —H, C₁₋₄ alkyl, C₁-C₄ haloalkyl, and cyclopropyl. In some embodiments of compounds of Formula (VI-a), Z is N, Y is S, and X is N. In some embodiments of compounds of Formula (VI-a), R² is —H and R³ is selected from the group consisting of optionally substituted C₁₋₄ alkyl and C₃-C₆ cycloalkyl.

In some embodiments of compounds of Formula (VI-a), R² is —H and R³ is optionally substituted C₁₋₄ alkyl. In some embodiments of compounds of Formula (VI-a), R³ is selected from methyl, ethyl, or cyclopropyl. In some embodiments of compounds of Formula (VI-a), R² is —H. In some embodiments of compounds of Formula (VI-a), R¹ is selected from the group consisting of —C(═O)NHOMe, —C(═O)NHN(Me)₂, and —CH₂OH.

In some embodiments of Formulas (I), (I-a), (I-b), (I-c), (I-d), (I-e), at least one of the optionally substituted moieties of A₂, A₄, and A₃ is substituted with ¹⁸F.

In some embodiments of Formulas (I), (I-a), (I-b), (I-c), (I-d), (I-e), at least one of the optionally substituted moieties of A₂, A₄, and A₃ is substituted with C₁-C₆ alkyl containing one or more ¹¹C.

In some embodiments of compounds of Formulas (I), (I-a), (I-b), (I-c), (I-d), (I-e) or their pharmaceutically acceptable salts; A₃ is selected from the group consisting of

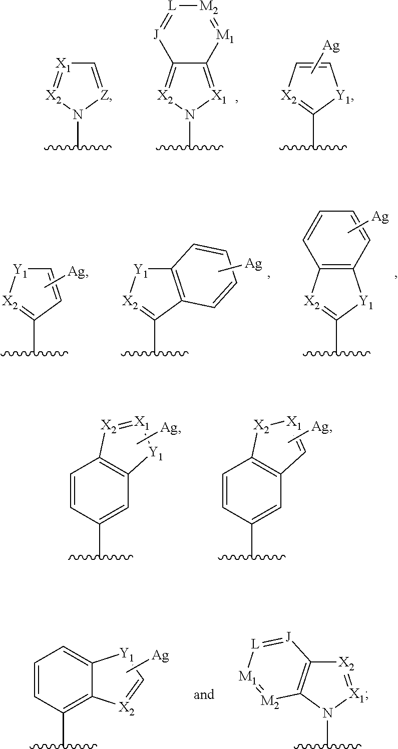

and A₉ is selected from the group consisting of H, C₆₋₁₀ aryl, 5-10 membered heteroaryl, 3-10 membered heterocyclyl, and C₃₋₁₀ carbocyclyl, C₁₋₄ alkyl; X₂, X₁, and Z are each independently selected from the group consisting of C(R⁴) and N; Y₁ is selected from the group consisting of NR⁵, O, and S; J, L, M₁ and M₂ are each independently selected from the group consisting of C(R⁴) and N; R⁴ is selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

In some embodiments of Formulas (I), (I-a), (I-b), (I-c), (I-d), (I-e), A₂ is —CH₂—.

In some embodiments of Formulas (I), (I-a), (I-b), (I-c), (I-d), (I-e), A₂ is —CH═CH—.

In some embodiments of Formulas (I), (I-a), (I-b), (I-c), (I-d), (I-e), A₂ is —O—.

In some embodiments of Formulas (I), (I-a), (I-b), (I-c), (I-d), (I-e), A₂ is S.

In some embodiments of Formulas (I), (I-a), (I-b), (I-c), (I-d), (I-e), A₂ is single bond.

In some embodiments of Formulas (I), (I-a), (I-b), (I-c), (I-d), (I-e), A₂ is phenyl.

In some embodiments of Formulas (I), (I-a), (I-b), (I-c), (I-d), (I-e), A₃ is optionally substituted C₆₋₁₀ aryl.

In some embodiments of Formulas (I), (I-a), (I-b), (I-c), (I-d), (I-e), A₂ is selected from the group consisting of optionally substituted 3-10 membered heterocyclyl, optionally substituted C₆₋₁₀ aryl, optionally substituted 5- or 7-10 membered heteroaryl, optionally substituted C₃₋₁₀ carbocyclyl, —S—, —S(═O)—, —SO₂—, —C(═S)—, —C(═O)—, —NR—, —CH═CH—, —C≡C—, —OC(O)NH—, —NHC(O)NH—, —NHC(O)O—, —NHC(S)NH—, —NHC(S)O—, and —NHC(S)—.

In some embodiments of Formulas (I), (I-a), (I-b), (I-c), (I-d), (I-e), A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, and —C≡C—,

In some embodiments of Formulas (I), (I-a), (I-b), (I-c), (I-d), (I-e), A₂ is selected from the group consisting of optionally substituted 3-10 membered heterocyclyl, optionally substituted C₆₋₁₀ aryl, optionally substituted 5-10 membered heteroaryl, and optionally substituted C₃₋₁₀ carbocyclyl.

In some embodiments of Formulas (I), (I-a), (I-b), (I-c), (I-d), (I-e), A₄ is single bond.

In some embodiments of Formulas (I), (I-a), (I-b), (I-c), (I-d), (I-e), A₃ is selected from the group consisting of

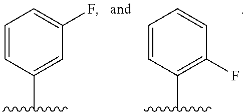 [SMILES: *c1cccc(F)c1 ; "and" ; *c1ccccc1F]

In some embodiments of Formulas (I), (I-a), (I-b), (I-c), (I-d), (I-e), A₃ is optionally substituted 5-10 membered heteroaryl.

In some embodiments of Formulas (I), (I-a), (I-b), (I-c), (I-d), (I-e), (II), (II), (IV), (V), (VI), and (VI-a), wherein at least one of the optionally substituted moieties of A₅, A₇, and A₆ is substituted with ¹⁸F.

In some embodiments of Formulas (I), (I-a), (I-b), (I-c), (I-d), (I-e), (II), (III), (IV), (V), (VI), and (VI-a), wherein at least one of the optionally substituted moieties of A₅, A₇, and A₆ is substituted with C₁-C₆ alkyl containing one or more ¹¹C.

In some embodiments of Formulas (I), (I-a), (I-b), (I-c), (I-d), (I-e), (II), (III), (IV), (V), (VI), and (VI-a), A₆ is phenyl.

In some embodiments of Formulas (I), (I-a), (I-b), (I-c), (I-d), (I-e), (II), (III), (IV), (V), (VI), and (VI-a), A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted —O—C₁₋₆ alkyl, and optionally substituted —O C₂₋₆ alkenyl.

In some embodiments of Formulas (I), (I-a), (I-b), (I-c), (I-d), (I-e), (II), (III), (IV), (V), (VI), and (VI-a), A₇ is —CH₂—.

In some embodiments of Formulas (I), (I-a), (I-b), (I-c), (I-d), (I-e), (II), (III), (IV), (V), (VI), and (VI-a), A₇ is —CH═CH—.

In some embodiments of Formulas (I), (I-a), (I-b), (I-c), (I-d), (I-e), (II), (III), (IV), (V), (V), and (VI-a), A₇ is —O—.

In some embodiments of Formulas (I), (I-a), (I-b), (I-c), (I-d), (I-e), (II), (III), (IV), (V), (VI), and (VI-a), A₇ is S.

In some embodiments of Formulas (I), (I-a), (I-b), (I-c), (I-d), (I-e), (II), (III), (IV), (V), (VI), and (VI-a), A₇ is single bond.

In some embodiments of Formulas (I), (I-a), (I-b), (I-c), (I-d), (I-e), (II), (III), (IV), (V), (VI), and (VI-a), A₇ is optionally substituted C₆₋₁₀ aryl.

In some embodiments of Formulas (I), (I-a), (I-b), (I-c), (I-d), (I-e), (II), (III), (IV), (V), (VI), and (VI-a), A₇ is phenyl.

In some embodiments of Formulas (I), (I-a), (I-b), (I-c), (I-d), (I-e), (II), (In), (IV), (V), (VI), and (VI-a), A₅ is —CH₂—.

In some embodiments of Formulas (I), (I-a), (I-b), (I-c), (I-d), (I-e), (II), (I) (IV), (V), (VI), and (VI-a), wherein A₅ is —CH₂— or —CH₂CH₂—; A₇ is a single bond; and

A₆ is selected from the group consisting of C₁-C₄ alkyl, optionally substituted phenyl, optionally substituted 5-10 membered heteroaryl.

In some embodiments of Formulas (I), (I-a), (I-b), (I-c), (I-d), (I-e), (II), (I)(IV), (V), (VI), and (VI-a), A₆ is optionally substituted phenyl.

In some embodiments of Formulas (I), (I-a), (I-b), (I-c), (I-d), (I-e), (II), (III) (IV), (V), (V), and (VI-a), wherein A₆ is unsubstituted phenyl.

In some embodiments of Formulas (I), (I-a), (I-b), (I-c), (I-d), (I-e), (II), (III) (IV), (V), (VI), and (VI-a) wherein A₆ is phenyl optionally substituted with one or more C₁₋₄ alkyl, C₃₋₇carbocyclyl, halo, hydroxy, and C₁-C₆ alkoxy.

In some embodiments of Formulas (I), (I-a), (I-b), (I-c), (I-d), (I-e), (II), (III), (IV), (V), (VI), and (VI-a), wherein A₅ is a single bond, A₇ is a single bond; and A₆ is C₁-C₅ alkyl.

In some embodiments of Formulas (I), (I-a), (I-b), (I-c), (I-d), (I-e), (II), (III), (IV), (V), (VI), and (VI-a), R² is —H and optionally substituted C₁₋₄ alkyl.

In some embodiments of Formulas (I), (I-a), (I-b), (I-c), (I-d), (I-e), (II), (III), (IV), (V), (VI), and (VI-a), wherein R² is selected from the group consisting of C₁-C₄ alkyl optionally substituted with C-amido, and C₃-C₆ cycloalkyl.

In some embodiments of Formulas (I), (I-a), (I-b), (I-c), (I-d), (I-e), (II), (III), (IV), (V), (VI), and (VI-a), wherein R² is selected from the methyl or ethyl.

In some embodiments of Formulas (I), (I-a), (I-b), (I-c), (I-d), (I-e), (II), (III), (IV), (V), (VI), and (VI-a), wherein R² is benzyl.

In some embodiments of Formulas (I), (I-a), (I-b), (I-c), (I-d), (I-e), (II), (III), (IV), (V), (VI), and (VI-a), R⁶ is —H and optionally substituted C₁₋₄ alkyl.

In some embodiments of Formulas (I), (I-a), (I-b), (I-c), (I-d), (I-e), (II), (III), (IV), (V), (VI), and (VI-a), R⁶ is optionally substituted C₁₋₆ alkyl.

In some embodiments of Formulas (I), (I-a), (I-b), (I-c), (I-d), (I-e), (II), (III), (IV), (V), (VI), and (VI-a), R⁶ is methyl.

In some embodiments of Formula (I), A₁ is selected from the group consisting of optionally substituted 6-10 membered heterocyclyl; 5-membered heterocyclyl optionally substituted with one or more C₁ alkyl, C₃₋₇ carbocyclyl, halo, hydroxy, or C₁-C₆ alkoxy; optionally substituted 5-, 8-, or 9-membered heteroaryl; and optionally substituted C₃₋₁₀ carbocyclyl. In some embodiments of Formula (I), A₁ is selected from the group consisting of 5-membered heterocyclyl optionally substituted with one or more C₁₋₄ alkyl, C₃₋₇ carbocyclyl, halo, hydroxy, or C₁-C₆ alkoxy and optionally substituted 5-membered heteroaryl.

In some embodiments of Formula (I), A is optionally substituted 5-membered heteroaryl.

Some embodiments include a compound selected from the group consisting of compounds 38, 40, 41, 42, 60, 64, 65, 67, 72, 74, 106, 107, 108, and pharmaceutically acceptable salts thereof, as such compounds are described herein.

Some embodiments include a compound selected from the group consisting of compounds 15, 19-21, 23-24, 26, 28, 36, 46, 52, 55, 57, 79, and pharmaceutically acceptable salts thereof, as such compounds are described herein.

Some embodiments include a compound selected from the group consisting of compounds 78, 81, 84, 90, 92, 98 and pharmaceutically acceptable salts thereof, as such compounds are described herein.

Some embodiments include a compound selected from the group consisting of compounds 109, 110, 111, 113, and pharmaceutically acceptable salts thereof, as such compounds are described herein.

Some embodiments include a compound selected from the group consisting of compounds 1-14, 16-18, 22, 25, 27, 29-35, 37, 39, 45, 47-51, 53-54, 58-59, 61-63, 68-71, 73, 75-77, 80, 82-83, 85-88, 89, 91, 93-97, 99-104, 112, 112, 115, and pharmaceutically acceptable salts thereof. Various embodiments include the S-enantiomer, the R-enantiomer, or the racemate of the above compounds.

Additional compounds suitable for use as described herein and that can be made by using the methods described herein are presented in Table 1.

TABLE 1

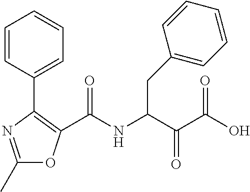 [SMILES: Cc1nc(-c2ccccc2)c(C(=O)NC(Cc2ccccc2)C(=O)C(=O)O)o1]

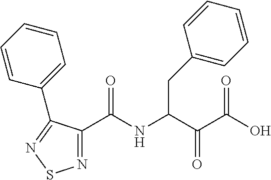 [SMILES: O=C(O)C(=O)C(Cc1ccccc1)NC(=O)c1nsnc1-c1ccccc1]

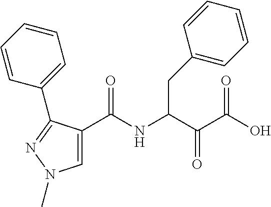 [SMILES: Cn1cc(C(=O)NC(Cc2ccccc2)C(=O)C(=O)O)c(-c2ccccc2)n1]

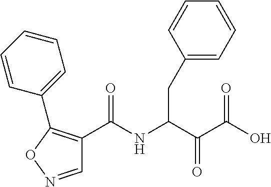 [SMILES: O=C(O)C(=O)C(Cc1ccccc1)NC(=O)c1cnoc1-c1ccccc1]

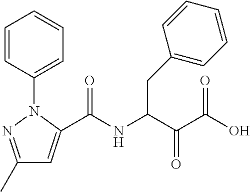 [SMILES: Cc1cc(C(=O)NC(Cc2ccccc2)C(=O)C(=O)O)n(-c2ccccc2)n1]

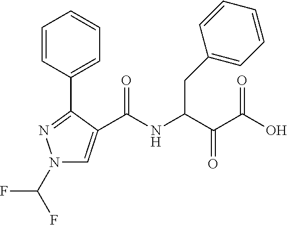 [SMILES: O=C(O)C(=O)C(Cc1ccccc1)NC(=O)c1cn(C(F)F)nc1-c1ccccc1]

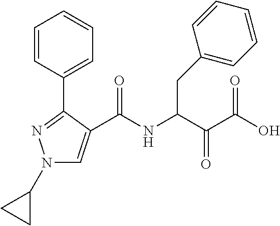 [SMILES: O=C(O)C(=O)C(Cc1ccccc1)NC(=O)c1cn(C2CC2)nc1-c1ccccc1]

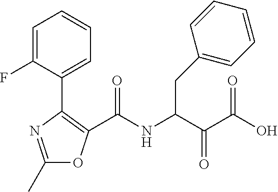 [SMILES: Cc1nc(-c2ccccc2F)c(C(=O)NC(Cc2ccccc2)C(=O)C(=O)O)o1]

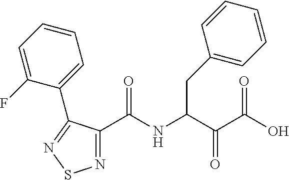 [SMILES: O=C(O)C(=O)C(Cc1ccccc1)NC(=O)c1nsnc1-c1ccccc1F]

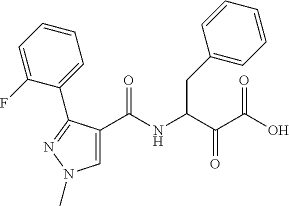 [SMILES: Cn1cc(C(=O)NC(Cc2ccccc2)C(=O)C(=O)O)c(-c2ccccc2F)n1]

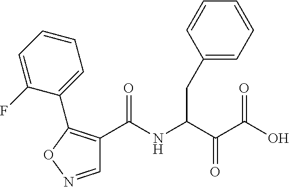 [SMILES: O=C(O)C(=O)C(Cc1ccccc1)NC(=O)c1cnoc1-c1ccccc1F]

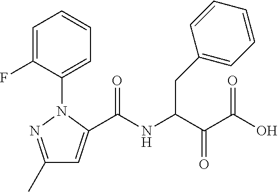 [SMILES: Cc1cc(C(=O)NC(Cc2ccccc2)C(=O)C(=O)O)n(-c2ccccc2F)n1]

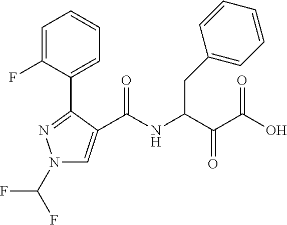 [SMILES: O=C(O)C(=O)C(Cc1ccccc1)NC(=O)c1cn(C(F)F)nc1-c1ccccc1F]

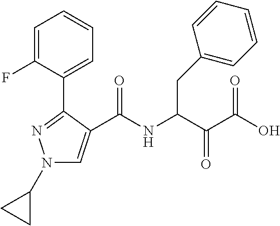 [SMILES: O=C(O)C(=O)C(Cc1ccccc1)NC(=O)c1cn(C2CC2)nc1-c1ccccc1F]

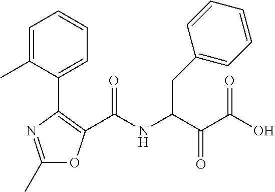 [SMILES: Cc1nc(-c2ccccc2C)c(C(=O)NC(Cc2ccccc2)C(=O)C(=O)O)o1]

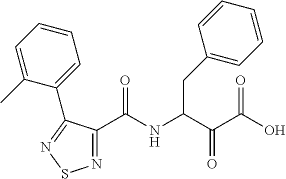 [SMILES: Cc1ccccc1-c1nsnc1C(=O)NC(Cc1ccccc1)C(=O)C(=O)O]

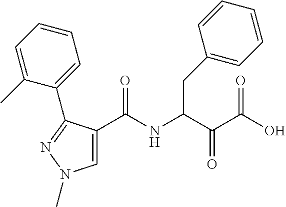 [SMILES: Cc1ccccc1-c1nn(C)cc1C(=O)NC(Cc1ccccc1)C(=O)C(=O)O]

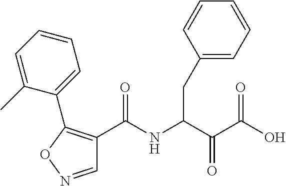 [SMILES: Cc1ccccc1-c1oncc1C(=O)NC(Cc1ccccc1)C(=O)C(=O)O]

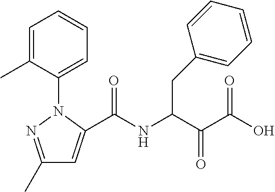 [SMILES: Cc1cc(C(=O)NC(Cc2ccccc2)C(=O)C(=O)O)n(-c2ccccc2C)n1]

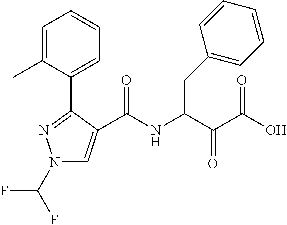 [SMILES: Cc1ccccc1-c1nn(C(F)F)cc1C(=O)NC(Cc1ccccc1)C(=O)C(=O)O]

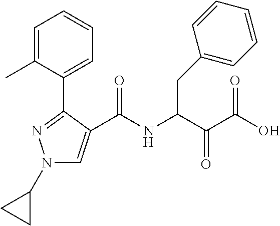 [SMILES: Cc1ccccc1-c1nn(C2CC2)cc1C(=O)NC(Cc1ccccc1)C(=O)C(=O)O]

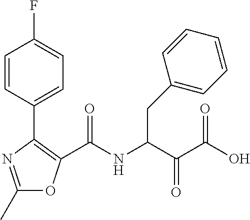 [SMILES: Cc1nc(-c2ccc(F)cc2)c(C(=O)NC(Cc2ccccc2)C(=O)C(=O)O)o1]

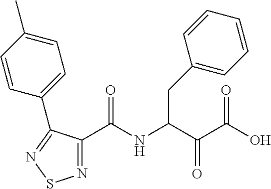 [SMILES: Cc1ccc(-c2nsnc2C(=O)NC(Cc2ccccc2)C(=O)C(=O)O)cc1]

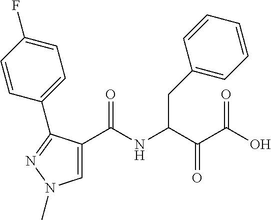 [SMILES: Cn1cc(C(=O)NC(Cc2ccccc2)C(=O)C(=O)O)c(-c2ccc(F)cc2)n1]

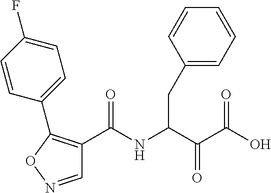 [SMILES: O=C(O)C(=O)C(Cc1ccccc1)NC(=O)c1cnoc1-c1ccc(F)cc1]

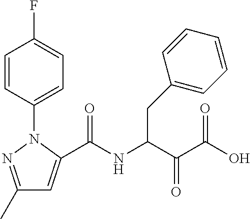 [SMILES: Cc1cc(C(=O)NC(Cc2ccccc2)C(=O)C(=O)O)n(-c2ccc(F)cc2)n1]

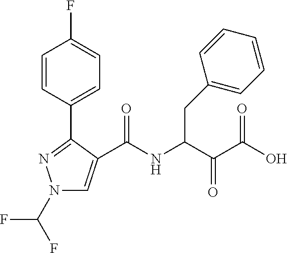 [SMILES: O=C(O)C(=O)C(Cc1ccccc1)NC(=O)c1cn(C(F)F)nc1-c1ccc(F)cc1]

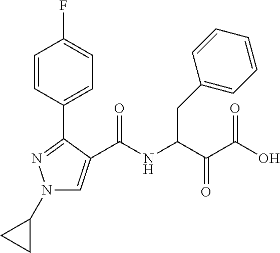 [SMILES: O=C(O)C(=O)C(Cc1ccccc1)NC(=O)c1cn(C2CC2)nc1-c1ccc(F)cc1]

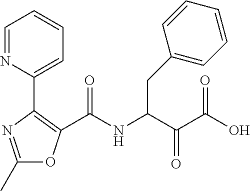 [SMILES: Cc1nc(-c2ccccn2)c(C(=O)NC(Cc2ccccc2)C(=O)C(=O)O)o1]

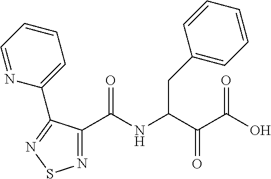 [SMILES: O=C(O)C(=O)C(Cc1ccccc1)NC(=O)c1nsnc1-c1ccccn1]

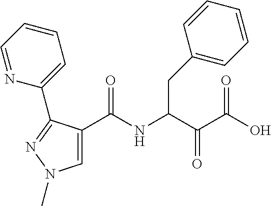 [SMILES: Cn1cc(C(=O)NC(Cc2ccccc2)C(=O)C(=O)O)c(-c2ccccn2)n1]

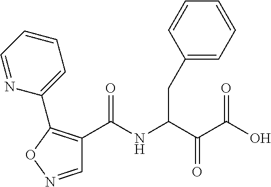 [SMILES: O=C(O)C(=O)C(Cc1ccccc1)NC(=O)c1cnoc1-c1ccccn1]

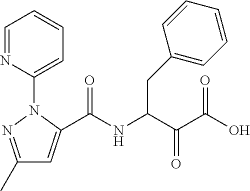 [SMILES: Cc1cc(C(=O)NC(Cc2ccccc2)C(=O)C(=O)O)n(-c2ccccn2)n1]

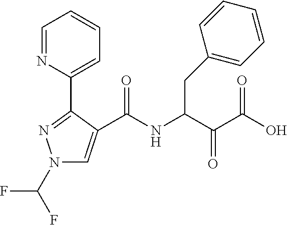 [SMILES: O=C(O)C(=O)C(Cc1ccccc1)NC(=O)c1cn(C(F)F)nc1-c1ccccn1]

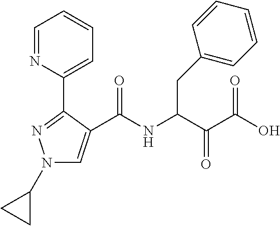 [SMILES: O=C(O)C(=O)C(Cc1ccccc1)NC(=O)c1cn(C2CC2)nc1-c1ccccn1]

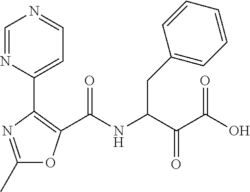 [SMILES: Cc1nc(-c2ccncn2)c(C(=O)NC(Cc2ccccc2)C(=O)C(=O)O)o1]

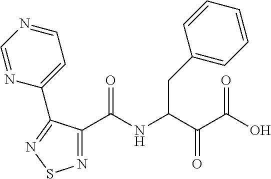 [SMILES: O=C(O)C(=O)C(Cc1ccccc1)NC(=O)c1nsnc1-c1ccncn1]

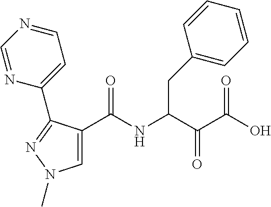 [SMILES: Cn1cc(C(=O)NC(Cc2ccccc2)C(=O)C(=O)O)c(-c2ccncn2)n1]

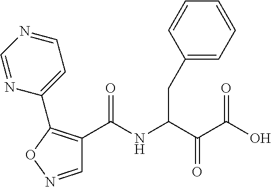 [SMILES: O=C(O)C(=O)C(Cc1ccccc1)NC(=O)c1cnoc1-c1ccncn1]

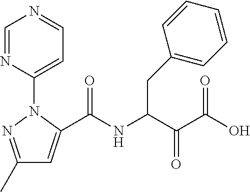 [SMILES: Cc1cc(C(=O)NC(Cc2ccccc2)C(=O)C(=O)O)n(-c2ccncn2)n1]

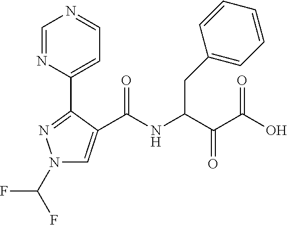 [SMILES: O=C(O)C(=O)C(Cc1ccccc1)NC(=O)c1cn(C(F)F)nc1-c1ccncn1]

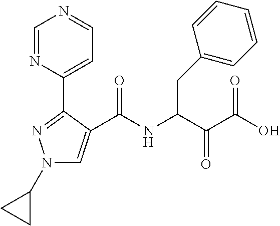 [SMILES: O=C(O)C(=O)C(Cc1ccccc1)NC(=O)c1cn(C2CC2)nc1-c1ccncn1]

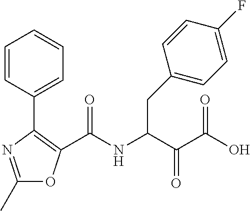 [SMILES: Cc1nc(-c2ccccc2)c(C(=O)NC(Cc2ccc(F)cc2)C(=O)C(=O)O)o1]

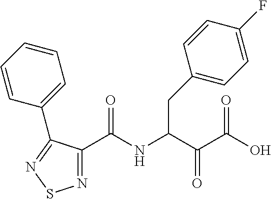 [SMILES: O=C(O)C(=O)C(Cc1ccc(F)cc1)NC(=O)c1nsnc1-c1ccccc1]

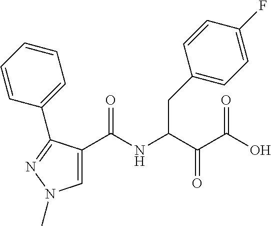 [SMILES: Cn1cc(C(=O)NC(Cc2ccc(F)cc2)C(=O)C(=O)O)c(-c2ccccc2)n1]

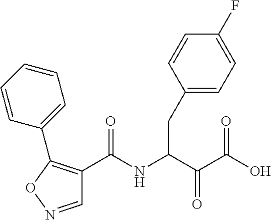 [SMILES: O=C(O)C(=O)C(Cc1ccc(F)cc1)NC(=O)c1cnoc1-c1ccccc1]

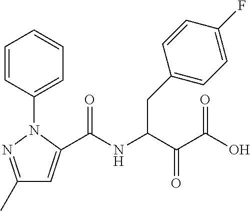 [SMILES: Cc1cc(C(=O)NC(Cc2ccc(F)cc2)C(=O)C(=O)O)n(-c2ccccc2)n1]

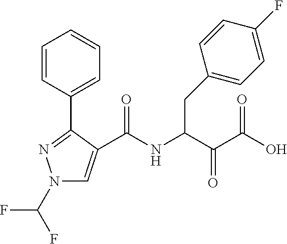 [SMILES: O=C(O)C(=O)C(Cc1ccc(F)cc1)NC(=O)c1cn(C(F)F)nc1-c1ccccc1]

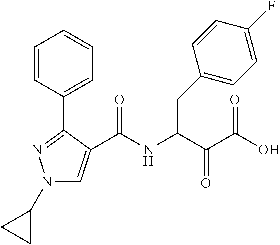 [SMILES: O=C(O)C(=O)C(Cc1ccc(F)cc1)NC(=O)c1cn(C2CC2)nc1-c1ccccc1]

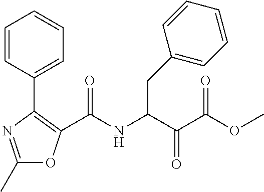

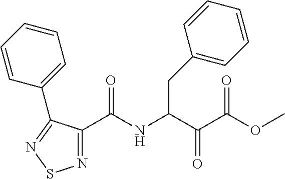 [SMILES: COC(=O)C(=O)C(Cc1ccccc1)NC(=O)c1nsnc1-c1ccccc1]

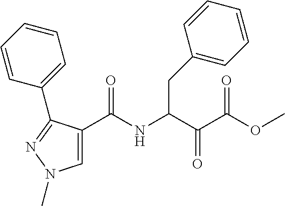 [SMILES: COC(=O)C(=O)C(Cc1ccccc1)NC(=O)c1cn(C)nc1-c1ccccc1]

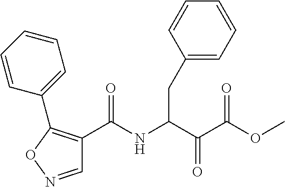 [SMILES: COC(=O)C(=O)C(Cc1ccccc1)NC(=O)c1cnoc1-c1ccccc1]

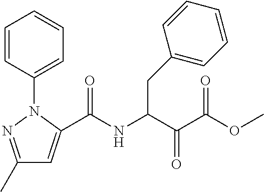

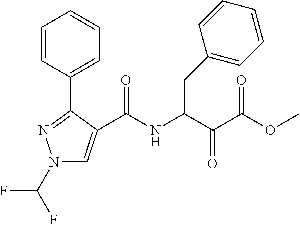 [SMILES: COC(=O)C(=O)C(Cc1ccccc1)NC(=O)c1cn(C(F)F)nc1-c1ccccc1]

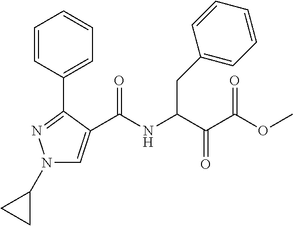 [SMILES: COC(=O)C(=O)C(Cc1ccccc1)NC(=O)c1cn(C2CC2)nc1-c1ccccc1]

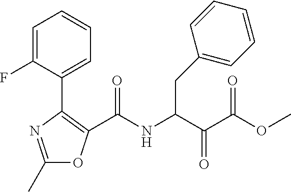 [SMILES: COC(=O)C(=O)C(Cc1ccccc1)NC(=O)c1oc(C)nc1-c1ccccc1F]

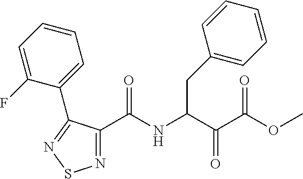 [SMILES: COC(=O)C(=O)C(Cc1ccccc1)NC(=O)c1nsnc1-c1ccccc1F]

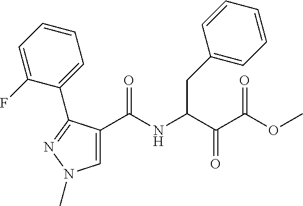 [SMILES: COC(=O)C(=O)C(Cc1ccccc1)NC(=O)c1cn(C)nc1-c1ccccc1F]

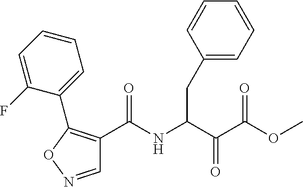 [SMILES: COC(=O)C(=O)C(Cc1ccccc1)NC(=O)c1cnoc1-c1ccccc1F]

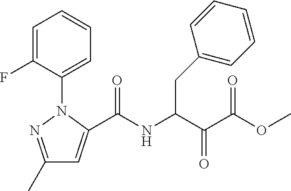 [SMILES: COC(=O)C(=O)C(Cc1ccccc1)NC(=O)c1cc(C)nn1-c1ccccc1F]

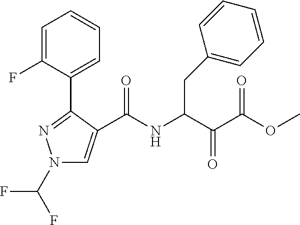 [SMILES: COC(=O)C(=O)C(Cc1ccccc1)NC(=O)c1cn(C(F)F)nc1-c1ccccc1F]

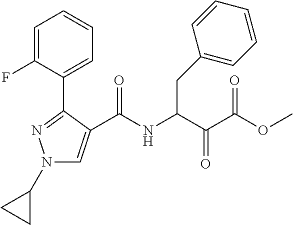 [SMILES: COC(=O)C(=O)C(Cc1ccccc1)NC(=O)c1cn(C2CC2)nc1-c1ccccc1F]

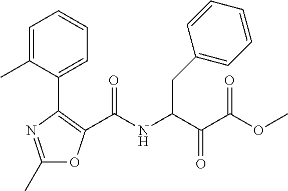

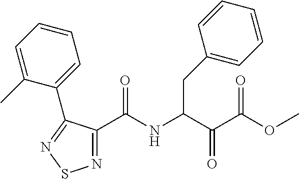

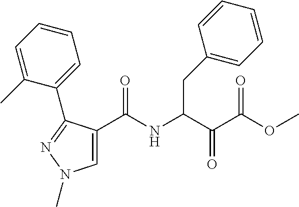

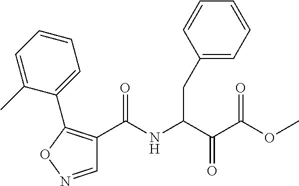

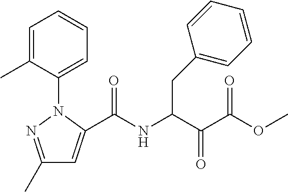

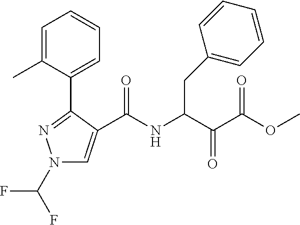 [SMILES: COC(=O)C(=O)C(Cc1ccccc1)NC(=O)c1cn(C(F)F)nc1-c1ccccc1C]

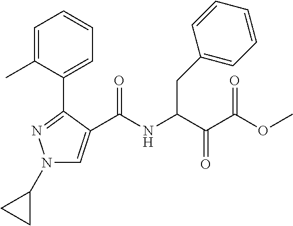 [SMILES: COC(=O)C(=O)C(Cc1ccccc1)NC(=O)c1cn(C2CC2)nc1-c1ccccc1C]

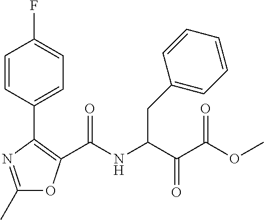

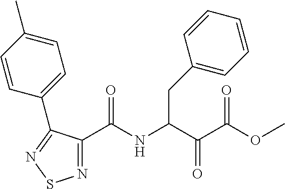 [SMILES: COC(=O)C(=O)C(Cc1ccccc1)NC(=O)c1nsnc1-c1ccc(C)cc1]

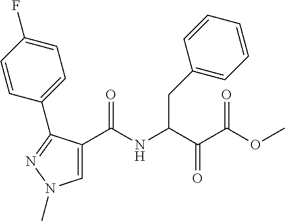 [SMILES: COC(=O)C(=O)C(Cc1ccccc1)NC(=O)c1cn(C)nc1-c1ccc(F)cc1]

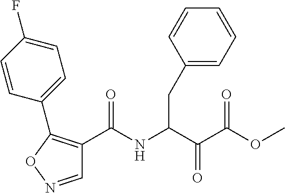 [SMILES: COC(=O)C(=O)C(Cc1ccccc1)NC(=O)c1cnoc1-c1ccc(F)cc1]

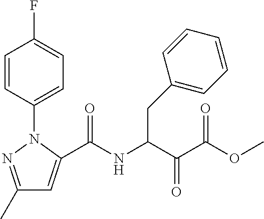

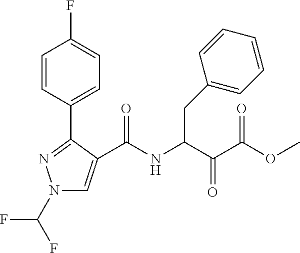 [SMILES: COC(=O)C(=O)C(Cc1ccccc1)NC(=O)c1cn(C(F)F)nc1-c1ccc(F)cc1]

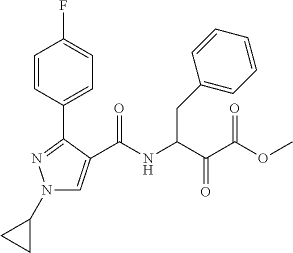 [SMILES: COC(=O)C(=O)C(Cc1ccccc1)NC(=O)c1cn(C2CC2)nc1-c1ccc(F)cc1]

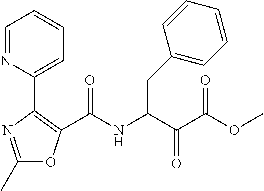

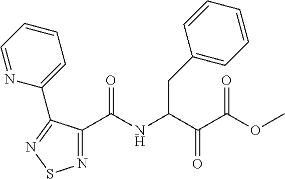 [SMILES: COC(=O)C(=O)C(Cc1ccccc1)NC(=O)c1nsnc1-c1ccccn1]

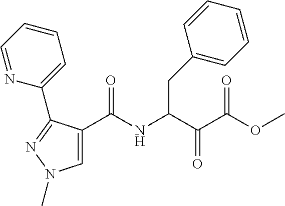 [SMILES: COC(=O)C(=O)C(Cc1ccccc1)NC(=O)c1cn(C)nc1-c1ccccn1]

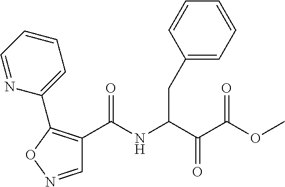 [SMILES: COC(=O)C(=O)C(Cc1ccccc1)NC(=O)c1cnoc1-c1ccccn1]

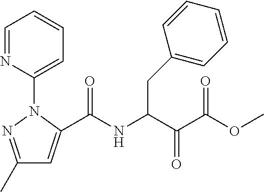

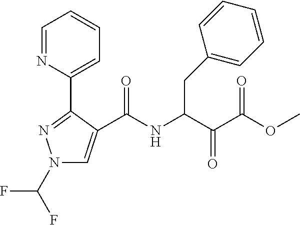 [SMILES: COC(=O)C(=O)C(Cc1ccccc1)NC(=O)c1cn(C(F)F)nc1-c1ccccn1]

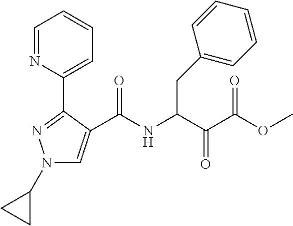 [SMILES: COC(=O)C(=O)C(Cc1ccccc1)NC(=O)c1cn(C2CC2)nc1-c1ccccn1]

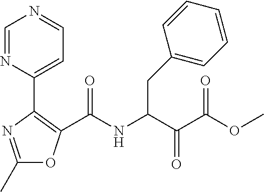

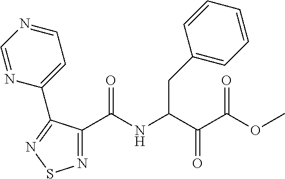 [SMILES: COC(=O)C(=O)C(Cc1ccccc1)NC(=O)c1nsnc1-c1ccncn1]

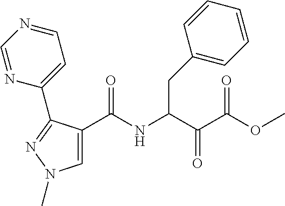 [SMILES: COC(=O)C(=O)C(Cc1ccccc1)NC(=O)c1cn(C)nc1-c1ccncn1]

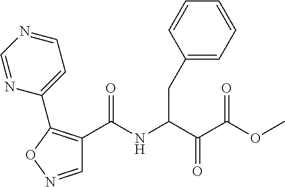 [SMILES: COC(=O)C(=O)C(Cc1ccccc1)NC(=O)c1cnoc1-c1ccncn1]

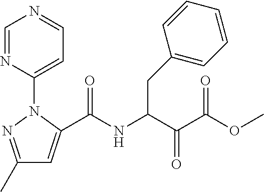

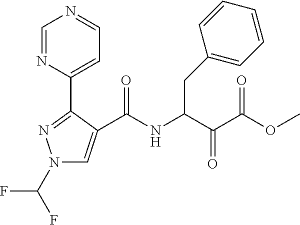 [SMILES: COC(=O)C(=O)C(Cc1ccccc1)NC(=O)c1cn(C(F)F)nc1-c1ccncn1]

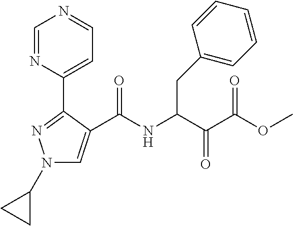 [SMILES: COC(=O)C(=O)C(Cc1ccccc1)NC(=O)c1cn(C2CC2)nc1-c1ccncn1]

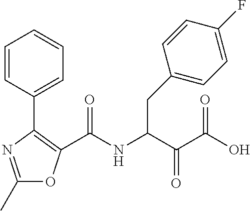 [SMILES: Cc1nc(-c2ccccc2)c(C(=O)NC(Cc2ccc(F)cc2)C(=O)C(=O)O)o1]

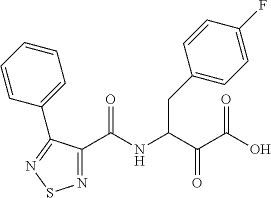 [SMILES: O=C(O)C(=O)C(Cc1ccc(F)cc1)NC(=O)c1nsnc1-c1ccccc1]

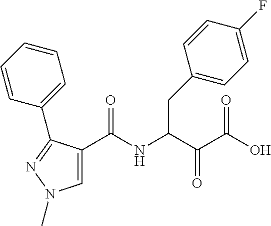 [SMILES: Cn1cc(C(=O)NC(Cc2ccc(F)cc2)C(=O)C(=O)O)c(-c2ccccc2)n1]

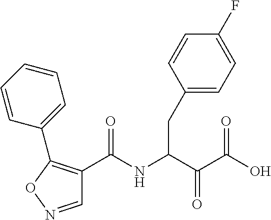 [SMILES: O=C(O)C(=O)C(Cc1ccc(F)cc1)NC(=O)c1cnoc1-c1ccccc1]

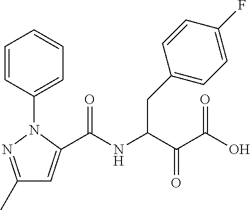 [SMILES: Cc1cc(C(=O)NC(Cc2ccc(F)cc2)C(=O)C(=O)O)n(-c2ccccc2)n1]

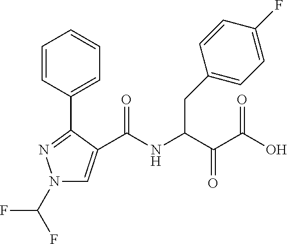 [SMILES: O=C(O)C(=O)C(Cc1ccc(F)cc1)NC(=O)c1cn(C(F)F)nc1-c1ccccc1]

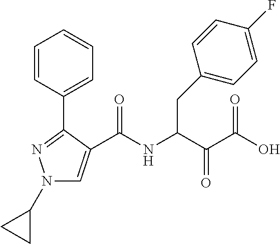 [SMILES: O=C(O)C(=O)C(Cc1ccc(F)cc1)NC(=O)c1cn(C2CC2)nc1-c1ccccc1]

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically, the artisan recognizes that such structures may only represent a very small portion of a sample of such compound(s). Such compounds are considered within the scope of the structures depicted, though such resonance forms or tautomers are not represented herein.

### Isotopically-Labeled Compounds

Isotopes may be present in the compounds described. Each chemical element as represented in a compound structure may include any isotope of said element. The isotopes may be isotopes of carbon, chlorine, fluorine, hydrogen, iodine, nitrogen, oxygen, phosphorous, sulfur, and technetium, including ¹¹C, ¹³C, ¹⁴C, ³⁶Cl, ¹⁸F, ²H, ³H, ¹²³I, ¹²⁵I, ¹³N, ¹⁵N, ¹⁵O, ¹⁷O, ¹⁸O, ³¹P, ³²P, ³⁵S, and ^{99m}Tc. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Isotopically-labeled compounds of the present embodiments are useful in drug and substrate tissue distribution and target occupancy assays. For example, isotopically labeled compounds are particularly useful in SPECT (single photon emission computed tomography) and in PET (positron emission tomography), as discussed further herein.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (ed. H. Bundgaard, Elsevier, 1985), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl)methyl group. Other examples of pro-drug ester groups can be found in, for example, T. Higuchi and V. Stella, in “Pro-drugs as Novel Delivery Systems”, Vol. 14, A.C.S. Symposium Series, American Chemical Society (1975); and “Bioreversible Carriers in Drug Design: Theory and Application”, edited by E. B. Roche, Pergamon Press: New York, 14-21 (1987) (providing examples of esters useful as prodrugs for compounds containing carboxyl groups). Each of the above-mentioned references is herein incorporated by reference in their entirety.

“Metabolites” of the compounds disclosed herein include active species that are produced upon introduction of the compounds into the biological milieu.

“Solvate” refers to the compound formed by the interaction of a solvent and a compound described herein, a metabolite, or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C-9 alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

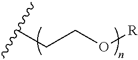

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

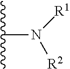

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

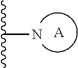

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

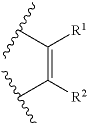

and R¹ and R² are defined as selected from the group consisting of hydrogen and alkyl, or RI and R² together with the atoms to which they are attached form an aryl or carbocyclyl, it is meant that R¹ and R² can be selected from hydrogen or alkyl, or alternatively, the substructure has structure:

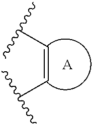

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

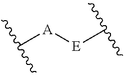

includes the substituent being oriented such that the A is attached at the leftmost attachment point of the molecule as well as the case in which A is attached at the rightmost attachment point of the molecule.

As used herein, the substructure:

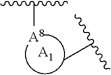

means that the A₈ atom can be in any ring atom position within the ring or ring system A₁. The substructure:

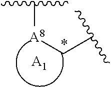

means that the A₈ atom is in the ring atom position immediately adjacent (i.e., alpha) to the point of attachment indicated by *.

As used herein, “isosteres” of a chemical group are other chemical groups that exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated include —SO₃H, —SO₂HNR, —PO₂(R)₂, —PO₃(R)₂, —CONHNHSO₂R, —COHNSO₂R, and —CONRCN, where R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 3-10 membered heterocyclyl, as defined herein. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carbocyclic and heterocyclic isosteres contemplated. The atoms of said ring structure may be optionally substituted at one or more positions with R as defined above.

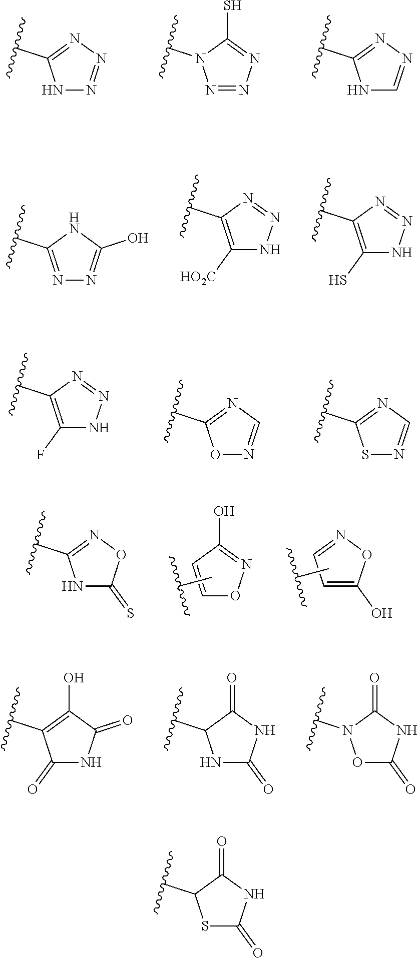

It is also contemplated that when chemical substituents are added to a carboxylic isostere, the compound retains the properties of a carboxylic isostere. It is contemplated that when a carboxylic isostere is optionally substituted with one or more moieties selected from R as defined above, then the substitution and substitution position is selected such that it does not eliminate the carboxylic acid isosteric properties of the compound. Similarly, it is also contemplated that the placement of one or more R substituents upon a carbocyclic or heterocyclic carboxylic acid isostere is not a substitution at one or more atom(s) that maintain(s) or is/are integral to the carboxylic acid isosteric properties of the compound, if such substituent(s) would destroy the carboxylic acid isosteric properties of the compound.

Other carboxylic acid isosteres not specifically exemplified in this specification are also contemplated.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved characteristics (such as higher binding affinity for a target molecule) is an approach that is well known in pharmaceutical chemistry.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “microbial infection” refers to the invasion of the host organism, whether the organism is a vertebrate, invertebrate, fish, plant, bird, or mammal, by pathogenic microbes. This includes the excessive growth of microbes that are normally present in or on the body of a mammal or other organism. More generally, a microbial infection can be any situation in which the presence of a microbial population(s) is damaging to a host mammal. Thus, a mammal is “suffering” from a microbial infection when excessive numbers of a microbial population are present in or on a mammal's body, or when the effects of the presence of a microbial population(s) is damaging the cells or other tissue of a mammal. Specifically, this description applies to a bacterial infection. Note that the compounds of preferred embodiments are also useful in treating microbial growth or contamination of cell cultures or other media, or inanimate surfaces or objects, and nothing herein should limit the preferred embodiments only to treatment of higher organisms, except when explicitly so specified in the claims.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated, however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Synthesis of Compounds of Formula I

In one embodiment, the method involves reacting an appropriately substituted intermediate (VI-a) with a nitrile group under acidic conditions followed by treatment with a base to provide a sodium salt (VI-b). This intermediate was treated with BOC-anhydride under basic conditions to provide the BOC-derivative (VI) which was subjected to esterification conditions to yield the intermediate (VII). The ester intermediate was hydrolyzed under acidic conditions to yield the amine (VIII). (Scheme 1). The amine (VIII) was subjected to amide coupling conditions with the carboxylic acid (IX) to yield the corresponding adduct (X). The resulting adduct (X) is subjected to oxidation conditions with DMP oxidation (with hypervalent iodine) or by an oxidizing agent such as PCC (pyridinium chlorochromate) to yield the α-ketoester product (I). Alternately, the adduct (X) was subjected to oxidation conditions using EDC and dichloroacetic acid or using IBX as the oxidizing agent to yield the α-ketoester product (I). Additionally, the intermediate (I) was hydrolyzed under acidic conditions to yield the carboxylic acid (XI). The skilled artisan will once again appreciate that there are many other oxidizing conditions and agents which are within the scope of this disclosure to oxidize the hydroxyl group. This synthetic route is generally shown in Scheme 2.

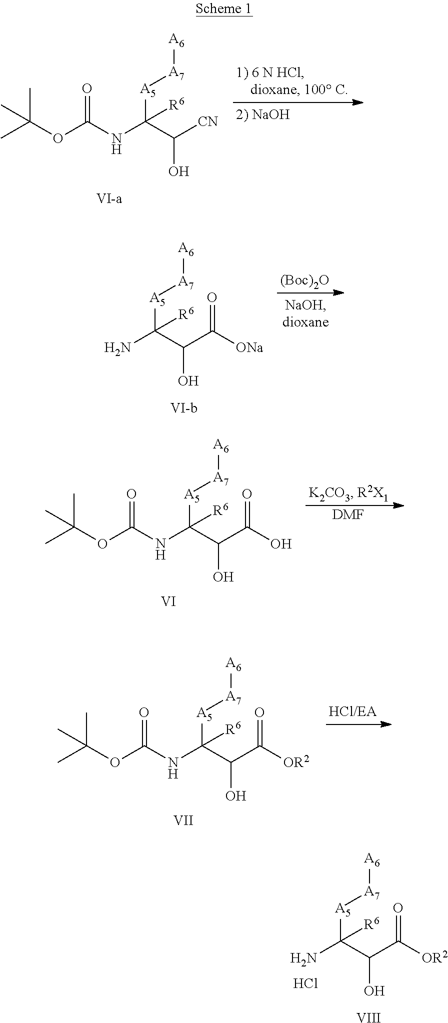

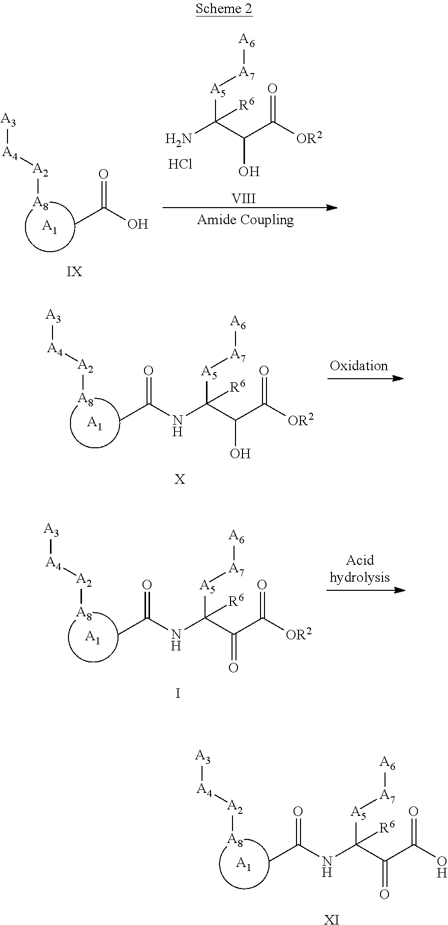

In one embodiment, the method involves reacting an appropriately substituted intermediate (XII) under suzuki coupling conditions with a substituted boronate ester intermediate (XIII) to yield a product which was hydrolyzed to yield the acid (XIV). The acid was coupled with the intermediate (XV) and then subjected to oxidation conditions with DMP oxidation (with hypervalent iodine) or by an oxidizing agent such as PCC (pyridinium chlorochromate) to yield the α-ketoamide product (II-a). This synthetic route is generally shown in Scheme 3.

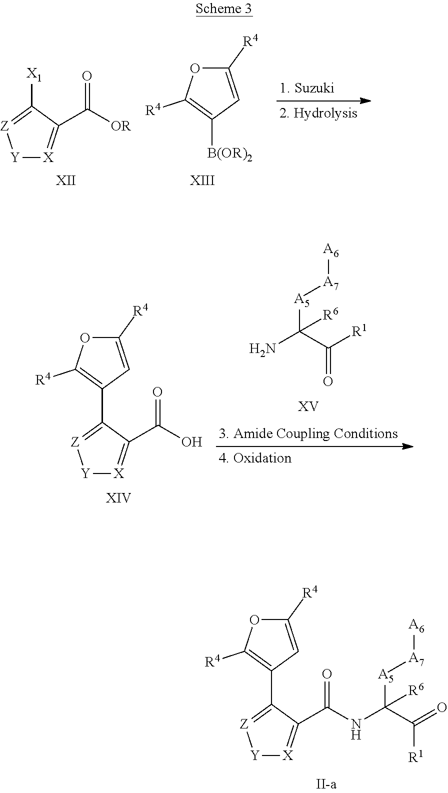

Alternately, the intermediate (XVI) was subjected to chlorination using NCS followed by hydrolysis of the ester to yield the intermediate (XVII). The intermediate (XVII) was coupled with the intermediate (XV) and then subjected to oxidation conditions with DMP oxidation (with hypervalent iodine) or by an oxidizing agent such as PCC (pyridinium chlorochromate) to yield the chloro-furan substituted α-ketoamide product (XVIII). This synthetic route is generally shown in Scheme 4.

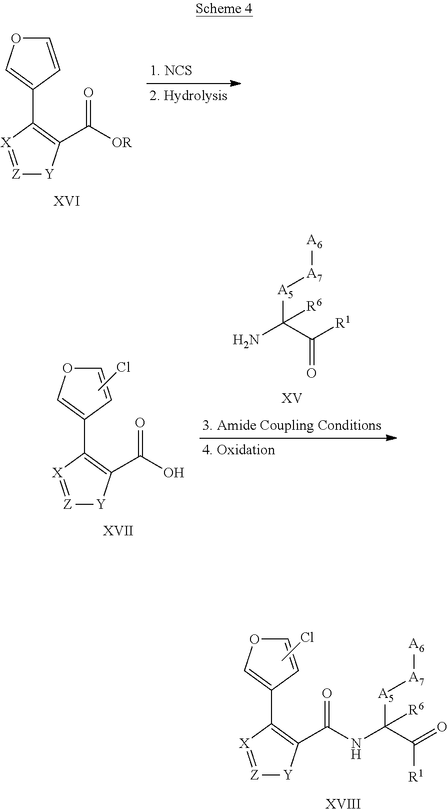

The above example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

### Uses of Isotopically-Labeled Compounds

Some embodiments provide a method of using isotopically labeled compounds and prodrugs of the present disclosure in: (i) metabolic studies (preferably with ¹⁴C), reaction kinetic studies (with, for example 2H or 3H); (ii) detection or imaging techniques [such as positron emission tomography (PET) or single-photon emission computed tomography (SPECT)] including drug or substrate tissue distribution assays; or (iii) in radioactive treatment of patients.

Isotopically labeled compounds and prodrugs of the embodiments thereof can generally be prepared by carrying out the procedures disclosed in the schemes or in the examples and preparations described below by substituting a readily available isotopically labeled reagent for a non-isotopically labeled reagent. An ¹⁸F or ¹¹C labeled compound may be particularly preferred for PET, and an ¹²³I labeled compound may be particularly preferred for SPECT studies. Further substitution with heavier isotopes such as deuterium (i.e., ²H) may afford certain therapeutic advantages resulting from greater metabolic stability, for example increased in vivo half-life or reduced dosage requirements.

### Synthesis of Isotopically Labeled Compounds

¹⁸F labeled compounds are synthesized as shown in the schemes below. In one embodiment, the method involves reacting the intermediate 44 with a ¹⁸F-labeling agent using conditions as described in Rotstein, et al., Spirocyclic hypervalent iodine(II)-mediated radiofluorination of non-activated and hindered aromatics, Nature Communications, 2014, Vol. 5, 4365-4371 and Rotstein, et al., Mechanistic Studies and Radiofluorination of Structurally Diverse Pharmaceuticals with Spirocyclic Iodonium(II) Ylides, Chemical Science, 2016, Vol. 7, 4407-4417, both of which are incorporated herein by reference in their entirety, to yield the ¹⁸F-labeled intermediate ethyl 3-(4-(fluoro-¹⁸F)phenyl)-1-methyl-1H-pyrazole-4-carboxylate (44-A) which is then transformed into the final α-ketoester or α-ketoacid product represented by the general structure XIX (Scheme 5).

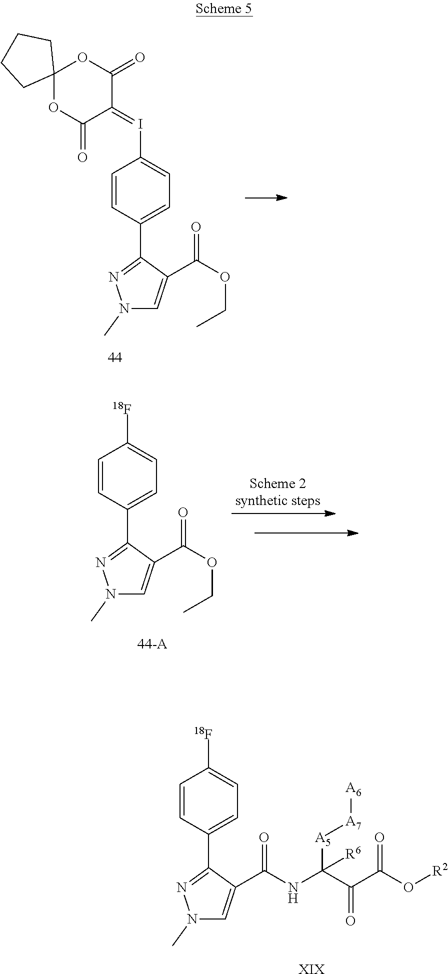

Alternately, ¹⁸F-labeled compound XXII is synthesized as shown in Scheme 6. In one embodiment, iodanylidene intermediate XX with a ¹⁸F label introduced using conditions as described in Rotstein, et al., Spirocyclic hypervalent iodine(III)-mediated radiofluorination of non-activated and hindered aromatics, Nature Communications, 2014, Vol. 5, 4365-4371 and Rotstein, et al., Mechanistic Studies and Radiofluorination of Structurally Diverse Pharmaceuticals with Spirocyclic Iodonium(III) Ylides, Chemical Science, 2016, Vol. 7, 4407-4417 is transformed to yield the labeled α-ketoester or α-ketoacid product XXII. In another embodiment, iodanylidene intermediate (XXI) is (Scheme 6) subjected to introduce the ¹⁸F-label followed by oxidation conditions with DMP oxidation (with hypervalent iodine) or by an oxidizing agent such as PCC (pyridinium chlorochromate) to yield the α-ketoester or α-ketoacid product (XXII).

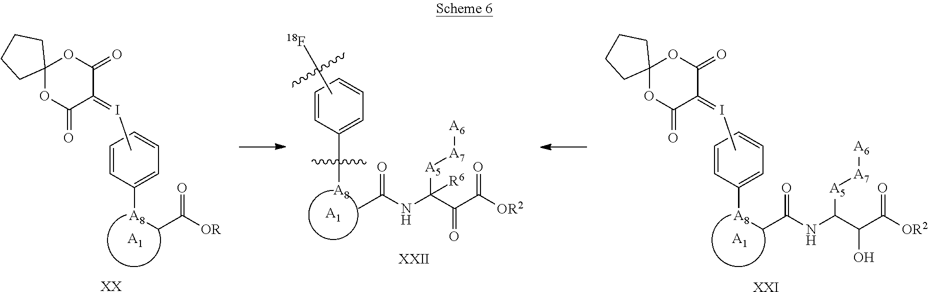

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising. (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, come embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modem Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 99.99 wt % of a compound of the present technology based on the total formulation, with the balance being one or more suitable pharmaceutical excipients. Preferably, the compound is present at a level of about 1 80 wt %. Representative pharmaceutical formulations are described below.

### FORMULATION EXAMPLES

The following are representative pharmaceutical formulations containing a compound of Formula I.

### Formulation Example 1—Tablet Formulation

The following ingredients are mixed intimately and pressed into single scored tablets.

| | Quantity per |
|---|---|
| Ingredient | tablet, mg |
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

### Methods of Treatment

The compounds disclosed herein or their tautomers and/or pharmaceutically acceptable salts thereof can effectively act as CAPN1, CAPN2, and/or CAPN9 inhibitors and treat conditions affected at least in part by CAPN1, CAPN2, and/or CAPN9. Some embodiments provide pharmaceutical compositions comprising one or more compounds disclosed herein and a pharmaceutically acceptable excipient. Some embodiments provide a method for treating a fibrotic disease with an effective amount of one or more compounds as disclosed herein.

In some embodiments, the subject is a human.

Further embodiments include administering a combination of compounds to a subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered i.v.

Some embodiments include combinations of a compound, composition or pharmaceutical composition described herein with any other pharmaceutical compound approved for treating fibrotic or myofibroblast differentiation associated diseases or disorders.

Some embodiments provide a method for inhibiting CAPN1, CAPN2, and/or CAPN9 and/or a method for treating a disease affected at least in part by CAPN1, CAPN2, and/or CAPN9 with an effective amount of one or more compounds as disclosed herein.

The compounds disclosed herein are useful in inhibiting CAPN1, CAPN2, and/or CAPN9 enzymes and/or treating disorders relating to fibrosis or myofibroblast differentiation.

Some embodiments provide a method for inhibiting CAPN1, CAPN2, and/or CAPN9 which method comprises contacting cells (including neurons/microglia/invading macrophages) with an effective amount of one or more compounds as disclosed herein.

Some embodiments provide a method for treating a fibrotic disease, which method comprises administering to a subject an effective amount of one or more compounds or a pharmaceutical composition disclosed herein comprising a pharmaceutically acceptable excipient.

Some embodiments provide a method for treating a disease affected at least in part by CAPN1, CAPN2, and/or CAPN9, which method comprises administering to a subject an effective amount of one or more compounds or a pharmaceutical composition disclosed herein comprising a pharmaceutically acceptable excipient.

Some embodiments provide a method for inhibiting CAPN1, CAPN2, and/or CAPN9 is provided wherein the method comprises contacting cells with an effective amount of one or more compounds disclosed herein. In some embodiments a method for inhibiting CAPN1, CAPN2, and/or CAPN9 is performed in-vitro or in-vivo.

Calpains are also expressed in cells other than neurons, microglia and invading macrophages. In particular, they are important in skeletal muscle and herein inhibition of calpains also refers to inhibition in these cells as well.

### Selective Inhibition

Some embodiments provide a method for competitive binding with calpastatin (CAST), the method comprising contacting a compound disclosed herein with CAPN1, CAPN2, and/or CAPN9 enzymes residing inside a subject. In such a method, the compound specifically inhibits one or more of the enzymes selected from the group consisting of: CAPN1, CAPN2, and CAPN9 by at least 2-fold, by at least 3-fold, by at least 4-fold, by at least 5-fold, by at least 10-fold, by at least 15-fold, by at least 20-fold, by at least 50-fold, by at least 100-fold, by at least 150-fold, by at least 200-fold, by at least 400-fold, or by at least 500-fold.

Some embodiments provide a method for selectively inhibiting CAPN1 in the presence of CAPN2 and CAPN9, which includes contacting cells (including neurons/microglia/invading macrophages) with an effective amount of one or more compounds disclosed herein.

Some embodiments provide a method for selectively inhibiting CAPN2 in the presence of CAPN1 and CAPN9, which includes contacting cells (including neurons/microglia/invading macrophages) with an effective amount of one or more compounds disclosed herein.

Some embodiments provide a method for selectively inhibiting CAPN9 in the presence of CAPN2 and CAPN1, which includes contacting cells (including neurons/microglia/invading macrophages) with an effective amount of one or more compounds disclosed herein.

Some embodiments provide a method for selectively inhibiting CAPN1 and CAPN2 in the presence of CAPN9, which includes contacting cells (including neurons/microglia/invading macrophages) with an effective amount of one or more compounds disclosed herein.

Some embodiments provide a method for selectively inhibiting CAPN1 and CAPN9 in the presence of CAPN2, which includes contacting cells (including neurons/microglia/invading macrophages) with an effective amount of one or more compounds disclosed herein.

Some embodiments provide a method for selectively inhibiting CAPN2 and CAPN9 in the presence of CAPN1, which includes contacting cells (including neurons/microglia/invading macrophages) with an effective amount of one or more compounds disclosed herein.

Some embodiments provide a method for treating a fibrotic disease, which method comprises administering to a subject an effective amount of one or more compounds which specifically inhibits CAPN1, CAPN2, and/or CAPN9, said compounds or a pharmaceutical composition comprising one or more compounds disclosed herein and a pharmaceutically acceptable excipient.

Some embodiments provide a method for treating a disease affected at least in part by CAPN1, CAPN2, and/or CAPN9, which method comprises administering to a subject an effective amount of one or more compounds which specifically inhibits CAPN1, CAPN2, and/or CAPN9, said compounds being selected from compounds disclosed herein or a pharmaceutical composition comprising one or more compounds disclosed herein and a pharmaceutically acceptable excipient.

Some embodiments provide a method for treating a fibrotic disease, which method comprises administering to a subject an effective amount of one or more compounds which selectively inhibits CAPN1, CAPN2, and/or CAPN9, said compounds being selected from compounds disclosed herein or a pharmaceutical composition comprising one or more compounds disclosed herein and a pharmaceutically acceptable excipient.

Some embodiments provide a method for treating a disease affected at least in part by CAPN1, CAPN2, and/or CAPN9, which method comprises administering to a subject an effective amount of one or more compounds which selectively inhibits CAPN1, CAPN2, and/or CAPN9, said compounds being selected from compounds disclosed herein or a pharmaceutical composition comprising one or more compounds disclosed herein and a pharmaceutically acceptable excipient.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:5.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:10.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:20.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:50.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:100.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:200.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:250.

Some embodiments provide a method for treating a fibrotic disease, which method comprises administering to a subject an effective amount of one or more compounds which specifically inhibits two or more enzymes selected from the group consisting of CAPN1, CAPN2, and CAPN9 in a ratio of at least 1:1:500.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:5.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:10.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:20.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:50.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:100.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:200.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:250.

Some embodiments provide a method for treating a fibrotic disease, which method comprises administering to a subject an effective amount of one or more compounds which selectively inhibits two or more enzymes selected from the group consisting of CAPN1, CAPN2, and CAPN9 in a ratio of at least 1:1:500.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:5.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:10.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:20.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:50.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:100.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:200.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:250.

Some embodiments provide a method for treating a disease affected at least in part by CAPN1, CAPN2, and/or CAPN9, which method comprises administering to a subject an effective amount of one or more compounds which specifically inhibits two or more enzymes selected from the group consisting of CAPN1, CAPN2, and CAPN9 in a ratio of at least 1:1:500.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:5.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:10.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:20.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:50.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:100.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:200.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:250.

Some embodiments provide a method for treating a disease affected at least in part by CAPN1, CAPN2, and/or CAPN9, which method comprises administering to a subject an effective amount of one or more compounds which selectively inhibits two or more enzymes selected from the group consisting of CAPN1, CAPN2, and CAPN9 in a ratio of at least 1:1:500.

Some embodiments provide a method for prophylactic therapy or treatment of a subject having a fibrotic disorder wherein said method comprising administering an effective amount of one or more compounds disclosed herein to the subject in need thereof.

Some embodiments provide a method for prophylactic therapy or treatment of a subject having a disorder affected by CAPN1, CAPN2, and/or CAPN9 wherein said method comprising administering an effective amount of one or more compounds disclosed herein to the subject in need thereof.

Some embodiments provide a method for inhibiting myofibroblast differentiation (e.g., Epithelial/Endothelial-to-Mesenchymal Transition (EpMT/EnMT)) is provided wherein the method comprises contacting cells with an effective amount of one or more compounds disclosed herein. In one aspect, the method for inhibiting myofibroblast differentiation (e.g., Epithelial/Endothelial-to-Mesenchymal Transition (EpMT/EnMT)) is performed in-vitro or in-vivo.

Some embodiments provide a method for treating a disease or condition selected from the group consisting of or that produces a symptom selected from the group consisting of: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis diseases, wherein which method comprises administering to a subject an effective amount of one or more compounds disclosed herein to a subject in need thereof.

Some embodiments provide a method for treating liver fibrosis.

Some embodiments provide a method for treating cardiac fibrosis.

Some embodiments provide a method for treating fibrosis in rheumatoid arthritis diseases.

Some embodiments provide a method for treating a condition affected by CAPN1, CAPN2, and/or CAPN9, which is in both a therapeutic and prophylactic setting for subjects. Both methods comprise administering of one or more compounds disclosed herein to a subject in need thereof.

Some embodiments provide a method for treating stiff skin syndrome.

Preferred embodiments include combinations of a compound, composition or pharmaceutical composition described herein with other CAPN1, CAPN2, and/or CAPN9 inhibitor agents, such as anti-CAPN1, CAPN2, AND/OR CAPN9 antibodies or antibody fragments, CAPN1, CAPN2, and/or CAPN9 antisense, iRNA, or other small molecule CAPN1, CAPN2, and/or CAPN9 inhibitors.

Some embodiments include combinations of a compound, composition or pharmaceutical composition described herein to inhibit myofibroblast differentiation (e.g., Epithelial/Endothelial-to-Mesenchymal Transition (EpMT/EnMT)).

Some embodiments include combinations of one or more of these compounds which are inhibitors of one or more (or all three) CAPN1, CAPN2, and/or CAPN9, alone or in combination with other TGFβ signaling inhibitors, could be used to treat or protect against or reduce a symptom of a fibrotic, sclerotic or post inflammatory disease or condition including: liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, postvasectomy pain syndrome, and rheumatoid arthritis.

Some embodiments include a combination of the compounds, compositions and/or pharmaceutical compositions described herein with an additional agent, such as anti-inflammatories including glucocorticoids, analgesics (e.g. ibuprofen), aspirin, and agents that modulate a Th2-immune response, immunosuppressants including methotrexate, mycophenolate, cyclophosphamide, cyclosporine, thalidomide, pomalidomide, leflunomide, hydroxychloroquine, azathioprine, soluble bovine cartilage, vasodilators including endothelin receptor antagonists, prostacyclin analogues, nifedipine, and sildenafil, IL-6 receptor antagonists, selective and non-selective tyrosine kinase inhibitors, Wnt-pathway modulators, PPAR activators, caspase-3 inhibitors, LPA receptor antagonists, B cell depleting agents, CCR2 antagonists, pirfenidone, cannabinoid receptor agonists, ROCK inhibitors, miRNA-targeting agents, toll-like receptor antagonists, CTGF-targeting agents, NADPH oxidase inhibitors, tryptase inhibitors, TGFD inhibitors, relaxin receptor agonists, and autologous adipose derived regenerative cells.

### Indications

In some embodiments, the compounds and compositions comprising the compounds described herein can be used to treat a host of conditions arising from fibrosis or inflammation, and specifically including those associated with myofibroblast differentiation. Example conditions include liver fibrosis (alcoholic, viral, autoimmune, metabolic and hereditary chronic disease), renal fibrosis (e.g., resulting from chronic inflammation, infections or type II diabetes), lung fibrosis (idiopathic or resulting from environmental insults including toxic particles, sarcoidosis, asbestosis, hypersensitivity pneumonitis, bacterial infections including tuberculosis, medicines, etc.), interstitial fibrosis, systemic scleroderma (autoimmune disease in which many organs become fibrotic), macular degeneration (fibrotic disease of the eye), pancreatic fibrosis (resulting from, for example, alcohol abuse and chronic inflammatory disease of the pancreas), fibrosis of the spleen (from sickle cell anemia, other blood disorders), cardiac fibrosis (resulting from infection, inflammation and hypertrophy), mediastinal fibrosis, myelofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic reperfusion injury associated fibrosis, injection fibrosis, cirrhosis, diffuse parenchymal lung disease, post-vasectomy pain syndrome, and rheumatoid arthritis diseases or disorders.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
DCM=dichloromethane
DIEA=N,N-Diisopropylethylamine
DIPEA=N,N-Diisopropylethylamine
DMF=N,N-dimethylformamide
DMP=Dess Martin Periodinane
DNs=dinitrosulfonyl
ESBL=extended-spectrum f-lactamase
EtOAc=ethyl acetate
EA=ethyl acetate
FCC=Flash Column Chromatography
HATU=2-(7-aza-1H-benzotriazole-1-yl)-1,1,3,3-tetramethyluronium hexafluorophosphate
MeCN=acetonitrile
NMR=nuclear magnetic resonance
PE=Petroleum Ether
Prep=preparatory
Py=pyridine
Sat.=saturated aqueous
TBDMSCl=tert-butyldimethylsilyl chloride
TBS=tert-butyldimethylsilyl
TFA=trifluoroacetic acid
THF=tetrahydrofuran
TLC=thin layer chromatography

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### COMPOUNDS 1, 12, 14, 18, 22, 28, 54, 94, 99, 100, 101, AND 102 N-(4-AMINO-3,4-DIOXO-1-PHENYLBUTAN-2-YL)-1-METHYL-3-(QUINOLIN-7-YL)-1H-PYRAZOLE-4-CARBOXAMIDE (1)

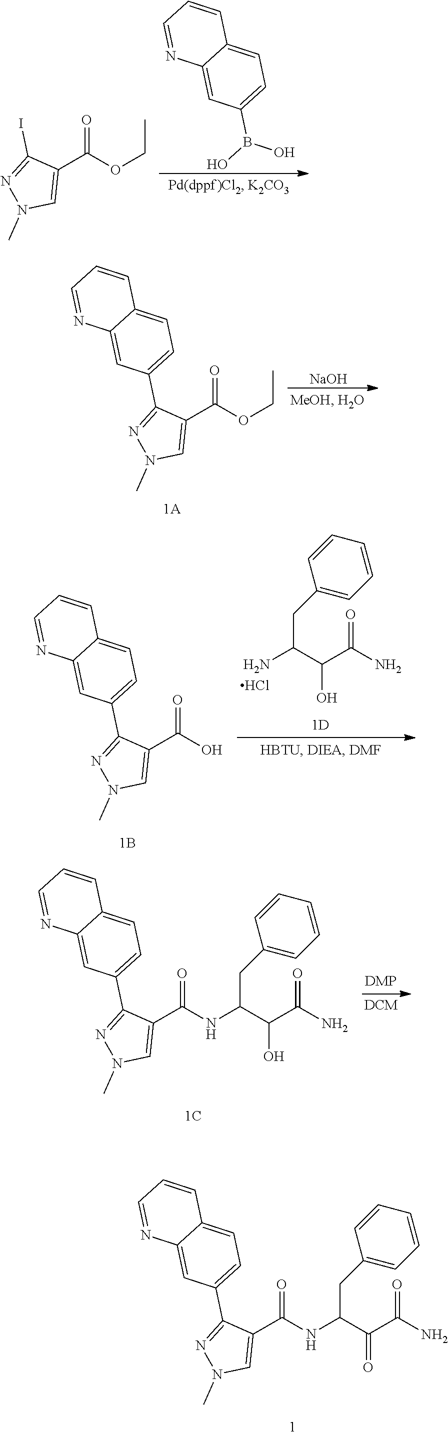

To a solution of ethyl 3-iodo-1-methyl-1H-pyrazole-4-carboxylate (0.5 g, 1.79 mmol) and 7-quinolylboronic acid (463 mg, 2.68 mmol) in dioxane (15 mL) and H₂O (1 mL) was added K₂CO₃ (494 mg, 3.57 mmol), then Pd(dppf)Cl₂ (261 mg, 357.06 umol) was added under N₂ atmosphere, the mixture was stirred at 80° C. for 17 h under N₂ atmosphere. The reaction mixture was concentrated to remove solvent, then diluted with EA (30 mL) and filtered, washed with EA (30 mL×2), the filtrate was concentrated to give a residue. The residue was purified by flash silica gel chromatography (ISCO®; 4 g SepaFlash® Silica Flash Column, Eluent of 0-70% Ethyl acetate/Petroleum ether gradient @ 20 mUmin). Compound 1A (0.48 g, yield: 91.4%) as yellow oil was obtained. ¹H NMR (400 MHz, CDCl₃) δ 8.94 (dd, J=1.8, 4.2 Hz, 1H), 8.56-8.49 (m, 1H), 8.18 (d, J=8.6 Hz, 1H), 8.04-7.94 (m, 2H), 7.85 (d, J=8.4 Hz, 1H), 7.44-7.37 (m, 1H), 4.26 (q, J=7.1 Hz, 2H), 4.01 (s, 3H), 1.30-1.24 (m, 3H). MS (ESI) m/z (M+H)⁺ 282.2.

To a solution of compound 1A (0.48 g, 1.71 mmol) in MeOH (10 mL) was added the solution of NaOH (341 mg, 8.53 mmol) in H₂O (2 mL), the mixture was stirred at 50° C. for 18 h. The reaction mixture was concentrated to remove MeOH, diluted with water (10 mL), extracted with EA (20 mL), the aqueous phase was acidized with 1N HCl to pH ˜3, the precipitate was formed, the solid was filtered and lyophilized. Compound 1B (0.22 g, yield: 50.9%) as yellow solid was obtained, which was used into the next step without further purification. ¹H NMR (400 MHz, DMSO-d₆) δ 9.10 (dd, J=1.4, 4.7 Hz, 1H), 8.77 (d, J=7.9 Hz, 1H), 8.65 (s, 1H), 8.42 (s, 1H), 8.23-8.11 (m, 2H), 7.81 (dd, J=4.6, 8.4 Hz, 1H), 3.97 (s, 3H). MS (ESI) m/z (M+H)⁺ 254.2.

To a mixture of compound 1B (210 mg, 829.20 umol), Intermediate 1D (230 mg, 997.01 umol, HCl) in DMF (6 mL) was added DIEA (4.13 mmol, 720 uL), and then added HBTU (377 mg, 994.09 umol). The mixture was stirred at 25° C. for 1.5 h. The reaction mixture was added in H₂O (40 mL, 0° C.), a quantity of yellow precipitate was formed, and then stirred at 0° C. for 15 min. The solid were washed with H₂O (10 mL×2) and lyophilized. The residue was triturated in DCM (3 mL) and PE (20 mL), and then filtered. Compound 1C (190 mg, yield: 50.8%) was obtained as a yellow solid. ¹H NMR (400 MHz, DMSO-d₆) 8.90 (s, 1H), 8.39-8.30 (m, 2H), 8.19-8.07 (m, 1H), 7.95-7.83 (m, 2H), 7.81-7.72 (m, 1H), 7.56-7.46 (m, 1H), 7.41-7.11 (m, 7H), 5.92-5.74 (m, 1H), 4.58-4.41 (m, 1H), 4.12-4.03 (m, 1H), 3.93 (s, 3H), 3.85 (br d, J=4.3 Hz, 1H), 3.19-2.74 (m, 2H). MS (ESI) m/z (M+H)⁺ 430.2.

To a solution of compound 1C (0.19 g, 442.41 umol) in DMSO (10 mL) and DCM (60 mL) was added DMP (751 mg, 1.77 mmol), the mixture was stirred at 25° C. for 1.5 h. The reaction mixture was diluted with DCM (20 mL), then quenched with saturated Na₂S₂O₃ (60 mL) and saturated NaHCO₃ (60 mL), extracted with DCM (50 mL×2), the organic layers were washed with water (100 mL×2) and brine (100 mL×2), dried over Na₂SO₄, filtered and concentrated to give a residue. The residue was triturated in CH₃CN (3 mL) and isopropyl ether (3 mL), then filtered and lyophilized. Compound 1 (30 mg, yield: 15.5%) as light yellow solid was obtained. ¹H NMR (400 MHz, DMSO-d₆) δ 8.89 (br s, 1H), 8.42-8.26 (m, 2H), 8.12 (br s, 1H), 8.00-7.43 (m, 5H), 7.33-6.76 (m, 6H), 5.43-4.51 (m, 1H), 3.94 (s, 3H), 3.21 (d, J=14.1 Hz, 1H), 2.96-2.84 (m, 1H). MS (ESI) m/z (M+H)⁺ 428.1.

### N-(4-AMINO-3,4-DIOXO-1-PHENYLBUTAN-2-YL)-3-(2,3-DIMETHOXYPHENYL)-1-METHYL-1H-PYRAZOLE-4-CARBOXAMIDE (12)

Compounds 12, 14, 18, 22, 28, 54, 94, 99, 100, 101, and 102 were prepared as in Example 1 using the corresponding boronic acid or boronate ester, respectively. Compound 12 (88 mg, yield: 66.5%) as a light yellow solid was obtained: ¹H NMR (400 MHz, DMSO-d₆) δ 8.15 (s, 1H), 8.02 (s, 1H), 7.83-7.73 (m, 2H), 7.30-7.11 (m, 5H), 7.09-6.98 (m, 2H), 6.72 (dd, J=1.5, 7.3 Hz, 1H), 5.42-5.15 (m, 1H), 3.88 (s, 3H), 3.81 (s, 3H), 3.42 (s, 3H), 3.10 (dd, J=3.5, 14.1 Hz, 1H), 2.74 (dd, J=9.5, 13.6 Hz, 1H). MS (ESI) m/z (M+H)⁺ 437.2.

### N-(4-AMINO-3,4-DIOXO-1-PHENYLBUTAN-2-YL)-1-METHYL-3-(QUINOLIN-YL)-1H-PYRAZOLE-4-CARBOXAMIDE (14)

Compound 14 (90 mg, yield: 53.7%) as a white solid was obtained: ¹H NMR (400 MHz, DMSO-d₆) δ 8.64 (dd, J=1.9, 4.1 Hz, 1H), 8.36 (dd, J=1.8, 8.4 Hz, 1H), 8.16 (s, 1H), 7.99 (dd, J=1.5, 8.2 Hz, 1H), 7.89 (s, 1H), 7.82 (d, J=7.5 Hz, 1H), 7.69 (s, 1H), 7.65-7.55 (m, 2H), 7.47 (dd, J=4.1, 8.3 Hz, 1H), 7.19-7.11 (m, 3H), 6.92 (dd, J=2.0, 7.3 Hz, 2H), 5.13-5.05 (m, 1H), 3.94-3.85 (m, 3H), 2.94 (dd, J=4.0, 13.9 Hz, 1H), 2.59-2.50 (m, 1H). MS (ESI) m/z (M+H)⁺ 428.2.

### N-(4-AMINO-3,4-DIOXO-1-PHENYLBUTAN-2-YL)-1-(DIFLUOROMETHYL)-3-(QUINOLIN-8-YL)-1H-PYRAZOLE-4-CARBOXAMIDE (18)

Compound 18 (80 mg, yield: 54.7%) as a white solid was obtained: ¹H NMR (400 MHz, DMSO-d₆) δ 8.67-8.60 (m, 1H), 8.56 (dd, J=1.8, 4.2 Hz, 1H), 8.42 (d, J=7.5 Hz, 1H), 8.38-8.33 (m, 1H), 8.03 (dd, J=1.3, 8.4 Hz, 1H), 7.95-7.77 (m, 2H), 7.76-7.69 (m, 2H), 7.65-7.59 (m, 1H), 7.46 (dd, J=4.2, 8.4 Hz, 1H), 7.26-7.16 (m, 3H), 7.10 (d, J=6.8 Hz, 2H), 5.22-5.05 (m, 1H), 3.02 (dd, J=3.6, 14.0 Hz, 1H), 2.64 (dd, J=9.7, 13.9 Hz, 1H). MS (ESI) m/z (M+H)⁺ 464.1.

### N-(4-AMINO-3,4-DIOXO-1-PHENYLBUTAN-2-YL)-1-(DIFLUOROMETHYL)-3-(ISOQUINOLIN-8-YL)-1H-PYRAZOLE-4-CARBOXAMIDE (22)

Compound 22 (90 mg, yield: 53.1%) as a white solid was obtained: ¹H NMR (400 MHz, DMSO-d₆) δ 9.14-9.06 (m, 1H), 8.81 (s, 1H), 8.51 (d, J=5.5 Hz, 1H), 8.29 (br s, 1H), 8.09-7.79 (m, 3H), 7.76 (t, J=7.8 Hz, 1H), 7.70 (d, J=9.0 Hz, 1H), 7.57 (d, J=7.0 Hz, 1H), 7.51 (br s, 1H), 7.25-7.12 (m, 5H), 5.36-5.07 (m, 1H), 3.16 (d, J=4.5 Hz, 1H), 2.83 (dd, J=9.2, 13.9 Hz, 1H). MS (ESI) m/z (M+H)⁺ 464.1.

### N-(4-AMINO-3,4-DIOXO-1-PHENYLBUTAN-2-YL)-1-METHYL-3-(2-METHYLFURAN-3-YL)-1H-PYRAZOLE-4-CARBOXAMIDE (28)

Compound 28 (170 mg, yield: 85.5%) as a white solid was obtained: ¹H NMR (400 MHz, DMSO-d₆) δ 8.12-7.99 (m, 3H), 7.77 (s, 1H), 7.40 (d, J=2.0 Hz, 1H), 7.29-7.15 (m, 5H), 6.48 (d, J=1.8 Hz, 1H), 5.38-5.13 (m, 1H), 3.83 (s, 3H), 3.12 (dd, J=3.9, 13.8 Hz, 1H), 2.79 (dd, J=9.7, 13.9 Hz, 1H), 2.19 (s, 3H). MS (ESI) m/z (M+H)⁺ 381.1.

### N-(4-AMINO-3,4-DIOXO-1-PHENYLBUTAN-2-YL)-3-(ISOQUINOLIN-YL)-1-METHYL-1H-PYRAZOLE-4-CARBOXAMIDE (54)

Compound 54 (15 mg, yield: 14.5%) as a white solid was obtained: ¹H NMR (400 MHz, DMSO-d₆) δ 9.17-9.03 (m, 1H), 8.44 (d, J=6.0 Hz, 1H), 8.35 (d, J=7.5 Hz, 1H), 7.94-7.92 (m, 1H), 7.82 (d, J=5.7 Hz, 1H), 7.82-7.79 (m, 1H), 7.74-7.61 (m, 2H), 7.46-7.28 (m, 2H), 7.26-6.97 (m, 6H), 5.16-5.11 (m, 0.5H), 4.47-4.31 (m, 0.5H), 3.99-3.92 (m, 3H), 3.19-2.70 (m, 2H). MS (ESI) m/z (M+H)⁺ 428.1.

### N-(4-AMINO-3,4-DIOXO-1-PHENYLBUTAN-2-YL)-2-(DIFLUOROMETHYL)-4-(1H-INDAZOL-7-YL)OXAZOLE-5-CARBOXAMIDE (94)

Intermediate derivatives 7-(4,4,5,5-tetramethyl-1,3,2-dioxaborolan-2-yl)-1-((2-(trimethylsilyl)ethoxy)methyl)-1H-indazole and ethyl 1-(difluoromethyl)-3-iodo-1H-pyrazole-4-carboxylate were subjected to conditions as described for compound 12 to yield compound 94. Compound 94 (63 mg, yield: 40.9%) as a pale-yellow solid was obtained: ¹H NMR (400 MHz, DMSO-d₆) δ 12.94 (br s, 1H), 8.91 (d, J=7.5 Hz, 1H), 8.63 (s, 1H), 8.19-8.12 (m, 2H), 8.01-7.84 (m, 2H), 7.81 (d, J=7.8 Hz, 1H), 7.75 (d, J=7.3 Hz, 1H), 7.31 (d, J=4.3 Hz, 4H), 7.26-7.22 (m, 1H), 7.10 (t, J=7.7 Hz, 1H), 5.42-5.34 (m, 1H), 3.21 (dd, J=3.9, 13.9 Hz, 1H), 2.85 (dd, J=9.9, 13.9 Hz, 1H). MS (ESI) m/z (M+H)⁺⁼453.1.

### N-(4-AMINO-3,4-DIOXO-1-PHENYLBUTAN-2-YL)-1-METHYL-3-(2-METHYL-2H-INDAZOL-7-YL)-1H-PYRAZOLE-4-CARBOXAMIDE (99)

Intermediate derivatives (2-methyl-2H-indazol-7-yl)boronic acid and ethyl 3-iodo-1-methyl-1H-pyrazole-4-carboxylate were subjected to conditions as described for compound 12 to yield compound 99. Compound 99 (70 mg, yield: 23.4%) as a white solid was obtained: ¹H NMR (400 MHz, DMSO-d₆) δ 8.41 (s, 1H), 8.15 (s, 1H), 8.00 (s, 1H), 7.93 (d, J=7.6 Hz, 1H), 7.80-7.74 (m, 2H), 7.18-7.05 (m, 5H), 6.82-6.78 (m, 2H), 5.25-5.18 (m, 1H), 4.09 (s, 3H), 3.92-3.87 (m, 3H), 3.01-2.95 (m, 1H), 2.47-2.41 (m, 1H). MS (ESI) m/z (M+H)⁺ 431.1.

### N-(4-AMINO-3,4-DIOXO-1-PHENYLBUTAN-2-YL)-3-(1-ISOPROPYL-1H-INDAZOL-4-YL)-1-METHYL-1H-PYRAZOLE-4-CARBOXAMIDE (100)

Intermediate derivatives 1-isopropyl-4-(4,4,5,5-tetramethyl-1,3,2-dioxaborolan-2-yl)-1H-indazole and ethyl 1-(difluoromethyl)-3-iodo-1H-pyrazole-4-carboxylate were subjected to conditions as described for compound 12 to yield compound 100. Compound 100 (60 mg, yield: 48.41%) as a white solid was obtained. MS (ESI) m/z (M+H)⁺⁼459.2. ¹H NMR (400 MHz, DMSO-d₆) δ 8.31 (d, J=7.2 Hz, 1H), 8.09-8.05 (m, 2H), 8.04 (br. s, 1H), 7.79 (br. s, 1H), 7.60 (d, J=7.2 Hz, 1H), 7.30-7.14 (m, 7H), 5.31-5.20 (m, 1H), 5.03-4.91 (m, 1H), 3.92 (s, 3H), 3.16-3.04 (m, 1H), 2.83-2.71 (m, 1H), 1.45 (d, J=6.4 Hz, 6H).

### N-(4-AMINO-3,4-DIOXO-1-PHENYLBUTAN-2-YL)-3-(BENZO[b]THIOPHEN-7-YL)-1-METHYL-1H-PYRAZOLE-4-CARBOXAMIDE (101)

Compound 101 (50 mg, yield: 11.58%) as a white solid was obtained: ¹H NMR (400 MHz, DMSO-d₆) 7.96 (s, 1H), 7.92 (dd, J=1.1, 7.9 Hz, 1H), 7.62 (d, J=5.5 Hz, 1H), 7.51-7.47 (m, 2H), 7.44-7.38 (m, 1H), 7.23-7.19 (m, 3H), 7.00 (dd, J=2.9, 6.7 Hz, 2H), 6.97-6.92 (m, 1H), 6.58 (br d, J=6.8 Hz, 1H), 6.20 (br s, 1H), 5.37 (ddd, J=4.8, 7.0, 8.5 Hz, 1H), 3.99-3.93 (m, 3H), 3.17 (dd, J=4.9, 13.9 Hz, 1H), 2.81 (dd, J=8.7, 13.9 Hz, 1H). MS (ESI) m/z (M+H)+=433.1.

### N-(4-AMINO-3,4-DIOXO-1-PHENYLBUTAN-2-YL)-3-(BENZO[b]THIOPHEN-4-YL)-1-METHYL-1H-PYRAZOLE-4-CARBOXAMIDE (102)

Compound 102 (100 mg, yield: 71.0%) as a white solid was obtained: ¹H NMR (DMSO-d₆, 400 MHz): δ 8.19 (s, 1H), 8.15 (d, J=7.5 Hz, 1H), 8.02 (s, 1H), 8.00-7.94 (m, 1H), 7.79 (s, 1H), 7.67 (d, J=5.5 Hz, 1H), 7.37-7.15 (m, 8H), 5.31-5.14 (m, 1H), 3.95 (s, 3H), 3.11 (dd, J=3.8, 13.8 Hz, 1H), 2.77 (dd, J=9.7, 13.9 Hz, 1H). MS (ESI) m/z (M+H)⁺ 433.1.

### Example 2

### COMPOUNDS 4, 10, 13, 25, 37, 49, AND 63 N-(4-AMINO-3,4-DIOXO-1-PHENYLBUTAN-2-YL)-1-(DIFLUOROMETHYL)-3-(ISOQUINOLIN-1-YL)-1H-PYRAZOLE-4-CARBOXAMIDE (3)

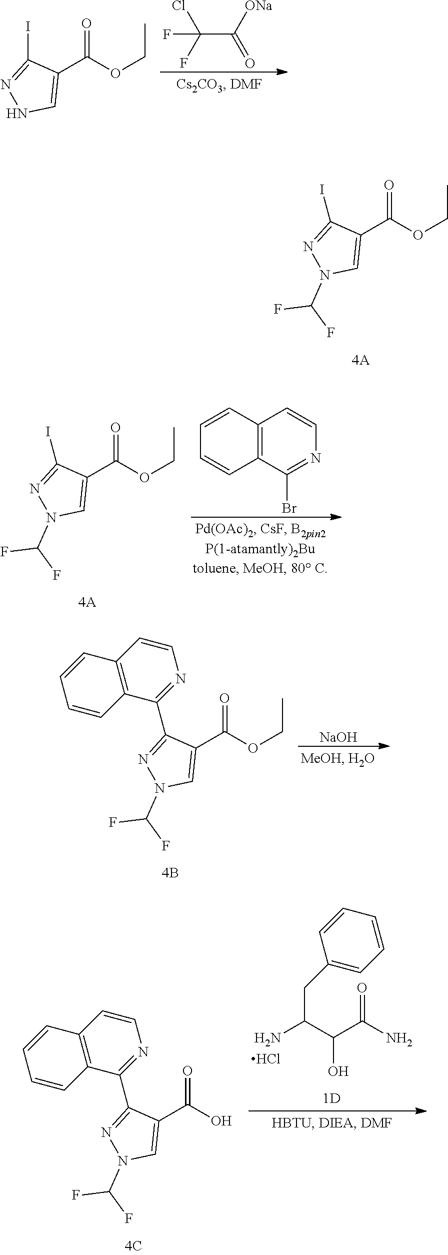

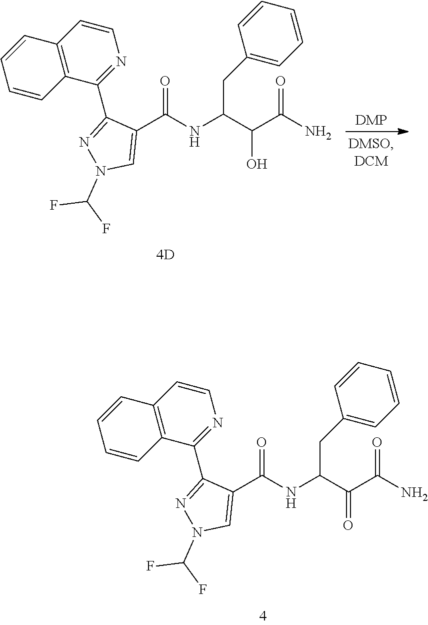 [SMILES: NC(=O)C(O)C(Cc1ccccc1)NC(=O)c1cn(C(F)F)nc1-c1nccc2ccccc12 ; NC(=O)C(=O)C(Cc1ccccc1)NC(=O)c1cn(C(F)F)nc1-c1nccc2ccccc12]

To a solution of ethyl 3-iodo-1H-pyrazole-4-carboxylate (20 g, 75.18 mmol) in DMF (100 mL) was added sodium 2-chloro-2,2-difluoroacetate (22.92 g, 150.36 mmol) and Cs₂CO₃ (48.99 g, 150.36 mmol). The mixture was stirred at 100° C. for 16 h. The reaction mixture was concentrated, the residue was diluted with H₂O (200 mL) and extracted with EtOAc (100 mL×3). The combined organic layers were washed with brine (200 mL), dried over Na₂SO₄, filtered and concentrated under reduced pressure to give a residue. The residue was purified by flash silica gel chromatography (ISCO®; X g SepaFlash® Silica Flash Column, eluent of 0%-10%-20% Ethyl acetate/Petroleum ether gradient). Compound 4A (9.1 g, yield: 38.30%) was obtained as a white solid. ¹H NMR (400 MHz, CDCl₃) δ 8.47-7.95 (m, 1H), 7.44-6.95 (m, 1H), 4.53-4.17 (m, 2H), 1.54-1.17 (m, 3H).

To a solution of compound 4A (500 mg, 1.58 mmol), 1-bromoisoquinoline (329 mg, 1.58 mmol), CsF (480 mg, 3.16 mmol), and B₂pin₂ (603 mg, 2.37 mmol) in toluene (8 mL) and MeOH (8 mL) was added Pd(OAc)₂ (35.52 mg, 158.21 umol) and P(1-adamantyl)₂Bu (57 mg, 158.98 umol) in one portion under N₂ atmosphere. The mixture was stirred at 80° C. for 16 hr under N₂ atmosphere. The reaction mixture was filtered and concentrated, the residue was diluted with H₂O (10 mL) and extracted with EA (10 mL×3). The organic layers were dried over Na₂SO₄, filtered and concentrated to give a residue. The residue was purified by flash silica gel chromatography (PE:EA=5:1 to 2:1). Compound 4B (80 mg, yield: 12.1%) was obtained as a yellow solid. ¹H NMR (400 MHz, CDCl₃) δ 8.64 (d, J=5.7 Hz, 1H), 8.54 (s, 1H), 7.90 (d, J=8.2 Hz, 1H), 7.83-7.75 (m, 2H), 7.74-7.68 (m, 1H), 7.55 (ddd, J=1.1, 7.0, 8.4 Hz, 1H), 7.48-7.29 (m, 1H), 4.01 (q, J=7.1 Hz, 2H), 0.86 (t, J=7.2 Hz, 3H). MS (ESI) m/z (M+H)⁺ 317.9.

To a solution of compound 4B (80 mg, 252.14 umol) in MeOH (10 mL) and H₂O (3 mL) was added NaOH (40 mg, 1.00 mmol). The mixture was stirred at 50° C. for 16 hr. The reaction mixture was concentrated, diluted with water (10 mL), extracted with MTBE (10 mL), then the aqueous phase were acidized with 2N HCl to pH ˜2-3, and lyophilized. Then the residue was stirred in the solution (DCM:MeOH=10:1), filtered and concentrated to give a residue. Compound 4C (39 mg, yield: 53.5%) was obtained as a brown solid. ¹H NMR (400 MHz, DMSO-d₆) δ 8.91 (s, 1H), 8.51 (d, J=5.7 Hz, 1H), 8.01 (t, J=8.5 Hz, 2H), 7.98-7.85 (m, 2H), 7.81-7.72 (m, 1H), 7.62 (t, J=7.7 Hz, 1H).

To a solution of compound 4C (64 mg, 221.27 umol) and Intermediate 1D (56 mg, 242.75 umol, HCl) in DMF (10 mL) was added HBTU (101 mg, 266.32 umol), then was added DIEA (114 mg, 882.06 umol, 153.64 uL) and stirred at 25° C. for 2 hr. The reaction mixture was diluted with water (40 mL), extracted with EA (30 mL×3), the organic layers were concentrated to give a residue. The residue was triturated in PE:EA (10:1, 20 mL) and collected by filtration. Compound 4D (80 mg, yield: 76.8%) was obtained as a pale-yellow solid. ¹H NMR (400 MHz, DMSO-d₆) δ 9.71-9.27 (m, 1H), 8.84-8.54 (m, 2H), 8.41-7.57 (m, 6H), 7.30 (br s, 1H), 7.16-6.62 (m, 6H), 6.17-5.76 (m, 1H), 4.52-4.23 (m, 1H), 3.93-3.75 (m, 1H), 2.85-2.67 (m, 2H). MS (ESI) m/z (M+H)⁺ 466.1.

To a solution of compound 4D (80 mg, 171.88 umol) in DMSO (10 mL) and DCM (50 mL) was added DMP (292 mg, 688.45 umol). The mixture was stirred at 25° C. for 3 hr. The reaction mixture was diluted with DCM (20 mL), quenched with saturated NaHCO₃ (25 mL) and saturated Na₂S₂O₃ (25 mL), the mixture was stirred 10 min. The organic layer was washed with water (40 mL×2), brine (40 mL×2), dried over Na₂SO₄, then filtered and concentrated to give a residue. The residue was purified by flash silica gel chromatography (PE:EA=1:1 to 0:1). Compound 4 (25 mg, yield: 29.9%) was obtained as a pale yellow solid. ¹H NMR (400 MHz, DMSO-d₆) δ 9.81 (d, J=7.3 Hz, 1H), 8.88 (s, 1H), 8.37 (d, J=5.5 Hz, 1H), 8.28 (d, J=9.0 Hz, 1H), 8.14-7.97 (m, 3H), 7.92 (d, J=6.0 Hz, 1H), 7.83 (br d, J=5.3 Hz, 2H), 7.72-7.66 (m, 1H), 7.06-6.92 (m, 5H), 5.46-5.36 (m, 1H), 3.15 (br dd, J=4.5, 14.0 Hz, 1H), 2.88 (dd, J=8.7, 14.0 Hz, 1H). MS (ESI) m/z (M+H)⁺ 464.1.

### N-(4-AMINO-3,4-DIOXO-1-PHENYLBUTAN-2-YL)-3-(ISOQUINOLIN-1-YL)-1-METHYL-1H-PYRAZOLE-4-CARBOXAMIDE (10)

Compounds 10, 13, 25, 37, 49, and 63 were prepared as in Example 2 using the corresponding carboxylic acid, respectively. Ethyl 3-iodo-1-methyl-1H-pyrazole-4-carboxylate was used to obtain compound 10 (55 mg, yield: 61.2%) as a pale yellow solid was obtained: ¹H NMR (400 MHz, DMSO-d₆) δ 10.21 (d, J=7.3 Hz, 1H), 8.61 (d, J=8.2 Hz, 1H), 8.37 (s, 1H), 8.32 (d, J=6.0 Hz, 1H), 8.12-8.02 (m, 2H), 7.90-7.80 (m, 3H), 7.69 (t, J=7.8 Hz, 1H), 7.05-6.88 (m, 5H), 5.47 (d, J=4.9 Hz, 1H), 4.01 (s, 3H), 3.17 (dd, J=4.7, 13.8 Hz, 1H), 2.91 (dd, J=7.3, 14.3 Hz, 1H). MS (ESI) m/z (M+H)⁺ 428.2.

### N-(4-AMINO-3,4-DIOXO-1-PHENYLBUTAN-2-YL)-1-METHYL-3-(QUINOXALIN-2-YL)-1H-PYRAZOLE-4-CARBOXAMIDE (13)

Ethyl 3-iodo-1-methyl-1H-pyrazole-4-carboxylate was used to obtain compound 13 (20 mg, yield: 76.2%) as a white solid was obtained: ¹H NMR (400 MHz, DMSO-d₆) δ 11.18 (d, J=8.2 Hz, 1H), 9.60 (s, 1H), 8.46 (s, 1H), 8.19 (s, 1H), 8.12 (d, J=8.2 Hz, 1H), 7.92-7.84 (m, 2H), 7.77 (dt, J=1.3, 7.7 Hz, 1H), 7.65 (d, J=8.4 Hz, 1H), 7.01-6.93 (m, 4H), 6.90-6.79 (m, 1H), 5.79-5.74 (m, 1H), 4.03 (s, 3H), 3.29-3.18 (m, 2H). MS (ESI) m/z (M+H)⁺ 429.1.

### N-(4-AMINO-3,4-DIOXO-1-PHENYLBUTAN-2-YL)-1-(DIFLUOROMETHYL)-3-(QUINOXALIN-2-YL)-1H-PYRAZOLE-4-CARBOXAMIDE (25)

Compound 25 (20 mg, yield: 52.2%) as a white solid was obtained: ¹H NMR (400 MHz, DMSO-d₆) δ 10.80 (d, J=8.2 Hz, 1H), 9.51 (s, 1H), 8.92 (s, 1H), 8.23-7.82 (m, 5H), 7.78 (dt, J=1.3, 7.6 Hz, 1H), 7.71-7.65 (m, 1H), 7.01-6.89 (m, 4H), 6.88-6.82 (m, 1H), 5.77-5.67 (m, 1H), 3.24-3.12 (m, 2H). MS (ESI) m/z (M+H)⁺ 465.1.

### N-(4-AMINO-3,4-DIOXO-1-PHENYLBUTAN-2-YL)-3-(6,7-DIMETHOXYQUINOLIN-4-YL)-1-METHYL-1H-PYRAZOLE-4-CARBOXAMIDE (37)

Compound 37 (15 mg, yield: 47.2%) as a pale yellow solid was obtained: ¹H NMR (400 MHz, DMSO-d₆) 8.62 (d, J=4.5 Hz, 1H), 8.35-8.23 (m, 1H), 7.71 (br d, J=6.8 Hz, 1H), 7.65 (br s, 1H), 7.49 (br s, 1H), 7.41 (s, 1H), 7.26-7.17 (m, 5H), 7.10 (d, J=6.8 Hz, 2H), 5.27-5.18 (m, 1H), 3.99 (s, 3H), 3.96 (s, 3H), 3.72 (s, 3H), 3.16-3.21 (m, 1H), 2.75-2.81 (m, 1H). MS (ESI) m/z (M+H)⁺ 488.2.

### N-(4-AMINO-3,4-DIOXO-1-PHENYLBUTAN-2-YL)-1-METHYL-3-(QUINAZOLIN-4-YL)-1H-PYRAZOLE-4-CARBOXAMIDE (49)

Ethyl 3-iodo-1-methyl-1H-pyrazole-4-carboxylate was used to obtain compound 49 (62 mg, yield: 61.3%) as a white solid was obtained: ¹H NMR (400 MHz, DMSO-d₆) 10.10 (d, J=7.5 Hz, 1H), 8.97 (s, 1H), 8.66 (d, J=8.4 Hz, 1H), 8.44 (s, 1H), 8.11 (s, 1H), 8.06 (d, J=3.5 Hz, 2H), 7.84 (s, 1H), 7.80-7.72 (m, 1H), 7.01 (s, 5H), 5.61-5.35 (m, 1H), 4.03 (s, 3H), 3.18 (dd, J=5.0, 14.2 Hz, 1H), 2.99 (dd, J=7.6, 14.0 Hz, 1H). MS (ESI) m/z (M+H)⁺ 429.1.

### N-(4-AMINO-3,4-DIOXO-1-PHENYLBUTAN-2-YL)-1-(DIFLUOROMETHYL)-3-(QUINAZOLIN-4-YL)-1H-PYRAZOLE-4-CARBOXAMIDE (63)

Compound 63 (28 mg, yield: 73.3%) as a pale yellow solid was obtained: H NMR (400 MHz, DMSO-d₆) δ 9.51 (d, J=7.5 Hz, 1H), 9.11 (s, 1H), 8.92 (s, 1H), 8.23 (d, J=8.6 Hz, 1H), 8.18-7.99 (m, 4H), 7.90-7.80 (m, 1H), 7.79-7.71 (m, 1H), 7.14-7.03 (m, 5H), 5.36 (dt, J=4.6, 7.9 Hz, 1H), 3.14 (dd, J=4.2, 13.9 Hz, 1H), 2.89 (dd, J=8.5, 14.0 Hz, 1H). MS (ESI) m/z (M+H)⁺ 465.1.

### Example 3

### N-(4-AMINO-3,4-DIOXO-1-PHENYLBUTAN-2-YL)-1-METHYL-3-(PIPERAZIN-1-YL)-1H-PYRAZOLE-4-CARBOXAMIDE HYDROCHLORIDE (2)

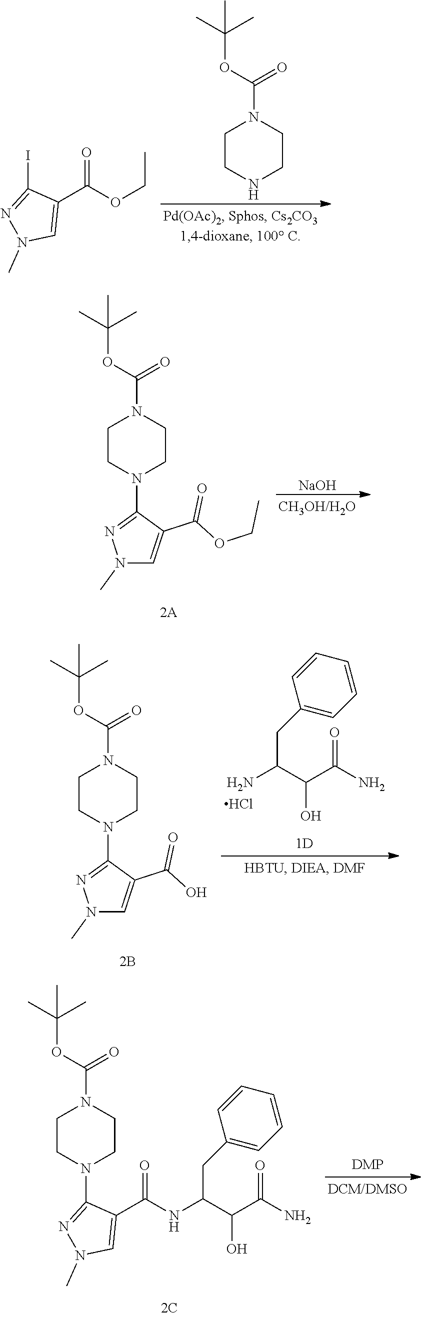

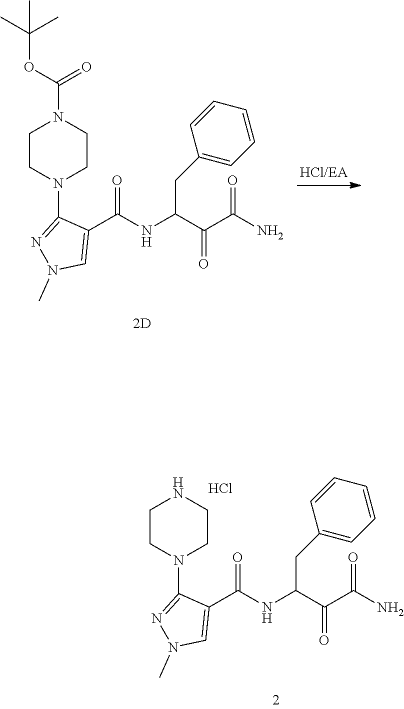 [SMILES: Cn1cc(C(=O)NC(Cc2ccccc2)C(=O)C(N)=O)c(N2CCN(C(=O)OC(C)(C)C)CC2)n1 ; Cn1cc(C(=O)NC(Cc2ccccc2)C(=O)C(N)=O)c(N2CCNCC2)n1 ; Cl]

To a solution of ethyl 3-iodo-1-methyl-1H-pyrazole-4-carboxylate (0.5 g, 1.79 mmol) and tert-butyl piperazine-1-carboxylate (665 mg, 3.57 mmol) in dioxane (20 mL) was added S-Phos (147 mg, 357.06 umol) and Cs₂CO₃ (1.16 g, 3.57 mmol), then Pd(OAc)₂ (40 mg, 178.53 umol) was added under N₂ atmosphere. The reaction was stirred at 100° C. for 17 h. The reaction mixture was filtered, washed with EA (30 mL×2), the filtrate was concentrated to give a residue. The residue was purified by flash silica gel chromatography (ISCO®; 4 g SepaFlash® Silica Flash Column, Eluent of 0-10% Ethyl acetate/Petroleum ethergradient @ 20 mUmin). Compound 2A (0.15 g, yield: 22.8%) as light yellow oil was obtained. ¹H NMR (400 MHz, CDCl₃) δ 7.75 (s, 1H), 4.24 (q, J=7.1 Hz, 2H), 3.76 (s, 3H), 3.61-3.54 (m, 4H), 3.30-3.20 (m, 4H), 1.47 (s, 9H), 1.32 (t, J=7.1 Hz, 3H). MS (ESI) m/z (M+H)⁺ 339.1.

Compound 2A was transformed into compound 2D as shown in Example 1. Compound 2D (0.10 g, yield: 72.2%) as yellow solid was obtained. ¹H NMR (400 MHz, DMSO-d₆) δ 8.30-7.84 (m, 4H), 7.30-7.17 (m, 3H), 7.07 (d, J=7.1 Hz, 2H), 5.57-5.44 (m, 1H), 3.76-3.67 (m, 3H), 3.28-3.08 (m, 6H), 2.86-2.70 (m, 4H), 1.43-1.38 (m, 9H). MS (ESI) m/z (M+H)⁺ 485.3.

To a solution of compound 2D (100 mg, 206.38 umol) in EtOAc (2 mL) was added HCl/EtOAc (4M, 4 mL), the mixture was stirred at 25° C. for 4 h. The reaction mixture was concentrated to give a residue. The residue was triturated in CH₃CN (10 mL×2), and then concentrated to give a residue. Compound 2 (75 mg, yield: 94.3%) as yellow solid was obtained. ¹H NMR (400 MHz, DMSO-d₆) 9.35 (br s, 2H), 8.17-8.06 (m, 2H), 7.87 (br s, 1H), 7.32-7.12 (m, 5H), 5.53-5.29 (m, 1H), 3.74 (s, 3H), 3.28-2.86 (m, 10H). MS (ESI) m/z (M+H)⁺ 385.2.

### Example 4

### COMPOUNDS 6-7 N-(4-AMINO-3,4-DIOXO-1-PHENYLBUTAN-2-YL)-3-(BENZO[D]THIAZOL-7-YL)-1-METHYL-1H-PYRAZOLE-4-CARBOXAMIDE (7)

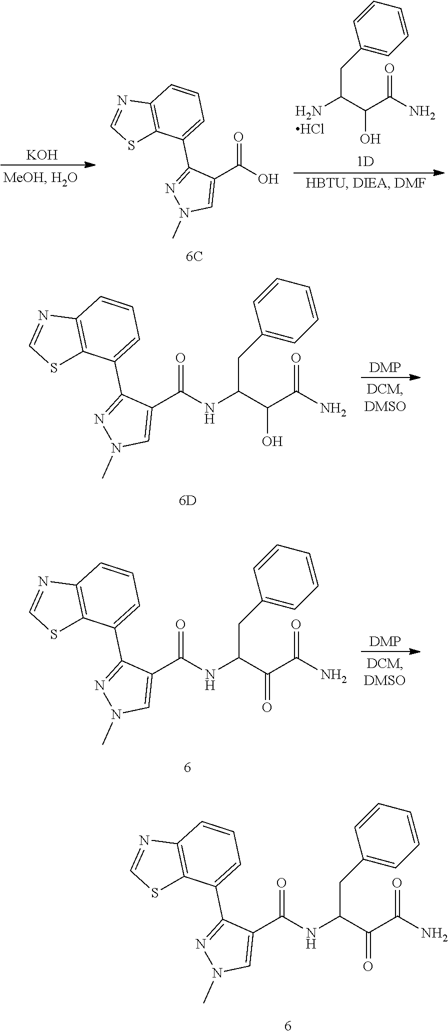

To a solution of 7-bromobenzo[d]thiazole (900 mg, 4.2 mmol) in dioxane (20 mL) was added KOAc (843 mg, 8.5 mmol), 4,4,4′,4′,5,5,5′,5′-octamethyl-2,2′-bi(1,3,2-dioxaborolane) (1.07 g, 4.2 mmol), Pd(dppf)Cl₂ (307 mg, 420 umol). Then the mixture was stirred at 90° C. for 12 h under N₂ atmosphere. The reaction was cooled to room temperature and the reaction was filtered. The filtered liquor was concentrated under reduced pressure to remove solvent. H₂O (20 mL) was added to the residue, the mixture was extracted with EA (20 mL×3). The combined organic layer was washed with brine (20 mL), dried over anhydrus Na₂SO₄, filtered and concentrated under reduced pressure to afford compound 6A (1.0 g, crude) as black oil which was used directly in next step.

Compound 6A was converted to compound 6 using procedures as described in Example 1. Compound 6 (50 mg, yield: 33%) as white solid was obtained. ¹H NMR (DMSO-d₆, 400 MHz): δ 9.36 (s, 1H), 8.60 (d, J=7.3 Hz, 1H), 8.14 (s, 1H), 8.10 (s, 1H), 8.03 (d, J=8.0 Hz, 1H), 7.83 (s, 1H), 7.78 (d, J=7.5 Hz, 1H), 7.48 (t, J=7.8 Hz, 1H), 7.33-7.27 (m, 4H), 7.26-7.20 (m, 1H), 5.41-5.22 (m, 1H), 3.97 (s, 3H), 3.18 (dd, J=3.8, 14.1 Hz, 1H), 2.83 (dd, J=10.2, 13.9 Hz, 1H). MS (ESI) m/z (M+H)⁺ 434.1.

### N-(4-AMINO-3,4-DIOXO-1-PHENYLBUTAN-2-YL)-3-(BENZO[D]THIAZOL-7-YL)-1-(DIFLUOROMETHYL)-1H-PYRAZOLE-4-CARBOXAMIDE (7)

Compounds 6A and 4A were converted to compound 7 using procedures as described in Example 1. Compound 7 (60 mg, yield: 51.6%) as yellow solid was obtained. ¹H NMR (DMSO-d₆, 400 MHz): δ 9.41 (s, 1H), 8.99 (d, J=7.5 Hz, 1H), 8.59 (s, 1H), 8.17-8.09 (m, 2H), 8.02-7.83 (m, 2H), 7.73 (d, J=7.5 Hz, 1H), 7.53 (t, J=8.0 Hz, 1H), 7.30 (s, 4H), 7.24 (br s, 1H), 5.42-5.32 (m, 1H), 3.21 (br dd, J=3.3, 13.9 Hz, 1H), 2.82 (dd, J=10.1, 13.5 Hz, 1H). MS (ESI) m/z (M+H)⁺ 470.1.

### Example 5

### Compounds 32, 62, 69, and 61

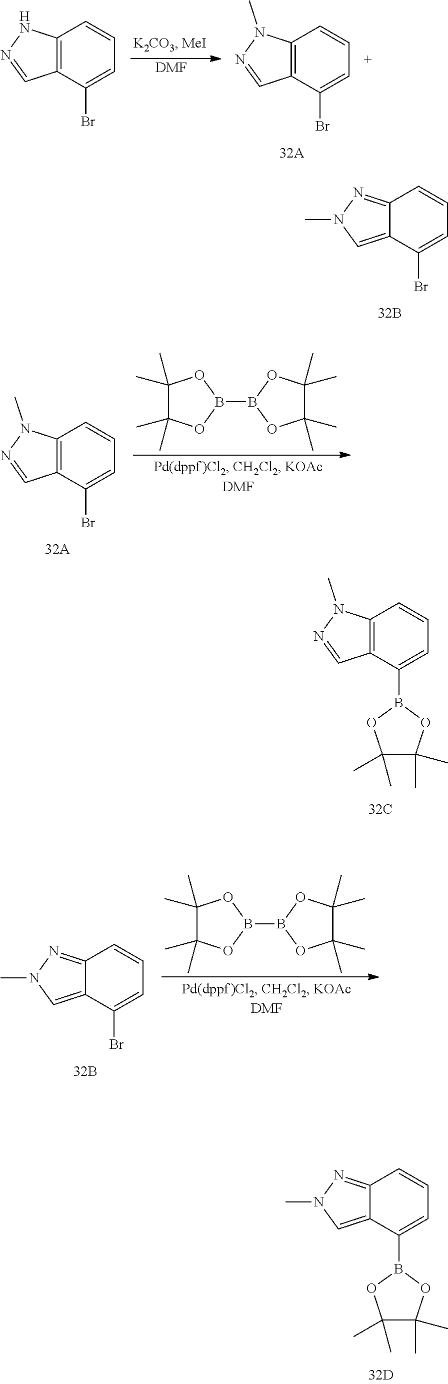

K₂CO₃ (5.26 g, 38.06 mmol) was added to a mixture of 4-bromo-1H-indazole (5 g, 25.38 mmol) in DMF (50 mL). 30 min later, MeI (18.2 g, 128.22 mmol, 8.0 mL) was added and the mixture was stirred at 25° C. for 3 h. The mixture was treated with H₂O (150 mL) and EA (50 mL). The organic layer was separated and the aqueous layer was extracted with EA (50 mL×2). The combined organic layer was washed brine (50 mL×2), dried over MgSO₄, filtered and concentrated. The residue was purified by flash column chromatography over silica gel (PE/EA=10/1 to 5/1) to afford a pair of isomers.

Isomer 1 (Compound 32A, R_{f}=0.54, PE/EA=5/1): 4-bromo-1-methyl-indazole (3.2 g, 59.8% yield) was obtained as white solid. ¹H NMR (DMSO-d₆, 400 MHz): δ 7.98 (d, J=0.9 Hz, 1H), 7.67-7.65 (m, 1H), 7.35-7.27 (m, 2H), 4.04 (s, 3H).

Isomer 2 (Compound 32B, R_{f}=0.24, PE/EA=5/1): 4-bromo-2-methyl-indazole (1.3 g, 24.3% yield) was obtained as colorless sticky oil. ¹H NMR (DMSO-d₆, 400 MHz): δ 8.37 (s, 1H), 7.60-7.57 (m, 1H), 7.26-7.21 (m, 1H), 7.13 (dd, J=7.3, 8.6 Hz, 1H), 4.16 (s, 3H).

KOAc (1.12 g, 11.37 mmol) was added to a mixture of compound 32A (1.2 g, 5.69 mmol) and 4,4,4′,4′,5,5,5′,5′-octamethyl-2,2′-bi(1,3,2-dioxaborolane) (2.17 g, 8.53 mmol) in DMF (25 mL), followed by Pd(dppf)Cl₂.CH₂Cl₂ (232 mg, 284.09 umol). Then nitrogen gas was bubbled through the mixture. The mixture was heated to 85° C. and stirred for 12 h. The mixture was treated with EA (75 mL) and brine (100 mL). The mixture was filtered through Celite. The filtrate was transferred to separating funnel. The organic layer was separated, dried over MgSO₄, filtered and concentrated. The residue was purified by silica gel column chromatography (petroleum ether/ethyl acetate=10/1 to 5/1) to afford compound 32C (1.5 g, 87.9% yield) as colorless sticky oil. ¹H NMR (DMSO-d₆, 400 MHz): δ 8.15 (d, J=0.8 Hz, 1H), 7.79 (d, J=8.5 Hz, 1H), 7.54-7.50 (m, 1H), 7.41 (dd, J=6.8, 8.5 Hz, 1H), 4.06 (s, 3H), 1.35 (s, 12H).

KOAc (1.2 g, 12.3 mmol) was added to mixture of compound 32B (1.3 g, 6.2 mmol) and 4,4,4′,4′,5,5,5′,5′-octamethyl-2,2′-bi(1,3,2-dioxaborolane) (2.4 g, 9.3 mmol) in DMF (20 mL). N₂ gas was bubbled through the mixture. Then Pd(dppf)Cl₂—CH₂Cl₂ (253 mg, 309.8 umol) was added. The mixture was stirred at 85° C. for 12 h under nitrogen atmosphere. The mixture was diluted with EA (50 mL) and brine (50 mL). The mixture was filtered through Celite. The filtrate was transferred to separating funnel. The organic layer was separated and the aqueous layer was extracted with EA (15 mL×2). The combined organic layer was washed with brine (35 mL), dried over MgSO₄, filtered and concentrated. The residue was purified by flash column chromatography over silica gel (PE/EA=5/1 to 2/1) to afford compound 32D (1.5 g, yield 94.4%) as white solid. MS (ESI) m/z (M+H)⁺ 259.2.

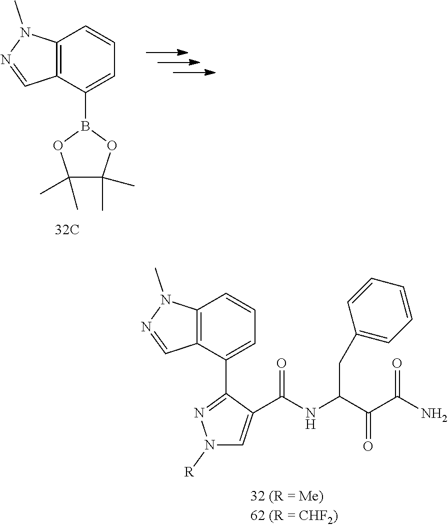

### N-(4-AMINO-3,4-DIOXO-1-PHENYLBUTAN-2-YL)-1-METHYL-3-(1-METHYL-1H-INDAZOL-4-YL)-1H-PYRAZOLE-4-CARBOXAMIDE (32)

Compounds 32C and ethyl 3-iodo-1-methyl-1H-pyrazole-4-carboxylate were converted to compound 32 using procedures as described in Example 1. Compound 32 (60 mg, yield: 60.0%) as pale yellow solid was obtained. ¹H NMR (DMSO-d₆, 400 MHz): δ 8.38 (br d, J=7.3 Hz, 1H), 8.09 (br d, J=9.5 Hz, 3H), 7.82 (br s, 1H), 7.61-7.53 (m, 1H), 7.35-7.19 (m, 7H), 5.38-5.25 (m, 1H), 4.05 (s, 3H), 3.96 (s, 3H), 3.15 (br dd, J=3.4, 13.7 Hz, 1H), 2.81 (br dd, J=10.2, 13.4 Hz, 1H). MS (ESI) m/z (M+H)⁺ 431.1.

### N-(4-AMINO-3,4-DIOXO-1-PHENYLBUTAN-2-YL)-1-(DIFLUOROMETHYL)-3-(1-METHYL-1H-INDAZOL-4-YL)-1H-PYRAZOLE-4-CARBOXAMIDE (62)

Compounds 32C and intermediate 4A were converted to compound 62 using procedures as described in Example 1. Compound 62 (96 mg, yield: 48.9%) as white solid was obtained. ¹H NMR (DMSO-d₆, 400 MHz): δ 8.52 (s, 1H), 8.46 (d, J=9.8 Hz, 1H), 8.18-7.70 (m, 3H), 7.69-7.51 (m, 2H), 7.42-7.33 (m, 2H), 7.31-7.19 (m, 5H), 5.45-5.28 (m, 1H), 4.11-4.04 (m, 3H), 3.21 (dd, J=4.4, 14.2 Hz, 1H), 2.89 (dd, J=9.4, 14.2 Hz, 1H). MS (ESI) m/z (M+H)⁺ 467.1.

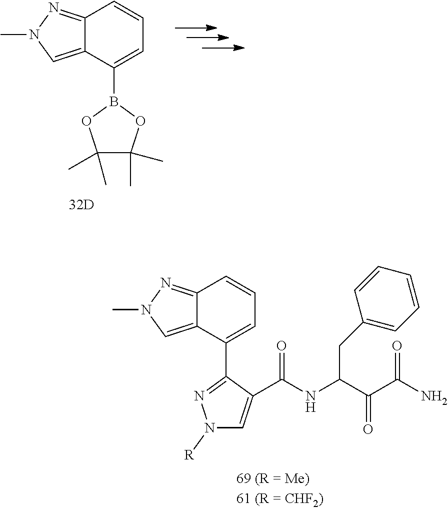

### N-(4-AMINO-3,4-DIOXO-1-PHENYLBUTAN-2-YL)-1-METHYL-3-(2-METHYL-2H-INDAZOL-4-YL)-1H-PYRAZOLE-4-CARBOXAMIDE (69)

Compounds 32D and ethyl 3-iodo-1-methyl-1H-pyrazole-4-carboxylate were converted to compound 69 using procedures as described in Example 1. Compound 69 (230 mg, yield: 69.7%) as white solid was obtained. ¹H NMR (400 MHz, DMSO-d₆) δ 8.39 (d, J=7.3 Hz, 1H), 8.36 (s, 1H), 8.10 (s, 1H), 8.06 (s, 1H), 7.85 (s, 1H), 7.53 (d, J=8.8 Hz, 1H), 7.32-7.22 (m, 6H), 7.15 (dd, J=7.2, 8.4 Hz, 1H), 5.33-5.28 (m, 1H), 4.17 (s, 3H), 3.95 (s, 3H), 3.16 (dd, J=3.9, 13.9 Hz, 1H), 2.81 (dd, J=9.9, 13.9 Hz, 1H). MS (ESI) m/z (M+H)⁺ 431.1.

### N-(4-AMINO-3,4-DIOXO-1-PHENYLBUTAN-2-YL)-1-(DIFLUOROMETHYL)-3-(2-METHYL-2H-INDAZOL-4-YL)-1H-PYRAZOLE-4-CARBOXAMIDE (61)

Compounds 32D and intermediate 4A were converted to compound 61 using procedures as described in Example 1. Compound 61 (250 mg, yield: 85.9%) as pale yellow solid was obtained. ¹H NMR (400 MHz, DMSO-d₆) δ 8.91 (d, J=7.5 Hz, 1H), 8.50 (s, 1H), 8.38 (s, 1H), 8.17-8.11 (m, 1H), 7.98-7.82 (m, 2H), 7.62 (d, J=8.5 Hz, 1H), 7.34-7.22 (m, 6H), 7.19 (dd, J=7.2, 8.4 Hz, 1H), 5.40-5.32 (m, 1H), 4.21-4.09 (m, 3H), 3.25-3.17 (m, 1H), 2.88-2.78 (m, 1H). MS (ESI) m/z (M+H)⁺⁼467.2.

### Example 6

### Compounds 33-34,77

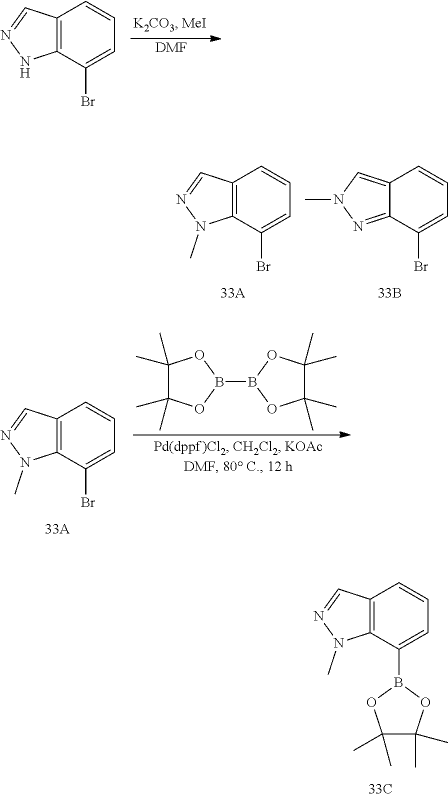

K₂CO₃ (3.51 g, 25.38 mmol) was added to a mixture of 7-bromo-1H-indazole (5 g, 25.38 mmol) in DMF (50 mL). 30 min later, MeI (18.05 g, 7.92 mL, 127.17 mmol) was added and the mixture was stirred at 25° C. for 3 h. The insoluble substance was removed by filter. The filtrate was concentrated in vacuum. The residue was treated with H₂O (50 mL) and EA (50 mL). The organic layer was separated, washed with brine (15 mL×2), dried over MgSO₄, filtered and concentrated. The residue was purified by silica gel chromatography (PE/EA=10/1 to 3/1) to afford a pair of isomers.

Isomer 1 (Compound 33A, R_{f}=0.54, PE/EA=5/1): 7-bromo-1-methyl-1H-indazole (2.85 g, 53.2% yield) was obtained as colorless oil, which turned white solid after standing by. ¹H NMR (DMSO-d₆, 400 MHz): δ 8.09 (s, 1H), 7.74 (dd, J=0.9, 7.9 Hz, 1H), 7.56 (dd, J=0.8, 7.4 Hz, 1H), 7.02-6.97 (m, 1H), 4.28 (s, 3H).

Isomer 2 (Compound 33B, R_{f}=0.18, PE/EA=5/1): 7-bromo-2-methyl-2H-indazole (1.85 g, 34.5% yield) was obtained as white solid. ¹H NMR (DMSO-d₆, 400 MHz): δ 8.47 (s, 1H), 7.69 (dd, J=0.7, 8.4 Hz, 1H), 7.49-7.44 (m, 1H), 6.91 (dd, J=7.3, 8.2 Hz, 1H), 4.17 (s, 3H).

KOAc (1.35 g, 13.74 mmol) was added to a mixture of compound 33A (1.45 g, 6.87 mmol) and 4,4,4′,4′,5,5,5′,5′-octamethyl-2,2′-bi(1,3,2-dioxaborolane) (2.62 g, 10.31 mmol) in DMF (25 mL). Nitrogen gas was bubbled through the mixture and Pd(dppf)Cl₂.CH₂Cl₂ (280 mg, 342.87 umol) was added. Then the mixture was heated to 85° C. and stirred for 12 h. The mixture was treated with EA (75 mL) and brine (100 mL). The mixture was filtered through Celite. The filtrate was transferred separating funnel. The organic layer was separated, dried over MgSO₄, filtered and concentrated. The residue was purified by silica gel column chromatography (petroleum ether/ethyl acetate=10/1 to 5/1) to afford compound 33C (1.7 g, 90.1% yield) as white solid. ¹H NMR (DMSO-d₆, 400 MHz): δ 7.99 (s, 1H), 7.89 (dd, J=1.0, 7.0 Hz, 1H), 7.82 (dd, J=1.3, 8.0 Hz, 1H), 7.13 (dd, J=7.0, 8.0 Hz, 1H), 4.31 (s, 3H), 1.41 (s, 12H). MS (ESI) m/z (M+H)⁺ 259.2.

### N-(4-AMINO-3,4-DIOXO-1-PHENYLBUTAN-2-YL)-1-METHYL-3-(1-METHYL-1H-INDAZOL-7-YL)-1H-PYRAZOLE-4-CARBOXAMIDE (33)

Compounds 33C and ethyl 3-iodo-1-methyl-1H-pyrazole-4-carboxylate were converted to compound 33 using procedures as described in Example 1. Compound 33 (70 mg, yield: 43.6%) as pale yellow solid was obtained. ¹H NMR (DMSO-d₆, 400 MHz): δ 8.37 (s, 1H), 8.06 (s, 1H), 8.02 (s, 1H), 7.93 (d, J=7.8 Hz, 1H), 7.82-7.70 (m, 2H), 7.26-7.17 (m, 3H), 7.13-7.06 (m, 4H), 5.26-5.17 (m, 1H), 3.95 (s, 3H), 3.46 (s, 3H), 3.10 (br dd, J=3.4, 13.9 Hz, 1H), 2.69 (br dd, J=9.8, 13.8 Hz, 1H). MS (ESI) m/z (M+H)⁺ 431.2.

### N-(4-AMINO-3,4-DIOXO-1-PHENYLBUTAN-2-YL)-1-(DIFLUOROMETHYL)-3-(1-METHYL-1H-INDAZOL-7-YL)-1H-PYRAZOLE-4-CARBOXAMIDE (34)

Compounds 33C and intermediate 4A were converted to compound 34 using procedures as described in Example 1. Compound 34 (30 mg, yield: 27.0%) as a white solid was obtained. ¹H NMR (400 MHz, DMSO-d₆) δ 8.81 (s, 1H), 8.10-8.00 (m, 2H), 7.92-7.43 (m, 4H), 7.22-7.07 (m, 7H), 5.30-5.22 (m, 1H), 3.52 (s, 3H), 3.15 (d, J=10.0 Hz, 1H), 2.79 (dd, J=9.4, 13.9 Hz, 1H). MS (ESI) m/z (M+H)⁺ 467.2), 4.21-4.09 (m, 3H), 3.25-3.17 (m, 1H), 2.88-2.78 (m, 1H). MS (ESI) m/z (M+H)⁺⁼467.2.

### N-(4-AMINO-3,4-DIOXO-1-PHENYLBUTAN-2-YL)-1-(DIFLUOROMETHYL)-3-(2-METHYL-2H-INDAZOL-7-YL)-1H-PYRAZOLE-4-CARBOXAMIDE (77)

Compounds 2-methyl-7-(4,4,5,5-tetramethyl-1,3,2-dioxaborolan-2-yl)-2H-indazole (prepared from intermediate 33B using same procedure as 33C) and intermediate 4A were converted to compound 77 using procedures as described in Example 1. Compound 77 (30 mg, yield: 42.6%) as a white solid was obtained. ¹H NMR (400 MHz, DMSO-d₆) δ 8.59 (s, 1H), 8.40-8.35 (m, 2H), 8.05-7.88 (m, 2H), 7.77-7.73 (m, 2H), 7.22-7.11 (m, 4H), 7.08-7.02 (m, 1H), 7.00-6.95 (m, 2H), 5.25-5.18 (m, 1H), 4.03 (s, 3H), 3.06-2.99 (m, 1H), 2.61-2.53 (m, 1H). MS (ESI) m/z (M+H)⁺ 467.2.

### Example 7

### Compounds 17, 31, 51, 70, 24, 26, and 55

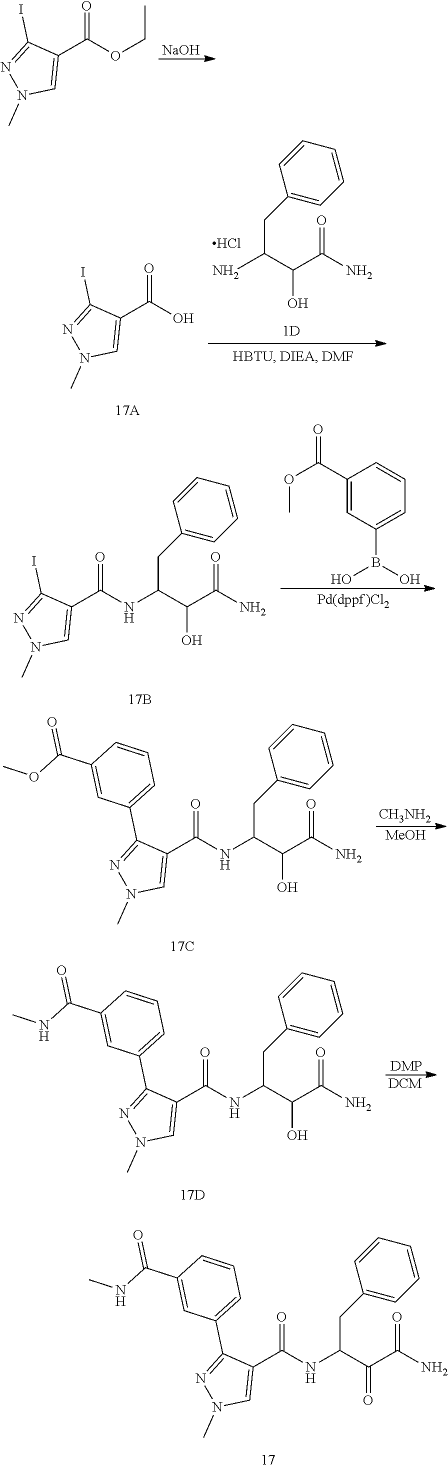

To a solution of ethyl 3-iodo-1-methyl-1H-pyrazole-4-carboxylate (1 g, 3.57 mmol) in MeOH (15 mL) was added the solution of NaOH (714 mg, 17.85 mmol) in H₂O (2 mL), the mixture was stirred at 50° C. for 1 h. The reaction mixture was concentrated to remove MeOH, then diluted with water (30 mL), acidified with 1N HCl to pH ˜3, the precipitate was formed, the solid was filtered and dried in vacuum. The residue was used into the next step without further purification. Compound 17A (850 mg, yield: 94.5%) as white solid was obtained. ¹H NMR (400 MHz, DMSO-d₆) δ 12.45 (s, 1H), 8.31-8.08 (m, 1H), 3.96-3.76 (m, 3H).

To a solution of compound 17A (0.85 g, 3.37 mmol) and Intermediate 1D (856 mg, 3.71 mmol, HCl) in DMF (20 mL) was added HBTU (1.53 g, 4.05 mmol) and DIEA (13.49 mmol, 2.35 mL), the mixture was stirred at 25° C. for 1 h. The reaction mixture was diluted with water (50 mL) at 0° C., the precipitate was formed, and the solid was filtered and dried in vacuum. The residue was used into the next step without further purification. Compound 17B (1.2 g, yield: 83.0%) as white solid was obtained. ¹H NMR (400 MHz, DMSO-d₆) δ 8.13 (s, 1H), 7.62 (d, J=9.0 Hz, 1H), 7.33 (s, 2H), 7.29-7.17 (m, 4H), 7.16-7.09 (m, 1H), 5.87 (d, J=6.0 Hz, 1H), 4.56-4.36 (m, 1H), 4.01 (dd, J=3.3, 5.7 Hz, 1H), 3.84 (s, 3H), 2.89-2.62 (m, 2H). MS (ESI) m/z (M+H)⁺ 429.0.

To a solution of compound 17B (1.2 g, 2.80 mmol) and (3-methoxycarbonylphenyl)boronic acid (756 mg, 4.20 mmol) in dioxane (30 mL) and H₂O (3 mL) was added K₂CO₃ (775 mg, 5.60 mmol), then Pd(dppf)Cl₂ (205 mg, 280.23 umol) was added under N₂ atmosphere, the mixture was stirred at 80° C. for 18 h. The reaction mixture was concentrated to remove solvent, diluted with EA (50 mL), filtered and washed with EA (20 mL×2), the filtrate was washed with water (50 mL×2), then dried over Na₂SO₄, filtered and concentrated to give a residue. The residue was purified by flash silica gel chromatography (ISCO®; 12 g SepaFlash® Silica Flash Column, Eluent of 0-100% Ethyl acetate/Petroleum ether gradient to EA:MeOH=10:1 @ 30 mL/min). Compound 17C (0.4 g, yield: 32.7%) as yellow solid was obtained. ¹H NMR (400 MHz, DMSO-d₆) δ 8.29 (t, J=1.7 Hz, 1H), 8.07 (s, 1H), 7.90-7.80 (m, 2H), 7.77-7.74 (m, 1H), 7.46-7.39 (m, 1H), 7.34-7.11 (m, 7H), 5.82 (d, J=5.7 Hz, 1H), 4.58-4.40 (m, 1H), 4.02 (dd, J=3.5, 5.7 Hz, 1H), 3.89 (s, 3H), 3.85 (s, 3H), 2.87-2.66 (m, 2H).

To a solution of compound 17C (120 mg, 274.94 umol) in MeOH (3 mL) was added CH₃NH₂ (549.88 umol, 8 mL), then the mixture was stirred at 45° C. for 40 h. The reaction mixture was concentrated to remove solvent, diluted with DCM (20 mL) and filtered, the solid was collected. The residue was purified by preparatory-HPLC (column: YMC-Actus Triart C18 100*30 mm*5 um; mobile phase: [water (0.05% HCl)-ACN]; B %: 10%-66%, 8.5 min). Compound 17D (60 mg, yield 49.8%) as white solid was obtained. ¹H NMR (400 MHz, DMSO-d₆) δ 8.43 (br d, J=4.6 Hz, 1H), 8.08 (d, J=18.1 Hz, 2H), 7.75 (dd, J=8.6, 11.0 Hz, 2H), 7.58 (d, J=7.7 Hz, 1H), 7.41-7.11 (m, 8H), 4.47 (br s, 1H), 4.02 (d, J=3.7 Hz, 1H), 3.89 (s, 3H), 2.82-2.65 (m, 5H). MS (ESI) m/z (M+H)⁺ 436.1.

To a solution of compound 17D (60 mg, 137.78 umol) in DMSO (3 mL) and DCM (50 mL) was added DMP (234 mg, 551.12 umol), the mixture was stirred at 25° C. for 1 h. The reaction mixture was diluted with DCM (20 mL) and quenched by addition Na₂S₂O₃ (sat, 30 mL) and NaHCO₃ (saturated 30 mL), the mixture was extracted with DCM (30 mL×2). The combined organic layers were washed with H₂O (50 mL), then washed with brine (50 mL×2), dried over Na₂SO₄, filtered and concentrated under reduced pressure to give a residue. The residue was triturated in CH₃CN, filtered and the solid was dried in vacuum. Compound 17 (15 mg, yield: 22.8%) as white solid was obtained. ¹H NMR (400 MHz, DMSO-d₆) δ 8.48-8.35 (m, 2H), 8.15-8.07 (m, 2H), 8.04 (s, 1H), 7.80 (s, 1H), 7.74 (td, J=1.5, 7.8 Hz, 1H), 7.64 (td, J=1.4, 8.0 Hz, 1H), 7.36 (t, J=7.8 Hz, 1H), 7.32-7.17 (m, 5H), 5.30-5.24 (m, 1H), 3.91 (s, 3H), 3.15 (dd, J=4.0, 13.9 Hz, 1H), 2.89-2.74 (m, 4H). MS (ESI) m/z (M+H)⁺ 434.2.

### N-(4-AMINO-3,4-DIOXO-1-PHENYLBUTAN-2-YL)-3-(BENZO[D]OXAZOL-7-YL)-1-METHYL-1H-PYRAZOLE-4-CARBOXAMIDE (31)

Compounds 7-(4,4,5,5-tetramethyl-1,3,2-dioxaborolan-2-yl)benzo[d]oxazole (prepared from 7-bromobenzo[d]oxazole using same procedure as 33C) and intermediate 17B were converted to compound 31 using procedures as described in Example 1. Compound 31 (60 mg, yield: 60.2%) as a white solid was obtained. ¹H NMR (400 MHz, DMSO-d₆) δ 8.61 (s, 1H), 8.44 (d, J=7.6 Hz, 1H), 8.21 (s, 1H), 8.03 (s, 1H), 7.80-7.74 (m, 2H), 7.47-7.43 (m, 1H), 7.39-7.20 (m, 6H), 5.26-5.19 (m, 1H), 3.96 (s, 3H), 3.17-3.10 (m, 1H), 2.86-2.79 (m, 1H). MS (ESI) m/z (M+H)⁺ 418.1.

### N-(4-AMINO-3,4-DIOXO-1-PHENYLBUTAN-2-YL)-3-(BENZO[D]THIAZOL-4-YL)-1-METHYL-1H-PYRAZOLE-4-CARBOXAMIDE (51)

Compounds benzo[d]thiazol-4-ylboronic acid (prepared from 4-bromobenzo[d]thiazole using same procedure as 33C) and intermediate 17B were converted to compound 51 using procedures as described in Example 1. Compound 51 (75 mg, yield: 69.6%) as a pale yellow solid was obtained. ¹H NMR (DMSO-d₆, 400 MHz): δ 9.19 (s, 1H), 8.22-8.12 (m, 2H), 7.99-7.90 (m, 2H), 7.73 (s, 1H), 7.51-7.42 (m, 2H), 7.27-7.15 (m, 3H), 7.13-7.06 (m, 2H), 5.22-5.06 (m, 1H), 3.92 (s, 3H), 3.11-2.94 (m, 1H), 2.80-2.63 (m, 1H). MS (ESI) m/z (M+H)⁺ 434.1.

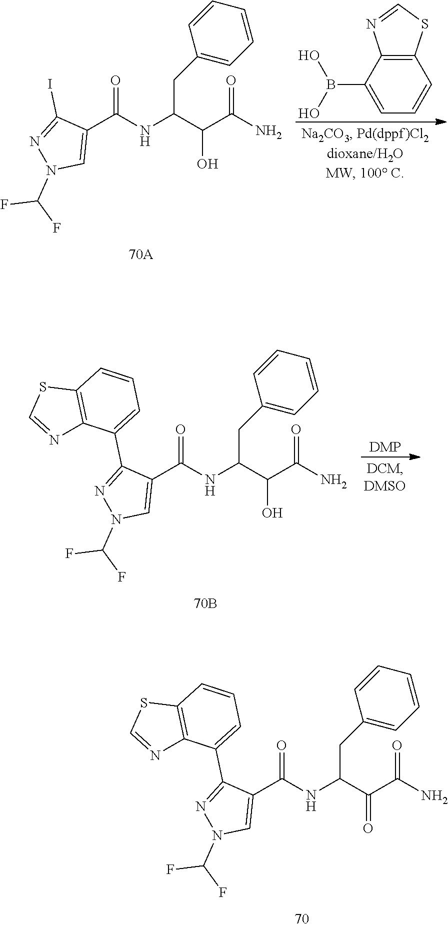 [SMILES: NC(=O)C(O)C(Cc1ccccc1)NC(=O)c1cn(C(F)F)nc1I ; OB(O)c1cccc2scnc12 ; NC(=O)C(O)C(Cc1ccccc1)NC(=O)c1cn(C(F)F)nc1-c1cccc2scnc12 ; NC(=O)C(=O)C(Cc1ccccc1)NC(=O)c1cn(C(F)F)nc1-c1cccc2scnc12]

### N-(4-AMINO-3,4-DIOXO-1-PHENYLBUTAN-2-YL)-3-(BENZO[D]THIAZOL-4-YL)-1-(DIFLUOROMETHYL)-1H-PYRAZOLE-4-CARBOXAMIDE (70)

Compounds benzo[d]thiazol-4-ylboronic acid (prepared from 4-bromobenzo[d]thiazole using same procedure as 33C) and intermediate 70A (prepared from 4A using same procedure as 17B) were converted to compound 70 using procedures as described in Example 1. Compound 70 (50 mg, yield: 48.5%) as a pale yellow solid was obtained. ¹H NMR (DMSO-d₆, 400 MHz): δ 9.14 (s, 1H), 8.61 (s, 1H), 8.52 (d, J=7.3 Hz, 1H), 8.20-8.16 (m, 1H), 8.11-7.87 (m, 2H), 7.79-7.69 (m, 1H), 7.50-7.44 (m, 2H), 7.27-7.13 (m, 5H), 5.16-5.07 (m, 1H), 3.04 (dd, J=3.7, 13.9 Hz, 1H), 2.72 (dd, J=9.7, 13.9 Hz, 1H). MS (ESI) m/z (M+H)⁺ 470.1.

### N-(4-AMINO-3,4-DIOXO-1-PHENYLBUTAN-2-YL)-1-(DIFLUOROMETHYL)-3-(2,5-DIMETHYLFURAN-3-YL)-1H-PYRAZOLE-4-CARBOXAMIDE (24)

Compounds 2-(2,5-dimethylfuran-3-yl)-4,4,5,5-tetramethyl-1,3,2-dioxaborolane and intermediate 70A (prepared from 4A using same procedure as 17B) were converted to compound 24 using procedures as described in Example 1. Compound 24 (140 mg, yield: 79.8%) as a light yellow solid was obtained. ¹H NMR (400 MHz, DMSO-d₆) δ 8.67-8.56 (m, 1H), 8.49 (s, 1H), 8.11 (s, 1H), 8.04-7.67 (m, 2H), 7.35-7.16 (m, 5H), 6.09 (s, 1H), 5.35-5.29 (m, 1H), 3.18 (dd, J=4.0, 14.1 Hz, 1H), 2.81 (dd, J=9.9, 13.9 Hz, 1H), 2.20 (d, J=12.1 Hz, 6H). MS (ESI) m/z (M+H)⁺ 431.1.

### N-(4-AMINO-3,4-DIOXO-1-PHENYLBUTAN-2-YL)-1-(DIFLUOROMETHYL)-3-(2-METHYLFURAN-3-YL)-1H-PYRAZOLE-4-CARBOXAMIDE (26)

Compounds 4,4,5,5-tetramethyl-2-(2-methylfuran-3-yl)-1,3,2-dioxaborolane and intermediate 70A (prepared from 4A using same procedure as 17B) were converted to compound 26 using procedures as described in Example 1. Compound 26 (128 mg, yield: 95.87%) as a pale yellow solid was obtained. ¹H NMR (400 MHz, DMSO-d) S 8.63 (d, J=7.3 Hz, 1H), 8.50 (s, 1H), 8.11-7.67 (m, 3H), 7.44 (d, J=1.8 Hz, 1H), 7.30-7.22 (m, 4H), 7.22-7.15 (m, 1H), 6.49 (d, J=1.8 Hz, 1H), 5.37-5.23 (m, 1H), 3.16 (dd, J=3.6, 14.0 Hz, 1H), 2.79 (br dd, J=10.1, 13.9 Hz, 1H), 2.25 (s, 3H). MS (ESI) m/z (M+H)⁺ 417.1.

### N-(4-AMINO-3,4-DIOXO-1-PHENYLBUTAN-2-YL-3-(2,5-DIMETHYLFURAN-3-YL)-1-METHYL-1H-PYRAZOLE-4-CARBOXAMIDE (55)

Compounds 2-(2,5-dimethylfuran-3-yl)-4,4,5,5-tetramethyl-1,3,2-dioxaborolane and intermediate 17B were converted to compound 55 using procedures as described in Example 1. Compound 55 (22 mg, yield: 26.5%) as a white solid was obtained. ¹H NMR (400 MHz, DMSO-d₆) δ 8.09-8.03 (m, 2H), 8.01 (d, J=7.3 Hz, 1H), 7.81 (s, 1H), 7.32-7.25 (m, 2H), 7.25-7.17 (m, 3H), 6.13-6.02 (s, 1H), 5.28 (m, 1H), 3.84 (s, 3H), 3.15 (dd, J=4.0, 13.9 Hz, 1H), 2.82 (dd, J=9.7, 13.9 Hz, 1H), 2.23-2.12 (m, 6H). MS (ESI) m/z (M+H)⁺ 395.2.

### Example 8

### Compounds 68 and 71

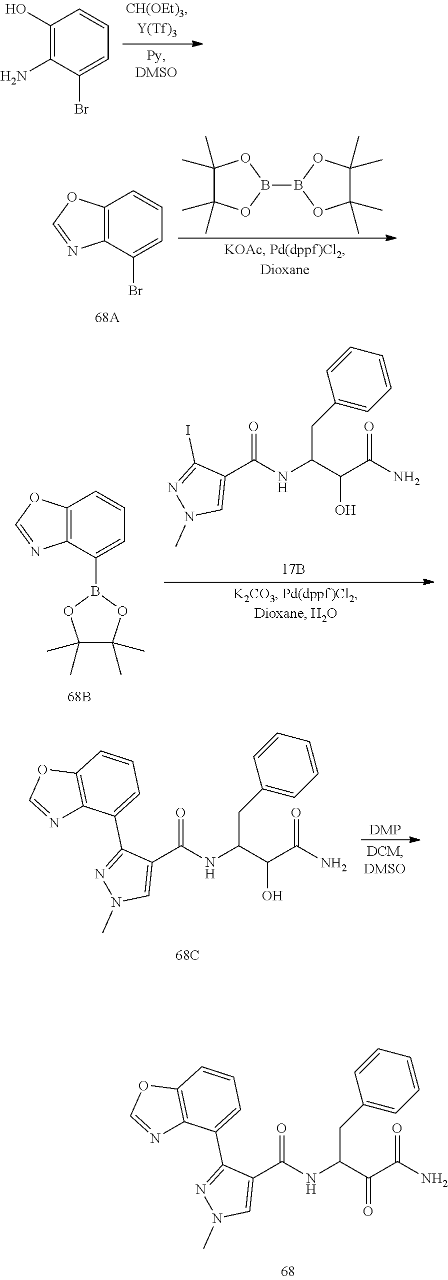

Yttrium tris (trifluoromethanesulfonate) (249 mg, 0.5 mmol) and Triethylorthoformate (15 mL, 93.1 mmol) were combined. To this mixture was added a solution of 2-amino-3-bromophenol (1.8 g, 9.31 mmol) in DMSO (20 mL) and Pyridine (1.5 mL, 18.6 mmol). The reaction mixture was stirred in a heat block at 60° C. for 18 h. The mixture was added H₂O (200 mL) and extracted with EA (50 mL). The organic phase was washed with brine (20 mL) and dried over Na₂SO₄, filtered and concentrated under vacuum. The product was purified by FCC (0-50% EA/PE) to afford compound 68A (1 g, yield 51.7%) as a red solid. ¹H NMR (400 MHz, DMSO-d₆) δ 8.96 (s, 1H), 7.90 (d, J=8.2 Hz, 1H), 7.73 (d, J=7.6 Hz, 1H), 7.53-7.44 (m, 1H). MS (ESI) m/z (M+H)⁺ 198.0.

### N-(4-AMINO-3,4-DIOXO-1-PHENYLBUTAN-2-YL)-3-(BENZO[D]OXAZOL-4-YL)-1-METHYL-1H-PYRAZOLE-4-CARBOXAMIDE (68)

Compounds 4-(4,4,5,5-tetramethyl-1,3,2-dioxaborolan-2-yl)benzo[d]oxazole (68B) (prepared from 68A using same procedure as 33C) and intermediate 17B were converted to compound 68 using procedures as described in Example 1. Compound 68 (10 mg, yield: 6.7%) as a white solid was obtained. ¹H NMR (400 MHz, DMSO-d₆) δ 8.52 (s, 1H), 8.15 (s, 1H), 7.73 (dd, J=1.6, 7.7 Hz, 1H), 7.69-7.46 (m, 3H), 7.45-7.37 (m, 2H), 7.25-7.15 (m, 3H), 7.08 (d, J=6.3 Hz, 2H), 5.26-5.21 (m, 1H), 3.94 (s, 3H), 3.22-3.10 (m, 1H), 2.83 (dd, J=8.5, 14.1 Hz, 1H). MS (ESI) m/z (M+H)⁺ 418.1.

### N-(4-AMINO-3,4-DIOXO-1-PHENYLBUTAN-2-YL)-3-(BENZO[D]OXAZOL-4-YL)-1-(DIFLUOROMETHYL)-1H-PYRAZOLE-4-CARBOXAMIDE (71)

Compounds 68B and intermediate 70A (prepared from 4A using same procedure as 17B) were converted to compound 71 using procedures as described in Example 1. Compound 71 (124 mg, yield: 77.99%) as a pale yellow solid was obtained. ¹H NMR (400 MHz, DMSO-d₆) δ 8.65 (s, 1H), 8.55 (s, 1H), 8.19 (s, 1H), 8.10-7.88 (m, 2H), 7.79 (dd, J=2.9, 6.4 Hz, 1H), 7.75-7.64 (m, 1H), 7.53-7.44 (m, 2H), 7.30-7.14 (m, 5H), 5.30-5.21 (m, 1H), 3.17-3.12 (m, 1H), 2.87 (dd, J=8.9, 14.2 Hz, 1H). MS (ESI) m/z (M+H)⁺ 454.1.

### Example 9

### COMPOUNDS 35 AND 50

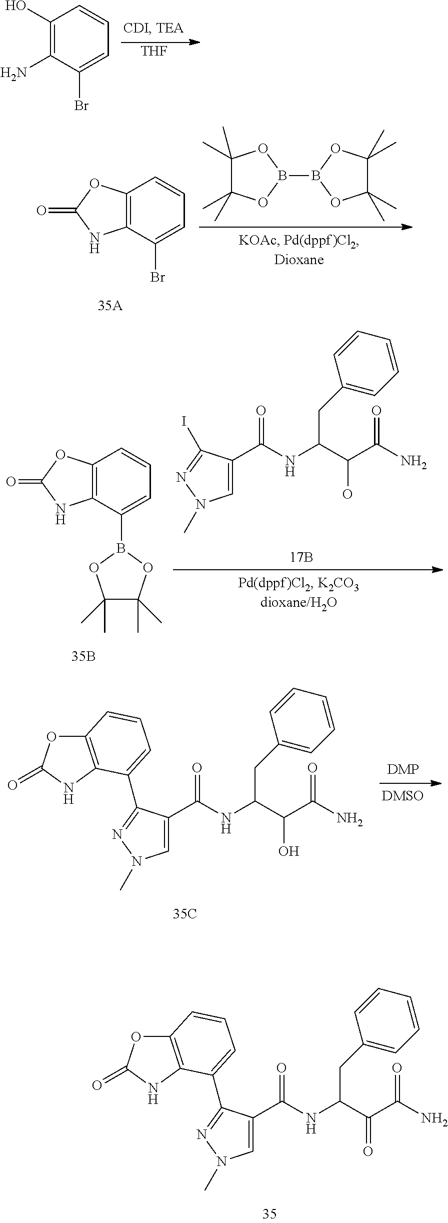

TEA (1.5 mL, 10.64 mmol) was added to the mixture of 2-amino-3-bromophenol (1 g, 5.32 mmol) and CDI (1.72 g, 10.64 mmol) in THF (20 mL). The mixture was stirred at 60° C. for 18 h. The reaction mixture was evaporated and diluted with dichloromethane (60 mL). The organic layer was washed with 1M hydrochloric acid (2×30 mL) and water (30 mL). The organic layer was dried over sodium sulfate, filtered and concentrated under vacuo. Compound 35A (1.1 g, 96.64% yield) was obtained as a red solid, which was used for next step directly. ¹H NMR (400 MHz, DMSO-d₆) δ 12.19 (br s, 1H), 7.37-7.29 (m, 2H), 7.08-7.01 (m, 1H).

### N-(4-AMINO-3,4-DIOXO-1-PHENYLBUTAN-2-YL)-1-METHYL-3-(2-OXO-2,3-DIHYDROBENZO[D]OXAZOL-4-YL)-1H-PYRAZOLE-4-CARBOXAMIDE (35)

Compounds 4-(4,4,5,5-tetramethyl-1,3,2-dioxaborolan-2-yl)benzo[d]oxazol-2(3H)-one (35B) (prepared from 35A using same procedure as 33C) and intermediate 17B were converted to compound 35 using procedures as described in Example 1. Compound 35 (18 mg, yield: 29.62%) as a yellow solid was obtained. ¹H NMR (400 MHz, DMSO-d₆) δ 9.47 (br s, 1H), 7.88 (s, 1H), 7.55 (d, J=8.3 Hz, 1H), 7.36-7.21 (m, 5H), 7.18 (d, J=8.0 Hz, 1H), 7.06 (br t, J=8.2 Hz, 2H), 6.96 (br d, J=6.8 Hz, H), 6.25 (br s, 1H), 5.49-5.40 (m, 1H), 4.01-3.93 (m, 3H), 3.30 (dd, J=4.8, 14.1 Hz, 1H), 2.93 (dd, J=9.0, 14.1 Hz, 1H). MS (ESI) m/z (M+H)⁺ 434.2.

### N-(4-AMINO-3,4-DIOXO-1-PHENYLBUTAN-2-YL)-1-(DIFLUOROMETHYL)-3-(2-OXO-2,3-DIHYDROBENZO[D]OXAZOL-4-YL)-1H-PYRAZOLE-4-CARBOXAMIDE (50)

Compounds 35B and intermediate 70A (prepared from 4A using same procedure as 17B) were converted to compound 50 using procedures as described in Example 1. Compound 50 (20 mg, yield: 22.8%) as a white solid was obtained. ¹H NMR (400 MHz, DMSO-d₆) δ 11.27 (s, 1H), 8.46 (s, 1H), 8.12-7.90 (m, 1H), 7.83-7.58 (m, 2H), 7.23-6.59 (m, 9H), 5.24 (s, 1H), 2.99-2.97 (m, 1H), 2.70-2.60 (m, 1H). MS (ESI) m/z (M+H)⁺ 470.1.

### Example 10

### Compound 16

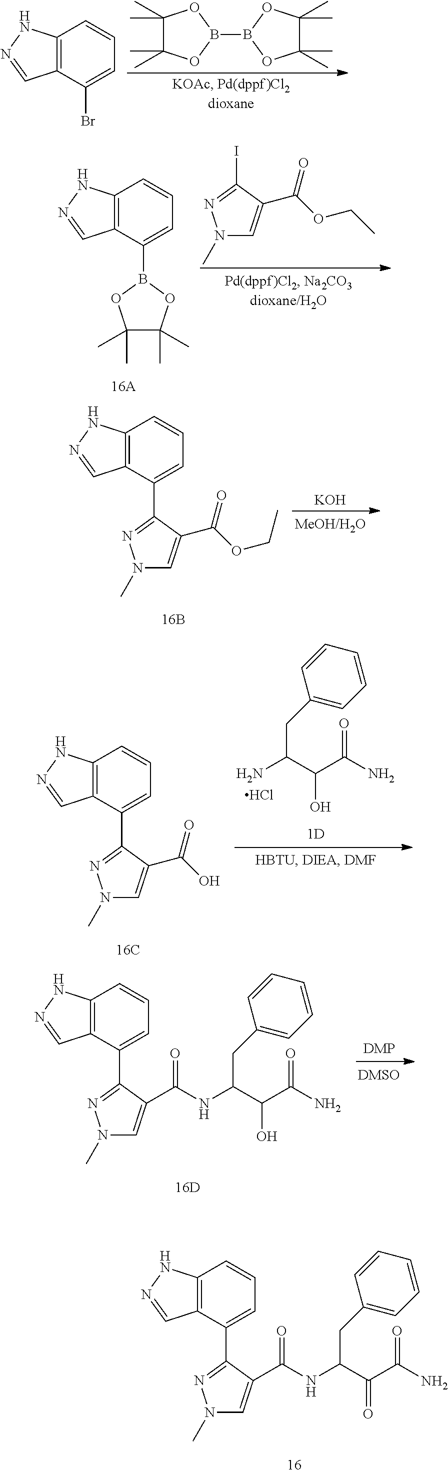

### N-(4-AMINO-3,4-DIOXO-1-PHENYLBUTAN-2-YL)-3-(1H-INDAZOL-4-YL)-1-METHYL-1H-PYRAZOLE-4-CARBOXAMIDE (16)

Compounds 4-(4,4,5,5-tetramethyl-1,3,2-dioxaborolan-2-yl)-1H-indazole (16A) (prepared from 4-bromo-1H-indazole using same procedure as 33C) and ethyl 3-iodo-1-methyl-1H-pyrazole-4-carboxylate were converted to compound 16 using procedures as described in Example 1. Compound 16 (60 mg, yield: 77.4%) as a white solid was obtained. ¹H NMR (DMSO-d₆,400 MHz): δ 13.05 (br s, 1H), 8.34 (d, J=7.3 Hz, 1H), 8.13-8.08 (m, 2H), 8.06 (s, 1H), 7.81 (s, 1H), 7.52-7.45 (m, 1H), 7.32-7.19 (m, 7H), 5.34-5.24 (m, 1H), 3.95 (s, 3H), 3.14 (dd, J=3.8, 14.1 Hz, 1H), 2.80 (dd, J=9.9, 13.9 Hz, 1H). MS (ESI) m/z (M+H)⁺ 417.1.

### Example 11

### Compound 39

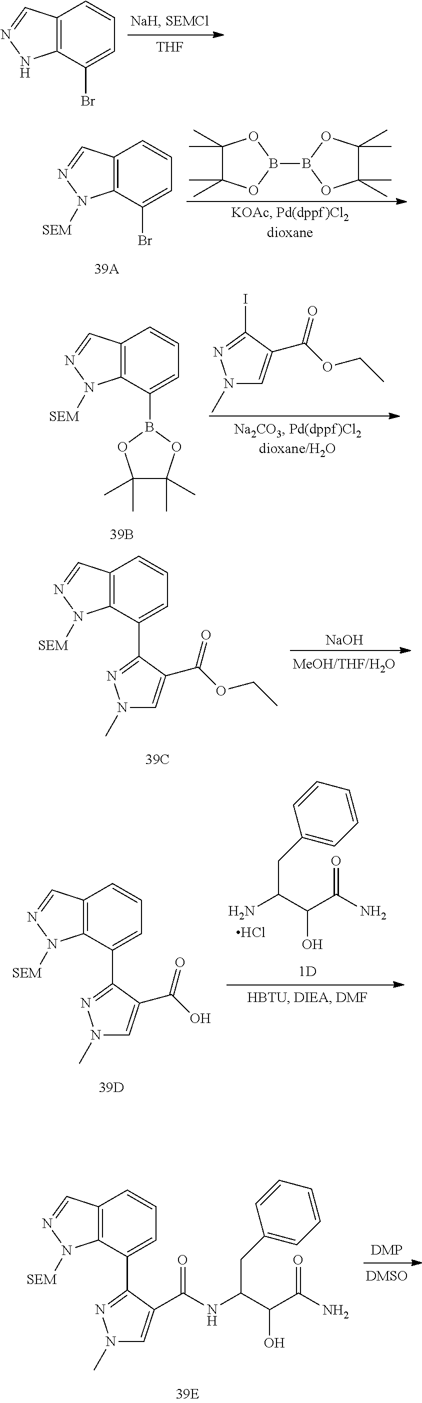

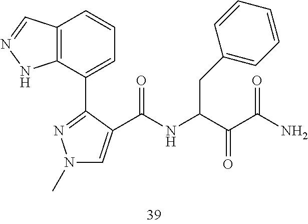 [SMILES: Cn1cc(C(=O)NC(Cc2ccccc2)C(=O)C(N)=O)c(-c2cccc3cn[nH]c23)n1]

### N-(4-AMINO-3,4-DIOXO-1-PHENYLBUTAN-2-YL)-3-(1H-INDAZOL-7-YL)-1-METHYL-1H-PYRAZOLE-4-CARBOXAMIDE (39)

NaH (406 mg, 10.2 mmol, 60% purity) was added to a mixture of 7-bromo-1H-indazole (1 g, 5.1 mmol) in THF (15 mL) at 0° C. The mixture was stirred at 0° C. for 1 h, then SEM-Cl (1.35 mL, 7.62 mmol) was added. After addition, the reaction temperature was allow to rise to room temperature (22° C.) slowly and the mixture was stirred for 15 h at 22° C. The mixture was quenched with the addition of saturated NH₄Cl (30 mL). Then the mixture was extracted with EA (3×25 mL). The combined organic layer was washed with brine (20 mL), dried over anhydrous MgSO₄, filtered and concentrated. The residue was purified by silica gel column chromatography (petroleum ether/ethyl acetate=1/0 to 8/1) to afford compound 39A (1.1 g, yield 66.2%) as yellow oil. ¹H NMR (400 MHz, DMSO-d₆) δ 8.26 (s, 1H), 7.85 (dd, J=0.9, 7.9 Hz, 1H), 7.70 (dd, J=0.9, 7.5 Hz, 1H), 7.13 (t, J=7.7 Hz, 1H), 5.99 (s, 2H), 3.52 (t, J=7.8 Hz, 2H), 0.78 (t, J=7.8 Hz, 2H), −0.13 (s, 9H).

Compounds 7-(4,4,5,5-tetramethyl-1,3,2-dioxaborolan-2-yl)-1-((2-(trimethylsilyl)ethoxy)methyl)-1H-indazole (39B) (prepared from 39A using same procedure as 33C) and ethyl 3-iodo-1-methyl-1H-pyrazole-4-carboxylate were converted to compound 39F using procedures as described in Example 1. Compound 39F (203 mg, yield: 70.49%) as a yellow solid was obtained. ¹H NMR (400 MHz, DMSO-ds) 8 8.31 (s, 1H), 8.19-8.16 (m, 1H), 7.86-7.80 (m, 1H), 7.71-7.50 (m, 2H), 7.25-7.13 (m, 6H), 7.01 (d, J=7.3 Hz, 2H), 5.31 (s, 2H), 5.28-5.19 (m, 1H), 3.94 (s, 3H), 2.74 (dd, J=8.5, 14.1 Hz, 1H), 0.90-0.83 (m, 3H), 0.57 (t, J=8.0 Hz, 2H), −0.14 (s, 9H).

HCl/EtOAc (4M, 4 mL) was added to the mixture of Compound 39F (160 mg, 0.3 mmol). The mixture was stirred at 30° C. for 3 h. The mixture was filtered and the filtered cake was concentrated under vacuo. Compound 39 (66 mg, 54.1% yield) was obtained as a white solid. ¹H NMR (400 MHz, DMSO-d₆) δ 12.74 (s, 1H), 8.44 (d, J=7.5 Hz, 1H), 8.11-8.04 (m, 3H), 7.81-7.73 (m, 2H), 7.68 (d, J=7.5 Hz, 1H), 7.28-7.22 (m, 4H), 7.21-7.16 (m, 1H), 7.02 (t, J=7.6 Hz, 1H), 5.33-5.26 (m, 1H), 3.97 (s, 3H), 3.14 (dd, J=3.9, 14.0 Hz, 1H), 2.85-2.75 (m, 1H). MS (ESI) m/z (M+H)⁺⁼417.1.

### Example 12

### Compounds 9, 47, and 48

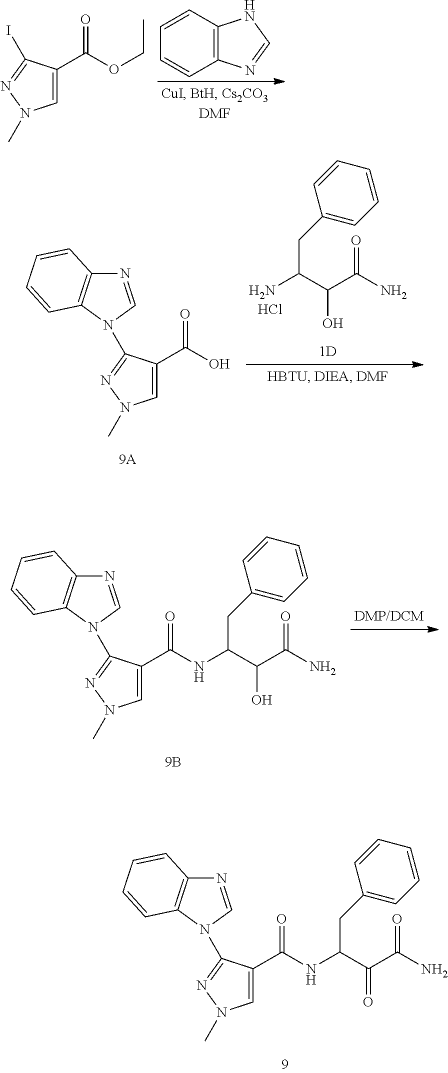

To a solution of ethyl 3-iodo-1-methyl-1H-pyrazole-4-carboxylate (4 g, 14.28 mmol) and 1H-benzo[d]imidazole (2 g, 16.93 mmol) in DMF (40 mL) was added Cs₂CO₃ (9.31 g, 28.57 mmol), 1H-benzotriazole (340 mg, 2.86 mmol) and CuI (272 mg, 1.43 mmol). The mixture was stirred at 110° C. for 48 h under N₂. The mixture was diluted with H₂O (100 mL), washed with EtOAc (150 mL). The aqueous phase was collected, adjusted to pH ˜4 with 1N HCl, washed with EtOAc (300 mL). The aqueous phase was collected and concentrated in vacuo. The residue was triturated with MeOH (40 mL). The solid was filtered off. The filtrate was collected and concentrated. The residue was purified by preparatory-HPLC (HCl) to give compound 9A (380 mg, yield: 10.74%) as white solid. MS (ESI) m/z (M+H)⁺ 242.9.

### N-(4-AMINO-3,4-DIOXO-1-PHENYLBUTAN-2-YL)-3-(1H-BENZO[D]IMIDAZOL-1-YL)-1-METHYL-1H-PYRAZOLE-4-CARBOXAMIDE (9)

Compounds 49A and intermediate 1D were converted to compound 9 using procedures as described in Example 1. Compound 9 (70 mg, yield: 46.85%) as a white solid was obtained. MS (ESI) m/z (M+H)⁺ 417.1. ¹HNMR (400 MHz, DMSO-d₆) δ 8.61 (d, J=7.6 Hz, 1H), 8.39 (s, 1H), 8.32 (s, 1H), 8.02 (br. s, 1H), 7.77 (br. s, 1H), 7.71-7.65 (m, 1H), 7.50-7.43 (m, 1H), 7.30-7.16 (m, 7H), 5.29-5.20 (m, 1H), 4.00-3.91 (m, 3H), 3.18-3.09 (m, 1H), 2.85-2.75 (m, 1H).

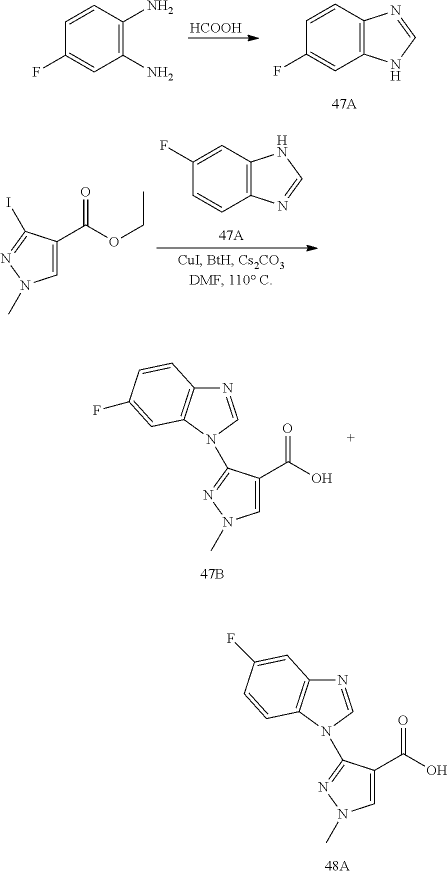

A mixture of 4-fluorobenzene-1,2-diamine (1 g, 7.93 mmol) and HCOOH (10 mL) was stirred at 90° C. for 2 h. The solution was adjusted to pH ˜7 with 5N NaOH. The mixture was extracted with EtOAc (50 mL×3). The organics were collected, dried with Na₂SO₄, filtered and concentrated to give compound 47A (1 g, crude) as brown solid, which was used directly for the next step without further purification.

Ethyl 3-iodo-1-methyl-1H-pyrazole-4-carboxylate and intermediate 47A were subjected to reaction conditions as for intermediate 9A and the reaction yielded products 47B and 48A. The product was purified by preparatory-HPLC (HCl) to give 400 mg of mixture as brown solid, which was repurified by SFC (column: AD (250 mm*30 mm, 5 um); mobile phase: [0.1% NH₃H₂O MEOH]; B %: 25%-25%, min) to give compound 47B (100 mg, yield: 2.61%) as white solid, compound 48A (100 mg, yield: 2.61%) as white solid, which was repurified by SFC to give 48A (90 mg). MS (ESI) m/z (M+H)⁺ 260.9.

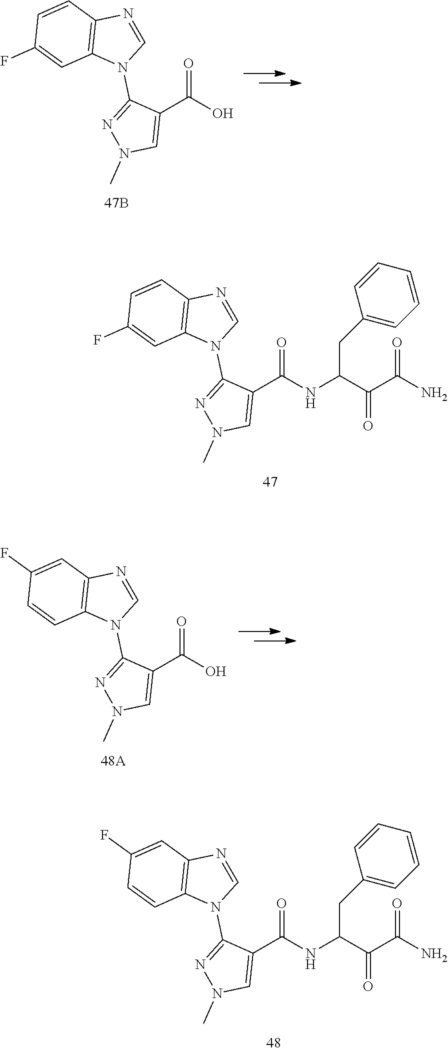 [SMILES: Cn1cc(C(=O)O)c(-n2cnc3ccc(F)cc32)n1 ; Cn1cc(C(=O)NC(Cc2ccccc2)C(=O)C(N)=O)c(-n2cnc3ccc(F)cc32)n1 ; Cn1cc(C(=O)O)c(-n2cnc3cc(F)ccc32)n1 ; Cn1cc(C(=O)NC(Cc2ccccc2)C(=O)C(N)=O)c(-n2cnc3cc(F)ccc32)n1]

### N-(4-AMINO-3,4-DIOXO-1-PHENYLBUTAN-2-YL)-3-(6-FLUORO-1H-BENZO[D]IMIDAZOL-1-YL)-1-METHYL-1H-PYRAZOLE-4-CARBOXAMIDE (47)

Compounds 47B and intermediate 1D were converted to compound 47 using procedures as described in Example 1. Compound 47 (50 mg, yield: 48.0%) as a white solid was obtained. ¹H NMR (400 MHz, DMSO-d₆) δ 8.42 (s, 1H), 8.36 (s, 1H), 8.33-8.27 (m, 1H), 7.72 (br s, 1H), 7.58-7.44 (m, 3H), 7.32-7.17 (m, 5H), 7.16-7.07 (m, 1H), 5.34-5.26 (m, 1H), 3.97 (s, 3H), 3.24-3.17 (m, 1H), 2.95-2.85 (m, 1H). MS (ESI) m/z (M+H)⁺ 435.2.

### N-(4-AMINO-3,4-DIOXO-1-PHENYLBUTAN-2-YL)-3-(5-FLUORO-1H-BENZO[D]IMIDAZOL-1-YL)-1-METHYL-1H-PYRAZOLE-4-CARBOXAMIDE (48)

Compounds 48A and intermediate 1D were converted to compound 48 using procedures as described in Example 1. Compound 48 (40 mg, yield: 28.2%) as a white solid was obtained. ¹H NMR (400 MHz, DMSO-d₆) δ 8.46-8.21 (m, 3H), 7.80-7.41 (m, 3H), 7.38-7.04 (m, 7H), 5.31 (br. s, 1H), 4.04-3.90 (m, 3H), 3.27-3.16 (m, 1H), 2.95-2.83 (m, 1H). MS (ESI) m/z (M+H)⁺ 435.2.

### Example 13

### Compounds 20 and 21

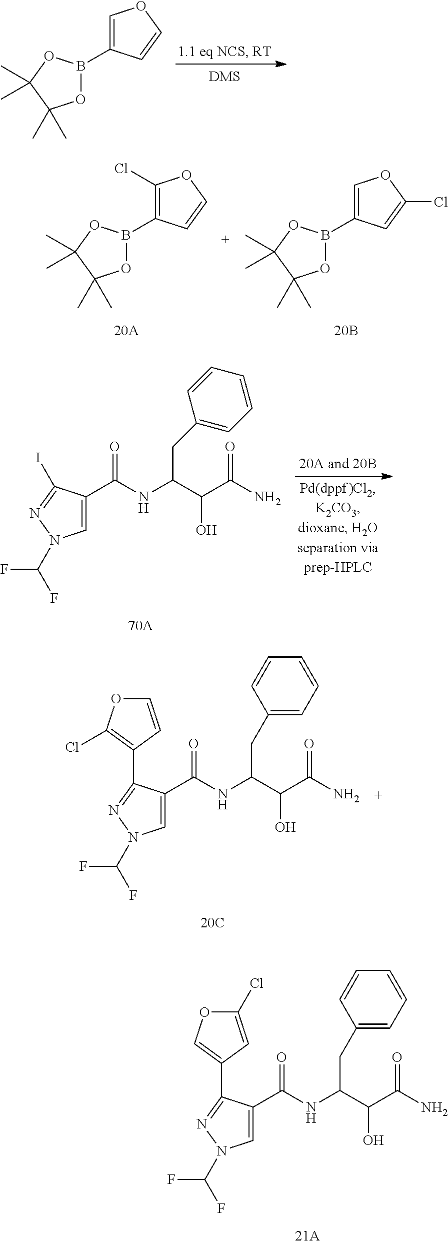 [SMILES: CC1(C)OB(c2ccoc2)OC1(C)C ; CC1(C)OB(c2ccoc2Cl)OC1(C)C ; CC1(C)OB(c2coc(Cl)c2)OC1(C)C ; NC(=O)C(O)C(Cc1ccccc1)NC(=O)c1cn(C(F)F)nc1I ; NC(=O)C(O)C(Cc1ccccc1)NC(=O)c1cn(C(F)F)nc1-c1ccoc1Cl ; NC(=O)C(O)C(Cc1ccccc1)NC(=O)c1cn(C(F)F)nc1-c1coc(Cl)c1]

To a solution of 2-(furan-3-yl)-4,4,5,5-tetramethyl-1,3,2-dioxaborolane (1 g, 5.15 mmol) in DMF (15 mL) was added NCS (723 mg, 5.41 mmol). The mixture was stirred at 25° C. for 4 h. resultant solution was treated with 10% Na₂S₂O₃ aqueous (50 mL) and was extracted with MTBE (50 mL×3). The combined organic phase was washed with brine (100 mL) and dried over Na₂SO₄. After removal of solvent under reduced pressure, the residue was purified by flash silica gel chromatography (ISCO®; 12 g SepaFlash® Silica Flash Column, Eluent of 0˜10% Ethyl acetate/Petroleum ethergradient @ 25 mUmin). Compound 20A (0.37 g, yield: 31.4%) was obtained as a colorless oil. Compound 20B (0.13 g, yield: 11.0%) was obtained as a colorless oil. The mixture of compound 20A and compound 20B. ¹H NMR (400 MHz, CDCl₃) δ 7.92 (s, 1H), 7.34 (d, J=2.0 Hz, 1H), 6.78 (d, J=2.0 Hz, 1H), 4.23 (q, J=7.1 Hz, 2H), 3.93 (s, 3H), 1.27 (t, J=7.2 Hz, 3H). MS (ESI) m/z (M+H)⁺ 254.9.

To a solution of compound 70A (400 mg, 861.69 umol) and compound 20A (216 mg, 945.38 umol) and compound 20B (80 mg, 350.14 umol) in dioxane (20 mL) and H₂O (2 mL) was added Pd(dppf)Cl₂ (70 mg, 95.67 umol) and K₂CO₃ (300 mg, 2.17 mmol) under N₂, and the mixture was stirred at 90° C. for 16 h under N₂ atmosphere. The reaction mixture was concentrated and the residue was diluted with EA (30 mL) and H₂O (40 mL), filtered, the filtrate was extracted with EA (20 mL×2), and then the organic phase was dried over Na₂SO₄, filtered and concentrated to give a residue. The residue was purified by preparatory-TLC (SiO₂, PE:EA=1:2.5). Then the residue was purified by preparatory-HPLC (HCl condition; column: YMC-Actus Triart C18 100*30 mm*5 um; mobile phase: [water (0.05% HCl)-ACN]; B %: 30%-60%, 10 min). Compound 20C (120 mg, yield: 31.6%) was obtained as a white solid. Compound 21A (45 mg, yield: 11.8%) was obtained as a white solid.

Compound 20C: ¹H NMR (400 MHz, DMSO-d₆) δ 8.61 (s, 0.3H), 8.54 (s, 0.7H), 8.21-7.71 (m, 2H), 7.69-7.62 (m, 1H), 7.31 (d, J=8.4 Hz, 1H), 7.25-7.09 (m, 6H), 6.65-6.57 (m, 1H), 5.86 (d, J=5.7 Hz, 0.7H), 5.75 (d, J=5.7 Hz, 0.3H), 4.50-4.36 (m, 1H), 4.03-3.96 (m, 0.7H), 3.87-3.83 (m, 0.3H), 2.91-2.69 (m, 2H). MS (ESI) m/z (M+H)⁺ 439.0.

Compound 21A: ¹H NMR (400 MHz, DMSO-d₆) δ 8.65 (s, 0.2H), 8.62 (s, 0.8H), 8.23-7.69 (m, 3H), 7.32 (d, J=7.7 Hz, 1H), 7.26-7.08 (m, 6H), 6.71-6.66 (m, 1H), 5.86 (d, J=5.7 Hz, 0.8H), 5.74 (d, J=6.0 Hz, 0.2H), 4.54-4.41 (m, 1H), 4.01 (dd, J=3.5, 5.7 Hz, 0.8H), 3.88 (d, J=5.3 Hz, 0.2H), 2.92-2.67 (m, 2H). MS (ESI) m/z (M+H)⁺ 439.0.

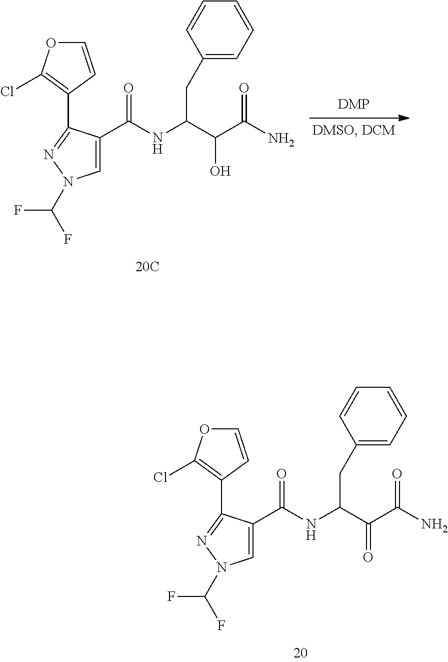 [SMILES: NC(=O)C(O)C(Cc1ccccc1)NC(=O)c1cn(C(F)F)nc1-c1ccoc1Cl ; NC(=O)C(=O)C(Cc1ccccc1)NC(=O)c1cn(C(F)F)nc1-c1ccoc1Cl]

### N-(4-AMINO-3,4-DIOXO-1-PHENYLBUTAN-2-YL)-3-(2-CHLOROFURAN-3-YL)-1-(DIFLUOROMETHYL)-1H-PYRAZOLE-4-CARBOXAMIDE (20)

Compounds 20C was converted to compound 20 using procedures as described in Example 1. Compound 20 (90 mg, yield: 70.6%) as a white solid was obtained. ¹H NMR (400 MHz, DMSO-d₆) δ 8.73 (d, J=7.5 Hz, 1H), 8.58 (s, 1H), 8.13-7.71 (m, 3H), 7.67 (d, J=2.2 Hz, 1H), 7.30-7.22 (m, 4H), 7.21-7.14 (m, 1H), 6.66 (d, J=2.2 Hz, 1H), 5.38-5.21 (m, 1H), 3.15 (dd, J=3.7, 13.9 Hz, 1H), 2.77 (dd, J=10.0, 13.8 Hz, 1H). MS (ESI) m/z (M+H)⁺ 437.0.

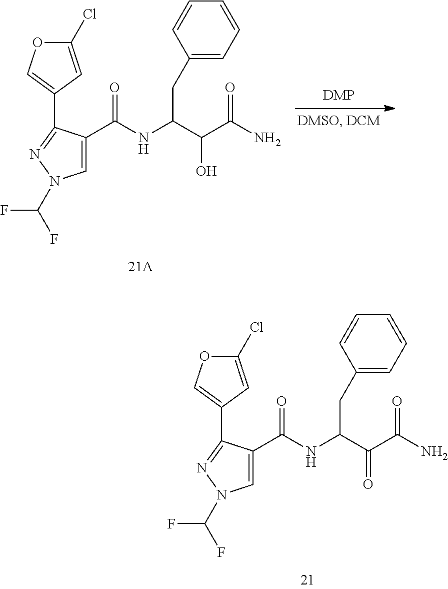 [SMILES: NC(=O)C(O)C(Cc1ccccc1)NC(=O)c1cn(C(F)F)nc1-c1coc(Cl)c1 ; NC(=O)C(=O)C(Cc1ccccc1)NC(=O)c1cn(C(F)F)nc1-c1coc(Cl)c1]

### N-(4-AMINO-3,4-DIOXO-1-PHENYLBUTAN-2-YL)-3-(5-CHLOROFURAN-3-YL)-1-(DIFLUOROMETHYL)-1H-PYRAZOLE-4-CARBOXAMIDE (21)

Compounds 21A was converted to compound 21 using procedures as described in Example 1. Compound 21 (30 mg, yield: 65.7%) as a white solid was obtained. ¹H NMR (400 MHz, DMSO-d₆) δ 8.81 (d, J=7.5 Hz, 1H), 8.62 (s, 1H), 8.16 (d, J=0.9 Hz, 1H), 8.10 (s, 1H), 8.03-7.71 (m, 2H), 7.26 (d, J=4.2 Hz, 4H), 7.20-7.16 (n, 1H), 6.74 (d, J=0.9 Hz, 1H), 5.36-5.23 (m, 1H), 3.17 (dd, J=3.9, 14.0 Hz, 1H), 2.80 (dd, J=10.3, 14.0 Hz, 1H). MS (ESI) m/z (M+H)⁺ 437.1.

### Example 14

### Compound 36

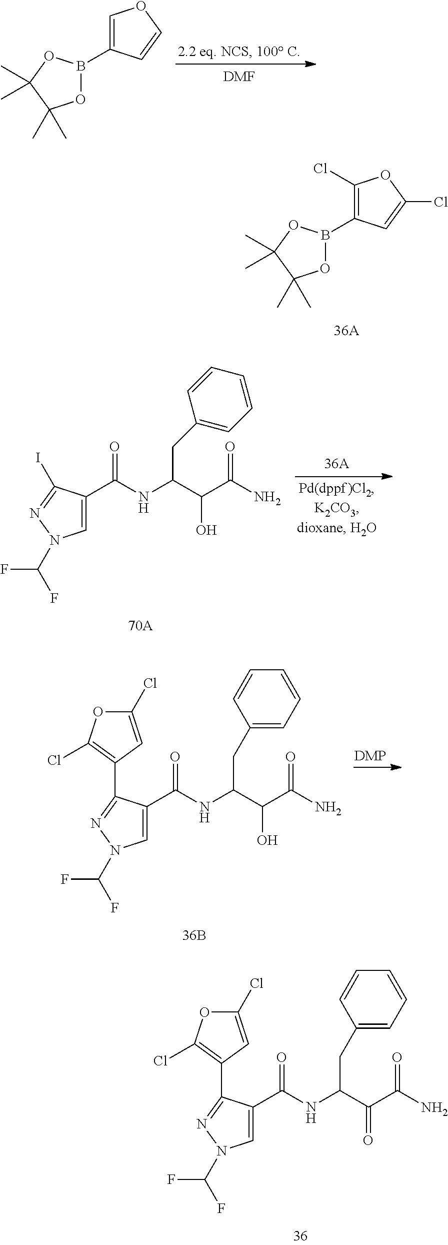

To a solution of 2-(furan-3-yl)-4,4,5,5-tetramethyl-1,3,2-dioxaborolane (1 g, 5.10 mmol.) in DMF (15 mL) was added NCS (1.50 g, 11.21 mmol). The mixture was stirred at 100° C. for 2 h. The resultant solution was treated with aq 10% Na₂S₂O₃ (50 mL) and was extracted with MTBE (50 mL×3). The combined organic phase was washed with brine (100 mL) and dried over Na₂SO₄. After removal of solvent under reduced pressure, the residue was purified by flash silica gel chromatography (ISCO®; 12 g SepaFlash® Silica Flash Column, Eluent of 010% Ethyl acetate/Petroleum ethergradient @20 mL/min). Compound 36A (0.5 g, yield: 37.0%) was obtained as a yellow oil. ¹H NMR (400 MHz, CDCl₃) δ 6.45-6.23 (m, 1H), 1.31 (s, 12H).

### N-(4-AMINO-3,4-DIOXO-1-PHENYLBUTAN-2-YL)-3-(2,5-DICHLOROFURAN-3-YL)-1-(DIFLUOROMETHYL)-1H-PYRAZOLE-4-CARBOXAMIDE (36)

Compounds 36A and intermediate 70A (prepared from 4A using same procedure as 17B) were converted to compound 36 using procedures as described in Example 1. Compound 36 (100 mg, yield: 71.7%) as a white solid was obtained. ¹H NMR (400 MHz, DMSO-d₆) δ 8.78 (d, J=7.5 Hz, 1H), 8.65 (s, 1H), 8.16-7.72 (m, 3H), 7.32-7.22 (m, 4H), 7.21-7.12 (m, 1H), 6.67 (s, 1H), 5.47-5.19 (m, 1H), 3.15 (dd, J=3.6, 13.8 Hz, 1H), 2.76 (dd, J=10.1, 13.9 Hz, 1H). MS (ESI) m/z (M+H)⁺ 471.0.

### Example 15

### Compounds 19 and 15

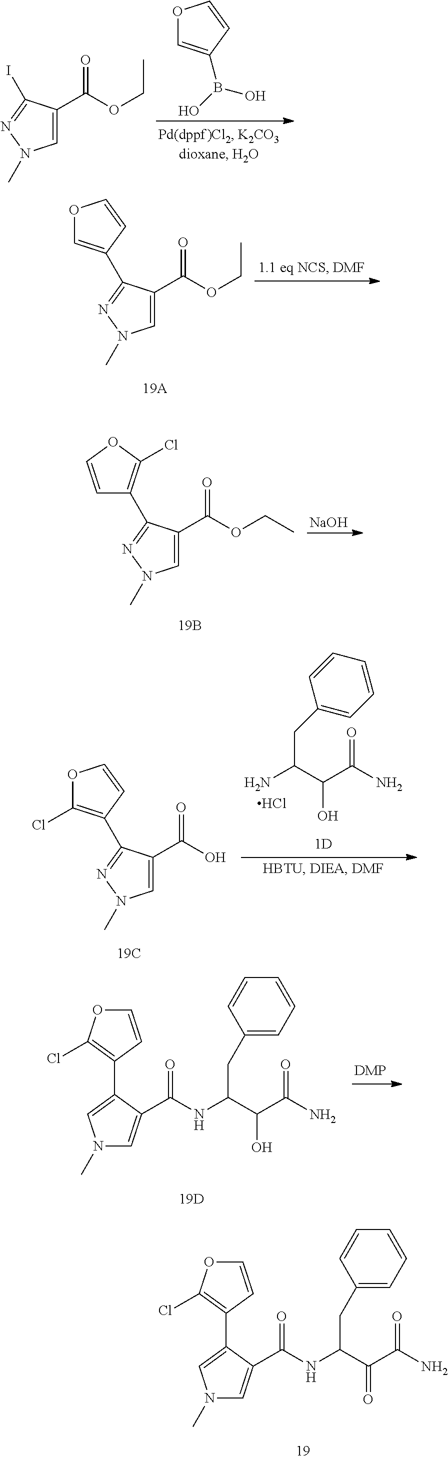

To a solution of 2-(furan-3-yl)-4,4,5,5-tetramethyl-1,3,2-dioxaborolane (500 mg, 1.79 mmol) and 3-furylboronic acid (250 mg, 2.23 mmol) in dioxane (20 mL) and H₂O (1 mL) was added K₂CO₃ (620 mg, 4.49 mmol) and Pd(dppf)Cl₂ (131 mg, 179.03 umol) under N₂. The mixture was stirred at 80° C. for 16 h under N₂. The reaction mixture was concentrated and the residue was diluted with EA (30 mL) and H₂O (30 mL), filtered. The filtrate was extracted with EA (20 mL), and then the organic phase was dried over Na₂SO₄, filtered and concentrated to give a residue. The residue was purified by flash silica gel chromatography (ISCO®; 24 g SepaFlash® Silica Flash Column, Eluent of 0-30% Ethyl acetate/Petroleum ethergradient @ 30 mUmin). Compound 19A (350 mg, yield: 88.8%) was obtained as a light yellow oil. ¹H NMR (400 MHz, CDCl₃) δ 8.39 (s, 1H), 7.91 (s, 1H), 7.44 (t, J=1.6 Hz, 1H), 6.95 (d, J=1.3 Hz, 1H), 4.30 (q, J=7.0 Hz, 2H), 3.92 (s, 3H), 1.35 (t, J=7.2 Hz, 3H). MS (ESI) m/z (M+H)⁺ 221.0.

To a solution of compound 19A (100 mg, 454.08 umol) in DMF (3 mL) was added NCS (68 mg, 509.24 umol). The mixture was stirred at 25° C. for 2 h. The reaction was diluted with H₂O (20 mL), extracted with EA (20 mL×2), the organic phase was dried over Na₂SO₄, filtered, and concentrated to give a residue. The residue was purified by preparatory-TLC (SiO₂, PE:EA=2: 1). Compound 19B (70 mg, yield: 60.5%) was obtained as a white solid. ¹H NMR (400 MHz, CDCl₃) δ 7.92 (s, 1H), 7.34 (d, J=2.0 Hz, 1H), 6.78 (d, J=2.0 Hz, 1H), 4.23 (q, J=7.1 Hz, 2H), 3.93 (s, 3H), 1.27 (t, J=7.2 Hz, 3H). MS (ESI) m/z (M+H)⁺ 254.9.

### N-(4-AMINO-3,4-DIOXO-1-PHENYLBUTAN-2-YL)-3-(2-CHLOROFURAN-3-YL)-1-METHYL-1H-PYRAZOLE-4-CARBOXAMIDE (19)

Compounds 19B was converted to compound 19 using procedures as described in Example 1. Compound 19 (40 mg, yield: 35.0%) as a white solid was obtained. ¹H NMR (400 MHz, DMSO-d₆) δ 8.32 (d, J=7.5 Hz, 1H), 8.14 (s, 1H), 8.06 (s, 1H), 7.80 (s, 1H), 7.64 (d, J=2.0 Hz, 1H), 7.32-7.24 (m, 4H), 7.23-7.19 (m, 1H), 6.66 (d, J=2.0 Hz, 1H), 5.33-5.25 (m, 1H), 3.89 (s, 3H), 3.15 (dd, J=3.9, 13.9 Hz, 1H), 2.82 (dd, J=9.9, 13.9 Hz, 1H). MS (ESI) m/z (M+H)⁺ 401.1.

### N-(4-AMINO-3,4-DIOXO-1-PHENYLBUTAN-2-YL)-3-(2,5-DICHLOROFURAN-3-YL)-1-METHYL-1H-PYRAZOLE-4-CARBOXAMIDE (15)

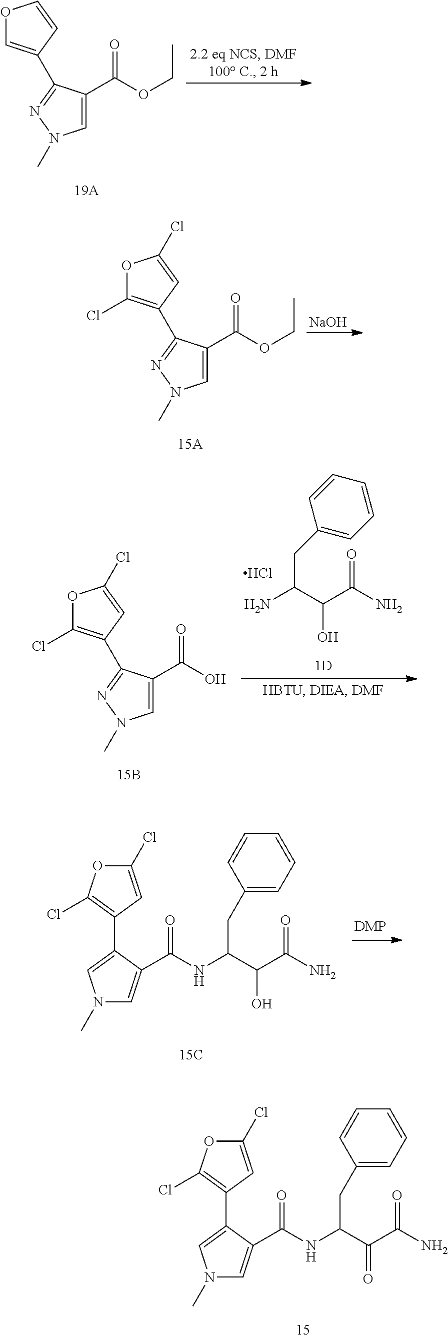

To a solution of compound 19A (50 mg, 227.04 umol) in DMF (2 mL) was added NCS (68 mg, 509.24 umol). The mixture was stirred at 100° C. for 1.5 h. The reaction was diluted with H₂O (20 mL), extracted with EA (20 mL×2), the organic phase was dried over Na₂SO₄, filtered, and concentrated to give a residue. The residue was purified by preparatory-TLC (SiO₂, PE:EA=2:1). Compound 15A (40 mg, yield 60.9%) was obtained as a white solid. ¹H NMR (400 MHz, CDCl₃) δ 7.93 (s, 1H), 6.63 (s, 1H), 4.34-4.18 (m, 2H), 3.95 (s, 3H), 1.31 (t, J=7.2 Hz, 3H). MS (ESI) m/z (M+H)⁺ 289.0.

Compounds 15A was converted to compound 15 using procedures as described in Example 1. Compound 15 (35 mg, yield: 47.2%) as a white solid was obtained. ¹H NMR (400 MHz, DMSO-d₆) δ 8.40 (d, J=7.5 Hz, 1H), 8.15 (s, 1H), 8.05 (s, 1H), 7.77 (s, 1H), 7.29-7.21 (m, 4H), 7.20-7.15 (m, 1H), 6.63 (s, 1H), 5.35-5.19 (m, 1H), 3.86 (s, 3H), 3.12 (dd, J=3.7, 13.9 Hz, 1H), 2.78 (dd, J=10.1, 13.9 Hz, 1H). MS (ESI) m/z (M+H)⁺ 435.0.

### Example 16

### COMPOUNDS 23, 3, 46, 52, AND 79

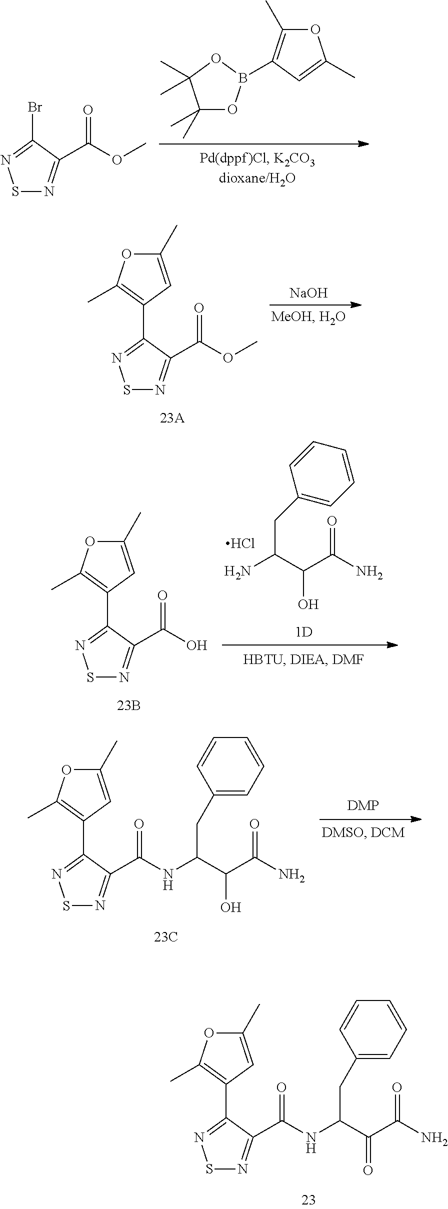

### N-(4-AMINO-3,4-DIOXO-1-PHENYLBUTAN-2-YL)-4-(2,5-DIMETHYLFURAN-3-YL)-1,2,5-THIADIAZOLE-3-CARBOXAMIDE (23)

Compounds methyl 4-bromo-1,2,5-thiadiazole-3-carboxylate and 2-(2,5-dimethylfuran-3-yl)-4,4,5,5-tetramethyl-1,3,2-dioxaborolane was converted to compound 23 using procedures as described in Example 1. Compound 23 (110 mg, yield: 65.02%) as a white solid was obtained. ¹H NMR (400 MHz, DMSO-d₆) δ 9.34 (d, J=7.9 Hz, 1H), 8.21 (s, 1H), 7.93 (s, 1H), 7.37-7.18 (m, 5H), 5.94 (s, 1H), 5.61-5.41 (m, 1H), 3.23 (dd, J=3.5, 14.1 Hz, 1H), 2.85 (dd, J=10.0, 14.0 Hz, 1H), 2.37 (s, 3H), 2.18 (s, 3H). MS (ESI) m/z (M+H)⁺ 399.1.

### N-(4-AMINO-3,4-DIOXO-1-PHENYLBUTAN-2-YL)-4-(4-FLUOROPHENYL)-1,2,5-THIADIAZOLE-3-CARBOXAMIDE (3)

Compounds methyl 4-bromo-1,2,5-thiadiazole-3-carboxylate and (4-fluorophenyl)boronic acid was converted to compound 3 using procedures as described in Example 1. Compound 3 (235 mg, yield: 68.1%) as a white solid was obtained. ¹H NMR (400 MHz, DMSO-d₆) δ 9.43 (d, J=7.7 Hz, 1H), 8.26-8.12 (m, 1H), 7.93 (s, 1H), 7.67-7.56 (m, 2H), 7.34-7.16 (m, 7H), 5.56-5.38 (m, 1H), 3.24 (dd, J=3.6, 14.0 Hz, 1H), 2.86 (dd, J=10.3, 14.0 Hz, H). MS (ESI) m/z (M+H)⁺ 399.1.

### N-(4-AMINO-3,4-DIOXO-1-PHENYLBUTAN-2-YL)-4-(2-METHYLFURAN-3-YL)-1,2,5-THIADIAZOLE-3-CARBOXAMIDE (46)

Compounds ethyl 4-chloro-1,2,5-thiadiazole-3-carboxylate and 4,4,5,5-tetramethyl-2-(2-methylfuran-3-yl)-1,3,2-dioxaborolane was converted to compound 46 using procedures as described in Example 1. Compound 46 (45 mg, yield: 42.84%) as a pale yellow solid was obtained. ¹H NMR (400 MHz, DMSO-d₆) δ 9.34 (d, J=7.7 Hz, 1H), 8.20 (s, 1H), 7.92 (s, 1H), 7.46 (d, J=2.0 Hz, 1H), 7.32-7.25 (m, 4H), 7.22 (qd, J=4.1, 8.7 Hz, 1H), 6.35 (d, J=2.0 Hz, 1H), 5.60-5.43 (m, 1H), 3.22 (dd, J=3.5, 13.9 Hz, 1H), 2.85 (dd, J=10.1, 14.1 Hz, 1H), 2.40 (s, 3H). MS (ESI) m/z (M+H)⁺ 385.1.

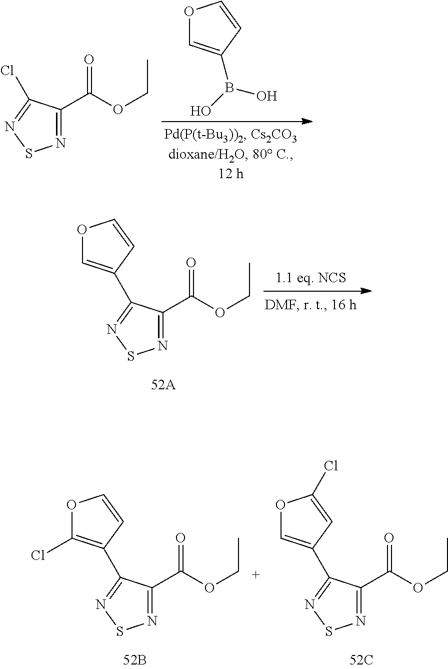

To a solution of ethyl 4-chloro-1,2,5-thiadiazole-3-carboxylate (3.0 g, 15.57 mmol) in dioxane (50 mL) and H₂O (5 mL) was added Cs₂CO₃ (15.2 g, 46.72 mmol) and 3-furylboronic acid (2.1 g, 18.69 mmol), the mixture was degassed and purged with N₂ for 3 times, then Pd(P(t-Bu)₃)₂ (796 mg, 1.56 mmol) was added. The mixture was stirred at 80° C. for 12 hours under N₂ and cooled to room temperature and concentrated, the residue was diluted with H₂O (100 mL) and extracted with EA (100 mL×3). The obtained organic phase was combined, washed with brine (50 mL×3) and dried over anhydrous Na₂SO₄ and filtered and the filtrate was concentrated to give a residue, which was purified by silica gel column chromatography (PE:EA=1:0 to 10:1) to give compound 52A (2 g, yield 57.3%) as a colorless oil. ¹H NMR (CDCl₃, 400 MHz) δ 8.44 (s, 1H), 7.51 (d, J=1.6 Hz, 1H), 7.03 (d, J=1.6 Hz, 1H), 4.50 (q, J=6.8 Hz, 2H), 1.49 (t, J=6.8 Hz, 3H).

To a solution of compound 52A (1.5 g, 6.69 mmol) in DMF (20 mL) was added NCS (1.0 g, 7.49 mmol). The mixture was stirred at 25° C. for 16 hours. The reaction was diluted with H₂O (60 mL) and extracted with EA (20 mL×3), the combined organic phase was washed with Na₂S₂O₃ (10% aq., 20 mL) and brine (20 mL×3) and concentrated to give a residue. The residue was purified by silica gel column chromatography (PE:EA=1:0 to 10:1) to give pure compound 52B (330 mg, yield: 19.5%) as a colorless oil and the mixture consist of compound 52A and compound 52C (500 mg). The mixture consist of compound 52A and compound 52C was purified by preparatory-TLC (PE:EA=100: 1, 5 times) to give compound 52C (135 mg, yield: 7.8%) as a white solid. Compound 52B: ¹H NMR (CDCl₃, 400 MHz) δ 7.43 (d, J=1.6 Hz, 1H), 6.85 (d, J=1.6 Hz, 1H), 4.60 (q, J=7.2 Hz, 2H), 1.42 (t, J=7.2 Hz, 3H). Compound 52C: ¹H NMR (CDCl₃, 400 MHz) δ 8.35 (s, 1H), 6.85 (s, 1H), 4.50 (q, J=7.2 Hz, 2H), 1.48 (t, J=7.2 Hz, 3H).

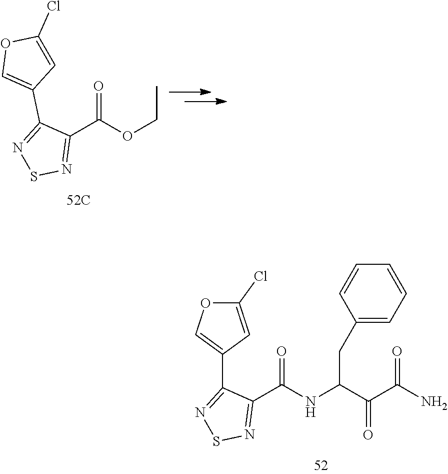 [SMILES: CCOC(=O)c1nsnc1-c1coc(Cl)c1 ; NC(=O)C(=O)C(Cc1ccccc1)NC(=O)c1nsnc1-c1coc(Cl)c1]

### N-(4-AMINO-3,4-DIOXO-1-PHENYLBUTAN-2-YL)-4-(5-CHLOROFURAN-3-YL)-1,2,5-THIADIAZOLE-3-CARBOXAMIDE (52)

Compounds ethyl 4-(5-chlorofuran-3-yl)-1,2,5-thiadiazole-3-carboxylate (52C) was converted to compound 52 using procedures as described in Example 1. Compound 52 (60 mg, yield: 62.8%) as a white solid was obtained. ¹H NMR (400 MHz, DMSO-d₆) δ 9.37 (d, J=7.7 Hz, 1H), 8.22 (s, 1H), 8.06 (d, J=1.1 Hz, 1H), 7.93 (s, 1H), 7.32-7.18 (m, 5H), 6.81 (d, J=1.1 Hz, 1H), 5.57-5.49 (m, 1H), 3.25 (dd, J=3.9, 14.0 Hz, 1H), 2.89 (dd, J=10.3, 14.0 Hz, 1H). MS (ESI) m/z (M+H)⁺ 405.0.

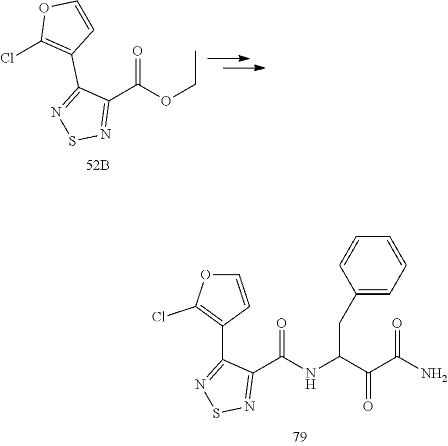 [SMILES: CCOC(=O)c1nsnc1-c1ccoc1Cl ; NC(=O)C(=O)C(Cc1ccccc1)NC(=O)c1nsnc1-c1ccoc1Cl]

### N-(4-AMINO-3,4-DIOXO-1-PHENYLBUTAN-2-YL)-4-(2-CHLOROFURAN-3-YL)-1,2,5-THIADIAZOLE-3-CARBOXAMIDE (79)

Compounds ethyl 4-(2-chlorofuran-3-yl)-1,2,5-thiadiazole-3-carboxylate (52B) was converted to compound 79 using procedures as described in Example 1. Compound 52 (50 mg, yield: 52.3%) as a pale yellow solid was obtained. ¹H NMR (400 MHz, DMSO-d₆) δ 9.37 (d, J=7.7 Hz, 1H), 8.20 (s, 1H), 7.92 (s, 1H), 7.73 (d, J=2.2 Hz, 1H), 7.32-7.17 (m, 5H), 6.59 (d, J=2.2 Hz, 1H), 5.56-5.47 (m, 1H), 3.29-3.18 (m, 1H), 2.88 (dd, J=10.0, 14.0 Hz, 1H). MS (ESI) m/z (M+H)⁺ 405.0.

### Example 17

### COMPOUNDS 85-86,57, AND 82

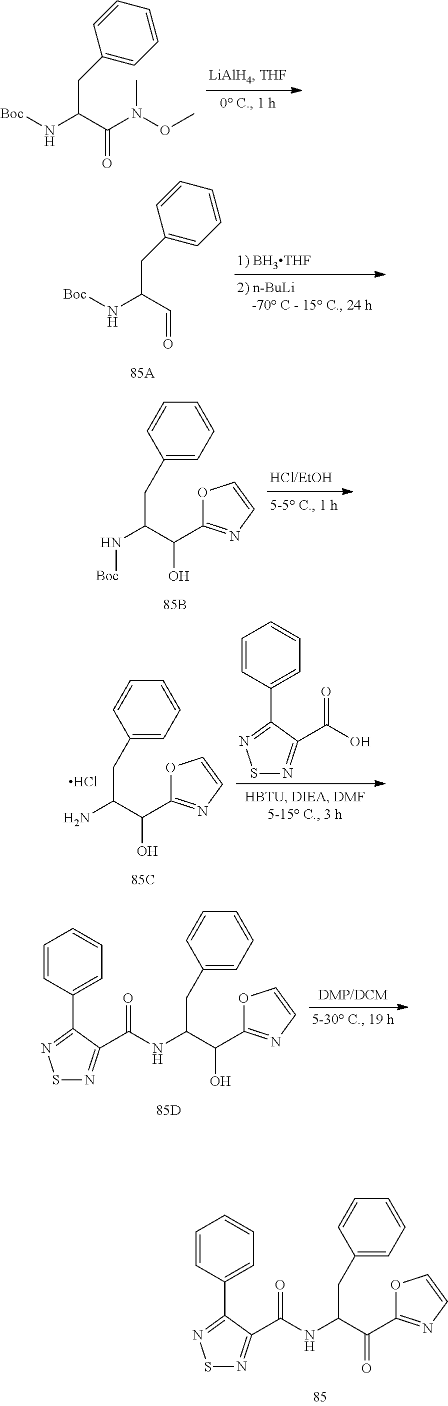

### N-(1-(OXAZOL-2-YL)-1-OXO-3-PHENYLPROPAN-2-YL)-4-PHENYL-1,2,5-THIADIAZOLE-3-CARBOXAMIDE (85)

To the mixture of LiAlH₄ (406.2 mg, 10.70 mmol) in THF (20 mL), solution of tert-butyl (1-(methoxy(methyl)amino)-1-oxo-3-phenylpropan-2-yl)carbamate (3 g, 9.73 mmol) in THF (20 mL) was added drop-wise at 0° C. under N₂ atmosphere. After addition, the mixture was stirred at 0° C. for 1 h. EtOAc (6 mL) was added drop-wise to the reaction mixture maintaining the temperature below 5° C., after that HCl (1M, 10 mL) was added. The reaction mixture was separated in a separation funnel and the aqueous was extracted with EtOAc (30 mL×2), the combined organic phase was washed with HCl (1M, 30 mL×3), sat. NaHCO₃ (30 mL) and brine (30 mL), dried over anhydrous Na₂SO₄. Filtered and the filtrate was concentrated to give compound 85A (2.3 g, yield: 94.8%) as a white solid. The product was used directly in next step. ¹H NMR (400 MHz, DMSO-d₆) 9.52 (s, 1H), 7.40-7.10 (m, 6H), 4.15-4.00 (m, 1H), 3.13-3.05 (m, 1H), 2.75-2.65 (m, 1H), 1.31 (s, 9H).

A solution comprised of oxazole (166.2 mg, 2.41 mmol) in THF (20 mL) was treated with BH₃.THF (1 M, 2.41 mL) under nitrogen and the mixture was stirred at 5-15° C. for 30 minutes and then cooled to −70° C. A solution comprised of n-BuLi (2.5M in cyclohexane, 1 mL) was added drop-wise and the mixture was stirred for 30 minutes at −70° C. A solution comprised of compound 85A (300 mg, 1.20 mmol) in THF (10 mL) was added and the mixture was stirred and allowed to warm to room temperature (5-15° C.) while the reaction proceeded to completion (24 h after). The mixture then was cooled to −78° C., quenched by slowly adding 5 percent acetic acid in ethanol (13.8 mL), allowed to warm to ambient temperature (5-15° C.) and stirred for 18 hours. The solvent was removed under reduced pressure, the residue was diluted with H₂O (15 mL) and extracted with EtOAc (20 mL×3). The organic phase was combined, washed with brine (30 mL) and concentrated to give a residue. The residue was purified by silica gel column chromatography (PE:EA=1:0 to 0:1) to give compound 85B (170 mg, yield: 24.4%) as a colorless oil. MS (ESI) m/z (M-Boc)⁺218.9.

The mixture of compound 85B (170 mg, 533.97 umol) in EtOAc (5 mL) was mixed with HC/EtOAc (4M, 10 mL) and stirred at room temperature (5-15° C.) for 1 h. The solvent was removed under reduced pressure to give compound 85C (150 mg, crude, HCl) as a white solid. The product was used directly in next step.

The mixture of 4-phenyl-1,2,5-thiadiazole-3-carboxylic acid (121.4 mg, 588.9 umol), compound 85C (150 mg, 588.90 umol, HCl), DIEA (0.3 mL, 1.77 mmol) and HBTU (245.67 mg, 647.79 umol) in DMF (10 mL) was stirred at 5-15° C. for 3 h. The reaction was diluted with H₂O (30 mL), extracted with EtOAc (30 mL×3). The organic phase was combined and washed with HCl (1M, 30 mL), sat. NaHCO₃aq. (30 mL), brine (30 mL×2) and concentrated to give a residue. The residue was purified by preparatory-HPLC (HCl system) purification to give compound 85D (50 mg, yield: 20.8%) as a white solid. ¹H NMR (400 MHz, DMSO-d₆) δ 9.02-8.83 (d, J=7.7 Hz, 1H), 8.07 (s, 1H), 7.52-7.16 (m, 12H), 4.88-4.74 (m, 1H), 4.64-4.49 (m, 1H), 3.20-2.77 (m, 2H). MS (ESI) m/z (M+H)⁺ 407.0.

To the mixture of compound 85D (50 mg, 123.01 umol) in DCM (20 mL), DMP (156.5 mg, 369.04 umol) was added and stirred at room temperature (5-15° C.). After 1.5 h, DMP (100 mg) was added and the reaction was stirred at 30° C. overnight (16 h). The reaction was diluted with DCM (20 mL), quenched with sat. Na₂S₂O₃ aqueous (30 mL) and separated. The organic phase was washed with sat. NaHCO₃ aqueous (20 mL) and brine (20 mL×3), dried over anhydrous Na₂SO₄. Filtered and the filtrate was concentrated. Compound 85 (40 mg, yield: 62.3%) was obtained as a pale yellow solid. ¹H NMR (400 MHz, DMSO-d₆) δ 9.68 (d, J=7.6 Hz, 1H), 8.50 (s, 1H), 7.66 (s, 1H), 7.58-7.52 (m, 2H), 7.49-7.42 (m, 1H), 7.41-7.22 (m, 7H), 5.74-5.66 (m, 1H), 3.41-3.36 (m, 1H), 3.06-2.95 (m, 1H). MS (ESI) m/z (M+H)⁺ 405.1. ¹H NMR (400 MHz, CDCl₃) δ 7.88 (s, 1H), 7.73-7.66 (m, 3H), 7.47-7.38 (m, 4H), 7.32-7.22 (m, 3H), 7.19-7.13 (m, 2H), 5.99 (dt, J=5.3, 7.8 Hz, 1H), 3.52 (dd, J=5.1, 13.9 Hz, 1H), 3.26 (dd, J=7.5, 14.1 Hz, 1H).

### N-(1-(BENZO[d]OXAZOL-2-YL)-1-OXO-3-PHENYLPROPAN-2-YL)-4-PHENYL-1,2,5-THIADIAZOLE-3-CARBOXAMIDE (86)

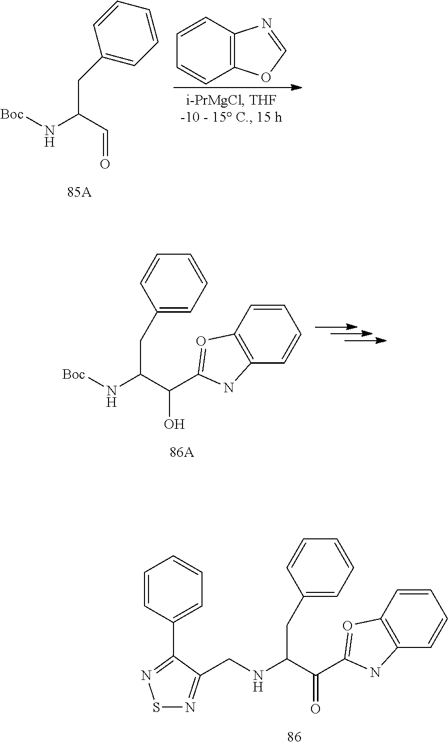

To a solution of 1, 3-benzoxazole (573.4 mg, 4.81 mmol) in THF (20 mL) at −10° C. was added i-PrMgCl (2.0 M, 1.60 mL), the reaction mixture was stirred at −10° C. for 1 h. Then compound 85A (400 mg, 1.60 mmol) was added as a solution in THF (20 mL) and the reaction mixture was stirred at −10° C. for 2 h followed by 12 h at 5-15° C. The reaction was concentrated and the residue was diluted with EtOAc (60 mL), washed with brine (30 mL×2) and concentrated to give a residue. The residue was diluted with EtOAc (100 mL) and washed with brine (30 mL×3), concentrated to give the crude product. The crude product was purified by silica gel column chromatography (PE:EA=1:0 to 5:1) to give compound 86A (270 mg, yield: 45%) as a yellow oil. ¹H NMR (400 MHz, CDCl₃) δ 7.77-7.63 (m, 1H), 7.52 (dt, J=2.6, 6.7 Hz, 1H), 7.41-7.30 (m, 4H), 7.26-7.13 (m, 3H), 5.11-4.88 (m, 2H), 4.53-4.19 (m, 2H), 3.08 (br. d, J=7.6 Hz, 1H), 3.00-2.83 (m, 1H), 1.43-1.27 (m, 9H). MS (ESI) m/z (M+Na⁺) 391.0.

Compound 86A was converted to compound 86 using procedures as described as for compound 85. Compound 86 (180 mg, yield: 78.53%) as a white solid was obtained. ¹H NMR (400 MHz, DMSO-d₆) δ 9.78 (d, J=7.3 Hz, 1H), 8.04 (d, J=8.1 Hz, 1H), 7.94 (d, J=8.3 Hz, 1H), 7.68 (t, J=7.5 Hz, 1H), 7.60-7.52 (m, 3H), 7.46-7.40 (m, 1H), 7.40-7.28 (m, 6H), 7.27-7.21 (m, 1H), 5.89-5.79 (m, 1H), 3.49 (dd, J=3.8, 14.1 Hz, 1H), 3.07 (dd, J=9.9, 14.1 Hz, 1H). MS (ESI) m/z (M+H)⁺ 455.0.

### N-(1-(OXAZOL-2-YLAMINO)-1-OXO-3-PHENYLPROPAN-2-YL)-4-PHENYL-1,2,5-THIADIAZOLE-3-CARBOXAMIDE (57)

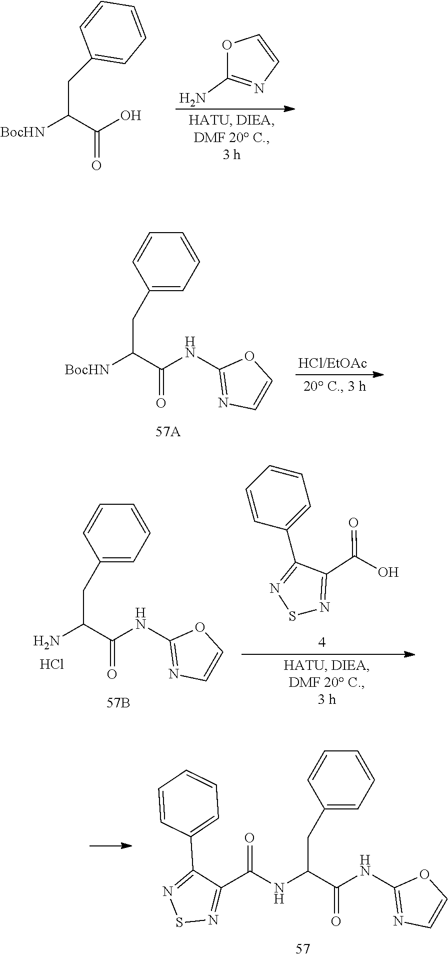

Compounds (tert-butoxycarbonyl)phenylalanine and oxazol-2-amine were coupled using conditions described for compound 85 to yield intermediate 57A which was converted to compound 57. Compound 57 (35 mg, yield: 11.2%) as a white solid was obtained. ¹H NMR (400 MHz, DMSO-d₆) δ 11.72 (br. s, 1H), 9.47 (br. d, J=7.7 Hz, 1H), 7.93 (s, 1H), 7.51 (d, J=7.3 Hz, 2H), 7.46-7.36 (m, 3H), 7.36-7.22 (m, 5H), 7.15 (s, 1H), 5.00-4.80 (m, 1H), 3.25-3.10 (m, 1H), 3.05-2.93 (m, 1H). MS (ESI) m/z (M+H)⁺ 420.2.

### N-(1-CYANO-2-PHENYLETHYL)-4-PHENYL-1,2,5-THIADIAZOLE-3-CARBOXAMIDE (82)

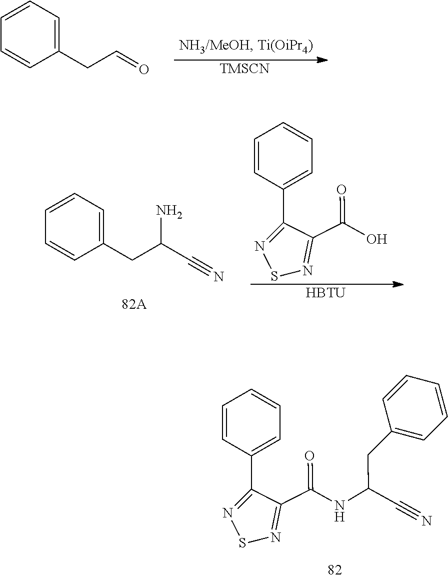

To a stirred solution of 2-phenylacetaldehyde (3 g, 24.97 mmol, 1.95 mL) in MeOH (70 mL) was added NH₃ in MeOH (30 mL) and Ti(i-PrO)₄ (10.64 g, 37.45 mmol, 11.05 mL) and the resulting solution was stirred at 15° C. for 2 h. To the reaction mixture was then added TMSCN (4.46 g, 44.94 mmol, 5.62 mL), then the reaction mixture was stirred at 15° C. for 16 h. Reaction mixture was quenched with water (150 mL), and the resulting white precipitate was filtered. The filtrate was concentrated under reduced pressure, extracted with ethyl acetate (50 mL×3) and the organic phase was washed with brine (100 mL). The organic layer was dried over Na₂SO₄, filtered and concentrated under reduced pressure. Compound 82A (2 g, yield: 54.8%) was obtained as a yellow oil, which was used into the next step without further purification. ¹H NMR (400 MHz, DMSO-d₆) δ 7.36-7.20 (m, 5H), 4.03-3.85 (m, 1H), 3.00-2.80 (m, 2H), 2.38 (br s, 2H)

Compound 82A was coupled with 4-phenyl-1,2,5-thiadiazole-3-carboxylic acid using conditions as described for compound 85 to yield compound 82. Compound 82 (130 mg, yield: 40.1%) as a white solid was obtained. H NMR (400 MHz, DMSO-d₆) δ 9.88 (br d, J=7.8 Hz, 1H), 7.61-7.46 (m, 3H), 7.45-7.39 (m, 2H), 7.38-7.20 (m, 5H), 5.25 (q, J=7.8 Hz, 1H), 3.30-3.07 (m, 2H).

### Example 18

### COMPOUNDS 41, 40, 38, 67, 40, 65, 42, 64, 74, 72,106, AND 107

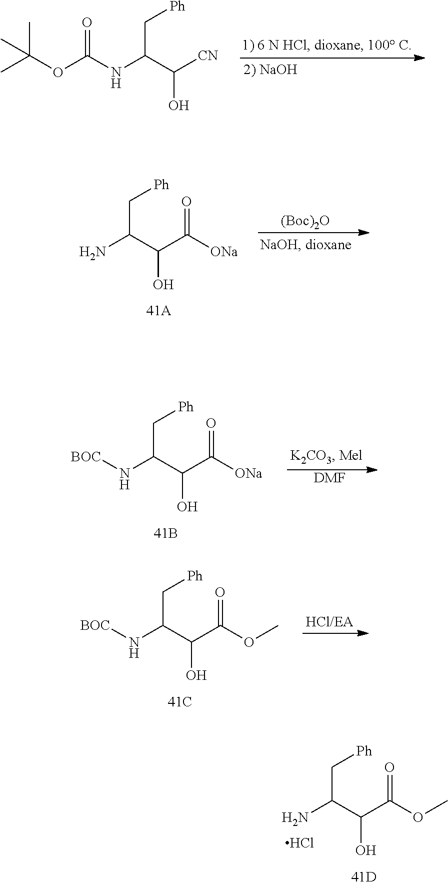

To a mixture of tert-butyl (1-cyano-1-hydroxy-3-phenylpropan-2-yl)carbamate (27 g, 97.7 mmol) in dioxane (150 mL) was added HCl (6 N, 360 mL). The mixture was stirred at 100° C. for 12 h. The hydrolysis reaction was allowed to cool to room temperature and then concentrated to 120 mL in vacuo. The aqueous phase was alkalized with NaOH (solid) till pH ˜11-12. The alkalized aqueous phase was used in next step without purification.

To a mixture of the alkalized aqueous solution compound 41A (97.7 mmol) in H₂O (120 mL)) was added dioxane (60 mL) and (Boc)₂₀ (45 mL, 195.9 mmol), which was stirred at 25° C. for 12 h while the pH was maintained between 10 and 11 with NaOH (2M). The mixture was concentrated under reduce pressure to move dioxane. After being alkalized to pH ˜12-13, the aqueous phase was washed with EA (80 mL×2) and acidified with 6N HCl till pH ˜2-3, and then extracted with EA (50 mL×3). The combined organic phases were washed with brine (50 mL), dried over Na₂SO₄, filtered and concentrated in vacuo to afford compound 41B (29.5 g, crude) as light red sticky liquid, which was used in next step without purification. ¹H NMR (DMSO-d₆, 400 MHz): δ 7.32-7.14 (m, 6H), 6.73-6.35 (m, 1H), 4.00-3.83 (m, 2H), 2.87-2.75 (m, 1H), 2.74-2.66 (m, 1H), 1.32-1.24 (m, 9H).

To a mixture of compound 41B (11 g, 37.3 mmol) in DMF (80 mL) was added K₂CO₃ (10.3 g, 74.5 mmol), followed by MeI (4.9 mL 78.9 mmol). The mixture was stirred at 25° C. for 2 h. The mixture was filtered. The filtrates was concentrated under reduced pressure and then diluted with H₂O (200 mL) and extracted with EA (50 mL×3). The combined organic phase was washed with brine (50 mL), dried over Na₂SO₄, filtered and concentrated in vacuo to afford compound 41C (8.56 g, 74.2% yield) as light yellow solid, which was used in next step without purification. ¹H NMR (DMSO-d₆, 400 MHz): δ 7.33-7.11 (m, 5H), 6.84-5.99 (m, 1H), 5.91-5.34 (m, 1H), 4.03-3.80 (m, 2H), 3.64-3.52 (m, 3H), 2.86-2.75 (m, 1H), 2.71-2.59 (m, 1H), 1.33-1.15 (m, 9H). MS (ESI) m/z (M+Na)⁺ 332.1, (M-Boc+H)⁺ 210.1.

To a mixture of compound 41C (4 g, 12.9 mmol) in EtOAc (10 mL) was added HCV/EtOAc (4M, 40 mL). The mixture was stirred at 25° C. for 3 h. The mixture was concentrated in vacuo. The residue was triturated with EA (20 mL). The solid was collected and dried in vacuum to afford compound 41D (2.68 g, 84.3% yield, HCl) as white solid. ¹H NMR (DMSO-d₆, 400 MHz): δ 8.27 (s, 3H), 7.41-7.17 (m, 5H), 6.71-6.34 (m, 1H), 4.53-3.93 (m, 1H), 3.77-3.60 (m, 1H), 3.59 (s, 2H), 3.27 (s, 1H), 3.11-2.82 (m, 2H).

### METHYL 3-(1-CYCLOPROPYL-3-PHENYL-1H-PYRAZOLE-4-CARBOXAMIDO)-2-OXO-4-PHENYLBUTANOATE (41)

### and

### 3-(1-CYCLOPROPYL-3-PHENYL-1H-PYRAZOLE-4-CARBOXAMIDO)-2-OXO-4-PHENYLBUTANOIC ACID (60)

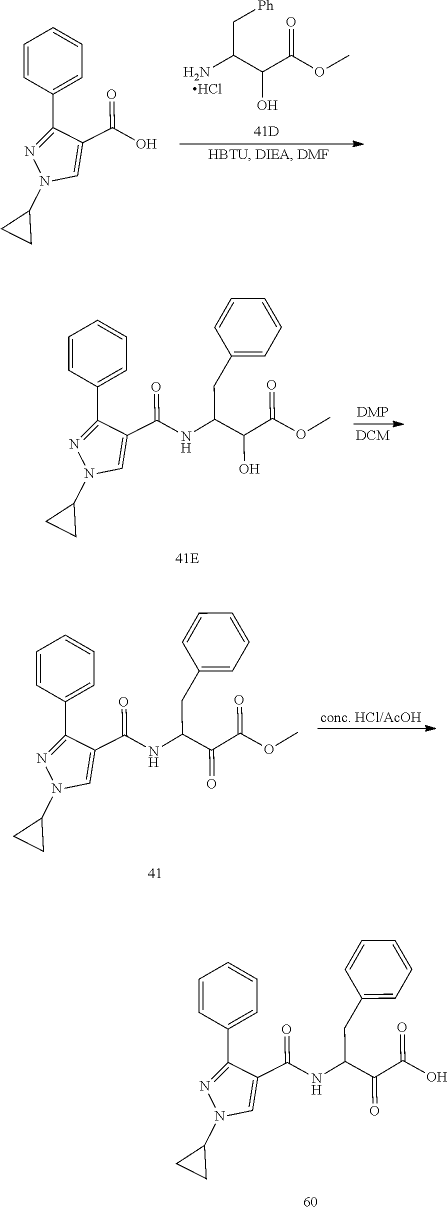 [SMILES: O=C(O)c1cn(C2CC2)nc1-c1ccccc1 ; Cl ; COC(=O)C(O)C(N)Cc1ccccc1 ; COC(=O)C(O)C(Cc1ccccc1)NC(=O)c1cn(C2CC2)nc1-c1ccccc1 ; COC(=O)C(=O)C(Cc1ccccc1)NC(=O)c1cn(C2CC2)nc1-c1ccccc1 ; O=C(O)C(=O)C(Cc1ccccc1)NC(=O)c1cn(C2CC2)nc1-c1ccccc1]

To a mixture of 1-cyclopropyl-3-phenyl-1H-pyrazole-4-carboxylic acid (0.3 g, 1.3 mmol) and intermediate 41D (387.5 mg, 1.6 mmol, HCl) in DMF (10 mL) was added HBTU (500 mg, 1.3 mmol) and DIEA (750 uL, 4.31 mmol). The mixture was stirred at 25° C. for 1 h. The mixture was concentrated, and then diluted with H₂O (100 mL) and extracted with EA (30 mL×3). The combined organic phase was washed with 1N HCl (30 mL), saturated NaHCO₃ (30 mL), brine (30 mL×3), dried over Na₂SO₄, filtered and concentrated in vacuo to afford compound 41E (0.55 g, 99.7% yield) as white solid, which was used in next step without purification. ¹H NMR (DMSO-d₆, 400 MHz): δ 8.10-7.99 (m, 1H), 7.96-7.67 (m, 1H), 7.57-7.45 (m, 2H), 7.33-7.13 (m, 8H), 5.96-5.55 (m, 1H), 4.52-4.33 (m, 1H), 4.16-4.07 (m, 1H), 3.83-3.73 (m, 1H), 3.63-3.51 (m, 3H), 2.97-2.68 (m, 2H), 1.14-0.96 (m, 4H). MS (ESI) m/z (M+H)⁺ 420.1.

To a mixture of compound 41E (0.54 g, 1.3 mmol) in DCM (50 mL) was added DMP (1.6 g, 3.9 mmol). The mixture was stirred at 25° C. for 50 min. The reaction was diluted with DCM (20 mL) and quenched by 40 mL of Sat. Na₂S₂O₃ solution and 40 mL of saturated NaHCO₃ solution and stirred for 5 min. After quenching the reaction, the reaction mixture was poured into separatory funnel and separated. The separated aqueous phase was extracted with DCM (30 mL×2). The combined organic phase was washed with brine (30 mL×2), dried over anhydrous Na₂SO₄, filtered and concentrated in vacuo to afford compound 41 (0.51 g, yield 93.6%) as pale yellow solid, which was used in next step without purification. ¹H NMR (DMSO-d₆, 400 MHz): S. 8.61 (d, J=6.8 Hz, 1H), 8.11 (s, 1H), 7.59-7.48 (m, 2H), 7.36-7.19 (m, 8H), 5.11-4.96 (m, 1H), 3.87-3.78 (m, 1H), 3.75 (s, 3H), 3.24-3.13 (m, 1H), 2.97-2.84 (m, 1H), 1.12-0.98 (m, 4H). MS (ESI) m/z (M+H)⁺ 418.2.

To a mixture of compound 41 (0.15 g, 359.3 umol) in AcOH (2 mL) was added HCl (12M, 2 mL) in one portion. The mixture was stirred at 40° C. for 1 h. The mixture was diluted with H₂O (50 mL), and extracted with EA (30 mL×3). The combined organic phase was washed with brine (30 mL), dried over Na₂SO₄, filtered and concentrated in vacuo. The residue was purified by preparatory-HPLC (HCl condition) to afford compound 60 (40 mg, yield 27.6%) as pale yellow solid. ¹H NMR (DMSO-d₆, 400 MHz): δ. 8.52 (d, J=7.3 Hz, 1H), 8.11 (s, 1H), 7.60-7.50 (m, 2H), 7.36-7.18 (m, 8H), 5.08-4.97 (m, 1H), 3.88-3.74 (m, 1H), 3.24-3.12 (m, 1H), 2.95-2.81 (m, 1H), 1.14-0.96 (m, 4H). MS (ESI) m/z (M+H)⁺ 404.1.

### METHYL 2-OXO-4-PHENYL-3-(4-PHENYL-1,2,5-THIADIAZOLE-3-CARBOXAMIDO)BUTANOATE (38)

### and

### 2-OXO-4-PHENYL-3-(4-PHENYL-1,2,5-THIADIAZOLE-3-CARBOXAMIDO)BUTANOIC ACID (67)

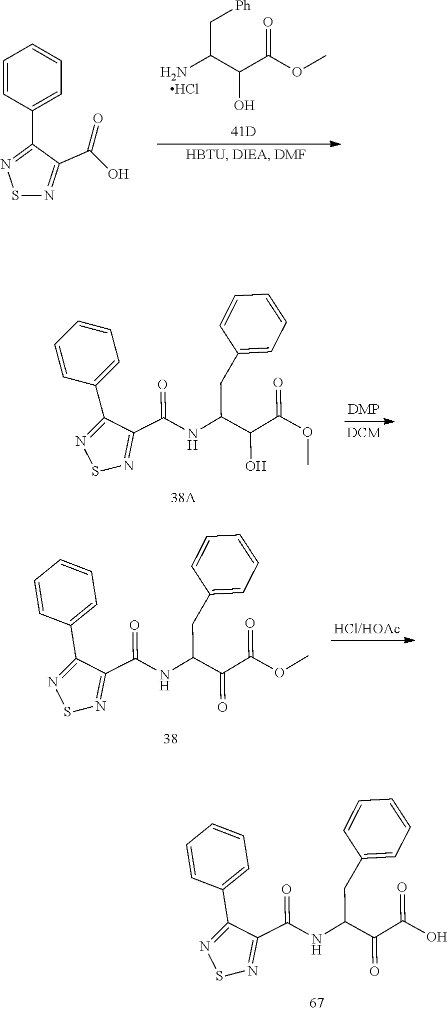 [SMILES: O=C(O)c1nsnc1-c1ccccc1 ; Cl ; COC(=O)C(O)C(N)Cc1ccccc1 ; COC(=O)C(O)C(Cc1ccccc1)NC(=O)c1nsnc1-c1ccccc1 ; COC(=O)C(=O)C(Cc1ccccc1)NC(=O)c1nsnc1-c1ccccc1 ; O=C(O)C(=O)C(Cc1ccccc1)NC(=O)c1nsnc1-c1ccccc1]

Compound 38 was prepared from 4-phenyl-1,2,5-thiadiazole-3-carboxylic acid and intermediate 41D using the same procedure as for compound 41. Compound 38 (0.440 g, yield 88.4%) was obtained as white solid, which was used in next step without purification. ¹H NMR (DMSO-d₆,400 MHz) 9.27 (br d, J=6.0 Hz, 1H), 7.64 (br d, J=7.0 Hz, 2H), 7.51-7.38 (m, 3H), 7.31-7.21 (m, 5H), 5.32 (ddd, J=5.0, 7.5, 9.1 Hz, 1H), 3.81 (s, 3H), 3.28 (dd, J=4.9, 14.2 Hz, 1H), 3.03-2.98 (m, 1H). MS (ESI) m/z (M+H)⁺ 396.1.

Compound 67 was prepared from compound 38 using the same procedure as for compound 60. Compound 67 (0.123 g, yield 82.89%) was obtained as white solid. ¹H NMR (DMSO-d₆0.400 MHz): δ 7.84 (br d, J=6.5 Hz, 1H), 7.63-7.59 (m, 2H), 7.53-7.42 (m, 3H), 7.35-7.24 (m, 5H), 5.40 (ddd, J=4.8, 7.8, 9.0 Hz, 1H), 3.38 (dd, J=4.8, 14.1 Hz, 1H), 3.04 (dd, J=9.0, 14.1 Hz, 1H). MS (ESI) m/z (M+H)⁺ 382.1.

### 2-OXO-3-4-(2-OXO-2,3-DIHYDROBENZO[D]OXAZOL-4-YL)-1,2,5-THIADIAZOLE-3-CARBOXAMIDO)-4-PHENYLBUTANOIC ACID (106)

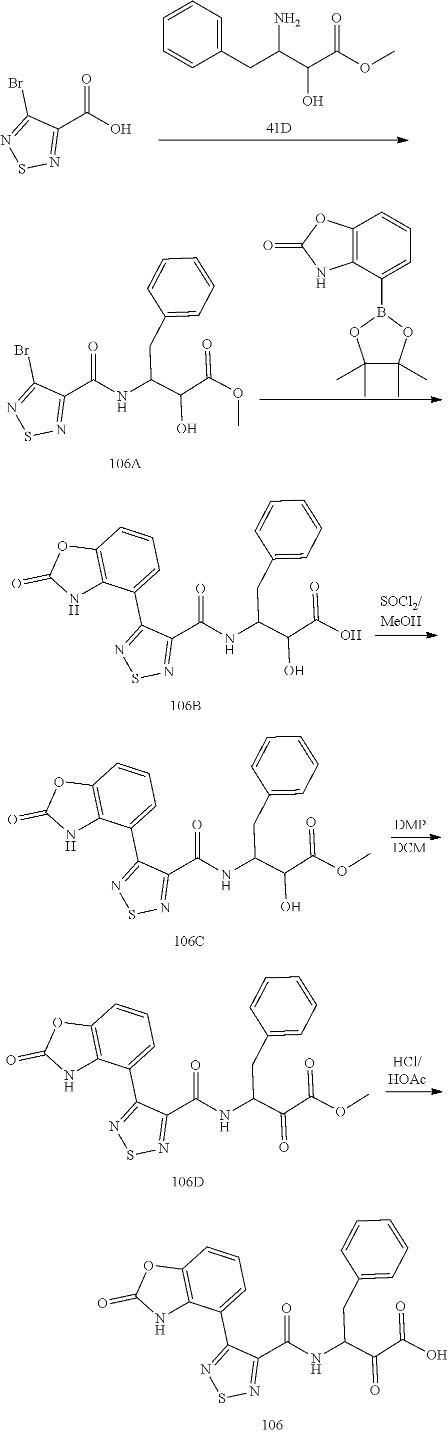

Compound 106A was prepared from 4-bromo-1,2,5-thiadiazole-3-carboxylic acid and intermediate 41D using the same procedure as for compound 41. Compound 106A (0.640 g, yield 33.42%) was obtained as white solid, which was used in next step without purification. MS (ESI) m/z (M+H)⁺ 401.7.

To a solution compound 106A (540 mg, 1.35 mmol) and 4-(4,4,5,5-tetramethyl-1,3,2-dioxaborolan-2-yl)benzo[d]oxazol-2(3H)-one (422.69 mg, 1.62 mmol) in dioxane (30 mL) and H₂O (10 mL) was added Pd(dppf)Cl₂ (98.72 mg, 134.92 umol), Na₂CO₃ (428.99 mg, 4.05 mmol). The mixture was stirred at 80° C. under N₂ for 5 h. The mixture was diluted with H₂O (150 mL), washed with EtOAc (150 mL×2). The aqueous phase was collected, adjusted to pH ˜4 with 1N HCl, extracted with EtOAc (100 mL×2). The organics were collected, dried with Na₂SO₄, filtered and concentrated. Compound 106B (40 mg, crude) was obtained as brown oil, which was used directly for the next step without further purification. MS (ESI) m/z (M+H)⁺ 440.9.

To a solution of compound 106B (490 mg, 1.11 mmol) in MeOH (20 mL) at 0° C. was added SOCl₂ (330.9 mg, 2.78 mmol) dropwise. The mixture was then heated to 60° C. and stirred for 1 h. The solvent was removed in vacuo. The residue was purified by preparatory-HPLC (HCl) to afford compound 106C (140 mg, 26.72% yield, 96.5% purity) as light yellow solid. ¹H NMR (400 MHz, DMSO-d₆) δ 8.63 (d, J=7.6 Hz, 1H), 7.35-7.34 (m, 1H), 7.33-7.20 (m, 5H), 6.99-6.98 (m, 1H), 5.84 (d, J=6.4 Hz, 1H), 4.49-4.43 (m, 1H), 4.14-4.12 (m, 1H), 3.50 (s, 3H), 2.94-2.78 (m, 2H).

Compound 106 was prepared from compound 106C using the same procedure as for compound 106. Compound 106 (0.040 g, yield 37.49%) was obtained as white solid. ¹H NMR (CD₃CN, 400 MHz): δ 9.35 (br. s, H), 7.88 (d, J=7.6 Hz, 1H), 7.36-7.23 (m, 7H), 7.17-7.05 (m, 1H), 5.49-5.36 (m, 1H), 3.41-3.33 (m, 1H), 3.09-3.00 (m, 1H). MS (ESI) m/z (M+H)⁺ 439.1.

### 3-(4-(2,2-DIFLUOROBENZO[D][1,3]DIOXOL-4-YL)-1,2,5-THIADIAZOLE-3-CARBOXAMIDO)-2-OXO-4-PHENYLBUTANOIC ACID (107)

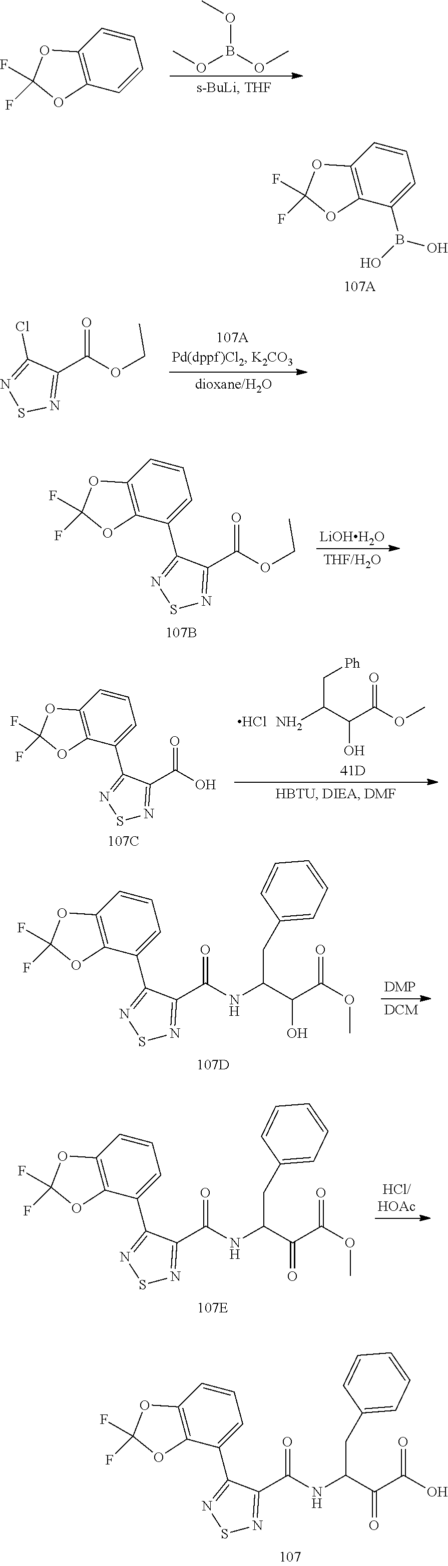

2,2-Difluoro-1,3-benzodioxole (3 g, 18.98 mmol) was dissolved in THF (60 mL) and the resulting solution cooled to −78° C. Sec-butyllithium (1.3M, 15 mL) was added dropwise and the reaction mixture stirred for 1.5 h at −78° C. Trimethyl borate (2.44 mL, 21.63 mmol) was added and the mixture was allowed to warm slowly to −30° C. for 1 h. The reaction mixture was quenched with 2N solution of HCl, adjusted to pH ˜2-3 and diluted with H₂O (30 mL). The reaction was extracted with EA (200 mL), two phases were separated and the organic layer was washed with brine (100 mL), dried over Na₂SO₄, filtered and evaporated to dryness. Compound 107A (3.25 g, yield 84.82%) was obtained as a white solid, which was used for next step without purification. ¹H NMR (400 MHz, DMSO-d₆) δ 8.44 (br s, 2H), 7.42 (dd, J=7.8, 15.3 Hz, 2H), 7.23-7.12 (m, 1H).

Cs₂CO₃ (7.26 g, 22.29 mmol) and palladium-tritert-butylphosphane (1.14 g, 2.23 mmol) was added to the mixture of ethyl 4-chloro-1,2,5-thiadiazole-3-carboxylate (1.43 g, 7.43 mmol) and compound 107A (1.5 g, 7.43 mmol) in 1,4-dioxane (15 mL) and H₂O (6 mL) under N₂ atmosphere. The mixture was stirred at 90° C. for 2 h. The mixture was filtered and concentrated under vacuum. The product was purified by Flash Column Chromatography (0-30% EA/PE). Compound 107B (726 mg, 2.31 mmol, 31.10% yield) was obtained as a yellow solid. ¹H NMR (400 MHz, DMSO-d₆) δ 7.58 (dd, J=1.0, 8.1 Hz, 1H), 7.49 (dd, J=1.0, 8.1 Hz, 1H), 7.37-7.35 (m, 1H), 4.28 (q, J=7.1 Hz, 2H), 1.17 (t, J=7.1 Hz, 3H).

LiOH.H₂O (485 mg, 11.55 mmol) was added to the mixture of Compound 107B (726 mg, 2.31 mmol) in THF (6 mL) and H₂O (2 mL) at 0° C., then the mixture was stirred for 1 h at 20° C. Then the mixture was adjusted to pH ˜1-2 by 1N HCl and extracted with EA (40 mL). The organic phase was washed with brine (10 mL), dried over Na₂SO₄, filtered and concentrated under vacuum. Compound 107C (629 mg, yield 95.13%) was obtained as a yellow solid, which was used for next step without purification. ¹H NMR (400 MHz, DMSO-d₆) δ 14.3 (br. s., 1H), 7.59 (dd, J=1.0, 8.0 Hz, 1H), 7.50 (dd, J=1.0, 8.0 Hz, 1H), 7.40-7.33 (m, 1H).

Compound 107 was prepared from compound 107C using the same procedure as for compound 67. Compound 107 (0.014 g, yield 15.12%) was obtained as white solid. ¹H NMR (CD₃CN. 400 MHz): δ 7.90 (br. d., J=6.3 Hz, 1H), 7.47-7.16 (m, 8H), 5.43-5.32 (m, 1H), 3.37 (dd, J=4.9, 14.2 Hz, 1H), 3.09 (br dd, J=8.5, 14.1 Hz, 1H). MS (ESI) m/z (M+H)⁺ 462.1.

### METHYL 3-(3-(2-FLUOROPHENYL)-1-METHYL-1H-PYRAZOLE-4-CARBOXAMIDO)-2-OXO-4-PHENYLBUTANOATE (40)

### and

### 3-(3-(2-FLUOROPHENYL)-1-METHYL-1H-PYRAZOLE-4-CARBOXAMIDO)-2-OXO-4-PHENYLBUTANOIC ACID (65)

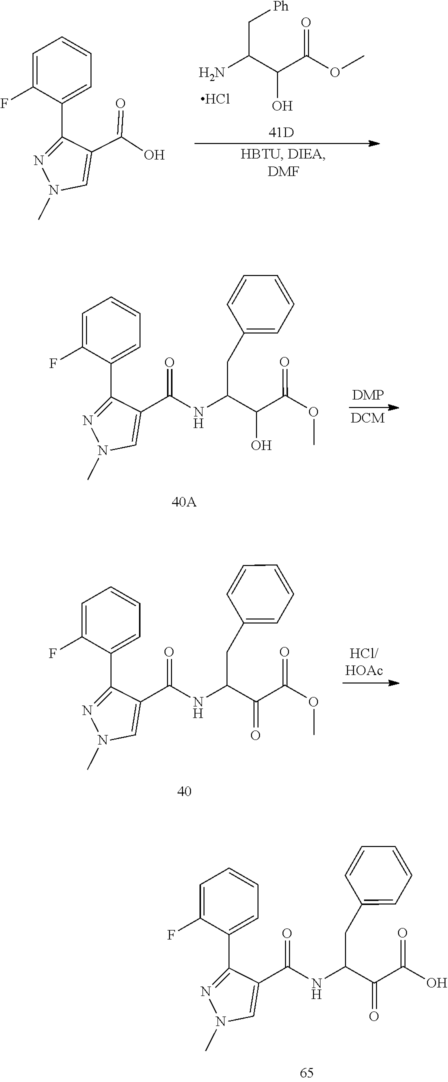 [SMILES: Cn1cc(C(=O)O)c(-c2ccccc2F)n1 ; Cl ; COC(=O)C(O)C(N)Cc1ccccc1 ; COC(=O)C(O)C(Cc1ccccc1)NC(=O)c1cn(C)nc1-c1ccccc1F ; COC(=O)C(=O)C(Cc1ccccc1)NC(=O)c1cn(C)nc1-c1ccccc1F ; Cn1cc(C(=O)NC(Cc2ccccc2)C(=O)C(=O)O)c(-c2ccccc2F)n1]

Compound 40 was prepared from 3-(2-fluorophenyl)-1-methyl-1H-pyrazole-4-carboxylic acid and intermediate 41D using the same procedure as for compound 41. Compound 40 (0.520 g, yield 87.1%) was obtained as yellow solid, which was used in next step without purification. ¹H NMR (DMSO-d₆, 400 MHz): δ 8.12 (br.s., 2H), 7.44-7.33 (m, 2H), 7.31-7.25 (m, 2H), 7.22-7.10 (m, 5H), 5.00 (br d, J=6.5 Hz, 1H), 3.91 (s, 3H), 3.75 (s, 3H), 3.17 (dd, J=5.3, 14.1 Hz, 1H), 2.94 (br.dd, J=8.9, 13.9 Hz, 1H). MS (ESI) m/z (M+H)⁺ 410.1.

Compound 65 was prepared from compound 40 using the same procedure as for compound 60. Compound 65 (60 mg, yield 40.5%) was obtained as white solid. ¹H NMR (DMSO-d₆, 400 MHz): δ 14.10 (s, 1H), 8.44 (d, J=7.0 Hz, 1H), 8.17 (s, 1H), 7.42-7.26 (m, 4H), 7.25-7.20 (m, 3H), 7.19-7.12 (m, 2H), 4.95 (ddd, J=4.8, 6.8, 9.5 Hz, 1H), 3.91 (s, 3H), 3.15 (dd, J=4.6, 13.9 Hz, 1H), 2.87 (dd, J=9.7, 13.9 Hz, 1H). MS (ESI) m/z (M+H)⁺ 396.2.

### METHYL 3-(4-(2-FLUOROPHENYL)-2-METHYLOXAZOLE-5-CARBOXAMIDO)-2-OXO-4-PHENYLBUTANOATE (42)

### and

### 3-(4-(2-FLUOROPHENYL)-2-METHYLOXAZOLE-5-CARBOXAMIDO)-2-OXO-4-PHENYLBUTANOIC ACID (64)

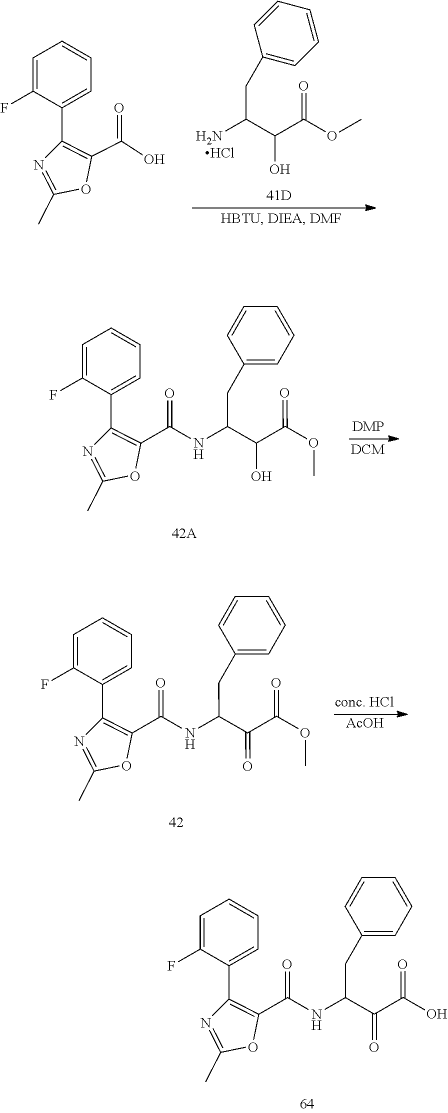

Compound 42 was prepared from 4-(2-fluorophenyl)-2-methyloxazole-5-carboxylic acid and intermediate 41D using the same procedure as for compound 41. Compound 42 (0.290 g, yield 67.0%) was obtained as light yellow solid, which was used in next step without purification. ¹H NMR (DMSO-d₆, 400 MHz): δ. 9.10 (d, J=7.1 Hz, 1H), 7.51-7.38 (m, 2H), 7.34-7.17 (m, 7H), 5.19-5.05 (m, 1H), 3.81-3.54 (m, 3H), 3.24-3.15 (m, 1H), 3.03-2.92 (m, 1H), 2.59-2.52 (m, 3H). MS (ESI) m/z (M+H)⁺ 411.1.

Compound 64 was prepared from compound 42 using the same procedure as for compound 60. Compound 64 (40 mg, yield 50.4%) was obtained as white solid. ¹H NMR (CD₃CN-d₃, 400 MHz): δ 7.54-7.39 (m, 2H), 7.37-7.11 (m, 8H), 5.31-5.16 (m, 1H), 3.29 (dd, J=5.0, 14.1 Hz, 1H), 3.00 (dd, J=8.8, 14.1 Hz, 1H), 2.50 (s, 3H). MS (ESI) m/z (M+H)⁺ 397.2.

### METHYL-3-(3-(3-FLUOROPHENYL)-1-METHYL-1H-PYRAZOLE-4-CARBOXAMIDO)-2-OXO-4-PHENYLBUTANOATE (74)

### and

### 3-(3-(3-FLUOROPHENYL)-1-METHYL-1H-PYRAZOLE-4-CARBOXAMIDO)-2-OXO-4-PHENYLBUTANOIC ACID (72)

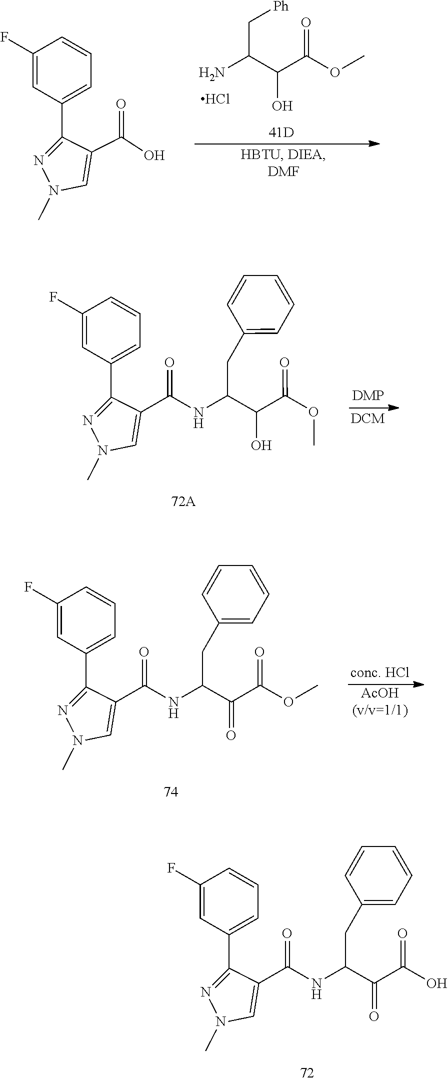

Compound 74 was prepared from 4-(2-fluorophenyl)-2-methyloxazole-5-carboxylic acid and intermediate 41D using the same procedure as for compound 41. Compound 74 (0.150 g, yield 75.3%) was obtained as light yellow solid, ¹H NMR (DMSO-d₆, 400 MHz): δ 8.73 (d, J=6.8 Hz, 1H), 8.06 (s, 1H), 7.45-7.29 (m, 4H), 7.28-7.20 (m, 4H), 7.14 (dt, J=2.1, 8.4 Hz, 1H), 5.06 (ddd, J=5.0, 6.8, 9.4 Hz, 1H), 3.91 (s, 3H), 3.76 (s, 3H), 3.20 (dd, J=4.9, 13.9 Hz, 1H), 2.91 (dd, J=9.5, 13.7 Hz, 1H). MS (ESI) m/z (M+H)+410.1.

Compound 72 was prepared from compound 74 using the same procedure as for compound 60. Compound 72 (50 mg, yield 64.7%) was obtained as white solid. ¹H NMR (DMSO-d₆, 400 MHz): δ 8.66 (br d, J=7.3 Hz, 1H), 8.07 (s, 1H), 7.44 (br d, J=8.0 Hz, 2H), 7.38-7.19 (m, 6H), 7.18-7.10 (m, 1H), 5.13-4.99 (m, 1H), 3.90 (s, 3H), 3.24-3.15 (m, 1H), 2.89 (dd, J=9.8, 14.1 Hz, 1H). MS (ESI) m/z (M+H)⁺ 396.2.

### Example 19

### COMPOUNDS 58, 75, 76, 73, 78, 81, 84, 88, 90, 91, 92, 98,105, AND 108

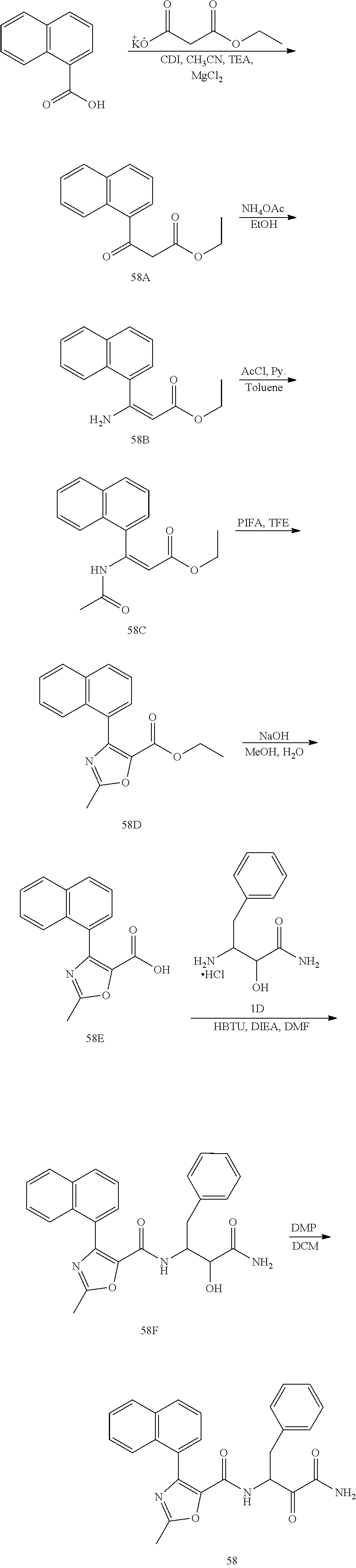

### N-(4-AMINO-3,4-DIOXO-1-PHENYLBUTAN-2-YL)-2-METHYL-4-(NAPHTHALEN-1-YL)OXAZOLE-5-CARBOXAMIDE (58)

(Flask A) A mixture of 1-naphthoic acid (25 g, 145.2 mmol) in CH₃CN (40 ml) was added CDI (28.3 g, 174.2 mmol), the mixture was stirred at 25° C. for 2 h. (Flask B) A mixture of ethyl potassium malonate (32.3 g, 191.7 mmol) in CH₃CN (200 mL) was added MgCl₂ (15.2, 64.0 mmol) and TEA (44.8 g, 435.6 mmol) in portions. The mixture was stirred 50° C. for 2 h. The solution in flask A was transferred to the slurry in flask B and the mixture was stirred at 70° C. for 12 h. The reaction mixture was quenched with HCl (3N, 600 mL) and the solution was concentrated under reduced pressure to remove solvent. The resulting concentrate extracted with MTBE (150 mL×3). The organic layer was washed with H₂O (150 mL×3), saturate NaHCO₃ (150 mL×3), and saturated NaCl (150 mL), dried over anhydrous Na₂SO₄, filtered and concentrated under reduced pressure to afford compound 58A (18 g, 46.9% yield) as colorless oil, which was used directly in next step. ¹H NMR (DMSO-d₆, 400 MHz) 8.59 (d, J=8.4 Hz, 1H), 8.19-8.15 (m, 2H), 8.03 (d, J=7.7 Hz, 1H), 7.68-7.58 (m, 3H), 4.31 (s, 2H), 4.09 (q, J=7.1 Hz, 2H), 1.11 (t, J=7.2 Hz, 3H). MS (ESI) Dm/z (M+H)⁺ 243.1.

To a mixture of compound 58A (18 g, 74.3 mmol, 1 eq) in EtOH (150 mL) was added NH₄OAc (45.8 g, 594.4 mmol) in one portion. The mixture was stirred at 90° C. for 24 h. The solvent was removed and concentrated under reduced pressure. EA (100 ml) and H₂O (50 mL) were added to the mixture, the organic layer was separated. The aqueous was extracted with EA (50 mL×2), the combined organic layer was washed with water (100 ml×2), saturate NaHCO₃ (100 mL×2), brine (100 mL×2). Then dried over anhydrous Na₂SO₄, filtered, concentrated under reduced pressure. The crude product was purified by column chromatography (SiO₂, Petroleum ether/Ethyl acetate=20/1 to 5/1) to afford compound 58B (16 g, 81.2% yield) as colorless oil. ¹H NMR (DMSO-d₆, 400 MHz) δ 8.21 (br. s, 1H), 8.13-8.06 (m, 1H), 8.02-7.95 (m, 2H), 7.61-7.42 (m, 5H), 4.51 (s, 1H), 4.08 (q, J=7.1 Hz, 2H), 1.21 (t, J=7.1 Hz, 3H). MS (ESI) m/z (M+H)⁺ 242.0.

Pyridine (10 mL, 124.3 mmol) was added to a stirred solution of compound 58B (3 g, 12.4 mmol) in toluene (20 mL) and the mixture reaction was cooled to 0° C. Acetyl chloride (6.7 mL, 93.3 mmol) was added dropwise, and the mixture was stirred for 6 h at 0° C. under an atmosphere of nitrogen. The compound 58B was monitored by LCMS, so additional acetyl chloride (20 mL, 279.8 mmol) was added into the reaction mixture and the mixture was stirred for 12 h at 0° C. under an atmosphere of nitrogen. The reaction was quenched with brine (30 ml), extracted with EA (50 ml×3) and dried over Na₂SO₄, and the solvent was evaporated in vacuo. The crude product was purified by column chromatography (SiO₂, PE/EA=20/1 to 5/1) to afford compound 58C (2.5 g, 66.4% yield) as white solid. ¹H NMR (DMSO-d₆, 400 MHz) δ 10.89 (s, 1H), 7.99-7.87 (m, 3H), 7.58-7.36 (m, 4H), 5.22-5.14 (m, 1H), 4.20 (q, J=7.1 Hz, 2H), 2.01 (s, 3H), 1.26 (t, J=7.2 Hz, 3H). MS (ESI) m/z (M+H)⁺ 284.1.

[Bis(trifluoroacetoxy)iodo]benzene (986.6 mg, 2.3 mmol) was added into a stirred solution of compound 58C (0.5 g, 1.8 mmol) in 2,2,2-trifluoroethanol (15 mL). The mixture was stirred for 30 min at 25° C. The reaction was quenched with saturated aqueous NaHCO₃ (20 ml) and the mixture diluted with EtOAc (20 ml) and extracted with EtOAc (20 ml×2). The organic layers were washed with water (15 ml×2), brine (15 ml), dried over Na₂SO₄, filtered and concentrated in vacuo The crude product was purified by column chromatography (SiO₂, Petroleum ether/Ethyl acetate=20/1 to 5/1) to afford compound 58D (380 mg, 74.2% yield) as pale yellow solid. ¹H NMR (DMSO-d₆, 400 MHz) δ 8.02 (dd, J=7.8, 14.6 Hz, 2H), 7.83 (d, J=8.3 Hz, 1H), 7.65-7.48 (m, 4H), 4.09 (q, J=7.0 Hz, 2H), 2.62 (s, 3H), 0.98 (t, J=7.0 Hz, 3H). MS (ESI) m/z (M+H)⁺ 282.0.

Compound 58D was hydrolyzed to yield intermediate 58E and this was reacted with intermediate 1D using the same procedure as described in Example 1 to yield compound 58. Compound 58 (0.140 g, yield 64.8%) was obtained as yellow solid, ¹H NMR (DMSO-4 400 MHz) δ 8.63 (d, J=7.5 Hz, 1H), 8.06 (s, 1H), 7.97 (br d, J=7.8 Hz, 2H), 7.86-7.76 (m, 2H), 7.58-7.42 (m, 4H), 7.32-7.18 (m, 5H), 5.37-5.27 (m, 1H), 3.15 (br dd, J=3.4, 13.9 Hz, 1H), 2.94 (br dd, J=9.8, 13.8 Hz, 1H), 2.61 (s, 3H). MS (ESI) m/z (M+H)⁺ 428.1.

### N-(4-AMINO-3,4-DIOXO-1-PHENYLBUTAN-2-YL)-4-(2-FLUORO-3-METHOXYPHENYL)-2-METHYLOXAZOLE-5-CARBOXAMIDE (75)

Compound 75 was prepared from 2-fluoro-3-methoxybenzoic acid using the same procedures as described for compound 58 to yield the compound 75. Compound 75 (0.160 g, yield 53.6%) was obtained as yellow solid, ¹H NMR (DMSO-d₆, 400 MHz) δ 8.71 (d, J=7.6 Hz, 1H), 8.03 (s, 1H), 7.78 (s, 1H), 7.30-7.05 (m, 7H), 6.97-6.89 (m, 1H), 5.37-5.27 (m, 1H), 3.80 (s, 3H), 3.13 (dd, J=3.9, 13.9 Hz, 1H), 2.93 (dd, J=9.8, 14.2 Hz, 1H), 2.51 (s, 3H). MS (ESI) m/z (M+H)⁺ 426.1.

### N-(4-AMINO-3,4-DIOXO-1-PHENYLBUTAN-2-YL)-4-(2,6-DIFLUOROPHENYL)-2-METHYLOXAZOLE-5-CARBOXAMIDE (76)

Compound 76 was prepared from 2,6-difluorobenzoic acid using the same procedures as described for compound 58 to yield the compound 76. Compound 76 (0.153 g, yield 53.8%) was obtained as yellow solid, ¹H NMR (DMSO-d₆, 400 MHz) δ 8.88 (d, J=7.3 Hz, 1H), 8.07 (s, 1H), 7.82 (s, 1H), 7.58-7.46 (m, 1H), 7.35-7.07 (m, 7H), 5.39-5.28 (m, 1H), 3.16 (dd, J=3.5, 14.1 Hz, 1H), 2.96 (dd, J=10.0, 14.2 Hz, 1H), 2.57 (s, 3H). MS (ESI) m/z (M+H)⁺ 414.1.

### N-(4-AMINO-1-(4-FLUOROPHENYL)-3,4-DIOXOBUTAN-2-YL)-4-(2-FLUOROPHENYL)-2-METHYLOXAZOLE-5-CARBOXAMIDE (73)

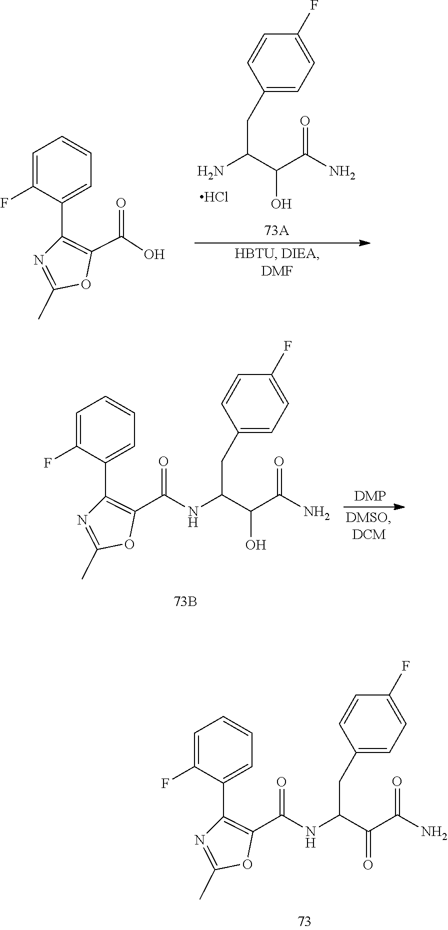 [SMILES: Cc1nc(-c2ccccc2F)c(C(=O)O)o1 ; Cl ; NC(=O)C(O)C(N)Cc1ccc(F)cc1 ; Cc1nc(-c2ccccc2F)c(C(=O)NC(Cc2ccc(F)cc2)C(O)C(N)=O)o1 ; Cc1nc(-c2ccccc2F)c(C(=O)NC(Cc2ccc(F)cc2)C(=O)C(N)=O)o1]

Compound 73 was prepared from 4-(2-fluorophenyl)-2-methyloxazole-5-carboxylic acid and intermediate 73A using the same procedures as described for Example 1 to yield the compound 73. Compound 73 (0.160 g, yield 73.08%) was obtained as white solid, ¹H NMR (DMSO-d₆, 400 MHz): δ 8.80 (d, J=7.3 Hz, 1H), 8.05 (s, 1H), 7.81 (s, 1H), 7.45 (q, J=7.3 Hz, 2H), 7.33-7.25 (m, 2H), 7.24-7.17 (m, 2H), 7.11 (t, J=8.8 Hz, 2H), 5.32 (s, 1H), 3.15 (dd, J=3.4, 13.9 Hz, 1H), 3.02-2.87 (m, 1H), 2.55 (s, 3H). MS (ESI) m/z (M+H)⁺ 414.1.

### N-(4-AMINO-3,4-DIOXO-1-PHENYLBUTAN-2-YL)-4-(2,5-DIMETHYLFURAN-3-YL)-2-METHYLOXAZOLE-5-CARBOXAMIDE (78)

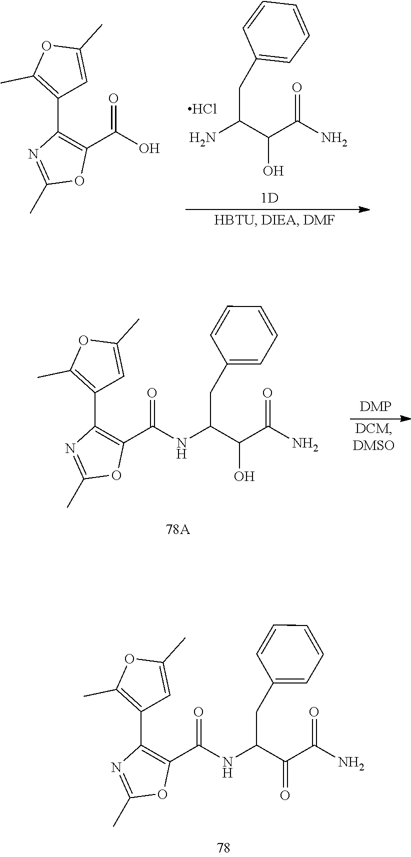 [SMILES: Cc1cc(-c2nc(C)oc2C(=O)O)c(C)o1 ; Cl ; NC(=O)C(O)C(N)Cc1ccccc1 ; Cc1cc(-c2nc(C)oc2C(=O)NC(Cc2ccccc2)C(O)C(N)=O)c(C)o1 ; Cc1cc(-c2nc(C)oc2C(=O)NC(Cc2ccccc2)C(=O)C(N)=O)c(C)o1]

Compound 78 was prepared from 4-(2,5-dimethylfuran-3-yl)-2-methyloxazole-5-carboxylic acid and intermediate 1D using the same procedures as described for compound 58 to yield the compound 78. Compound 78 (65 mg, yield 40.9%) was obtained as white solid, ¹H NMR (400 MHz, DMSO-d₆) δ 8.60 (d, J=7.3 Hz, 1H), 8.14-8.04 (m, 1H), 7.81 (s, 1H), 7.29-7.23 (m, 4H), 7.20-7.15 (m, 1H), 6.57 (s, 1H), 5.39-5.34 (m, 1H), 3.16 (dd, J=3.8, 13.8 Hz, 1H), 2.95 (dd, J=9.8, 13.9 Hz, 1H), 2.46 (s, 3H), 2.36 (s, 3H), 2.19-2.12 (m, 3H). MS (ESI) m/z (M+H)⁺ 396.1.

### N-(4-AMINO-3,4-DIOXO-1-PHENYLBUTAN-2-YL)-4-(2,5-DICHLOROFURAN-3-YL)-2-METHYLOXAZOLE-5-CARBOXAMIDE (81)

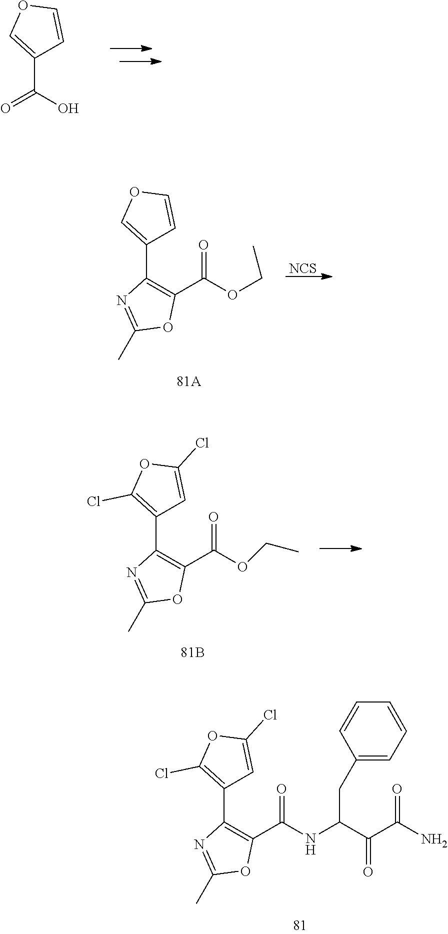

Compound 81A was prepared from furan-3-carboxylic acid using the same procedures as described for compound 58D to yield the compound 81A. Compound 81A (1.28 g, yield 64.2%) was obtained as white solid, ¹H NMR (400 MHz, CDCl₃) δ 8.37 (s, 1H), 7.48 (s, 1H), 7.13-7.07 (m, 1H), 4.43 (q, J=7.3 Hz, 2H), 2.55 (s, 3H), 1.43 (t, J=7.1 Hz, 3H). MS (ESI) m/z (M+H)⁺ 221.9.

To a solution of compound 81A (300 mg, 1.36 mmol) in DMF (3 mL) was added NCS (580 mg, 4.34 mmol). The mixture was stirred at 100° C. for 6 br. The reaction was diluted with H₂O (30 mL), extracted with EA (20 mL×3), the organic phase was dried over Na₂SO₄, filtered, and concentrated to give a residue. The residue was purified by flash silica gel chromatography (PE:EA=10:1 to 5:1). Compound 81B (80 mg, yield: 20.3%) was obtained as a white solid. ¹H NMR (400 MHz, CDCl₃) δ 6.85 (s, 1H), 4.40 (q, J=7.2 Hz, 2H), 2.58 (s, 3H), 1.39 (br t, J=7.1 Hz, 3H).

Compound 81B was hydrolyzed to yield the intermediate acid which was reacted with intermediate 1D using the same procedure as described in Example 1 to yield compound 81. Compound 81 (68 mg, yield 88.3%) was obtained as pale yellow solid, ¹H NMR (400 MHz, DMSO-d₆) δ 8.93 (d, J=7.6 Hz, 1H), 8.10 (s, 1H), 7.83 (s, 1H), 7.27-7.24 (m, 4H), 7.19-7.15 (m, 1H), 7.00 (s, 1H), 5.42-5.31 (m, 1H), 3.22-3.13 (m, 1H), 2.96-2.89 (m, 1H), 2.50 (s, 3H). MS (ESI) m/z (M+H)⁺ 436.0.

### N-(4-AMINO-3,4-DIOXO-1-PHENYLBUTAN-2-YL)-2-METHYL-4-(2-METHYLFURAN-3-YL)OXAZOLE-5-CARBOXAMIDE (84)

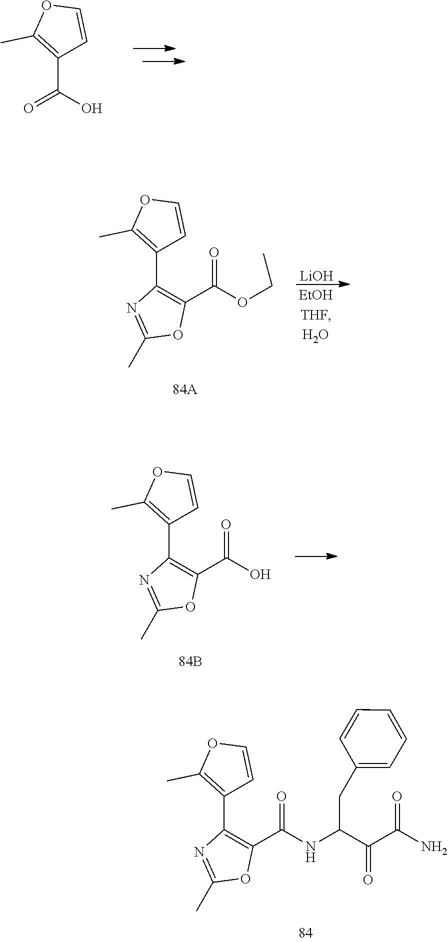

Compound 84 was prepared from 2-methylfuran-3-carboxylic acid via intermediates 84A and 84B using the same procedures as described for compound 58 to yield the compound 84. Compound 84 (60 mg, yield 37.52%) was obtained as white solid. MS (ESI) m/z (M+1)⁺ 382.1. ¹H NMR (DMSO-d₆, 400 MHz): δ 8.65 (d, J=7.2 Hz, 1H), 8.08 (br. s, 1H), 7.81 (br. s, 1H), 7.45 (d, J=2.0 Hz, 1H), 7.30-7.21 (m, 4H), 7.21-7.13 (m, 1H), 6.94 (d, J=2.0 Hz, 1H), 5.45-5.32 (m, 1H), 3.21-3.09 (m, 1H), 3.01-2.88 (m, 1H), 2.48 (s, 3H), 2.39 (s, 3H).

### N-(4-AMINO-3,4-DIOXO-1-PHENYLBUTAN-2-YL)-4-(BENZO[b]THIOPHEN-4-YL)-2-METHYLOXAZOLE-5-CARBOXAMIDE (88)

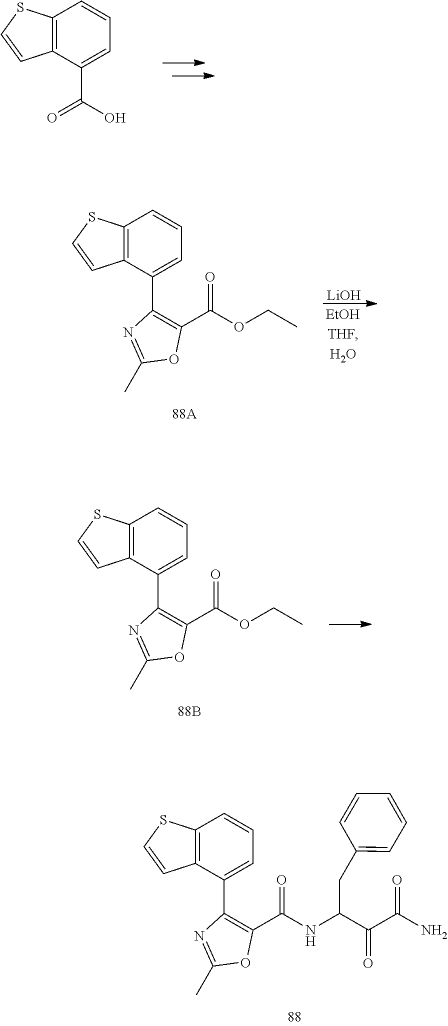

Compound 88 was prepared from benzo[b]thiophene-4-carboxylic acid via intermediates 88A and 88B using the same procedures as described for compound 58 to yield the compound 88. Compound 88 (110 mg, yield 92.6%) was obtained as yellow solid. ¹H NMR (400 MHz, DMSO-d₆) δ 8.73 (d, J=7.3 Hz, 1H), 8.07-7.98 (m, 2H), 7.80 (s, 1H), 7.71 (d, J=5.6 Hz, 1H), 7.52-7.47 (m, 1H), 7.37 (d, J=5.4 Hz, 1H), 7.32 (d, J=7.8 Hz, 1H), 7.30-7.16 (m, 5H), 5.38-5.28 (m, 1H), 3.14 (dd, J=3.5, 13.8 Hz, 1H), 2.92 (dd, J=9.9, 14.1 Hz, 1H), 2.56 (s, 3H). MS (ESI) m/z (M+H)⁺⁼434.1.

### N-(4-AMINO-3,4-DIOXO-1-PHENYLBUTAN-2-YL)-4-(2-CHLOROFURAN-3-YL)-2-METHYLOXAZOLE-5-CARBOXAMIDE (90)

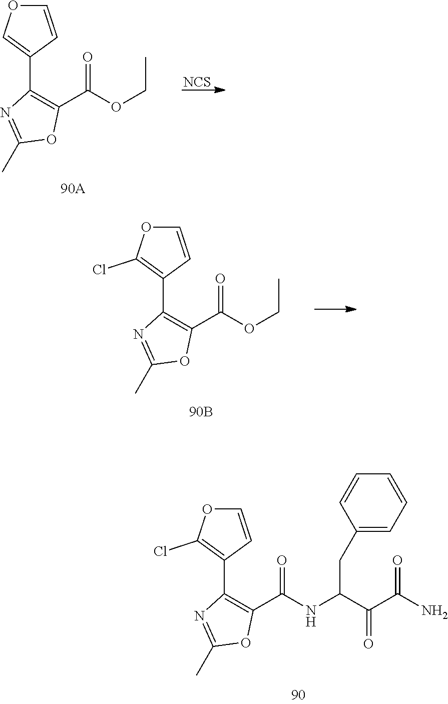

To a solution of compound 90A (400 mg, 1.81 mmol) in DMF (3 mL) was added NCS (266 mg, 1.99 mmol). The mixture was stirred at 15° C. for 16 h. Then the mixture was stirred at 25° C. for 16 h. The reaction was diluted with H₂O (40 mL), extracted with EA (30 mL×2), the organic phase was dried over Na₂SO₄, filtered, and concentrated to give a residue. The residue was purified by flash silica gel chromatography (PE:EA=10:1 to 4:1). Compound 90B (300 mg, yield: 64.9%) was obtained as a white solid. ¹H NMR (400 MHz, CDCl₃) δ 7.37 (d, J=2.0 Hz, 1H), 6.98 (d, J=2.2 Hz, 1H), 4.46-4.34 (m, 2H), 2.62-2.53 (m, 3H), 1.46-1.33 (m, 3H).

Compound 90B was hydrolyzed to yield the intermediate acid which was reacted with intermediate 1D using the same procedure as described in Example 1 to yield compound 90. Compound 90 (90 mg, yield 51.8%) was obtained as white solid, ¹H NMR (400 MHz, DMSO-d) 8 8.86 (d, J=7.6 Hz, 1H), 8.12 (s, 1H), 7.85 (s, 1H), 7.73-7.68 (m, 1H), 7.32-7.26 (m, 4H), 7.25-7.17 (m, 1H), 7.07-6.99 (m, 1H), 5.43-5.38 (m, 1H), 3.19 (dd, J=3.8, 14.1 Hz, 1H), 2.97 (dd, J=10.0, 13.9 Hz, 1H), 2.53 (s, 3H). MS (ESI) m/z (M+H)⁺ 402.1.

### N-(4-AMINO-3,4-DIOXO-1-PHENYLBUTAN-2-YL)-4-(BENZO[b]THIOPHEN-7-YL)-2-METHYLOXAZOLE-5-CARBOXAMIDE (91)

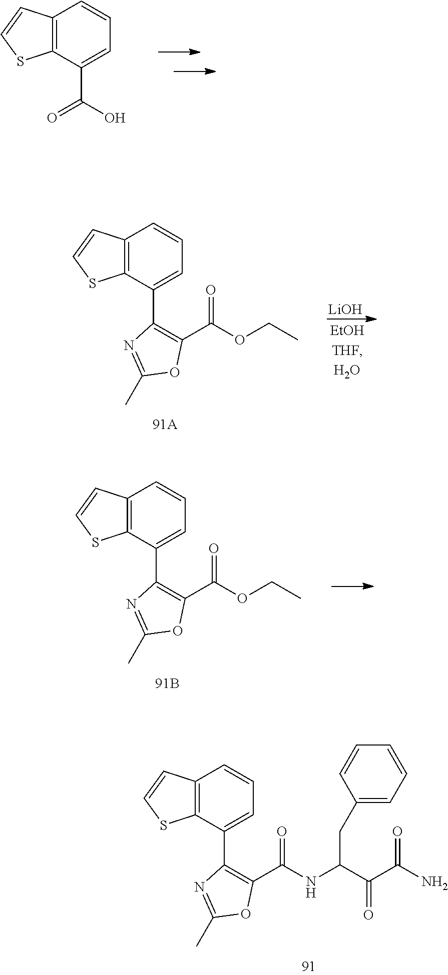

Compound 91 was prepared from benzo[b]thiophene-7-carboxylic acid via intermediates 91A and 91B using the same procedures as described for compound 58 to yield the compound 91. Compound 91 (15 mg, yield 49.6%) was obtained as white solid. ¹H NMR (400 MHz, DMSO-d₆) δ 8.90 (d, J=7.5 Hz, 1H), 8.12 (s, 1H), 8.02 (d, J=7.3 Hz, 1H), 7.93-7.85 (m, 2H), 7.75 (d, J=5.8 Hz, 1H), 7.48 (d, J=5.8 Hz, 1H), 7.39 (d, J=7.8 Hz, 1H), 7.31-7.28 (m, 3H), 7.25-7.16 (m, 2H), 5.45-5.41 (m, 1H), 3.20 (dd, J=3.9, 13.9 Hz, 1H), 2.98 (dd, J=9.8, 13.8 Hz, 1H), 2.62 (s, 3H). MS (ESI) m/z (M+H)⁺ 434.1.

### N-(4-AMINO-3,4-DIOXO-1-PHENYLBUTAN-2-YL)-4-(5-CHLOROFURAN-3-YL)-2-METHYLOXAZOLE-5-CARBOXAMIDE (92)

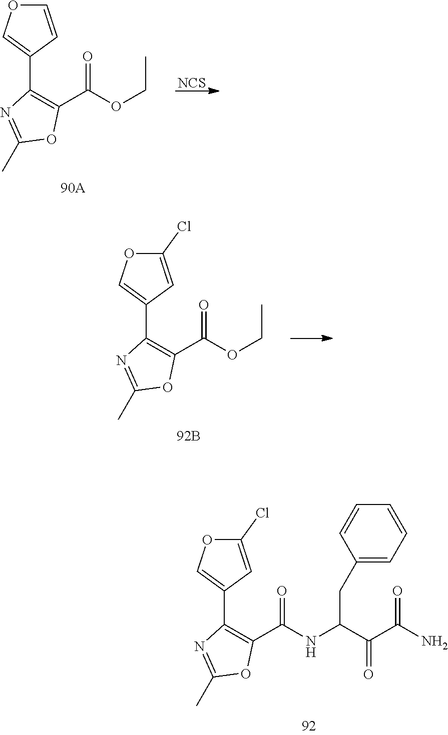

To a solution of compound 90A (400 mg, 1.81 mmol) in DMF (3 mL) was added NCS (266 mg, 1.99 mmol). The mixture was stirred at 15° C. for 16 h. Then the mixture was stirred at 25° C. for 16 h. The reaction was diluted with H₂O (40 mL), extracted with EA (30 mL×2), the organic phase was dried over Na₂SO₄, filtered, and concentrated to give a residue. The residue was purified by flash silica gel chromatography (PE:EA=10:1 to 4:1). Compound 92B (55 mg, yield: 11.9%) was obtained as a white solid. ¹H NMR (400 MHz, CDCl₃) δ 8.22 (s, 1H), 6.93 (d, J=1.0 Hz, 1H), 4.46-4.40 (m, 2H), 2.54 (s, 3H), 1.42 (t, J=7.1 Hz, 3H).

Compound 92B was hydrolyzed to yield the intermediate acid which was reacted with intermediate 1D using the same procedure as described in Example 1 to yield compound 92. Compound 92 (45 mg, yield 61.3%) was obtained as white solid, ¹H NMR (400 MHz, DMSO-d₆) δ 8.90 (d, J=7.6 Hz, 1H), 8.33 (d, J=1.0 Hz, 1H), 8.15 (s, 1H), 7.87 (s, 1H), 7.34-7.27 (m, 4H), 7.24-7.16 (m, 1H), 7.02 (d, J=1.0 Hz, 1H), 5.45-5.41 (m, 1H), 3.21 (dd, J=3.9, 13.9 Hz, 1H), 3.00 (dd, J=9.9, 14.1 Hz, 1H), 2.53 (s, 3H). MS (ESI) m/z (M+H)⁺ 402.1.

### N-(4-AMINO-3,4-DIOXO-1-PHENYLBUTAN-2-YL)-4-(5-CHLORO-2-METHYLFURAN-3-YL)-2-METHYLOXAZOLE-5-CARBOXAMIDE (98)

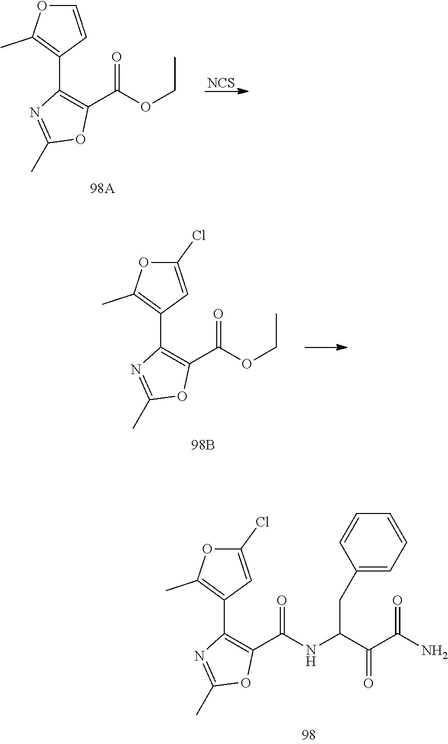

To a solution of compound 98A (100 mg, 0.42 mmol) in DMF (5 mL) was added NCS (57 mg, 0.42 mmol). The mixture was stirred at 20° C. for 12 h. The mixture was washed with H₂O (20 mL), extracted with EtOAc (15 mL×2). The organics were collected and concentrated. The residue was purified by column (PE:EA=10:1) to give compound 2 (60 mg, yield: 52.34%) as colorless solid. ¹H NMR (DMSO-d₆, 400 MHz): δ 6.94 (s, 1 h), 4.34-4.27 (m, 2H), 2.53 (s, 3H), 2.50 (s, 3H), 1.31-1.27 (m, 3H).

Compound 98B was hydrolyzed to yield the intermediate acid which was reacted with intermediate 1D using the same procedure as described in Example 1 to yield compound 98. Compound 98 (80 mg, yield 40.15%) was obtained as light yellow solid, MS (ESI) m/z (M+1)+416.1. ¹H NMR (DMSO-d₆, 400 MHz): 8.77 (d, J=7.6 Hz, 1H), 8.09 (br. s, 1H), 7.82 (br. s, 1H), 7.29-7.22 (m, 4H), 7.20-7.13 (m, 1H), 6.89 (s, 1H), 5.41-5.32 (m, 1H), 3.20-3.12 (m, 1H), 3.00-2.89 (m, 1H), 2.49 (s, 3H), 2.41 (s, 3H).

### 2-(5-(ETHOXYCARBONYL)-2-METHYLOXAZOL-4-YL)-N,N,N-TRIMETHYLBENZENAMINIUM (105)

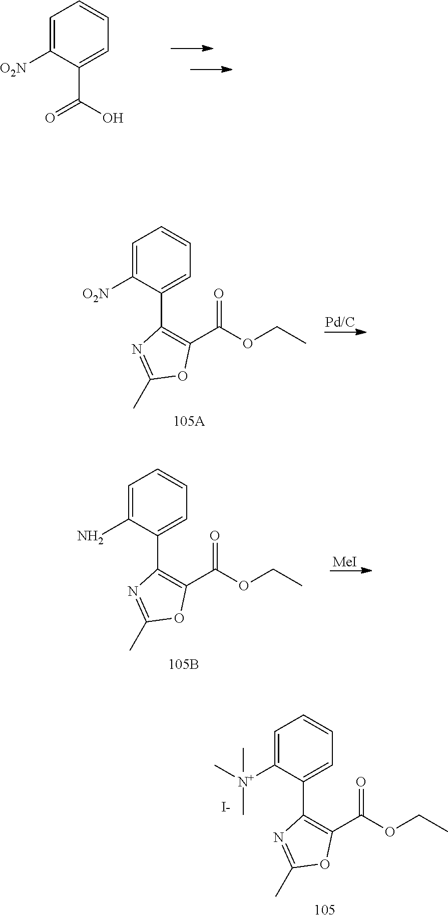 [SMILES: O=C(O)c1ccccc1[N+](=O)[O-] ; "MeI" ; CCOC(=O)c1oc(C)nc1-c1ccccc1[N+](=O)[O-] ; CCOC(=O)c1oc(C)nc1-c1ccccc1N ; CCOC(=O)c1oc(C)nc1-c1ccccc1[N+](C)(C)C ; [I-]]

2-nitrobenzoic acid was subjected to conditions as described for compound 58 to yield the compound 105A. Compound 105A (480 mg, 60.4% yield) was obtained as a yellow oil. ¹H NMR (400 MHz, DMSO-d₆) δ 8.14-8.09 (m, 1H), 7.87-7.80 (m, 1H), 7.78-7.70 (m, 2H), 4.21-4.13 (m, 2H), 2.58 (s, 3H), 1.17-1.11 (m, 3H).

To a solution of compound 105A (200 mg, 724.00 umol) in EtOH (20 mL) was added Pd/C (45 mg, 72.40 umol, 10% purity) and NH₃.H₂O (2.17 mmol, 270 uL, 30% purity). The mixture was stirred at 25° C. for 1 hr under H₂ balloon (15 psi). The mixture was filtered and concentrated. The residue was purified by column chromatography (SiO₂, Petroleum ether/Ethyl acetate=2/1 to 0/1). Compound 105B (75 mg, 42.1% yield) was obtained as a yellow solid. ¹H NMR (400 MHz, CDCl₃) δ 7.58-7.48 (m, 1H), 7.22-7.16 (m, 1H), 6.79-6.72 (m, 2H), 4.67 (br s, 2H), 4.37-4.30 (m, 2H), 2.58 (s, 3H), 1.34-1.28 (m, 3H).

To a solution of Compound 105B (120 mg, 487.29 umol) and MeI (2.77 g, 19.49 mmol, 1.21 mL) in acetone (3 mL) was added K₂CO₃ (300 mg, 2.17 mmol). The mixture was stirred at 40° C. for 48 h, and added MeI (2.77 g, 19.49 mmol, 1.21 mL). The mixture was stirred at 40° C. for 48 h, The reaction was filtered, the filtrate was concentrated, The residue was purified by preparatory-TLC (SiO₂, DCM:EA=1:1). Compound 105 (40 mg, 27.2% yield) was obtained as a yellow solid. ¹H NMR (400 MHz, DMSO-d₆) δ 8.10 (d, J=8.3 Hz, 1H), 7.76 (t, J=7.5 Hz, 1H), 7.64 (t, J=7.4 Hz, 1H), 7.48 (d, J=7.3 Hz, 1H), 4.13 (q, J=7.2 Hz, 2H), 3.74-3.47 (m, 9H), 2.61 (s, 3H), 1.06 (t, J=7.0 Hz, 3H). MS (ESI) m/z (M+H)⁺ 433.1.

### 3-(4-(2,6-DIFLUOROPHENYL)-2-METHYLOXAZOLE-5-CARBOXAMIDO)-2-OXO-4-PHENYLBUTANOIC ACID (108)

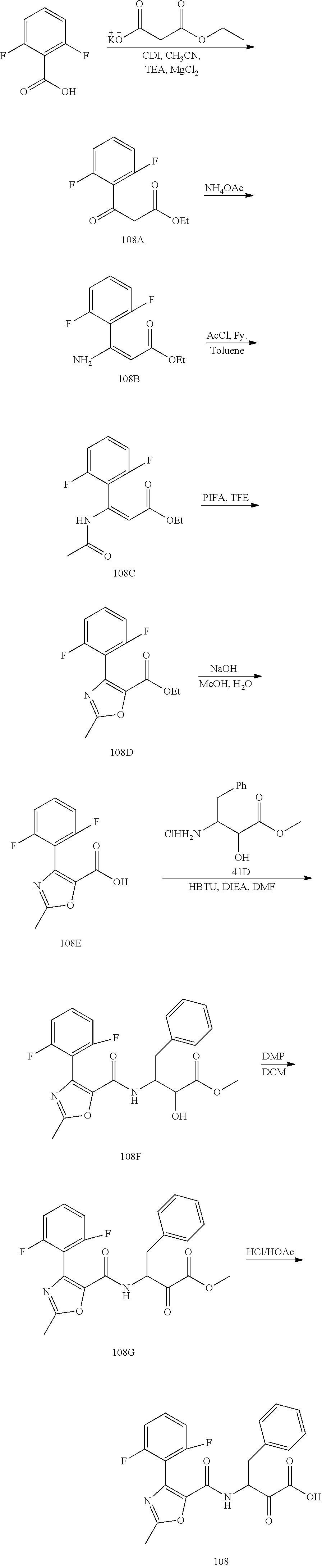

Compound 108 was prepared from 2,6-difluorobenzoic acid using the same procedures as described for compound 58 to yield the compound 108. Compound 108 (0.025 g, yield 25.17%) was obtained as white solid, ¹H NMR (DMSO-d₆, 400 MHz) δ₁ 7.52-7.39 (m, 2H), 7.32-7.27 (m, 2H), 7.26-7.19 (m, 3H), 7.04 (br t, J=8.0 Hz, 2H), 5.29-5.20 (m, 1H), 3.30 (br dd, J=4.9, 14.2 Hz, 1H), 3.00 (br dd, J=9.0, 14.1 Hz, 1H), 2.51 (s, 3H). MS (ESI) m/z (M+H)⁺ 415.2.

### Example 20

### COMPOUNDS 80,83,87,89,95,96,97, AND 115

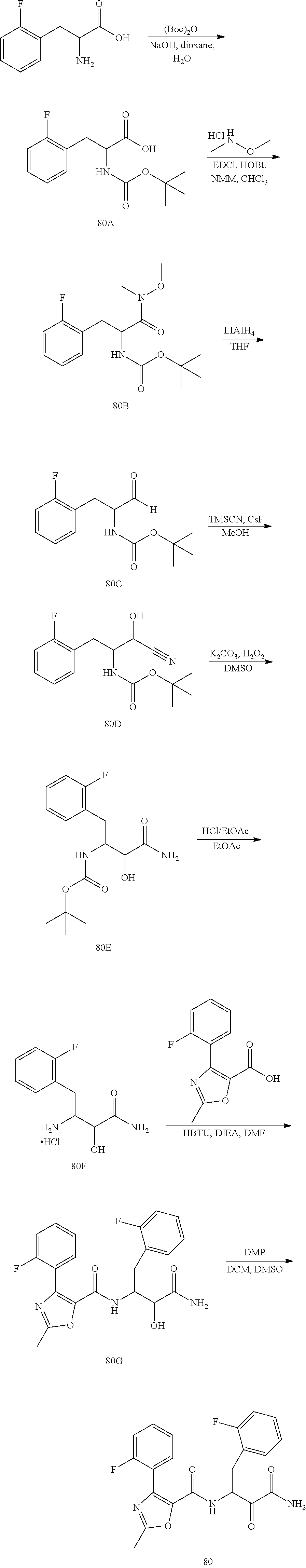

### N-(4-AMINO-1-(2-FLUOROPHENYL)-3,4-DIOXOBUTAN-2-YL)-4-(2-FLUOROPHENYL)-2-METHYLOXAZOLE-5-CARBOXAMIDE (80)

To a solution of 2-amino-3-(2-fluorophenyl)propanoic acid (5.77 g, 31.50 mmol) in dioxane (45 mL) was added NaOH (1.95 g, 48.82 mmol) in H₂O (12 mL) and Boc₂O (8.66 g, 39.69 mmol, 9.12 mL) in dioxane (15 mL). The mixture was stirred at 25° C. for 20 h. The reaction was concentrated under reduced pressure and H₂O (60 mL) was added to the mixture. The aqueous was treated with HCl (0.5M) until pH ˜3 and the reaction was extracted with EA (50 mL×3). The combined organic layer was washed with H₂O (50 mL) and brine (50 mL), dried over anhydrous Na₂SO₄, filtered, concentrated to give a residue. Compound 80A (8.58 g, yield: 96.2%) was obtained as a yellow solid which was used into the next step without further purification. ¹H NMR (400 MHz, DMSO-d₆) δ 12.66 (br s, 1H), 7.35-7.22 (m, 2H), 7.17-7.07 (m, 3H), 4.19-4.07 (m, 1H), 3.13 (br dd, J=4.9, 13.9 Hz, 1H), 2.81 (br dd, J=10.5, 13.7 Hz, 1H), 1.30 (s, 9H).

To a mixture of compound 80A (8.58 g, 30.29 mmol) and N-methoxymethanamine (4.14 g, 42.41 mmol, HCl), HOBt (4.50 g, 33.32 mmol) in CHCl₃ (100 mL) was added NMM (12.25 g, 121.16 mmol, 13.32 mL) dropwise. Then EDCI (8.13 g, 42.41 mmol) was added to the mixture and the mixture was stirred at 25° C. for 18 h. The reaction was concentrated under reduced pressure. H₂O (100 mL) and EA (100 mL) were added to the mixture, the organic layer was separated. The aqueous layer was extracted with EA (60 mL×2). The combined organic layer was washed with HCl (0.5M, 100 mL), saturated NaHCO₃ (100 mL), dried over anhydrous Na₂SO₄, filtered, concentrated under reduced pressure to give a residue. Compound 80B (9.26 g, yield: 91.7%) was obtained as a yellow solid, which was used into the next step without further purification. ¹H NMR (400 MHz, DMSO-d₆) δ 7.38-7.19 (m, 2H), 7.17-6.99 (m, 3H), 4.66 (br s, 1H), 3.67 (br s, 3H), 3.13-3.02 (m, 3H), 2.95 (br dd, J=4.5, 13.6 Hz, 1H), 2.76-2.61 (m, 1H), 1.27 (s, 9H). MS (ESI) m/z (M+Na)+348.9.

To a solution of LiAlH₄ (1.18 g, 31.21 mmol) in THF (50 mL) was added dropwise a solution of compound SOB (9.26 g, 28.37 mmol) in THF (100 mL) at 0° C. under N₂ atmosphere. After addition, the mixture was stirred at 0° C. for 2 h. The reaction mixture was added EA (100 mL) and HCl (1M, 100 mL) at 0° C. The organic layer was separated and the aqueous layer was extracted with EA (100 mL×2). The combined organic layer was washed with HCl (1M, 100 mL), H₂O (100 mL), brine (100 mL). The combined organic layer was dried over anhydrous Na₂SO₄, filtered, concentrated under reduced pressure to give a residue. Compound 80C (5.65 g, yield: 74.5%) was obtained as a yellow oil, which was used into the next step without further purification. ¹H NMR (400 MHz, DMSO-d₆) δ 9.50 (s, 1H), 7.37 (br d, J=7.3 Hz, 1H), 7.31-7.22 (m, 2H), 7.16-7.08 (m, 2H), 4.03 (q, J=6.8 Hz, 1H), 3.13 (br dd, J=4.6, 13.9 Hz, 1H), 2.74 (br dd, J=10.1, 13.6 Hz, 1H), 1.32 (s, 9H).

To a solution of compound 80C (2 g, 7.48 mmol) and CsF (568 mg, 3.74 mmol) in MeOH (50 mL) was added drop wised trimethylsilylformonitrile (890.76 mg, 8.98 mmol, 1.12 mL) at 0° C. The mixture was warmed to 20° C. and stirred for 5 h. The reaction mixture was concentrated, then diluted with H₂O (30 mL), extracted with EA (30 mL×3), the combined organic layers were dried over Na₂SO₄, filtered and concentrated to give a residue. Compound 80D (2.62 g, crude) was obtained as a yellow oil, which was used into the next step without further purification. ¹H NMR (400 MHz, DMSO-d₆) δ 7.23 (br d, J=7.6 Hz, 2H), 7.15-7.03 (m, 3H), 4.63-4.28 (m, 1H), 3.93-3.75 (m, 1H), 3.12-2.93 (m, 1H), 2.78-2.58 (m, 1H), 1.25 (s, 4.5H), 1.22 (s, 4.5H).

To a solution of compound 80D (530 mg, 1.80 mmol) in DMSO (10 mL) was added K₂CO₃ (498 mg, 3.60 mmol) and H₂O₂ (3.06 g, 27.01 mmol, 2.60 mL, 30% purity) was added dropwise to the mixture. The mixture was stirred at 20° C. for 3 h. The reaction was quenched with saturated Na₂S₂O₃ (20 mL) and diluted with H₂O (30 mL). The mixture was extracted with EA (40 mL×3) and the combined organic layer was washed with H₂O (40 mL), brine (40 mL), dried over anhydrous Na₂SO₄, filtered, concentrated to give a residue. Compound 80E (507 mg, yield: 90.1%) was obtained as a pale yellow solid, which was used into the next step without further purification. ¹H NMR (400 MHz, DMSO-d₆) δ 7.34-7.16 (m, 4H), 7.14-7.04 (m, 2H), 6.52-6.04 (m, 1H), 5.69 (dd, J=6.0, 12.6 Hz, 1H), 4.04 (br d, J=8.8 Hz, 1H), 3.94-3.74 (m, 1H), 2.90-2.61 (m, 2H), 1.24 (s, 9H).

To a solution of compound 80E (1.39 g, 4.45 mmol) in EtOAc (15 mL) was added HC/EtOAc (4M, 15 mL). The mixture was stirred at 25° C. for 2 h. The precipitate was filtered and filtered cake was washed with EA (20 mL). The solid was dried under reduced pressure. Compound 80F (933 mg, yield: 84.3%, HCl) was obtained as a pale yellow solid. ¹H NMR (400 MHz, DMSO-d₆) δ 8.17-7.90 (m, 3H), 7.55-7.43 (m, 2H), 7.43-7.23 (m, 2H), 7.21-7.07 (m, 2H), 6.74-6.36 (m, 1H), 4.23-3.77 (m, 1H), 3.72-3.53 (m, 1H), 2.92 (br d, J=7.1 Hz, 1H), 2.82 (br d, J=7.1 Hz, 1H).

Compound 80F and 4-(2-fluorophenyl)-2-methyloxazole-5-carboxylic acid were coupled using the same conditions as for intermediates 58E and 1D and then used procedures as described in Example 1 to yield compound 80. Compound 80 (95 mg, yield 60.7%) was obtained as white solid, ¹H NMR (400 MHz, DMSO-d) 8 8.77 (d, J=7.3 Hz, 1H), 8.01 (s, 1H), 7.75 (s, 1H), 7.50-7.38 (m, 2H), 7.32-7.17 (m, 4H), 7.16-7.06 (m, 2H), 5.39-5.29 (m, 1H), 3.22 (br dd, J=4.8, 14.3 Hz, 1H), 3.01 (dd, J=9.0, 13.9 Hz, 1H), 2.53 (s, 3H). MS (ESI) m/z (M+H)⁺ 414.1.

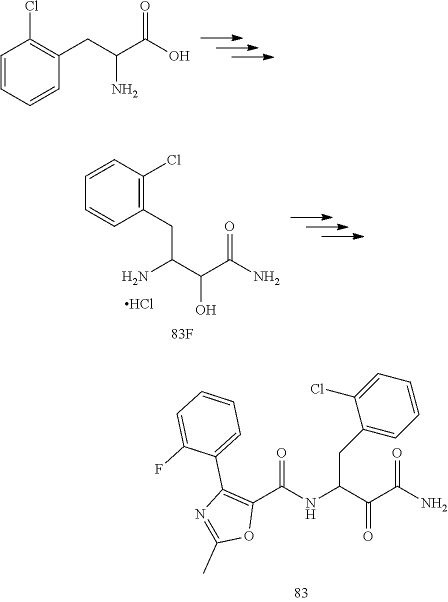 [SMILES: NC(Cc1ccccc1Cl)C(=O)O ; NC(=O)C(O)C(N)Cc1ccccc1Cl ; Cl ; Cc1nc(-c2ccccc2F)c(C(=O)NC(Cc2ccccc2Cl)C(=O)C(N)=O)o1]

### N-(4-AMINO-1-(2-CHLOROPHENYL)-3,4-DIOXOBUTAN-2-YL)-4-(2-FLUOROPHENYL)-2-METHYLOXAZOLE-5-CARBOXAMIDE (83)

Compound 2-amino-3-(2-chlorophenyl)propanoic acid was converted to intermediate 83F which was then coupled with 4-(2-fluorophenyl)-2-methyloxazole-5-carboxylic acid using the same conditions as for compound 80 and then further used procedures as described in Example 1 to yield compound 83. Compound 83 (120 mg, yield 36%) was obtained as white solid, ¹H NMR (DMSO-d₆, 400 MHz): δ 8.89 (d, J=7.6 Hz, 1H), 8.03 (s, 1H), 7.75 (s, 1H), 7.50-7.39 (m, 3H), 7.38-7.30 (m, 1H), 7.29-7.17 (m, 4H), 5.46-5.33 (m, 1H), 3.33-3.26 (m, 1H), 3.08 (dd, J=9.8, 14.2 Hz, 1H), 2.54 (s, 3H). MS (ESI) m/z (M+H)⁺ 430.1.

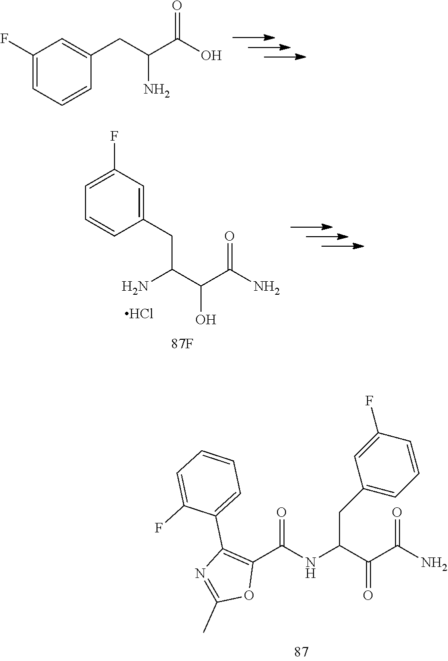 [SMILES: NC(Cc1cccc(F)c1)C(=O)O ; NC(=O)C(O)C(N)Cc1cccc(F)c1 ; Cl ; Cc1nc(-c2ccccc2F)c(C(=O)NC(Cc2cccc(F)c2)C(=O)C(N)=O)o1]

### N-(4-AMINO-1-(3-FLUOROPHENYL)-3,4-DIOXOBUTAN-2-YL)-4-(2-FLUOROPHENYL)-2-METHYLOXAZOLE-5-CARBOXAMIDE (87)

Compound 2-amino-3-(3-fluorophenyl)propanoic acid was converted to intermediate 87F which was then coupled with 4-(2-fluorophenyl)-2-methyloxazole-5-carboxylic acid using the same conditions as for compound 80 and then further used procedures as described in Example 1 to yield compound 87. Compound 87 (160 mg, yield 55%) was obtained as light yellow solid, ¹H NMR (DMSO-d₆, 400 MHz): δ 8.87 (d, J=7.5 Hz, 1H), 8.07 (s, 1H), 7.83 (s, 1H), 7.50-7.40 (m, 2H), 7.38-7.30 (m, 1H), 7.25-7.17 (m, 2H), 7.13-7.01 (m, 3H), 5.42-5.27 (m, 1H), 3.19 (dd, J=3.8, 14.1 Hz, 1H), 2.98 (dd, J=9.9, 13.9 Hz, 1H), 2.55 (s, 3H). MS (ESI) m/z (M+H)⁺ 414.1.

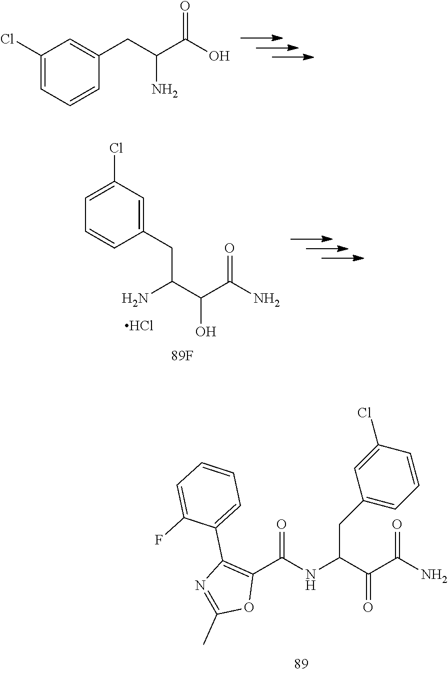 [SMILES: NC(Cc1cccc(Cl)c1)C(=O)O ; NC(=O)C(O)C(N)Cc1cccc(Cl)c1 ; Cl ; Cc1nc(-c2ccccc2F)c(C(=O)NC(Cc2cccc(Cl)c2)C(=O)C(N)=O)o1]

### N-(4-AMINO-1-(3-CHLOROPHENYL)-3,4-DIOXOBUTAN-2-YL)-4-(2-FLUOROPHENYL)-2-METHYLOXAZOLE-5-CARBOXAMIDE (89)

Compound 2-amino-3-(3-chlorophenyl)propanoic acid was converted to intermediate 89F which was then coupled with 4-(2-fluorophenyl)-2-methyloxazole-5-carboxylic acid using the same conditions as for compound 80 and then further used procedures as described in Example 1 to yield compound 89. Compound 89 (70 mg, yield 31.5%) was obtained as white solid. ¹H NMR (400 MHz, DMSO-d) 6 8.89 (d, J=7.6 Hz, 1H), 8.09 (s, 1H), 7.84 (s, 1H), 7.44 (q, J=7.3 Hz, 2H), 7.35-7.25 (m, 3H), 7.25-7.16 (m, 3H), 5.37-5.26 (m, 1H), 3.17 (dd, J=3.7, 13.9 Hz, 1H), 2.95 (dd, J=10.0, 13.9 Hz, 1H), 2.55 (s, 3H). MS (ESI) m/z (M+H)⁺ 430.1.

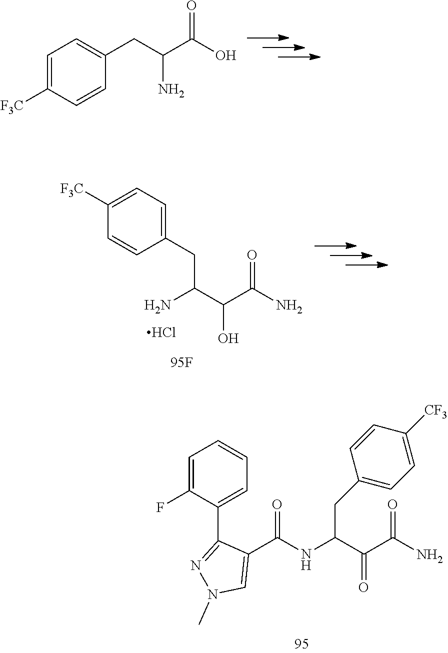 [SMILES: NC(Cc1ccc(C(F)(F)F)cc1)C(=O)O ; NC(=O)C(O)C(N)Cc1ccc(C(F)(F)F)cc1 ; Cl ; Cn1cc(C(=O)NC(Cc2ccc(C(F)(F)F)cc2)C(=O)C(N)=O)c(-c2ccccc2F)n1]

### N-(4-AMINO-3,4-DIOXO-1-(4-(TRIFLUOROMETHYL)PHENYL)BUTAN-2-YL)-3-(2-FLUOROPHENYL)-1-METHYL-1H-PYRAZOLE-4-CARBOXAMIDE (95)

Compound 2-amino-3-(4-(trifluoromethyl)phenyl)propanoic acid was converted to intermediate 95F which was then coupled with 3-(2-fluorophenyl)-1-methyl-1H-pyrazole-4-carboxylic acid using the same conditions as for compound 80 and then further used procedures as described in Example 1 to yield compound 95. Compound 96 (70 mg, yield 55.13%) was obtained as pale yellow solid. ¹H NMR (400 MHz, DMSO-d₆) δ 7.92 (s, 1H), 7.61 (d, J=8.0 Hz, 2H), 7.47-7.37 (m, 2H), 7.34 (d, J=8.0 Hz, 2H), 7.25-7.18 (m, 1H), 7.16-7.09 (m, 1H), 6.96 (br s, 1H), 6.69 (br d, J=6.8 Hz, 1H), 6.22 (br s, 1H), 5.40-5.32 (m, 1H), 3.91 (s, 3H), 3.31 (dd, J=4.6, 14.2 Hz, 1H), 2.97 (dd, J=8.9, 13.9 Hz, 1H). MS (ESI) m/z (M+H)⁺⁼463.1.

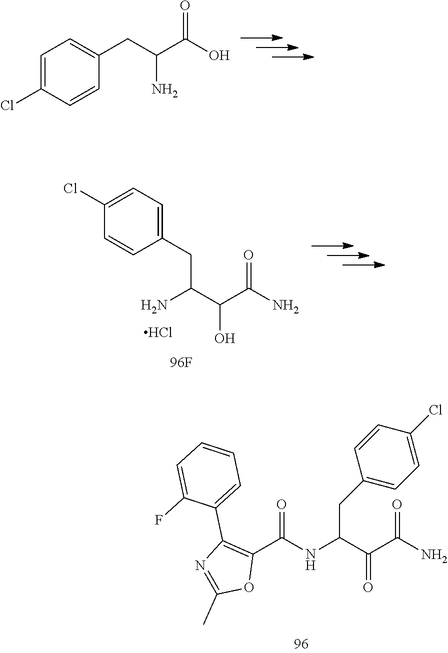 [SMILES: NC(Cc1ccc(Cl)cc1)C(=O)O ; NC(=O)C(O)C(N)Cc1ccc(Cl)cc1 ; Cl ; Cc1nc(-c2ccccc2F)c(C(=O)NC(Cc2ccc(Cl)cc2)C(=O)C(N)=O)o1]

### N-(4-AMINO-1-(4-CHLOROPHENYL)-3,4-DIOXOBUTAN-2-YL)-4-(2-FLUOROPHENYL)-2-METHYLOXAZOLE-5-CARBOXAMIDE (96)

Compound 2-amino-3-(4-chlorophenyl)propanoic acid was converted to intermediate 96F which was then coupled with 4-(2-fluorophenyl)-2-methyloxazole-5-carboxylic acid using the same conditions as for compound 80 and then further used procedures as described in Example 1 to yield compound 96. Compound 96 (120 mg, yield 77%) was obtained as white solid. ¹H NMR (DMSO-4 400 MHz): δ 8.86 (d, J=7.6 Hz, 1H), 8.09 (s, 1H), 7.84 (s, 1H), 7.48-7.41 (m, 2H), 7.38-7.33 (m, 2H), 7.31-7.26 (m, 2H), 7.24-7.18 (m, 2H), 5.37-5.26 (m, 1H), 3.16 (dd, J=3.7, 14.2 Hz, 1H), 2.94 (dd, J=10.0, 13.9 Hz, 1H), 2.56 (s, 3H). MS (ESI) m/z (M+H)⁺ 430.1.

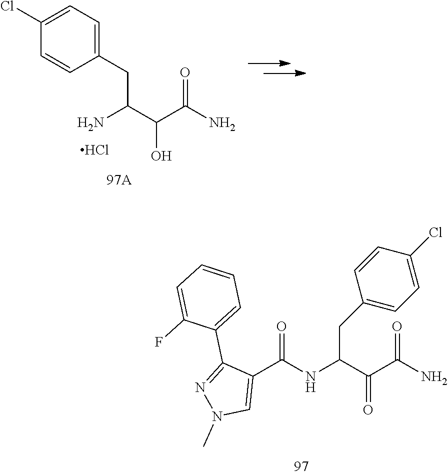 [SMILES: NC(=O)C(O)C(N)Cc1ccc(Cl)cc1 ; Cl ; Cn1cc(C(=O)NC(Cc2ccc(Cl)cc2)C(=O)C(N)=O)c(-c2ccccc2F)n1]

### N-(4-AMINO-1-(4-CHLOROPHENYL)-3,4-DIOXOBUTAN-2-YL)-3-(2-FLUOROPHENYL)-1-METHYL-1H-PYRAZOLE-4-CARBOXAMIDE (97)

Compound 3-amino-4-(4-chlorophenyl)-2-hydroxybutanamide was coupled with 3-(2-fluorophenyl)-1-methyl-1H-pyrazole-4-carboxylic acid using the same conditions as for compound 80 and then further used procedures as described in Example 1 to yield compound 97. Compound 97 (120 mg, yield 65.3%) was obtained as white solid. ¹H NMR (DMSO-d₆, 400 MHz): δ 8.28 (d, J=7.3 Hz, 1H), 8.18 (s, 1H), 8.01 (s, 1H), 7.78 (s, 1H), 7.41-7.30 (m, 4H), 7.28-7.24 (m, 2H), 7.19-7.09 (m, 2H), 5.29-5.11 (m, 1H), 3.91 (s, 3H), 3.11 (dd, J=3.7, 13.9 Hz, 1H), 2.80 (dd, J=10.1, 13.8 Hz, 1H). MS (ESI) m/z (M+H)⁺ 429.1.

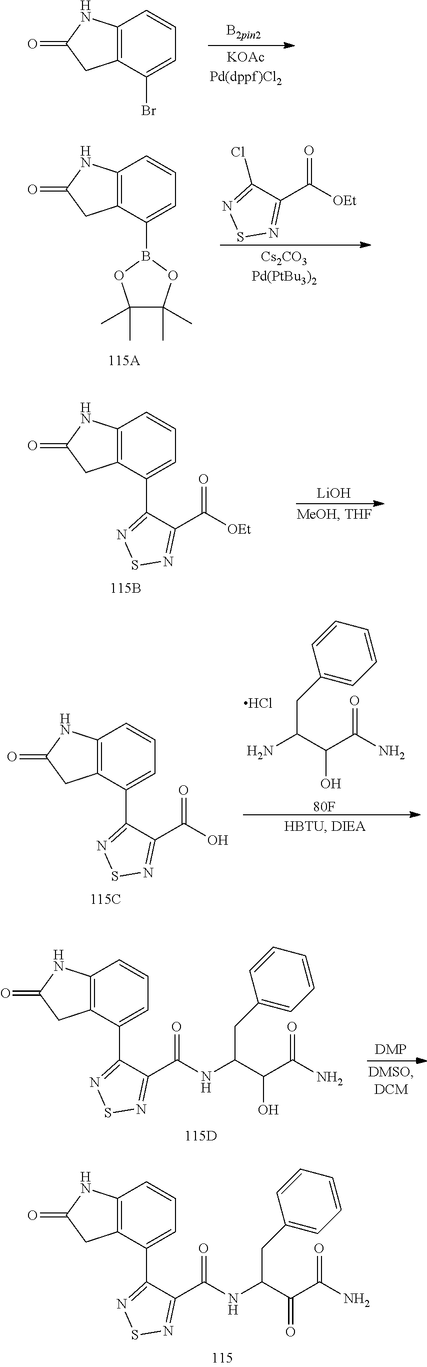

### N-(4-AMINO-3,4-DIOXO-1-PHENYLBUTAN-2-YL)-4-(2-OXOINDOLIN-4-YL)-1,2,5-THIADIAZOLE-3-CARBOXAMIDE (115)

A mixture of 4-bromoindolin-2-one (500.0 mg, 2.36 mmol), B₂pin₂ (898.2 mg, 3.54 mmol), KOAc (462.8 mg, 4.72 mmol), Pd(dppf)Cl₂ (172.5 mg, 235.80 umol) in dioxane (20 mL) was degassed and purged with N₂ for 3 times, and then the mixture was stirred at 80° C. for 12 h under N₂ atmosphere. The mixture was concentrated and the resulting residue was purified by column chromatography (SiO₂, Petroleum ether/Ethyl acetate=10/1 to 0:1). Compound 115A (600.0 mg, crude) was obtained as a yellow solid. The crude product was used in next step directly.

A mixture of ethyl 4-chloro-1,2,5-thiadiazole-3-carboxylate (446.0 mg, 2.32 mmol), compound 115A (600.0 mg, 2.32 mmol), palladium; tritert-butylphosphane (118.3 mg, 231.56 umol), Cs₂CO₃ (2.26 g, 6.95 mmol) in H₂O (5 mL) and dioxane (50 mL) was degassed and purged with N₂ for 3 times, and then the mixture was stirred at 80° C. for 1 hr under N₂ atmosphere. The mixture was concentrated and the resulting residue was purified by column chromatography (SiO₂, Petroleum ether/Ethyl acetate=5/1 to 0:1). Compound 115B (500.0 mg, 50.3% yield, 67.4% purity) was obtained as a yellow solid. MS (ESI) m/z (M+H)⁺ 290.0.

To a solution of compound 115B (480.0 mg, 1.66 mmol) in THF (10 mL) and MeOH (10 mL) was added LiOH.H₂O (2M, 4.15 mL). The mixture was stirred at 20° C. for 10 min. The mixture was concentrated, diluted with H₂O (50 mL), washed with DCM (50 mL), the water phase was added HCl (1M) until pH ˜3, then the mixture was extracted with EA (50 mL×2), dried over Na₂SO₄ and concentrated. Compound 115C (110.0 mg, crude) was obtained as a yellow solid. The crude product was used in next step directly.

Compound 115C was then coupled with intermediate 80F using the same conditions as for compound 80 and then further used procedures as described in Example 1 to yield compound 115. Compound 115 (30 mg, yield 34.3%; 91.1% purity) was obtained as white solid. ¹H NMR (DMSO-d₆, 400 MHz): δ 10.54 (s, 1H), 9.37 (d, J=7.6 Hz, 1H), 8.19 (s, 1H), 7.93 (s, 1H), 7.33-7.19 (m, 5H), 7.17-7.11 (m, 1H), 6.90 (d, J=8.0 Hz, 2H), 5.52-5.45 (m, 1H), 3.53 (s, 2H), 3.25-3.17 (m, 1H), 2.94-2.85 (m, 1H).

### Example 21

### COMPOUNDS 5 AND 8

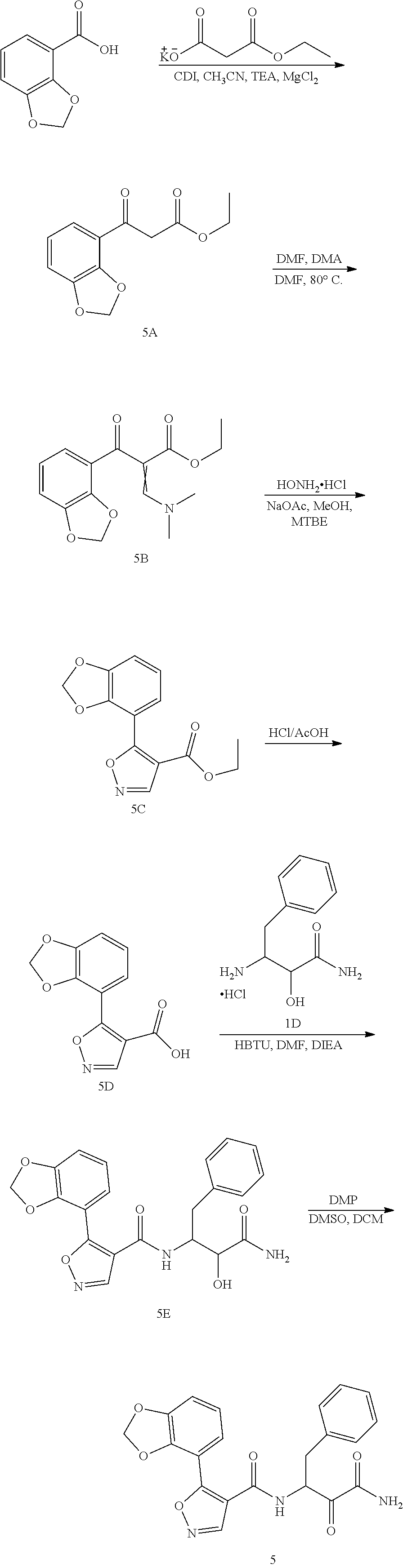

### N-(4-AMINO-3,4-DIOXO-1-PHENYLBUTAN-2-YL)-5-(BENZO[d][1,3]DIOXOL-4-YL)ISOXAZOLE-4-CARBOXAMIDE (5)

Flask 1: To a solution of benzo[d][1,3]dioxole-4-carboxylic acid (2 g, 12.04 mmol) in CH₃CN (15 mL) was added CDI (2.19 g, 13.48 mmol). The mixture was stirred at 25° C. for 4 h.

Flask 2: To a solution of ethyl potassium malonate (2.70 g, 15.89 mmol) in CH₃CN (25 mL) was added MgCl₂ (1.15 g, 12.04 mmol) in portions over 15 min. The mixture was stirred at 25° C. for 0.5 h, then TEA (3.65 g, 36.12 mmol) was added and the slurry was stirred for 0.5 h. The solution in flask 1 was transferred to the slurry in flask 2. The mixture was stirred at 25° C. for 18 h. The reaction mixture was quenched with 3N HCl (40 mL) and the solution was concentrated under reduce pressure. The resulting was extracted with MTBE (50 mL×2). The organic layer was washed with H₂O (50 mL), saturated NaHCO₃ (50 mL), saturated NaCl (50 mL), dried over anhydrous Na₂SO₄, filtered and concentrated under reduced pressure to give compound 5A (2.1 g, 73.9% yield) as a yellow oil, which was used for next step without purification.

A mixture of compound 5A (1.1 g, 4.66 mmol) and DMFDMA (2.47 mL, 18.63 mmol) in DMF (15 mL) was stirred at 80° C. for 3 h. The mixture was concentrated under vacuo to give compound 5B (1.2 g, 88.5% yield) as a brown oil, which was used for next step without purification.

NaOAc (676 mg, 8.24 mmol) was added to the mixture of compound 5B (1.20 g, 4.12 mmol) and hydroxylamine hydrochloride (573 mg, 8.24 mmol) in MeOH (7 mL) and MTBE (7 mL). The mixture was stirred at 25° C. for 17 h. The mixture was added saturated NH₄Cl (20 mL) and extracted with MTBE (20 mL×2). The combined organic phase was washed brine (10 mL), dried over Na₂SO₄, filtered and concentrated under vacuum. The product was purified by FCC (0-10% EA/PE) to give compound 5C (444 mg, 41.3% yield) as a white solid. ¹H NMR (400 MHz, DMSO-d₆) 6 9.07 (s, 1H), 7.29 (d, J=8.0 Hz, 1H), 7.17 (d, J=7.8 Hz, 1H), 7.03-6.97 (m, 1H), 6.13 (s, 2H), 4.24 (q, J=7.1 Hz, 2H), 1.21 (t, J=7.2 Hz, 3H).

HCl (12M, 5 mL) was added to the mixture of compound 5C (244 mg, 0.93 mmol) in AcOH (5 mL). The mixture was stirred at 118° C. for 4.5 h. The mixture was concentrated under vacuum. H₂O (50 mL) was added to the mixture, the mixture was extracted with DCM (50 mL). The organic phase was washed with brine (30 mL), dried over Na₂SO₄, filtered and concentrated under vacuo to give compound 5D (185 mg, 84.9% yield) as a yellow solid, which was used for next step without purification. ¹H NMR (400 MHz, DMSO-d₆) δ 8.96 (s, 1H), 7.28 (d, J=7.7 Hz, 1H), 7.11 (dd, J=1.0, 7.8 Hz, 1H), 6.96 (t, J=7.9 Hz, 1H), 6.14-6.06 (m, 2H).

Compound 5D and intermediate 1D were coupled using the same conditions as for intermediates 58E and 1D and then used procedures as described in Example 1 to yield compound 5. Compound 5 (40 mg, yield 20.8%) was obtained as pale-yellow solid. ¹H NMR (400 MHz, DMSO-d₆) δ 8.88 (br d, J=7.3 Hz, 1H), 8.81 (s, 1H), 8.08 (br s, 1H), 7.82 (br s, 1H), 7.30-7.17 (m, 5H), 7.07 (br dd, J=7.7, 15.7 Hz, 2H), 6.92-6.86 (m, 1H), 6.03-5.86 (m, 2H), 5.31 (br s, 1H), 3.15 (br dd, J=3.4, 13.6 Hz, 1H), 2.81 (br dd, J=10.3, 13.8 Hz, 1H). MS (ESI) m/z (M+H)⁺ 408.1.

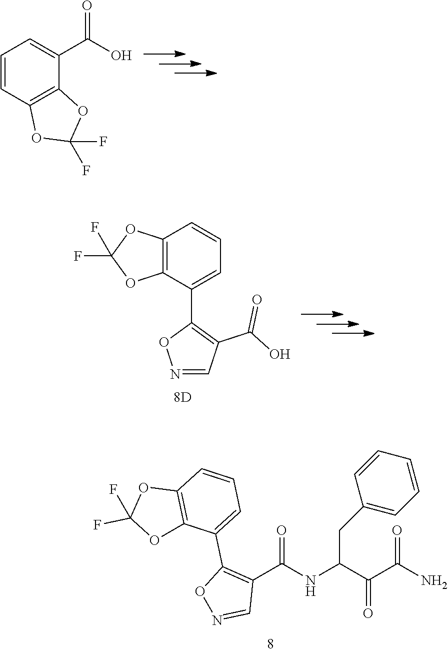 [SMILES: O=C(O)c1cccc2c1OC(F)(F)O2 ; O=C(O)c1cnoc1-c1cccc2c1OC(F)(F)O2 ; NC(=O)C(=O)C(Cc1ccccc1)NC(=O)c1cnoc1-c1cccc2c1OC(F)(F)O2]

### N-(4-AMINO-3,4-DIOXO-1-PHENYLBUTAN-2-YL)-5-(2,2-DIFLUOROBENZO[d][1,3]DIOXOL-4-YL)ISOXAZOLE-4-CARBOXAMIDE (8)

Compound 2,2-difluorobenzo[d][1,3]dioxole-4-carboxylic acid was converted to intermediate 8D using procedures as described for compound 5 and then intermediate 8D was coupled with intermediate 1D using procedures as described in compound 58 to yield compound. Compound 8 (60 mg, yield 54%) was obtained as white solid. ¹H NMR (DMSO-d₆, 400 MHz): δ. 9.06 (d, J=7.5 Hz, 1H), 8.97 (s, 1H), 8.10 (s, 1H), 7.85 (s, 1H), 7.65-7.47 (m, 2H), 7.36-7.14 (m, 6H), 5.38 (s, 1H), 3.24-3.07 (m, 1H), 2.89-2.75 (m, 1H). MS (ESI) m/z (M+H)⁺ 444.1.

### Example 22

### COMPOUNDS 11, 27, 30, 29, 45, AND 59

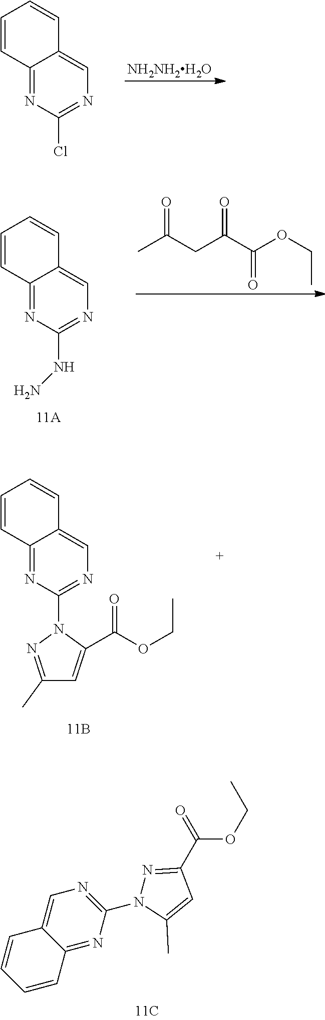 [SMILES: Clc1ncc2ccccc2n1 ; "NH2NH2·H2O" ; NNc1ncc2ccccc2n1 ; CCOC(=O)C(=O)CC(C)=O ; CCOC(=O)c1cc(C)nn1-c1ncc2ccccc2n1 ; CCOC(=O)c1cc(C)n(-c2ncc3ccccc3n2)n1]

To a mixture of 2-chloroquinazoline (1 g, 6.08 mmol) and K₂CO₃ (1.00 g, 7.24 mmol) was added NH₂NH₂.H₂O (5 mL, 85% purity). The mixture was stirred at 100° C. for 0.5 hr. The reaction mixture was ice cooled and the resulting crude crystals were collected by filtration. The crystals were washed with cold water, air dried to give a residue. The residue was triturated in PE (20 mL) and collected by filtration. Compound 11A (490 mg, yield: 50.4%) was obtained as a yellow solid.

To a solution of compound 11A (490 mg, 3.06 mmol) and ethyl 2,4-dioxopentanoate (484 mg, 3.06 mmol) was added HOAc (5 mL). The mixture was stirred at 100° C. for 16 h. The mixture was concentrated, diluted with EA (25 mL) and filtered. The organic layer was washed with NaHCO₃ (25 mL), brine (25 mL×3), dried over Na₂SO₄, then filtered and concentrated to give a residue. The residue was purified by preparatory-TLC (PE:EA=1:1). Compound 11B (180 mg, yield: 18.1%) was obtained as a yellow oil. Compound 11C (110 mg, yield: 11.3%) was obtained as a yellow oil.

Compound 11B: ¹H NMR (400 MHz, DMSO-d₆) δ 9.69 (s, 1H), 8.23 (d, J=8.4 Hz, 1H), 8.12-8.03 (m, 1H), 8.00-7.93 (m, 1H), 7.78 (dt, J=1.0, 7.6 Hz, 1H), 6.85 (s, 1H), 4.21-4.09 (m, 2H), 2.28 (s, 3H), 1.03 (t, J=7.2 Hz, 3H). MS (ESI) m/z (M+H)⁺ 282.9.

Compound 11C: ¹H NMR (400 MHz, DMSO-d₆) δ 9.79 (d, J=0.7 Hz, 1H), 8.29 (d, J=8.2 Hz, 1H), 8.16-8.05 (m, 2H), 7.83 (ddd, J=1.5, 6.4, 8.1 Hz, H), 6.83 (d, J=0.9 Hz, 1H), 4.32 (q, J=7.1 Hz, 2H), 2.68 (d, J=0.9 Hz, 3H), 1.32 (t, J=7.2 Hz, 3H). MS (ESI) m/z (M+H)⁺ 282.9.

### N-(4-AMINO-3,4-DIOXO-1-PHENYLBUTAN-2-YL)-3-METHYL-1-(QUINAZOLIN-2-YL)-1H-PYRAZOLE-5-CARBOXAMIDE (11)

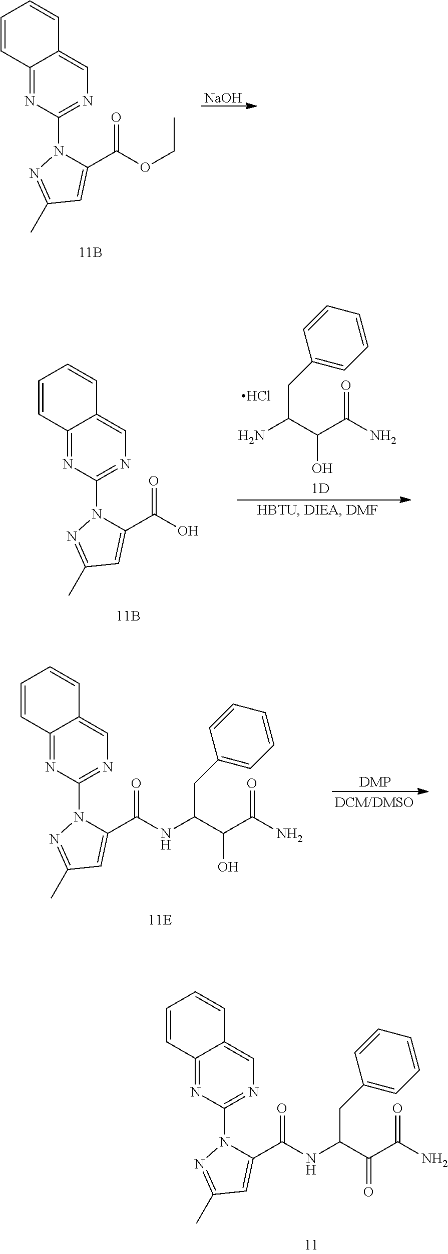

Compound 11B was subjected to procedures as used for converting intermediate 58D to compound 58 as described in Example 19 to yield compound 11. Compound 11 (45 mg, yield 41.4%) was obtained as pale yellow solid, ¹H NMR (400 MHz, DMSO-d₆) δ 9.51 (s, 1H), 9.11 (d, J=7.7 Hz, 1H), 8.19 (d, J=8.2 Hz, 1H), 8.09-7.98 (m, 2H), 7.88-7.79 (m, 2H), 7.75 (t, J=7.6 Hz, 1H), 7.28-7.16 (m, 5H), 6.58 (s, 1H), 5.43-5.15 (m, 1H), 3.13 (dd, J=3.1, 14.1 Hz, 1H), 2.83 (dd, J=9.9, 13.9 Hz, 1H), 2.28 (s, 3H). MS (ESI) m/z (M+H)⁺ 429.1.

### N-(4-AMINO-3,4-DIOXO-1-PHENYLBUTAN-2-YL)-5-METHYL-1-(QUINAZOLIN-2-YL)-1H-PYRAZOLE-3-CARBOXAMIDE (27)

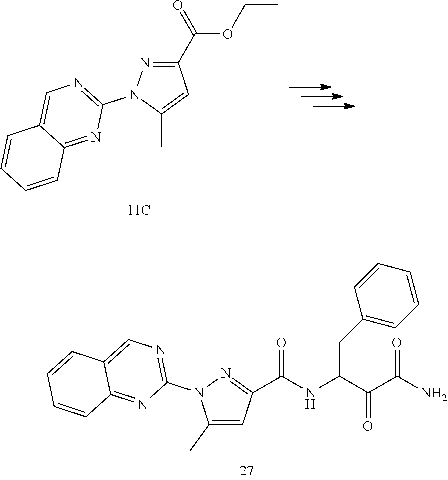 [SMILES: CCOC(=O)c1cc(C)n(-c2ncc3ccccc3n2)n1 ; Cc1cc(C(=O)NC(Cc2ccccc2)C(=O)C(N)=O)nn1-c1ncc2ccccc2n1]

Compound 11C was subjected to procedures as used for converting intermediate 58D to compound 58 as described in Example 19 to yield compound 27. Compound 27 (28 mg, yield 77.1%) was obtained as pale yellow solid, ¹H NMR (400 MHz, DMSO-d₆) δ 9.76 (s, 1H), 8.48 (d, J=7.5 Hz, 1H), 8.26 (d, J=8.2 Hz, 1H), 8.15-7.98 (m, 3H), 7.89-7.73 (m, 2H), 7.27-7.19 (m, 4H), 7.19-7.11 (m, 1H), 6.68 (s, 1H), 5.56-5.29 (m, 1H), 3.24-3.00 (m, 2H), 2.64 (s, 3H). MS (ESI) m/z (M+H)⁺ 429.2.

### N-(4-AMINO-3,4-DIOXO-1-PHENYLBUTAN-2-YL)-3-METHYL-1-(5-PHENYLPYRIMIDIN-2-YL)-1H-PYRAZOLE-5-CARBOXAMIDE (30)

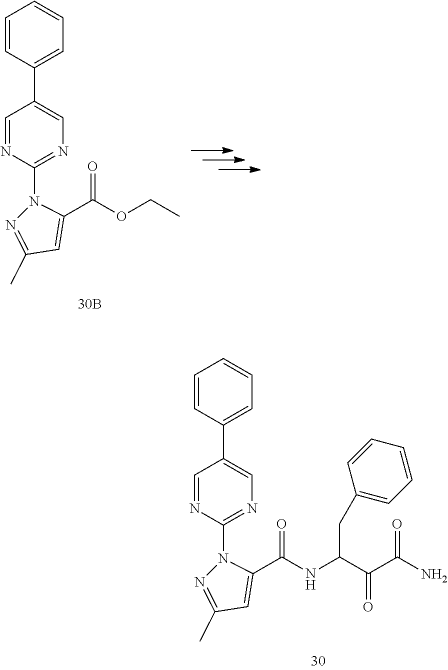 [SMILES: CCOC(=O)c1cc(C)nn1-c1ncc(-c2ccccc2)cn1 ; Cc1cc(C(=O)NC(Cc2ccccc2)C(=O)C(N)=O)n(-c2ncc(-c3ccccc3)cn2)n1]

Compound 30B was prepared from 2-chloro-5-phenylpyrimidine using procedures as described for compound 11. Then, compound 30B was subjected to procedures as used for converting intermediate 58D to compound 58 as described in Example 19 to yield compound 30. Compound 30 (130 mg, yield 82.9%) was obtained as white solid, ¹H NMR (400 MHz, DMSO-d₆) δ 9.07 (d, J=7.2 Hz, 1H), 9.01 (s, 2H), 8.06 (s, 1H), 7.84-7.79 (m, 3H), 7.58-7.44 (m, 3H), 7.28-7.21 (m, 4H), 7.15-7.10 (m, 1H), 6.58 (s, 1H), 5.29-5.21 (m, 1H), 3.18-3.10 (m, 1H), 2.88-2.78 (m, 1H), 2.26 (s, 3H). MS (ESI) m/z (M+H)⁺ 455.1.

### N-(4-AMINO-3,4-DIOXO-1-PHENYLBUTAN-2-YL)-5-METHYL-1-(5-PHENYLPYRIMIDIN-2-YL)-1H-PYRAZOLE-3-CARBOXAMIDE (29)

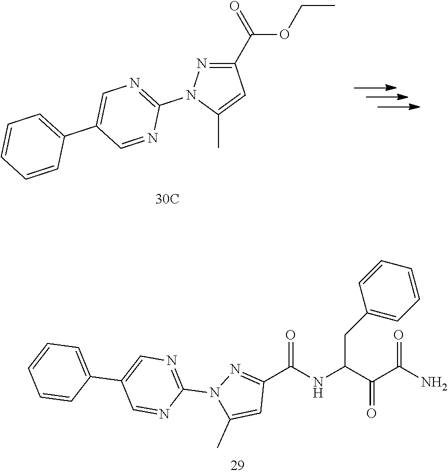 [SMILES: CCOC(=O)c1cc(C)n(-c2ncc(-c3ccccc3)cn2)n1 ; Cc1cc(C(=O)NC(Cc2ccccc2)C(=O)C(N)=O)nn1-c1ncc(-c2ccccc2)cn1]

Compound 30C was prepared from 2-chloro-5-phenylpyrimidine using procedures as described for compound 11. Then, compound 30C was subjected to procedures as used for converting intermediate 58D to compound 58 as described in Example 19 to yield compound 29. Compound 29 (50 mg, yield 33.8%) was obtained as white solid, ¹H NMR (400 MHz, DMSO-d₆) δ 9.25 (s, 2H), 8.12 (br s, 1H), 7.90-7.47 (m, 7H), 7.33-7.15 (m, 5H), 6.69 (s, 1H), 5.56-5.42 (m, 1H), 3.35-3.12 (m, 2H), 2.65 (s, 3H). MS (ESI) m/z (M+H)⁺ 455.2.

### N-(4-AMINO-3,4-DIOXO-1-PHENYLBUTAN-2-YL)-3-METHYL-1-(4-PHENYLPYRIMIDIN-2-YL)-1H-PYRAZOLE-5-CARBOXAMIDE (45)

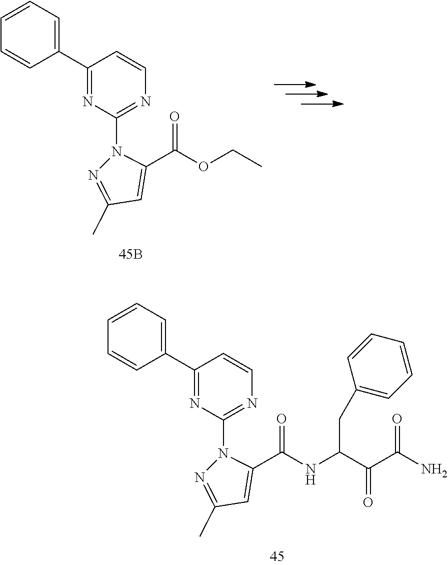 [SMILES: CCOC(=O)c1cc(C)nn1-c1nccc(-c2ccccc2)n1 ; Cc1cc(C(=O)NC(Cc2ccccc2)C(=O)C(N)=O)n(-c2nccc(-c3ccccc3)n2)n1]

Compound 45B was prepared from 2-chloro-4-phenylpyrimidine using procedures as described for compound 11. Then, compound 45B was subjected to procedures as used for converting intermediate 58D to compound 58 as described in Example 19 to yield compound 45. Compound 45 (110 mg, yield 73.5%) was obtained as white solid, ¹H NMR (DMSO-d₆, 400 MHz): h 9.06 (d, J=7.3 Hz, 1H), 8.78 (d, J=5.3 Hz, 1H), 8.12-8.05 (m, 3H), 8.00 (d, J=5.3 Hz, 1H), 7.83 (s, 1H), 7.58-7.46 (m, 3H), 7.25-7.13 (m, 5H), 6.55 (s, 1H), 5.44-5.36 (m, 1H), 3.11 (dd, J=3.9, 14.0 Hz, 1H), 2.76 (dd, J=9.9, 13.9 Hz, 1H), 2.28 (s, 3H). MS (ESI) m/z (M+H)⁺ 455.2.

### N-(4-AMINO-3,4-DIOXO-1-PHENYLBUTAN-2-YL)-5-METHYL-1-(4-PHENYLPYRIMIDIN-2-YL)-1H-PYRAZOLE-3-CARBOXAMIDE (59)

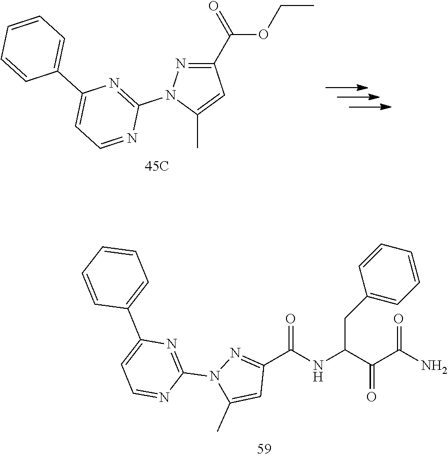 [SMILES: CCOC(=O)c1cc(C)n(-c2nccc(-c3ccccc3)n2)n1 ; Cc1cc(C(=O)NC(Cc2ccccc2)C(=O)C(N)=O)nn1-c1nccc(-c2ccccc2)n1]

Compound 45C was prepared from 2-chloro-4-phenylpyrimidine using procedures as described for compound 11. Then, compound 45C was subjected to procedures as used for converting intermediate 58D to compound 58 as described in Example 19 to yield compound 59. Compound 59 (25 mg, yield 11.9%) was obtained as yellow solid, ¹H NMR (DMSO-d₆, 400 MHz): δ 8.99 (d, J=5.3 Hz, 1H), 8.29-8.25 (m, 2H), 8.10 (br d, J=5.3 Hz, 2H), 7.82 (br s, 1H), 7.65-7.58 (m, 4H), 7.31-7.24 (m, 4H), 7.23-7.17 (m, 1H), 6.72-6.68 (m, 1H), 5.49 (dt, J=4.9, 8.1 Hz, 1H), 3.29 (dd, J=4.9, 14.2 Hz, 1H), 3.16 (br d, J=5.5 Hz, 1H), 2.71-2.69 (m, 3H). MS (ESI) m/z (M+H)⁺ 455.1.

### Example 23

### COMPOUNDS 43-44 [METHYL 4-(4-((7,9-DIOXO-6,10-DIOXASPIRO[4.5]DECAN-8-YLIDENE)-λ³-IODANYL)PHENYL)-1,2,5-THIADIAZOLE-3-CARBOXYLATE (43)

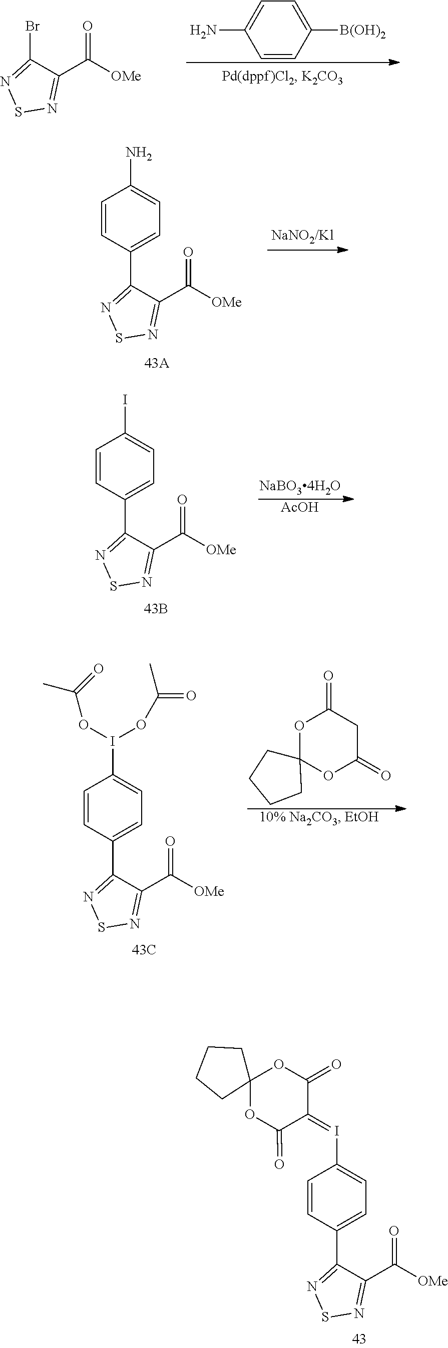

To a solution of methyl 4-bromo-1,2,5-thiadiazole-3-carboxylate (2 g, 8.97 mmol) and (4-aminophenyl)boronic acid (1.60 g, 11.66 mmol) in dioxane (25 mL) and H₂O (2 mL) was added K₂CO₃ (3.72 g, 26.90 mmol), Pd(dppf)Cl₂ (656 mg, 896.67 umol) was added under N₂ atmosphere, the mixture was stirred at 80° C. for 18 h under N₂ atmosphere. The reaction mixture was concentrated to remove solvent, then diluted with EA (50 mL) and filtered; the organic layers were concentrated to give a residue. The residue was purified by flash silica gel chromatography (ISCO®; 12 g SepaFlash® Silica Flash Column, Eluent of 0-30% Ethyl acetate/Petroleum ethergradient @ 30 mUmin). Compound 43A (1.3 g, yield: 61.6%) as light yellow solid was obtained. ¹H NMR (400 MHz, DMSO-d₆) 7.47-7.33 (m, 2H), 6.65-6.56 (m, 2H), 5.64 (s, 2H), 3.94-3.85 (m, 3H). MS (ESI) m/z (M+H)⁺ 236.1.

A solution of TsOH.H₂O (2.63 g, 13.81 mmol) in H₂O (20 mL) was added a suspension of compound 43A (1.3 g, 5.53 mmol) in CH₃CN (30 mL) at 0° C., the mixture was stirred for 30 min, then a solution of NaNO₂ (572 mg, 8.29 mmol) in H₂O (10 mL) and KI (1.38 g, 8.29 mmol) in H₂O (10 mL) was added dropwise to the mixture at 0° C., After addition, the mixture was stirred at 25° C. for 16 h. The mixture was quenched by the addition of saturated Na₂SO₃ (˜20 mL) at 0° C. The mixture was concentrated in vacuum to remove CH₃CN. The reaction was filtered, the filter cake was dried in vacuo. The residue was purified by flash silica gel chromatography (ISCO®; 12 g SepaFlash® Silica Flash Column, Eluent of 0-10% Ethyl acetate/Petroleum ethergradient @ 30 mUmin). Compound 43B (1.4 g, yield: 73.2%) as white solid was obtained. ¹H NMR (400 MHz, CDCl₃) δ 7.90-7.77 (m, 2H), 7.52-7.37 (m, 2H), 4.02-3.92 (s, 3H).

Sodium perborate tetrahydrate (4 g, 26.00 mmol) was added in portions to a solution of compound 43B (900 mg, 2.60 mmol) in AcOH (15 mL), the mixture was stirred at 50° C. for 10 h. The reaction mixture was diluted with DCM (50 mL), filtered, the filtrate was diluted with water (100 mL), and extracted three times with DCM (40 mL×2). The combined organic extracts were dried with Na₂SO₄, filtered, and concentrated to give a residue. The residue was triturated in DCM:PE (1:15) (20 mL×3). Filtered and the cake was obtained. Compound 43C (590 mg, yield: 48.9%) as light yellow solid was obtained. ¹H NMR (400 MHz, CDCl₃) δ 8.26-8.15 (m, 2H), 7.89-7.84 (m, 2H), 4.05-3.98 (m, 3H), 2.10-2.00 (m, 6H).

To a solution of compound 43C (590 mg, 1.27 mmol) in EtOH (20 mL) was added the solution of Na₂CO₃ (539 mg, 5.08 mmol) in H₂O (10 mL), then 6,10-dioxaspiro[4.5]decane-7,9-dione (281 mg, 1.65 mmol) was added, the mixture was stirred at 20° C. for 1 h. The reaction mixture was then diluted with water (80 mL), and extracted with DCM (50 mL×3). The combined organic extracts were dried with anhydrous Na₂SO₄, filtered, and concentrated to give a residue. The residue was purified by flash silica gel chromatography (ISCO®; 4 g SepaFlash® Silica Flash Column, Eluent of 0-100% Ethyl acetate/Petroleum ethergradient @ 20 mUmin). The product (Part of Methyl ester was changed to Ethyl ester) was dissolved in MeOH (20 mL), then a solution of Na₂CO₃ (100 mg) in H₂O (2 mL) was added, the mixture was stirred at 20° C. for 4 h. The reaction mixture was then diluted with water (50 mL), and extracted with DCM (30 mL×3). The combined organic layers were dried with anhydrous Na₂SO₄, filtered, and concentrated to give the desired product. Compound 43 (130 mg, yield: 19.9%) as light yellow solid was obtained. ¹H NMR (400 MHz, CDCl₃) δ 8.02-7.92 (m, 2H), 7.86-7.75 (m, 2H), 4.04-3.90 (m, 3H), 2.24-2.15 (m, 4H), 1.85-1.78 (m, 4H). MS (ESI) m/z (M+Na)⁺537.0.

### ETHYL 3-(4-((7,9-DIOXO-6,10-DIOXASPIRO[4.5]DECAN-8-YLIDENE)-λ³-IODANYL)PHENYL)-1-METHYL-1H-PYRAZOLE-4-CARBOXYLATE (44)

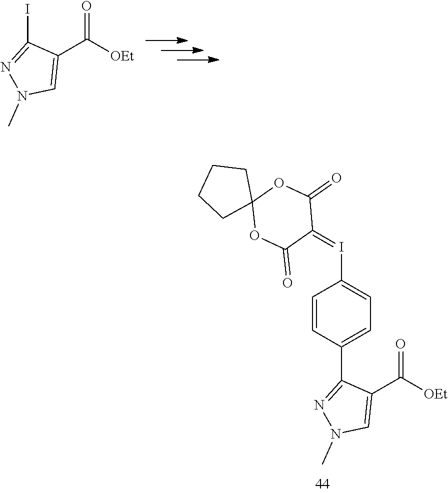 [SMILES: CCOC(=O)c1cn(C)nc1I ; CCOC(=O)c1cn(C)nc1-c1ccc(I=C2C(=O)OC3(CCCC3)OC2=O)cc1]

Compound ethyl 3-iodo-1-methyl-1H-pyrazole-4-carboxylate was converted to the compound 44 using procedures described for compound 43. Compound 44 (120 mg, yield 57.5%) was obtained as pale yellow solid, ¹H NMR (400 MHz, CDCl₃) δ 7.98 (s, 1H), 7.91 (s, 4H), 4.25 (q, J=7.1 Hz, 2H), 3.97 (s, 3H), 2.16 (t, J=7.4 Hz, 4H), 1.82-1.77 (m, 4H), 1.29 (t, J=7.2 Hz, 3H). MS (ESI) m/z (M+Na)⁺546.9.

### Example 24

### COMPOUNDS 56 AND 66 ETHYL-4-(4-((7,9-DIOXO-6,10-DIOXASPIRO[4.5]DECAN-8-YLIDENE)-λ³-IODANYL)PHENYL)-2-METHYLOXAZOLE-5-CARBOXYLATE (56)

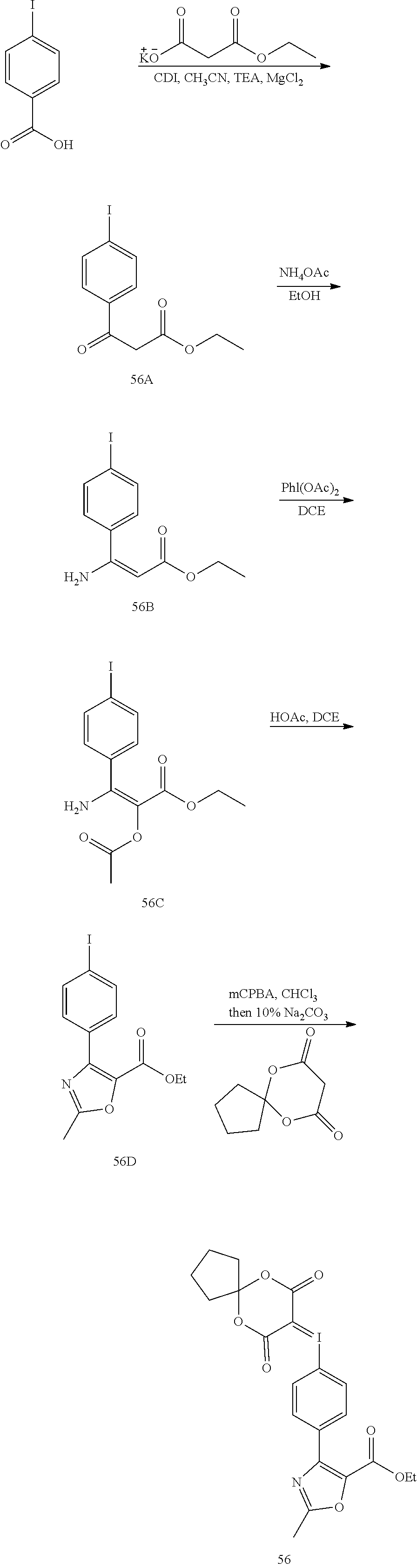

(Flask A) To a solution of 4-iodobenzoic acid (25 g, 100.80 mmol) in CH₃CN (300 mL) was added CDI (18.5 g, 114.09 mmol), the mixture was stirred at 20° C. for 2 h. At the same time, in Flask B to a solution of potassium; 3-ethoxy-3-oxo-propanoate (22.30 g, 131.04 mmol) in CH₃CN (300 mL) was added MgCl₂ (10.6 g, 111.33 mmol) and TEA (301.75 mmol, 42 mL), the mixture was stirred at 20° C. for 2 h. The solution of flask A was then transferred to flask B, the mixture was stirred for 18 h at 20° C. The reaction mixture was diluted with H₂O (200 mL), adjusted to pH ˜4 with HC (4M), extracted with EA (300 mL×3) and the organic layers were combined and washed with NaHCO₃ (aq) (500 mL), brine (500 mL). And then the organic phase was dried over anhydrous sodium sulfate, filtered and concentrated to give a residue. Compound 56A (31.5 g, yield: 98.2%) as yellow oil was obtained, which was used into the next step without further purification. ¹H NMR (400 MHz, CDCl₃) δ 7.91-7.73 (m, 2H), 7.70-7.42 (m, 2H), 4.30-4.15 (m, 2H), 3.97-3.89 (m, 2H), 1.30-1.19 (m, 3H).

To a solution of compound 56A (31.5 g, 99.02 mmol) in EtOH (300 mL) was added NH₄OAc (20 g, 259.46 mmol), then the mixture was stirred at 85° C. for 18 h. The reaction mixture was concentrated to remove solvent, then diluted with water (150 mL) and extracted with EA (100 mL×3), the organic layers were washed with saturated NaHCO₃ (100 mL×2), dried over Na₂SO₄, filtered and concentrated to give a residue. The residue was purified by flash silica gel chromatography (ISCO®; 220 g SepaFlash® Silica Flash Column, Eluent of 0-10% Ethyl acetate/Petroleum ethergradient @ 100 mUmin). Compound 56B (26 g, yield: 71.5%) as light yellow solid was obtained. ¹H NMR (400 MHz, DMSO-d₆) δ 7.86-7.75 (m, 2H), 7.44-7.34 (m, 2H), 4.77 (s, 1H), 4.05 (q, J=7.1 Hz, 2H), 1.19 (t, J=7.2 Hz, 3H). MS (ESI) m/z (M+H)⁺ 317.9.

To a solution of compound 56B (2 g, 6.31 mmol) in DCE (20 mL) was added PhI(OAc)₂ (2.44 g, 7.57 mmol) in portions at 0° C., then the mixture was stirred at 20° C. for 1 h. The mixture was cooled to 0° C., washed with saturated NaHCO₃ (80 mL), the aqueous phase was extracted with DCM (30 mL), the organic layer was collected, washed with H₂O (50 mL), then dried over Na₂SO₄, filtered and concentrated to give a residue. The residue was purified by flash silica gel chromatography (ISCO®; 20 g SepaFlash® Silica Flash Column, Eluent of 0-10% Ethyl acetate/Petroleum ethergradient @ 30 mUmin). Compound 56C (220 mg, yield: 8.2%) as light yellow oil was obtained. ¹H NMR (400 MHz, DMSO-d₆) δ 7.84-7.80 (m, 2H), 7.16-7.12 (m, 2H), 4.13-4.06 (m, 2H), 1.88 (s, 3H), 1.19-1.15 (m, 3H). MS (ESI) m/z (M+H)⁺ 376.0.

The solution of compound 56C (220 mg, 586.42 umol) in AcOH (2 mL) and DCE (1 mL) was stirred at 90° C. for 1 h. The solvent was removed in vacuo. The residue was dissolved in EtOAc (30 mL), washed with saturated NaHCO₃ (30 mL). The organics were collected and concentrated to give a residue. The residue was purified by preparatory-TLC (PE:EA=5:1). Compound 56D (110 mg, yield: 52.5%) as light yellow solid was obtained. ¹H NMR (400 MHz, CDCl₃) δ 7.86-7.72 (m, 4H), 4.39 (q, J=7.1 Hz, 2H), 2.57 (s, 3H), 1.38 (t, J=7.2 Hz, 3H)

To a solution of compound 56D (0.4 g, 1.12 mmol) in CHCl₃ (8 mL) was added m-CPBA (314 mg, 1.46 mmol, 80% purity), the mixture was stirred at 20° C. for 18 h. The mixture was concentrated to get rid of most of solvent to give a residue. The residue was dissolved in EtOH (15 mL), and the reaction was added Na₂CO₃ (475 mg, 4.48 mmol) in H₂O (10 mL), and then added 6,10-dioxaspiro[4.5]decane-7,9-dione (248 mg, 1.46 mmol) quickly. The reaction mixture was then stirred at 20° C. for 2 h. The residue was diluted with water (100 mL) and extracted with EA (50 mL×2). The combined organic extracts were washed with brine (100 mL) and dried with anhydrous Na₂SO₄, filtered and concentrated to give a residue. The residue was purified by flash silica gel chromatography (ISCO®; 12 g SepaFlash® Silica Flash Column, Eluent of 0-100% Ethyl acetate/Petroleum ethergradient @ 30 mUmin). Compound 56 (190 mg, yield: 30.7%) was obtained as white solid. ¹H NMR (400 MHz, CDCl₃) δ 8.25-8.09 (m, 2H), 7.97-7.85 (m, 2H), 4.40 (q, J=7.1 Hz, 2H), 2.59 (s, 3H), 2.21-2.12 (m, 4H), 1.84-1.75 (m, 4H), 1.39 (t, J=7.2 Hz, 3H). MS (ESI) m/z (M+Na)⁺ 548.1.

### ETHYL-4-(2-((7,9-DIOXO-6,10-DIOXASPIRO[4.5]DECAN-8-YLIDENE)-λ³-IODANYL)PHENYL)-2-METHYLOXAZOLE-5-CARBOXYLATE (66)

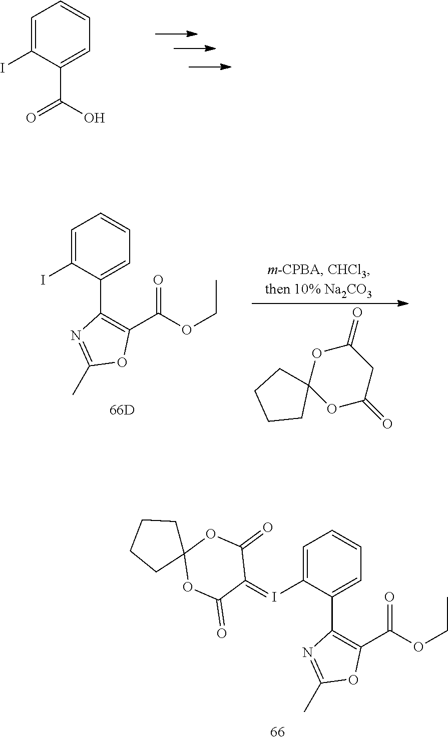 [SMILES: O=C(O)c1ccccc1I ; CCOC(=O)c1oc(C)nc1-c1ccccc1I ; O=C1CC(=O)OC2(CCCC2)O1 ; CCOC(=O)c1oc(C)nc1-c1ccccc1I=C1C(=O)OC2(CCCC2)OC1=O]

Compound 2-iodobenzoic acid was converted to intermediate 66D using the same procedures as described for synthesis of intermediate 58D. Further, intermediate 66D was treated with 6,10-dioxaspiro[4.5]decane-7,9-dione using the same conditions as described for compound 56 to obtain final compound 66. Compound 66 (90 mg, yield 15.3%) was obtained as white solid, ¹H NMR (CDCl₃, 400 MHz): δ 8.77 (dd, J=1.8, 7.8 Hz, 1H), 7.67 (dd, J=1.1, 8.2 Hz, 1H), 7.61-7.55 (m, 1H), 7.54-7.48 (m, 1H), 4.46 (q, J=7.1 Hz, 2H), 2.66 (s, 3H), 2.29-2.21 (m, 4H), 1.85 (td, J=3.9, 7.1 Hz, 4H), 1.43 (t, J=7.2 Hz, 3H). MS (ESI) m/z (M+H)⁺ 548.0.

### Example 25

### COMPOUNDS 103, 114, AND 112 N-(4-AMINO-3,4-DIOXO-1-PHENYLBUTAN-2-YL)-3-(1-ISOPROPYL-2-OXO-2,3-DIHYDRO-1H-BENZO[D]IMIDAZOL-4-YL)-1-METHYL-1H-PYRAZOLE-4-CARBOXAMIDE (103)

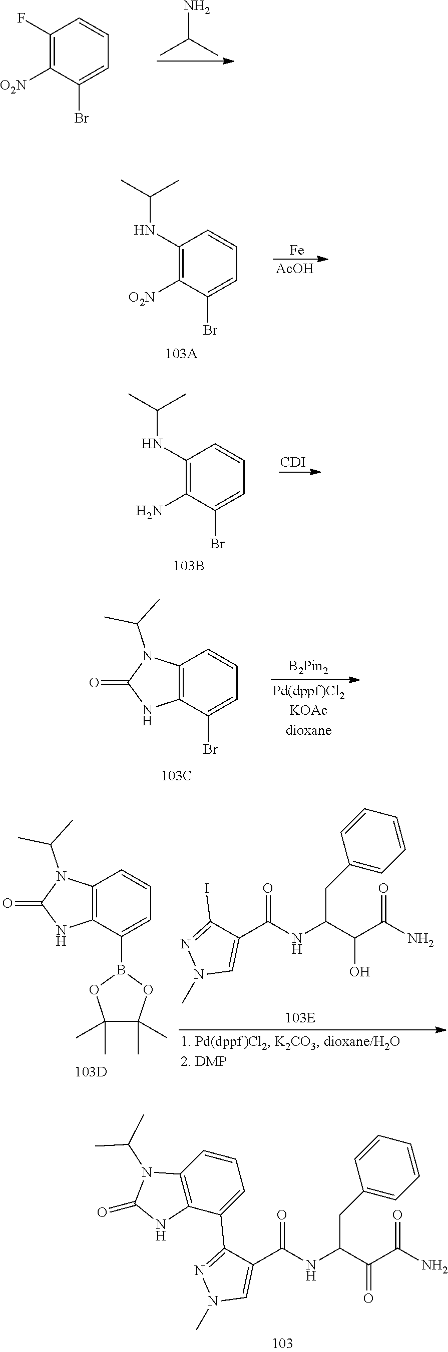

The solution of 1-bromo-3-fluoro-2-nitrobenzene (4.5 g, 20.45 mmol) and isopropyl amine (1.21 g, 20.45 mmol) in EtOH (20 mL) was stirred at 50° C. for 48 h. The solvent was removed in vacuo. The residue was purified by column (PE:EA=10:1) to give compound 103A (5 g, yield: 94.34%) as brown oil.

To a solution of compound 103A (5 g, 19.30 mmol) in AcOH (60 mL) was added Fe (5.39 g, 96.49 mmol). The mixture was stirred at 60° C. for 1 h. The solvent was removed in vacuo. The residue was washed with saturated NaHCO₃ (200 mL), extracted with EtOAc (100 mL×2). The organics were collected, washed with brine (200 mL), dried with Na₂SO₄, filtered, and concentrated to give compound 103B (4.4 g, crude) as brown oil, which was used directly for the next step without further.

To a solution of compound 103B (4.4 g, 19.20 mmol) in THF (60 mL) was added TEA (5.4 mL, 38.41 mmol), CDI (6.23 g, 38.41 mmol). The mixture was stirred at 20° C. for 12 h. The mixture was washed with H₂O (50 mL), extracted with EtOAc (50 mL×2). The organics were collected and concentrated. The residue was purified by column (PE:EA=2:1) to give compound 103C (2.5 g, yield: 51.03%) as brown solid.

To a solution of compound 103C (400 mg, 1.57 mmol) and 4,4,4′,4′,5,5,5′,5′-octamethyl-2,2′-bi(1,3,2-dioxaborolane) (B₂Pin₂) (398 mg, 1.57 mmol) in dioxane (10 mL) was added Pd(dppf)Cl₂ (115 mg, 156.79 umol), KOAc (462 mg, 4.70 mmol). The mixture was stirred at 90° C. for 12 h under N₂. The solution was filtered. The filtrate was collected and concentrated. The residue was purified by column (PE:EA=2:1) to give compound 103D (398 mg, yield: 84.00%) as light brown solid)

Compounds 103D and intermediate 103E were converted to compound 103 using procedures as described in Example 1. Compound 103 (70 mg, yield: 64.6%) as a white solid was obtained. ¹H NMR (DMSO-d₆, 400 MHz): 9.96 (br s, 1H), 8.07 (s, 1H), 7.92-7.46 (m, 3H), 7.35-7.11 (m, 8H), 6.97-6.92 (m, 1H), 5.37-5.31 (m, 1H), 4.65-4.57 (m, 1H), 3.94 (s, 3H), 3.21-3.16 (m, 1H), 2.90-2.84 (m, 1H), 1.49 (d, J=7.2 Hz, 6H). MS (ESI) m/z (M+H)⁺ 475.2.

### N-(4-AMINO-3,4-DIOXO-1-PHENYLBUTAN-2-YL)-3-(3-ISOPROPYL-2-OXO-2,3-DIHYDROBENZO[D]OXAZOL-7-YL)-1-METHYL-1H-PYRAZOLE-4-CARBOXAMIDE (114)

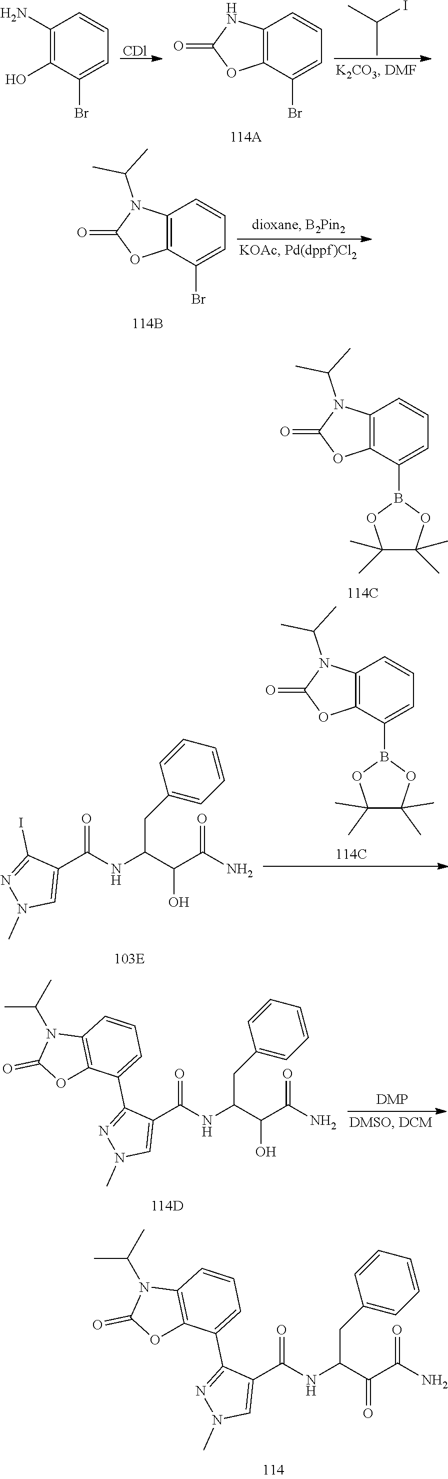

To a solution of 2-amino-6-bromophenol (3 g, 15 mmol) in THF (20 mL) was added CDI (5.2 g, 32 mmol), TEA (4.5 mL, 32 mmol). Then the mixture was stirred at 60° C. for 18 h. The reaction was concentrated under reduced pressure to remove solvent. H₂O (15 mL) and EA (20 mL) were added to the reaction and the organic layer was separated. The aqueous layer was extracted with EA (15 mL), the combined organic layer was washed with HCl (1M, 20 mL x 2), brine (20 mL), dried over anhydrous Na₂SO₄, filtered and concentrated under reduced pressure to afford compound 114A (2.5 g, yield 73%) as brown solid, which was used directly in next step. ¹H NMR (DMSO-ds, 400 MHz): δ 11.96 (br s, 1H), 7.30-7.23 (m, 1H), 7.13-7.04 (m, 2H).

To a solution of compound 114A (1.2 g, 5 mmol) in DMF (20 mL) was added Cs₂CO₃ (3.7 g, 11 mmol), 2-iodopropane (1.5 mL, 15 mmol) at 0° C. Then the mixture was stirred at 15° C. for 2 h. The reaction was concentrated under reduced pressure to remove solvent. H₂O (20 mL) and EA (20 mL) were added to the reaction and the organic layer was separated. The aqueous layer was extracted with EA (20 mL×2), the combined organic layer was washed with brine (20 mL), dried over anhydrous Na₂SO₄, filtered and concentrated under reduced pressure to afford compound 114B (1.4 g, yield 97.5%) as brown solid, which was used directly in next step. ¹H NMR (DMSO-d₆, 400 MHz): δ7.46-7.40 (m, 1H), 7.33 (dd, J=0.8, 8.3 Hz, 1H), 7.19-7.12 (m, 1H), 4.53-4.39 (m, 1H), 1.45 (d, J=6.8 Hz, 6H).

Compounds 103E and intermediate 114C were converted to compound 114 using procedures as described in Example 1. Compound 114 (78 mg, yield: 77.55%) as a white solid was obtained. ¹H NMR (DMSO-d₆, 400 MHz): δ 8.33-8.22 (m, 2H), 8.00-7.93 (m, 1H), 7.77 (s, 1H), 7.38 (d, J=7.8 Hz, 1H), 7.31-7.18 (m, 5H), 7.15 (t, J=7.9 Hz, 1H), 7.10-7.06 (m, 1H), 5.30-5.19 (m, 1H), 4.50 (quin, J=6.9 Hz, 1H), 3.97-3.88 (m, 3H), 3.13 (dd, J=3.9, 13.9 Hz, 1H), 2.90-2.73 (m, 1H), 1.47 (d, J=6.8 Hz, 6H). MS (ESI) m/z (M+H)⁺ 476.1.

### N-(4-AMINO-3,4-DIOXO-1-PHENYLBUTAN-2-YL)-3-(2,2-DIMETHYLBENZO[D][1,3]DIOXOL-4-YL)-1-METHYL-1H-PYRAZOLE-4-CARBOXAMIDE (112)

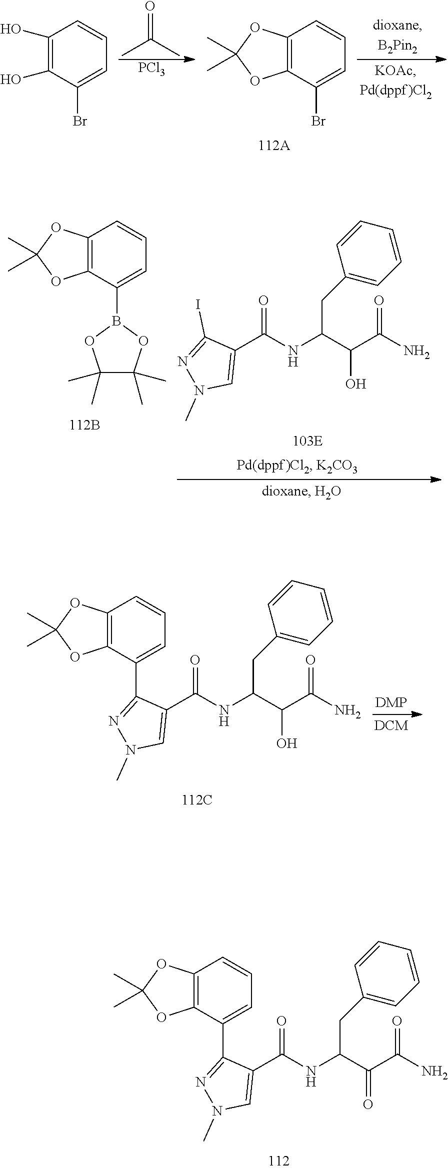

To a cold (0° C.) solution of 3-bromobenzene-1,2-diol (2 g, 10.58 mmol), acetone (1 mL, 12.70 mmol) in toluene (11 mL) was added dropwise PCl₃ (581 mg, 4.23 mmol), then the mixture was stirred at 80° C. for 48 h. The mixture was quenched with H₂O (20 mL) and extracted with DCM (10 mL×2). The organic phase was dried over Na₂SO₄, filtered and concentrated under vacuum. The product was purified by Flash Column Chromatography (0-50% EA/PE). Compound 112A (1 g, yield 41.26%) was obtained as a white liquid. ¹H NMR (DMSO-d₆, 400 MHz): 6.98 (dd, J=1.0, 8.3 Hz, 1H), 6.85 (dd, J=1.0, 7.8 Hz, 1H), 6.80-6.71 (m, 1H), 1.76-1.60 (m, 7H)

To a mixture of compound 112A (300 mg, 1.31 mmol) and B₂Pin₂ (665 mg, 2.62 mmol) in dioxane (5 mL) was added KOAc (386 mg, 3.93 mmol) and Pd(dppf)Cl₂ (96 mg, 130.96 umol) in one portion. The mixture was stirred at 90° C. for 18 h under N₂ atmosphere. The mixture was filtered and concentrated under vacuum. Compound 112B (400 mg, crude) was obtained as a black oil, which was used for next step without purification

Compounds 103E and intermediate 112B were converted to compound 114 using procedures as described in Example 1. Compound 112 (41 mg, yield: 74.51%) as a pale-yellow solid was obtained. ¹H NMR (DMSO-d₆, 400 MHz): δ 8.05 (br s, 2H), 7.99 (br s, 1H), 7.75 (br s, 1H), 7.30-7.06 (m, 5H), 6.72 (br s, 3H), 5.25 (br s, 1H), 3.85 (s, 3H), 3.08 (br d, J=13.2 Hz, 1H), 2.82-2.72 (m, 1H), 1.42 (br d, J=11.2 Hz, 6H).

### Example 26

### COMPOUNDS 93 AND 104 N-(1-OXO-3-PHENYL-1-(1H-TETRAZOL-5-YL)PROPAN-2-YL)-4-PHENYL-1,2,5-THIADIAZOLE-3-CARBOXAMIDE (93)

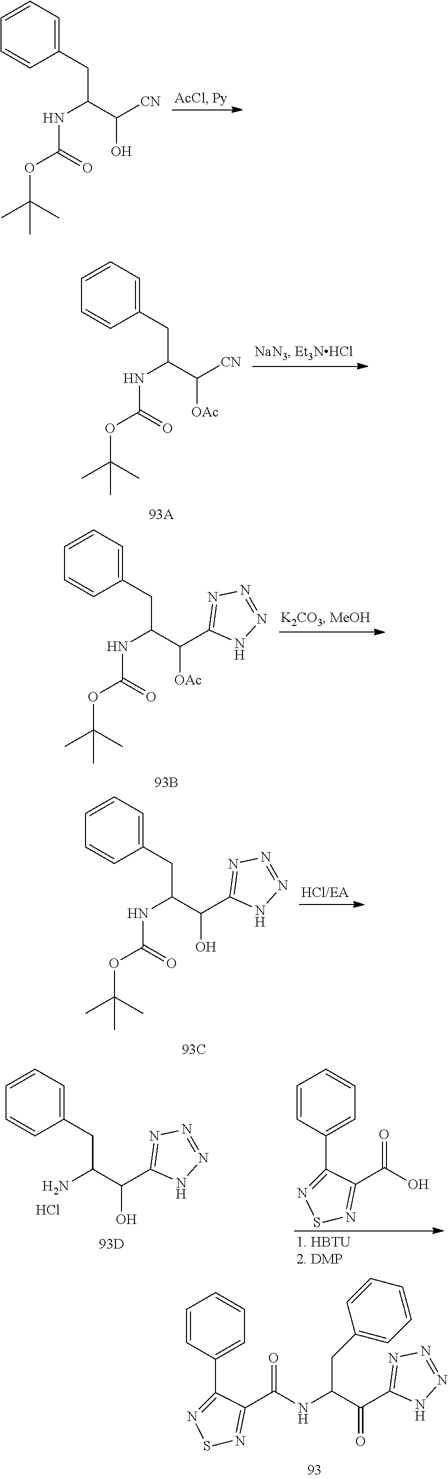

To a solution of tert-butyl (1-cyano-1-hydroxy-3-phenylpropan-2-yl)carbamate (1 g, 3.62 mmol) in DCM (15 mL) was added Pyridine (6.19 mmol, 0.5 mL), then acetyl chloride (5.61 mmol, 0.4 mL) was added dropwise, the mixture was stirred at 10° C. for 20 h. The reaction mixture was diluted with DCM (20 mL) and water (50 mL), the aqueous phase was extracted with DCM (20 mL×2), the organic layers were washed with 1N HCl (30 mL), sat. NaHCO₃ (30 mL) and brine (50 mL), dried over Na₂SO₄, filtered and concentrated to give a residue. Compound 93A (1 g, yield: 86.7%) as light yellow oil was obtained, which was used into the next step without further purification. ¹H NMR (400 MHz, CDCl₃) δ 7.36-7.27 (m, 3H), 7.22-7.16 (m, 2H), 5.42-5.33 (m, 1H), 4.70 (d, J=8.5 Hz, 1H), 4.32 (br s, 1H), 3.10-2.83 (m, 2H), 2.16 (s, 3H), 1.40 (s, 9H). MS (ESI) m/z (M+Na)⁺341.1.

To a mixture of compound 93A (500 mg, 1.57 mmol), Et₃N.HCl (432 mg, 3.14 mmol) in toluene (15 mL) was added NaN₃ (250 mg, 3.85 mmol), the mixture was stirred at 110° C. for 18 h. The reaction mixture was diluted with toluene (20 mL) and extracted with water (50 mL×3), the combined water layers were acidized with concentrated HCl to pH ˜2, and extracted with EA (30 mL×2), the organic layers were washed with brine (50 mL), dried over Na₂SO₄, filtered and concentrated to give a residue. The residue was triturated in EA (2 mL) and PE (20 mL) twice, filtered and dried in vacuo. Compound 93B (500 mg, yield: 74.4%) as light yellow solid was obtained. ¹H NMR (400 MHz, DMSO-d₆) 5 7.33-7.16 (m, 6H), 7.02 (d, J=9.0 Hz, 1H), 6.01-5.89 (m, 1H), 4.23-4.16 (m, 1H), 2.86-2.64 (m, 2H), 2.21-2.10 (m, 3H), 1.26-1.18 (m, 9H). MS (ESI) m/z (M+H)⁺ 362.2.

To a solution of compound 93B (400 mg, 1.11 mmol) in MeOH (15 mL) was added K₂CO₃ (610 mg, 4.41 mmol) in H₂O (3 mL), the mixture was stirred at 15° C. for 4 h. The reaction mixture was concentrated to remove MeOH, diluted with water (20 mL), extracted with EA (20 mL), the aqueous layer was acidized with concentrated HCl to pH ˜2, extracted with EA (20 mL×2), the organic layers were dried over Na₂SO₄, filtered and concentrated to give a residue. Compound 93C (420 mg, crude) was obtained as light yellow solid, which was used into the next step without further purification. ¹H NMR (400 MHz, DMSO-d₆) δ 7.30-7.16 (m, 6H), 6.56 (d, J=9.0 Hz, 1H), 6.37 (br d, J=4.0 Hz, 1H), 5.02 (t, J=4.5 Hz, 1H), 3.99-3.92 (m, 1H), 2.98-2.57 (m, 2H), 1.24 (s, 9H). MS (ESI) m/z (M+Na)⁺342.2.

To a solution of compound 93C (420 mg, 1.32 mmol) in EA (3 mL) was added HC/EtOAc (4M, 3 mL), the mixture was stirred at 15° C. for 2 h. The reaction mixture was concentrated to give a residue. The residue was triturated in EA (3 mL) and PE (20 mL), filtered and dried in vacuo. Compound 93D (300 mg, yield: 89.2%, HCl) as light yellow solid was obtained. ¹H NMR (400 MHz, DMSO-d₆) δ 8.26 (br s, 3H), 7.39-7.12 (m, 6H), 5.03 (t, J=4.5 Hz, 1H), 3.82 (s, 1H), 3.08-2.91 (m, 2H). MS (ESI) m/(M+Na)⁺276.2

Compounds 93D and 4-phenyl-1,2,5-thiadiazole-3-carboxylic acid were converted to compound 93 using procedures as described in Example 17. Compound 93 (15 mg, yield: 37.7%) as a white solid was obtained. ¹H NMR (400 MHz, DMSO-d₆) δ 9.33 (br dd, J=7.3, 16.8 Hz, 1H), 7.66-7.56 (m, 2H), 7.49-7.42 (m, 1H), 7.42-7.34 (m, 2H), 7.33-7.06 (m, 5H), 5.74-5.67 (m, 1H), 3.16-3.10 (m, 2H). MS (ESI) m/z (M+H)⁺ 406.1.

### N-(1-OXO-3-PHENYL-1-(H-1,2,4-TRIAZOL-3-YL)PROPAN-2-YL)-4-PHENYL-1,2,5-THIADIAZOLE-3-CARBOXAMIDE (104)

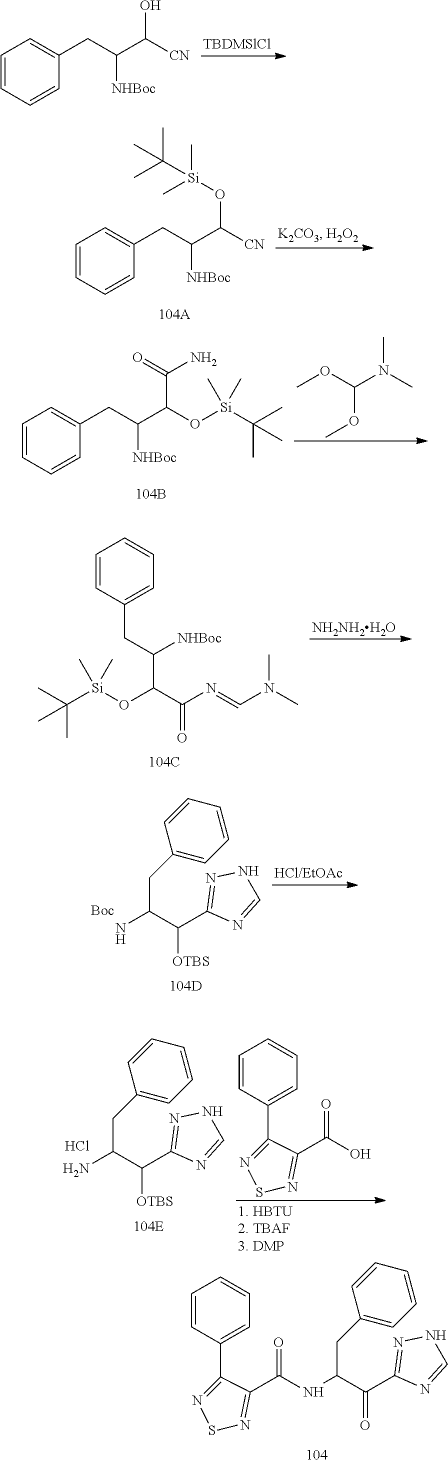

To a solution of tert-butyl (1-cyano-1-hydroxy-3-phenylpropan-2-yl)carbamate (500 mg, 1.81 mmol) in DMF (5 mL) was added imidazole (246 mg, 3.62 mmol) and TBDMSiCl (2.90 mmol, 0.35 mL) at 0° C. The mixture was stirred at 25° C. for 12 h. The mixture was diluted with EA (200 mL), washed with brine (200 mL), dried over Na₂SO₄, filtered and concentrated. The residue was purified by column chromatography (SiO₂, Petroleum ether/Ethyl acetate=10/1 to 1/1). Compound 104A (2.9 g) was obtained as a colorless oil. ¹H NMR (400 MHz, CDCl₃) δ 7.36-7.14 (m, 6H), 4.75-4.61 (m, 1H), 4.10-3.97 (m, 1H), 3.20-2.70 (m, 2H), 1.38 (s, 9H), 1.00-0.83 (m, 9H), 0.26-0.08 (m, 6H).

To a solution of compound 104A (450 mg, 1.15 mmol) and K₂CO₃ (318 mg, 2.30 mmol) in DMSO (10 mL) was added H₂O₂ (23.04 mmol, 2.21 mL, 30% purity) at 0° C., the mixture was stirred at 15° C. for 20 h. The reaction mixture was quenched with saturated Na₂S₂O₃ (20 mL) slowly at ice water, diluted with water (30 mL), extracted with EtOAc (30 mL×3), the organic layers were washed with brine (30 mL×2), dried over Na₂SO₄, filtered and concentrated to give a residue. Compound 104B (400 mg, crude) was obtained as colorless oil, which was used into the next step without further purification.

A solution of compound 104B (400 mg, 978.94 umol) in 1,1-dimethoxy-N,N-dimethyl-methanamine (75.28 mmol, 10 mL) was stirred at 30° C. for 1 h. The reaction mixture was diluted with water (50 mL) at ice water, extracted with EA (20 mL×3), the organic layers were washed with brine (30 mL×2), dried over Na₂SO₄, filtered and concentrated to give a residue. Compound 104C (420 mg, crude) was obtained as light yellow oil, which was used into the next step without further purification.

To a solution of compound 104C (410 mg, 884.22 umol) in CH₃COOH (5 mL) was added NH₂NH₂—H₂O (884.22 umol, 0.43 mL), the mixture was stirred at 85° C. for 1.5 h. The reaction mixture was diluted with water (60 mL) at ice water, extracted with EA (30 mL×3), the organic layers were washed with brine (80 mL×2), dried over Na₂SO₄, filtered and concentrated to give a residue. Compound 104D (400 mg, crude) was obtained as light yellow oil, which was used into the next step without further purification. MS (ESI) m/z (M+H)⁺ 433.3.

To a solution of compound 104D (400 mg, 924.58 umol) in EA (3 mL) was added HCl/EtOAc (4M, 4.62 mL), the mixture was stirred at 15° C. for 2 h. The reaction mixture was concentrated to give a residue. Compound 104E (350 mg, crude, HCl) was obtained as yellow solid, which was used into the next step without further purification. MS (ESI) m/z (M+H)⁺ 333.2.

Compounds 104E and 4-phenyl-1,2,5-thiadiazole-3-carboxylic acid were coupled using peptide coupling conditions as in Example 17 and then deprotection using TBAF followed by oxidation using procedure for Example 17 to obtain compound 104. Compound 104 (40 mg, yield: 53.5%) as a white solid was obtained. ¹H NMR (400 MHz, CD₃CN) 8 8.45 (s, 1H), 7.83 (d, J=7.1 Hz, 1H), 7.66-7.55 (m, 2H), 7.49-7.36 (m, 3H), 7.34-7.16 (m, 6H), 5.92-5.87 (m, 1H), 3.45 (dd, J=4.6, 14.2 Hz, 1H), 3.12 (dd, J=8.6, 13.9 Hz, 1H). MS (ESI) m/z (M+H)⁺ 405.1.

### Example 27

### COMPOUNDS 113, 110, AND 109 N-(4-(METHOXYAMINO)-3,4-DIOXO-1-PHENYLBUTAN-2-YL)-2-(3-PHENYL-1H-PYRAZOL-1-YL)NICOTINAMIDE (113)

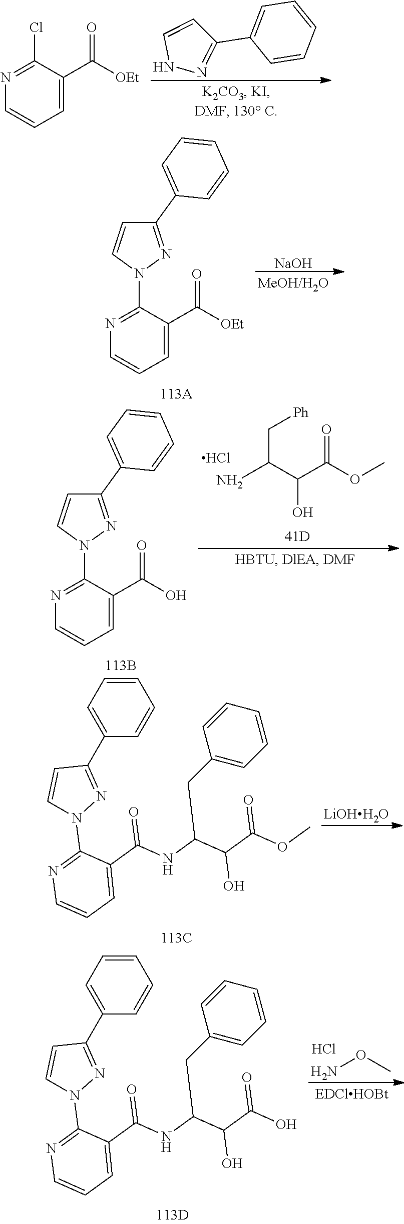

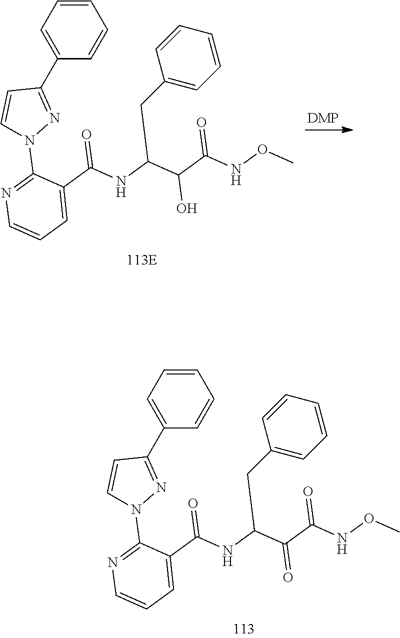 [SMILES: CONC(=O)C(O)C(Cc1ccccc1)NC(=O)c1cccnc1-n1ccc(-c2ccccc2)n1 ; CONC(=O)C(=O)C(Cc1ccccc1)NC(=O)c1cccnc1-n1ccc(-c2ccccc2)n1]

To a solution of ethyl 2-chloronicotinate (2 g, 10.78 mmol) and 3-phenyl-1H-pyrazole (2.33 g, 16.16 mmol) in DMF (30 mL) was added K₂CO₃ (4.47 g, 32.33 mmol) and KI (1.79 g, 10.78 mmol). The mixture was stirred at 130° C. for 16 h. The reaction was filtered, the filtrate was added H₂O (100 mL), extracted with EA (30 mL×2), the organic phase was washed with brine (100 mL), filtered, and concentrated. The residue was purified by flash silica gel chromatography (ISCO®; 40 g SepaFlash® Silica Flash Column, Eluent of 15% Ethyl acetate/Petroleum ethergradient @ 40 mUmin). Compound 13A (1 g, yield: 28.5%) was obtained as a white solid. ¹H NMR (400 MHz, CDCl₃) δ 8.49 (dd, J=1.7, 4.6 Hz, 1H), 8.42 (d, J=2.4 Hz, 1H), 7.94 (dd, J=1.7, 7.6 Hz, 1H), 7.89-7.78 (m, 2H), 7.40 (t, J=7.3 Hz, 2H), 7.33 (br d, J=7.3 Hz, 1H), 7.29-7.23 (m, 1H), 6.79 (d, J=2.7 Hz, 1H), 4.45-4.25 (m, 2H), 1.14 (t, J=7.2 Hz, 3H). MS (ESI) m/z (M+H)⁺ 294.1.

To a solution of compound 113A (1 g, 3.41 mmol) in MeOH (20 mL) was added NaOH (410 mg, 10.25 mmol) in H₂O (5 mL). The mixture was stirred at 15° C. for 16 h. The reaction was diluted with H₂O (20 mL) and the mixture was concentrated under reduced pressure to remove solvent. The aqueous layer was washed with MTBE (20 mL) and the aqueous layer was treated with HCl (1N) until pH ˜4. The aqueous layer was extracted with EA (20 mL×3), the combined organic layer was dried over anhydrous Na₂SO₄, filtered and concentrated under reduced pressure. Compound 113B (600 mg, yield: 66.4%) was obtained as a white solid, which was used directly in next step. ¹H NMR (400 MHz, DMSO-d₆) δ 14.19-12.15 (m, 1H), 8.64-8.48 (m, 2H), 8.06 (dd, J=1.7, 7.6 Hz, 1H), 7.95-7.84 (m, 2H), 7.52-7.39 (m, 3H), 7.38-7.29 (m, 1H), 7.04 (d, J=2.7 Hz, 1H)

To a solution of compound 113B (250 mg, 942 umol) in DMF (10 mL) was added intermediate 41D (280 mg, 1 mmol, HCl), HBTU (428 mg, 1 mmol), DIEA (500 uL, 2 mmol). Then the mixture was stirred at 15° C. for 6 h. The reaction was concentrated under reduced pressure to remove solvent. H₂O (10 mL) was added to the reaction and the precipitation was filtered. The filtered cake was concentrated under reduced pressure to afford compound 113C (420 mg, yield: 97.6%) as light yellow solid, which was used directly in next step. ¹H NMR (DMSO-d₆,400 MHz): δ 8.66-8.53 (m, 1H), 8.53-8.36 (m, 2H), 7.91-7.75 (m, 3H), 7.69-7.46 (m, 1H), 7.44-7.37 (m, 2H), 7.37-7.25 (m, 3H), 7.22-7.14 (m, 3H), 7.03-6.95 (m, 1H), 5.77-5.53 (m, 1H), 4.62-4.36 (m, 1H), 4.33-3.87 (m, 1H), 3.55-3.51 (m, 3H), 2.84-2.75 (m, 1H), 2.69-2.62 (m, 1H).

To a solution of compound 113C (300 mg, 657 umol) in MeOH (10 mL) was added a solution of LiOH.H₂O (140 mg, 3 mmol) in H₂O (2 mL). Then the mixture was stirred at 15° C. for 8 h. The reaction was diluted with H₂O (20 mL) and the mixture was concentrated under reduced pressure. The aqueous layer was washed with MTBE (20 mL) and the aqueous layer was treated with HCl (1N) until pH ˜3. The precipitation was filtered and concentrated under reduced pressure to afford compound 113D (270 mg, yield: 92.8%) as white solid, which was used directly in next step. ¹H NMR (DMSO-d₆, 400 MHz): δ 812.62 (br s, 1H), 8.68-8.54 (m, 1H), 8.53-8.40 (m, 1H), 8.39-8.31 (m, 1H), 7.93-7.74 (m, 3H), 7.52-7.47 (m, 1H), 7.42-7.35 (m, 2H), 7.34-7.24 (m, 3H), 7.23-7.16 (m, 3H), 7.03-6.97 (m, 1H), 5.56-5.17 (m, 1H), 4.63-4.40 (m, 1H), 4.32-3.80 (m, 1H), 2.90-2.63 (m, 2H).

To a solution of compound 113D (220 mg, 497.21 umol) and 0-methyhydroxylamine (330 mg, 3.95 mmol, HCl) in DMF (5 mL) and DCM (15 mL) was added EDCI (760 mg, 3.96 mmol), HOBt (540 mg, 4.00 mmol) and TEA (4.96 mmol, 0.69 mL), the mixture was stirred at 30° C. for 20 h. The reaction mixture was concentrated to remove solvent, then diluted with water (80 mL), extracted with EA (30 mL×3), the organic layers were washed with water (50 mL) and brine (50 mL), dried over Na₂SO₄, filtered and concentrated to give a residue. The residue was purified by column chromatography (SiO₂, Petroleum ether/Ethyl acetate=5/1 to 0/1 to EA/MeOH=5/1). Compound 113E (70 mg, yield: 28.8%) as white solid was obtained. ¹H NMR (400 MHz, Methanol-d₄) δ 8.53 (dd, J=1.8, 4.8 Hz, 1H), 8.41 (d, J=2.4 Hz, 1H), 8.01-7.81 (m, 3H), 7.45-7.33 (m, 3H), 7.31-7.17 (m, 6H), 6.88 (d, J=2.7 Hz, 1H), 4.67-4.57 (m, 1H), 4.01 (d, J=2.2 Hz, 1H), 3.66 (s, 3H), 2.93-2.80 (m, 2H). MS (ESI) m/z (M+H)⁺ 472.3.

To a solution of compound 113E (60 mg, 127.25 umol) in DMSO (3 mL) and DCM (40 mL) was added DMP (170 mg, 400.81 umol), the mixture was stirred at 20° C. for 2 h. The reaction mixture was quenched with Na₂S₂O₃ (30 mL) and NaHCO₃ (30 mL), extracted with DCM (30 mL×2), the organic layers were washed with water (50 mL) and brine (50 mL), dried over Na₂SO₄, filtered and concentrated to give a residue which was purified by preparatory-TLC (plate 1, DCM:i-Pr₂O=13:1). Compound 113 (23 mg, yield: 38.5%) as white solid was obtained. ¹H NMR (400 MHz, CD₃CN) 8 8.50 (dd, J=1.7, 4.9 Hz, 1H), 8.43 (d, J=2.7 Hz, 1H), 7.77 (d, J=8.6 Hz, 3H), 7.42-7.33 (m, 4H), 7.16 (s, 3H), 7.08 (s, 2H), 6.86 (d, J=2.4 Hz, 1H), 5.67-5.56 (m, 1H), 3.66 (s, 3H), 3.16 (dd, J=5.7, 14.6 Hz, 1H), 2.89 (dd, J=7.5, 14.1 Hz, 1H). MS (ESI) m/z (M+H)⁺ 470.2.

### N-(4-(METHOXYAMINO)-3,4-DIOXO-1-PHENYLBUTAN-2-YL)-2-(3-PHENYL-1H-PYRAZOL-1-YL)NICOTINAMIDE (110)

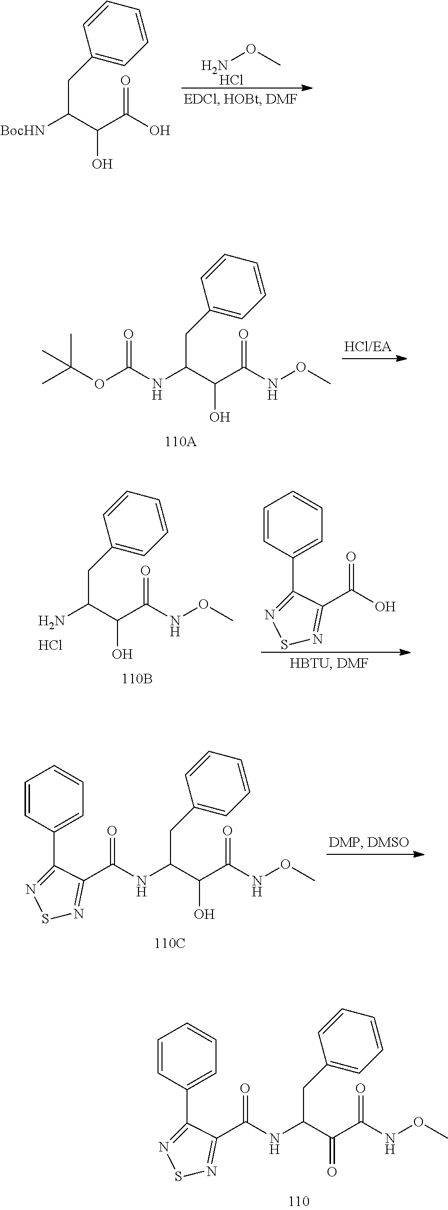

A mixture of 3-((tert-butoxycarbonyl)amino)-2-hydroxy-4-phenylbutanoic acid (600 mg, 2.03 mmol), O-methylhydroxylamine (340 mg, 4.07 mmol, HC %) EDCI (900 mg, 4.69 mmol), DIEA (1.11 g, 8.61 mmol, 1.50 mL) and HOBt (300 mg, 2.22 mmol) in DMF (10 mL) was degassed and purged with N₂ for 3 times, and then the mixture was stirred at 20° C. for 16 h under N₂ atmosphere. The reaction mixture was diluted with H₂O (100 mL), extracted with EA (50 mL×3) and washed with NaHCO₃(aq) (100 mL). The organic layers were washed with brine (100 mL), dried over Na₂SO₄, filtered and concentrated under reduced pressure to give a residue. The crude product compound 110A (620 mg, crude) as yellow solid, which was used into the next step without further purification. ¹H NMR (400 MHz, DMSO-d₆) δ 11.09 (d, J=14.9 Hz, 1H), 7.28-7.06 (m, 5H), 6.69-6.04 (m, 1H), 5.82-5.53 (m, 1H), 3.96-3.72 (m, 2H), 3.52 (d, J=12.7 Hz, 3H), 2.79-2.68 (m, 1H), 2.61 (br d, J=6.6 Hz, 1H), 1.25 (s, 4.5H), 1.23 (s, 4.5H).

To a solution of compound 110A (800 mg, 2.47 mmol) in EA (8 mL) was added HCV/EtOAc (4M, 8 mL). The mixture was stirred at 20° C. for 1.5 h. The reaction was concentrated to give a residue. The residue was triturated in EA:MTBE=1:1 (20 mL), filtered, the cake was obtained. Compound 110B (550 mg, yield: 85.5%, HCl) was obtained as a white solid. 1H NMR (400 MHz, DMSO-d₆) δ 11.44 (s, 0.5H), 11.41 (s, 0.5H), 8.15-7.92 (m, 3H), 7.35-7.13 (m, 5H), 6.67 (br s, 0.5H), 6.45 (br s, 0.5H), 4.25 (d, J=2.2 Hz, 0.5H), 3.87 (br s, 0.5H), 3.72-3.56 (m, 1H), 3.54 (s, 1.5H), 3.46 (s, 1.5H), 2.89-2.74 (m, 2H).

To a solution of 4-phenyl-1,2,5-thiadiazole-3-carboxylic acid (200 mg, 969.83 umol) and compound 110B (300 mg, 1.15 mmol, HCl) in DMF (10 mL) was added HBTU (440 mg, 1.16 mmol) and DIEA (593.60 mg, 4.59 mmol, 0.8 mL). The mixture was stirred at 20° C. for 1 h. The reaction mixture was diluted with H₂O (50 mL), extracted with EA (30 mL×3). The organic layers were washed with brine (100 mL), dried over Na₂SO₄, filtered and concentrated under reduced pressure to give a residue. The residue was purified by preparatory-TLC (SiO₂, DCM:MeOH=15:1). Compound 110C (300 mg, yield: 73.5%) was obtained as a white solid. ¹H NMR (400 MHz, DMSO-d₆) δ 11.32 (s, 0.5H), 11.19 (s, 0.5H), 8.95 (d, J=9.0 Hz, 0.5H), 8.57 (d, J=9.3 Hz, 0.5H), 7.55 (d, J=7.3 Hz, 1H), 7.47-7.23 (m, 9H), 6.08 (d, J=6.0 Hz, 0.5H), 6.00 (d, J=6.3 Hz, 0.5H), 4.62-4.41 (m, 1H), 4.11 (dd, J=4.4, 5.9 Hz, 0.5H), 4.02 (dd, J=3.3, 6.3 Hz, 0.5H), 3.60 (s, 1.5H), 3.52 (s, 1.5H), 2.97-2.88 (m, 1H), 2.85-2.77 (m, 1H). MS (ESI) m/z (M+H)⁺ 413.1.

To a solution of compound 110C (150 mg, 363.67 umol) in DCM (30 mL) and DMSO (3 mL) was added DMP (500 mg, 1.18 mmol, 364.96 uL). The mixture was stirred at 20° C. for 2 h. The reaction mixture was diluted with DCM (20 mL), quenched with saturated NaHCO₃ (40 mL) and saturated Na₂S₂O₃ (40 mL), the mixture was stirred 5 min. The organic layer was washed with water (40 mL×2), brine (40 mL×2), dried over Na₂SO₄, filtered and then concentrated to give a residue. The residue was purified by preparatory-TLC (SiO₂, DCM:i-PrOH=10:1). Compound 110 (20 mg, yield: 11.7%) was obtained as a white solid. ¹H NMR (400 MHz, DMSO-d₆) δ 9.15 (br s, 1H), 8.05-7.92 (m, 1H), 7.71 (br d, J=7.5 Hz, 1H), 7.65 (br d, J=7.3 Hz, 1H), 7.50-7.39 (m, 3H), 7.33-7.21 (m, 5H), 5.48 (br s, 1H), 3.72 (br s, 3H), 3.26 (br dd, J=3.8, 14.3 Hz, 1H), 3.01-2.90 (m, 1H). MS (ESI) m/z (M+H)⁺ 411.1.

### N-(4-(2,2-DIMETHYLHYDRAZINEYL)-3,4-DIOXO-1-PHENYLBUTAN-2-YL)-4-PHENYL-1,2,5-THIADIAZOLE-3-CARBOXAMIDE (109)

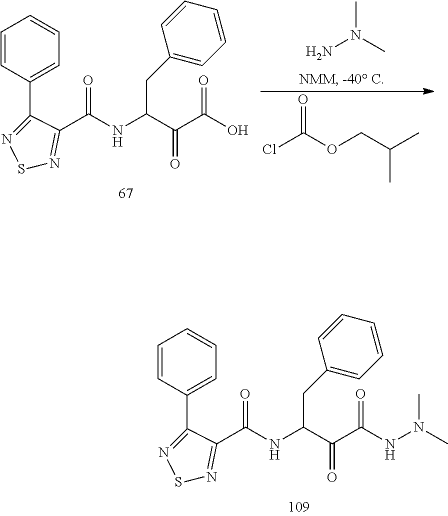

To a solution of compound 67 (150 mg, 393.28 umol) in THF (3 mL) was added 4-methylmorpholine (59.67 mg, 589.92 umol, 64.86 uL), isobutyl carbonochloridate (59.09 mg, 432.61 umol, 56.81 uL) at −40° C. The mixture was stirred at −40° C. for 30 min. A solution of 1,1-dimethyhydrazine (79.20 mg, 1.32 mmol, 0.1 mL) in THF (3 mL) was added at −40° C. The mixture was stirred at −40° C. for 1.5 h. The reaction mixture was quenched with H₂O (2 mL) and partitioned between EtOAc (20 mL) and H₂O (20 mL). The organic phase was separated, washed with NaHCO₃ (20 mL), dried over Na₂SO₄, filtered and concentrated under reduced pressure to give a residue. The residue was purified by prep-TLC (SiO₂, EA). Compound 109 (15 mg, yield: 8.3%) was obtained as a white solid. ¹H NMR (400 MHz, DMSO-d₆) δ 9.50 (s, 1H), 9.06 (br d, J=8.0 Hz, 1H), 7.73-7.54 (m, 2H), 7.48-7.38 (m, 3H), 7.32-7.24 (m, 5H), 5.63-5.13 (m, 1H), 3.35-3.21 (m, 1H), 3.02-2.95 (m, 1H), 2.60-2.52 (m, 6H). MS (ESI) m/z (M+H)⁺ 424.1

### Example 28 COMPOUND 111

### N-(4-HYDROXY-3-OXO-1-PHENYLBUTAN-2-YL)-4-PHENYL-1,2,5-THIADIAZOLE-3-CARBOXAMIDE (111)

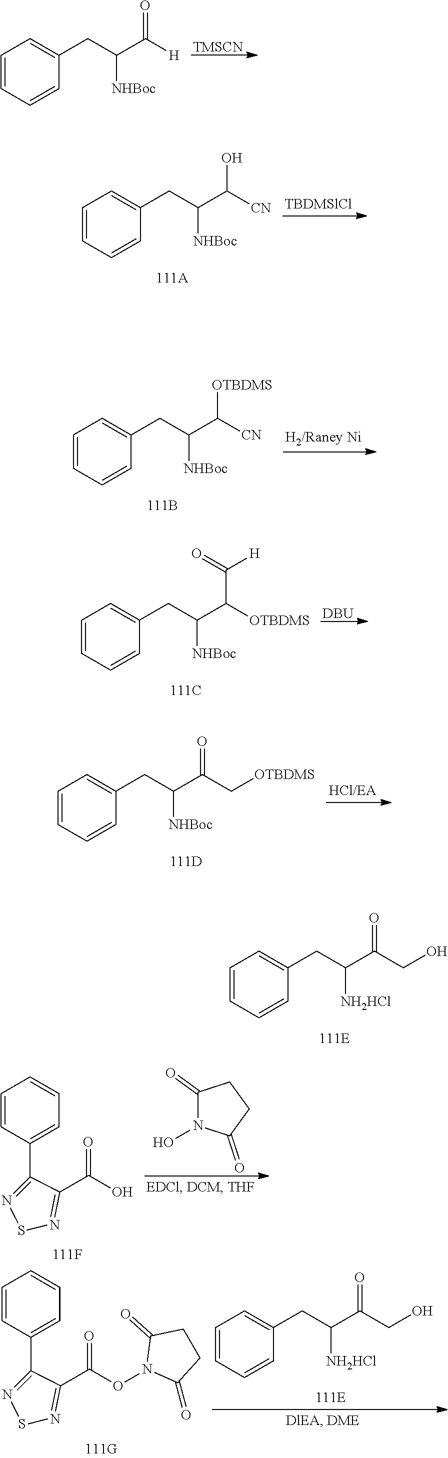

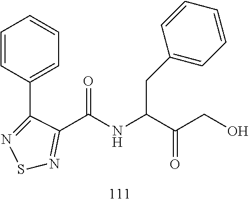 [SMILES: O=C(NC(Cc1ccccc1)C(=O)CO)c1nsnc1-c1ccccc1]

To a solution of tert-butyl (1-oxo-3-phenylpropan-2-yl)carbamate (8.3 g, 33.29 mmol) in MeOH (200 mL) was added TMSCN (66.59 mmol, 8.33 mL) and CsF (2.53 g, 16.65 mmol). The mixture was stirred at 25° C. for 0.5 hr. The mixture was concentrated and diluted with EA (200 mL), washed with H₂O (200 mL), brine (200 mL), dried over Na₂SO₄ and concentrated. The residue was purified by column chromatography (SiO₂, Petroleum ether/Ethyl acetate=10/1 to 1:1). Compound 111A (9.4 g, crude) was obtained as a yellow oil. ¹H NMR (400 MHz, DMSO-d₆) δ 7.34-6.70 (m, 6H), 4.60-4.31 (m, 1H), 3.87-3.76 (m, 1H), 3.05-2.89 (m, 1H), 2.74-2.54 (m, 1H), 1.35-1.12 (m, 9H).

To a solution of compound 111A (9.4 g, 34.02 mmol) in DMF (100 mL) was added imidazole (4.63 g, 68.03 mmol) and TBDMSCl (8.20 g, 54.43 mmol) at 0° C. The mixture was stirred at 25° C. for 12 h. The mixture was concentrated and diluted with EA (200 mL), washed with H₂O (200 mL), brine (200 mL), dried over Na₂SO₄ and concentrated and the resulting residue was purified by column chromatography (SiO₂, Petroleum ether/Ethyl acetate=20/1 to 1:1). Compound 111B (9 g, 67.7% yield) was obtained as a colorless oil. ¹H NMR (400 MHz, DMSO-d₆) δ 7.36-7.14 (m, 6H), 4.79-4.62 (m, 1H), 3.90-3.68 (m, 1H), 3.01-2.86 (m, 1H), 2.79-2.56 (m, 1H), 1.37-1.15 (m, 9H), 0.99-0.82 (m, 9H), 0.23-0.09 (m, 6H).

To a solution of compound 111B (7 g, 17.92 mmol) in H₂O (60 mL) and EtOH (240 mL) was added Raney-Ni (3.07 g, 35.84 mmol) and H₂SO₄ (1 M, 35.84 mL). The mixture was stirred at 25° C. for 2 h under H₂ (4 psi). The mixture was filtered and the filtrate was added NaHCO₃ (aqueous) until pH=8, then the mixture was concentrated and diluted with EA (200 mL), washed with H₂O (200 mL), brine (200 mL), dried over Na₂SO₄ and concentrated and the resulting residue was purified by column chromatography (SiO₂, Petroleum ether/Ethyl acetate=10/1). Compound 111C (7 g, crude) was obtained as a colorless oil. The crude product was used in next step directly.

To a solution of compound 111C (3 g, 7.62 mmol) in DCM (200 mL) was added DBU (19.06 mmol, 2.87 mL). The mixture was stirred at 20° C. for 3 h. The combined mixture was washed with H₂O (200 mL), brine (200 mL), dried over Na₂SO₄ and concentrated. The residue was purified by column chromatography (SiO₂, Petroleum ether/Ethyl acetate=1/0 to 5:1). The residue was purified by preparatory-HPLC (basic condition). Compound 111D (400.0 mg, 13.3% yield) was obtained as a colorless oil. ¹H NMR (400 MHz, CDCl₃) δ 7.24-7.11 (m, 3H), 7.07-7.01 (m, 2H), 5.04-4.91 (m, 1H), 4.80-4.69 (m, 1H), 4.29-4.04 (m, 2H), 3.15-2.81 (m, 2H), 1.31 (s, 9H), 0.84 (s, 9H), 0.08-−0.04 (m, 6H).

To a solution of compound 111D (350.0 mg, 889.25 umol) in EA (5 mL) was added HCl/EtOAc (4M, 4.45 mL). The mixture was stirred at 25° C. for 0.5 h. The mixture was concentrated. The crude product was triturated with EA (10 mL), the cake was dried in vacuum. Compound 111E (160.0 mg, crude, HCl) was obtained as a white solid. The crude product was used in next step directly.

To a solution of compound 111F (300.0 mg, 1.45 mmol) in DCM (10 mL) and THF (10 mL) was added 1-hydroxypyrrolidine-2,5-dione (184.2 mg, 1.60 mmol) and EDCI (334.6 mg, 1.75 mmol). The mixture was stirred at 25° C. for 2 h. The mixture was concentrated, diluted with EA (20 mL), washed with HCl (1M, 20 mL), saturated NaHCO₃ (aqueous, 20 mL), brine (20 mL), dried over Na₂SO₄ and concentrated. Compound 111G (440.0 mg, 94.6% yield) was obtained as a white solid. MS (ESI) m/z (M+Na)⁺ 326.0.

To a solution of compound 111G (80.0 mg, 263.77 umol) in DME (10 mL) was added DIEA (791.31 umol, 140 uL) and 3-amino-1-hydroxy-4-phenyl-butan-2-one (111E) (56.9 mg, 263.77 umol, HCl). The mixture was stirred at 25° C. for 1 hr. The mixture was concentrated. The residue was purified by preparatory-HPLC (basic condition). Compound 111 (15.0 mg, 15.3% yield) was obtained as a white solid. ¹H NMR (400 MHz, DMSO-d₆) δ 9.42-9.33 (m, 1H), 7.54-7.22 (m, 10H), 5.43-5.34 (m, 1H), 5.04-4.94 (m, 1H), 4.46-4.19 (m, 2H), 3.27-3.17 (m, 1H), 2.91-2.80 (m, 1H). MS (ESI) m/z (M+H)⁺ 368.1.

### Biological Data

### Example 29

Calpain 1, 2, and 9 activity and inhibition thereof was assessed by means of a continuous fluorescence assay. The SensoLyte 520 Calpain substrate (Anaspec Inc) was optimized for detecting calpain activity. This substrate contains an internally quenched 5-FAM/QXLTM 520 FRET pair. Calpains 1, 2, and 9 cleave the FRET substrate into two separate fragments resulting in an increase of 5-FAM fluorescence that is proportional to calpain activity

Assays were typically setup in black 384-well plates using automated liquid handling as follows. Calpain assay base buffer typically contains 50 mM Tris, pH 7.5, 100 mM NaCl and 1 mM DTT. Inhibitors were serially diluted in DMSO and used to setup 2× mixtures with calpains in the aforementioned buffer. After incubation at ambient temperature (25C), the reaction was initiated by adding a 2× mix of the fluorescent peptide substrate and CaCl2) (required for in-situ calpain activation) in the same buffer. Reaction progress curve data were typically collected for 10 min using excitation/emission wavelengths of 490 nm/520 nm on SpectraMax i3× or the FLIPR-Tetra plate readers (Molecular Devices Inc). Reaction rates were calculated from progress curve slopes typically over 1-5 min. Dose response curves (rate vs. log inhibitor concentration) were typically fit to a 4-parameter logistic function to extract IC50 values.

Calpain activity in SH-SY5Y cells and inhibition thereof were assessed by means of a homogeneous, fluorescence assay that uses the cell-permeable and pro-fluorescent calpain substrate Suc-LLVY-AMC (Sigma-Aldrich Inc). Upon intracellular calpain cleavage of Suc-LLVY-AMC, fluorescent amino-methyl-coumarin (AMC) is released into the media resulting in a continuous increase in fluorescence signal that is proportional to intra-cellular calpain activity.

Assays were typically setup by seeding SH-SY5Y cells in black 384-well plates at 40 k/per well in RPMI-1640 containing 1% serum followed by 37C overnight incubation. Next morning, cells were pre-incubated for 30 min with serially diluted compounds followed by addition of 100 uM of Suc-LLVY-AMC substrate. The continuous increase in AMC fluorescence is monitored using a FLIPR Tetra plate reader (Molecular Devices Inc) and slopes measured to report calpain activity. Dose response curves (slopes vs. log inhibitor concentration) were typically fit to a 4-parameter logistic function to extract IC50 values.

Calpain activity in SH-SY5Y cells and inhibition thereof were also assessed by a western blot based assay that measures a calpain-specific breakdown product of the alpha chain of non-erythrocytic spectrin (SBDP-150). Addition of the calcium ionophore A23187 was used to induce calpain activity and SBDP-150 formation.

These assays were typically setup by seeding SH-SY5Y cells in 96-well plates at 150 k/per well in DMEM containing 10% serum, followed by 37C incubation for 24 hrs. The cells were then pre-incubated for 60 min with serially diluted compounds followed by addition of 25 uM A23187 and further incubation for 90 min. Total cellular protein was extracted in RIPA buffer, boiled in gel loading buffer and run on SDS-PAGE gel. The gel was processed via Western Blotting (dry transfer) to quantify SBDP-150 (AA6 antibody, Enzo Inc) and either GAPDH or HSP90 as loading controls. Normalized SBDP-150 levels vs. log inhibitor concentration were plotted to get dose response curves that are typically fit to a 4-parameter logistic function to extract IC50 values.

### Calpain Inhibition

TABLE 2
Calpain inhibition assay

Column A: Human Calpain 1/NS1 IC50
Column B: Human Calpain 2/NS1 IC50
Column C: Human Calpain 9/NS1 IC50
Column D: SH-SY5Y Spectrin IC50
Column E: SH-SY5Y + AMC IC50

| | Column | Column | Column | Column | Column |
|---|---|---|---|---|---|
| Compound No. | A | B | C | D | E |
| 1 | A | A | A | F | F |
| 2 | C | B | C | F | E |
| 3 | A | A | A | E | D |
| 4 | A | A | A | F | D |
| 5 | A | A | A | E | D |
| 6 | A | A | A | D | D |
| 7 | A | A | A | D | D |
| 8 | A | A | A | E | F |
| 9 | A | A | A | E | F |
| 10 | B | A | A | F | F |
| 11 | A | A | A | E | D |
| 12 | B | A | A | F | F |
| 13 | A | A | A | F | F |
| 14 | A | A | A | F | F |
| 15 | A | A | A | E | D |
| 16 | A | A | A | E | E |
| 17 | A | A | A | F | E |
| 18 | A | A | A | F | D |
| 19 | A | A | A | F | D |
| 20 | A | A | A | D | D |
| 21 | A | A | A | D | D |
| 22 | A | A | A | D | D |
| 23 | A | A | A | D | D |
| 24 | A | A | A | E | D |
| 25 | A | A | A | E | ND |
| 26 | A | A | A | D | D |
| 27 | C | B | B | F | D |
| 28 | A | A | A | F | D |
| 29 | B | A | B | F | D |
| 30 | A | A | A | D | F |
| 31 | A | A | A | D | D |
| 32 | A | A | A | E | E |
| 33 | B | A | A | F | ND |
| 34 | A | A | A | E | D |
| 35 | A | A | A | D | D |
| 36 | A | A | A | D | D |
| 37 | A | A | A | F | F |
| 38 | A | A | A | D | D |
| 39 | A | A | A | D | E |
| 40 | A | A | A | F | D |
| 41 | A | A | A | F | D |
| 42 | A | A | A | D | D |
| 45 | A | A | A | E | D |
| 46 | A | A | A | D | D |
| 47 | A | A | A | E | F |
| 48 | A | A | A | D | E |
| 49 | A | A | A | F | E |
| 50 | A | A | A | E | E |
| 51 | A | A | A | E | F |
| 52 | A | A | A | E | F |
| 53 | ND | ND | ND | ND | ND |
| 54 | A | A | A | F | F |
| 55 | A | A | A | F | D |
| 57 | C | C | C | F | F |
| 58 | A | A | A | D | D |
| 59 | C | B | C | F | D |
| 60 | A | A | A | E | E |
| 61 | A | A | A | D | D |
| 62 | A | A | A | D | D |
| 63 | A | A | A | E | D |
| 64 | A | A | A | F | D |
| 65 | A | A | A | F | D |
| 67 | A | A | A | D | D |
| 68 | A | A | A | E | D |
| 69 | A | A | A | E | E |
| 70 | A | A | A | D | D |
| 71 | A | A | A | D | D |
| 72 | A | A | A | F | D |
| 73 | A | A | A | D | ND |
| 74 | A | A | A | F | ND |
| 75 | A | A | A | E | ND |
| 76 | A | A | A | E | ND |
| 77 | A | A | A | E | ND |
| 78 | A | A | A | E | D |
| 79 | A | A | A | D | D |
| 80 | A | A | A | D | D |
| 81 | A | A | A | E | D |
| 82 | C | C | C | F | F |
| 83 | A | A | A | F | D |
| 84 | A | A | A | E | D |
| 85 | C | C | C | F | D |
| 86 | C | C | C | F | F |
| 87 | A | A | A | E | D |
| 88 | A | A | A | F | D |
| 89 | A | A | A | D | D |
| 90 | A | A | A | E | D |
| 91 | A | A | A | D | D |
| 92 | A | A | A | E | D |
| 93 | C | C | C | F | F |
| 94 | A | A | A | D | D |
| 95 | A | A | A | E | F |
| 96 | A | A | A | D | D |
| 97 | A | A | A | D | D |
| 98 | A | A | A | E | D |
| 99 | A | A | A | F | F |
| 100 | A | A | A | E | E |
| 101 | A | A | A | D | E |
| 102 | A | A | A | D | F |
| 103 | A | A | A | D | E |
| 104 | C | C | C | F | F |
| 106 | A | A | A | ND | E |
| 107 | A | A | A | ND | E |
| 108 | A | A | A | ND | D |
| 109 | A | A | A | ND | E |
| 110 | A | A | A | ND | D |
| 111 | B | B | C | ND | F |
| 112 | A | A | A | ND | F |
| 113 | A | A | A | ND | E |
| 114 | A | A | A | ND | F |
| 115 | A | A | A | ND | D |

A: <3 uM;
B: 3-10 uM;
C: >10 uM;
D: <10 uM;
E: 10-25 uM;
F: >25 uM
ND: Not Determined

### Carbon Tetrachloride-Induced Liver Fibrosis in Mice or Rats

Carbon tetrachloride-induced liver fibrosis is a widely used and accepted model for evaluating novel antifibrotic therapies. The methods for inducing liver fibrosis by carbon tetrachloride administration is described in Lee, J Clin Invest, 1995 and Tsukamoto, Semin Liver Dis, 1990. Briefly, male C57B/6 mice are challenged with 1 mg/kg carbon tetrachloride (Sigma Aldrich, diluted 1:7 in corn or olive oil) administered by intraperitoneal injection twice weekly for a period of 4 weeks. Mice are euthanized on day 28. In an alternative implementation, Wistar rats are administered carbon tetrachloride by intraperitoneal injection three times per week for 8-12 weeks. Rats are euthanized at the termination of the experiment, 8-12 after study initiation.

Blood is collected by cardiac puncture and processed into serum for evaluation of liver enzymes (including ALT, AST, ALP, etc) at several timepoints throughout the study and at termination of the study. The liver tissues from all animals are collected and fixed by immersion in 10% neutral buffered formalin, processed, paraffin embedded, sectioned, mounted, and stained with Masson's Trichrome (Tri) or Picrosirius Red (PSR) using standard histological methods for evaluation of fibrosis severity.

### Mouse Unilateral Ureteral Obstruction Kidney Fibrosis Model

Female C57B/6 mice (Harlan, 4-6 weeks of age) will be given free access to food and water and allowed to acclimate for at least 7 days prior to test initiation. After acclimation, mice are anesthetized and undergo unilateral ureteral obstruction (UUO) surgery or sham to left kidney. Briefly, a longitudinal, upper left incision is performed to expose the left kidney. The renal artery is located and 6/0 silk thread is passed between the artery and the ureter. The thread is looped around the ureter and knotted 3 times insuring full ligation of ureter. The kidney is returned to abdomen, the abdominal muscle is sutured and the skin is stapled closed. All animals are euthanized 4, 8, 14, 21, or 28 days after UUO surgery. Following sacrifice blood is collected via cardiac puncture, the kidneys are harvested and one half of the kidney is frozen at −80° C. and the other half is fixed in 10% neutral buffered formalin for histopathological assessment of kidney fibrosis.

### Bleomycin Dermal Fibrosis Model

Bleomycin (Calbiochem, Billerica Mass.) is dissolved in phosphate buffered saline (PBS) at 10 ug/ml, and sterilized by filtration. Bleomycin or PBS control is injected subcutaneously into two locations on the shaved back of C57/BL6 or S129 mice (Charles River/Harlan Labs, 20-25 g) once daily for 28 days while under isoflourane anesthesia (5% in 100% 02). After 28 days, mice are euthanized and 6 mm-full thickness punch biopsies are obtained from each injection site. Dermal fibrosis is assessed by standard histopathology and hydroxyproline biochemical assays.

### Example 30: Targeting Calpains

### Inhibition of EpMT

For assessment of in vitro EMT, NMuMG cells (ATCC) are grown to confluence in 10% serum (Fetal Bovine Serum) growth media (Dubecco's Modified Eagles Medium supplemented with 10 ug/mL insulin) and then are followed by 24 h starvation in 0.5% serum media+/− drug inhibitors. Cells are then treated with recombinant human TGFb1 (R&D Systems 5 ng/mL)+/− drug inhibitors in 0.5% serum media. For time points greater than 24 h, the aforementioned media is refreshed every 24 hours. Cell lysates were analyzed for aSMA protein expression by western blot.

Miettinen et al. (1994). “TGF-beta induced transdifferentiation of mammary epithelial cells to mesenchymal cells: involvement of type I receptors.” J Cell Biol 127(6 Pt 2):2021-36.

Lamouille et al. (2014). “Molecular mechanisms of epithelial-mesenchymal transition.” Nat Rev Mol Cell Biol 15(3):178-96.

For assessment of in vitro FMT, Normal Human Lung Fibroblasts (NHLF) cells (Lonza) were grown in Fibroblast Growth Media-2 (Lonza CC-3131/with CC1-4126 bullet kit) and then were followed by 24 h starvation in serum/growth factor free Fibroblast Basal Media-2 (Lonza CC-3131)+/− drug inhibitors. Cells were then treated with TGFb1 (5 ng/mL) Fibroblast Basal Media+/− drug inhibitors. Cell lysates are analyzed for aSMA protein expression by western blot.

Further details may be found in Pegorier et al. (2010). “Bone Morphogenetic Protein (BMP)-4 and BMP-7 regulate differentially Transforming Growth Factor (TGF)-B1 in normal human lung fibroblasts (NHLF)” Respir Res 11:85, which is incorporated herein by reference in its entirety.

### Example 31: Human Treatment

The efficacy of treatment with a compound of a preferred embodiment compared with placebo in patients with idiopathic pulmonary fibrosis (IPF) and the safety of treatment with a compound of a preferred embodiment compared with placebo in patients with IPF is assessed. The primary outcome variable is the absolute change in percent predicted forced vital capacity (FVC) from baseline to Week 52. Other possible end-points would include, but are not limited to: mortality, progression free survival, change in rate of FVC decline, change in Sp02, and change in biomarkers (HRCT image analysis; molecular and cellular markers of disease activity). Secondary outcome measures include: composite outcomes of important IPF-related events; progression-free survival; the rate of death from any cause; the rate of death from IPF; categorical assessment of absolute change in percent predicted FVC from baseline to Week 52; change in Shortness-of-Breath from baseline to Week 52; change in percent predicted hemoglobin (Hb)-corrected carbon monoxide diffusing capacity (DLco) of the lungs from baseline to Week 52; change in oxygen saturation during the 6 minute walk test (6MWT) from baseline to Week 52; change in high-resolution computed tomography (HRCT) assessment from baseline to Week 52; change in distance walked in the 6MWT from baseline to Week 52. Patients eligible for this study include, but are not limited to: those patients that satisfy the following inclusion criteria: diagnosis of IPF; 40 to 80 years of age; FVC ≥50% predicted value; DLco ≥35% predicted value; either FVC or DLco ≤90% predicted value; no improvement in past year; a ratio of the forced expiratory volume in 1 second (FEV) to the FVC of 0.80 or more; able to walk 150 meters in 6 minutes and maintain saturation-83% while on no more than 6 L/min supplemental oxygen. Patients are excluded from this study if they satisfy any of the following criteria: unable to undergo pulmonary function testing; evidence of significant obstructive lung disease or airway hyper-responsiveness; in the clinical opinion of the investigator, the patient is expected to need and be eligible for a lung transplant within 52 weeks of randomization; active infection; liver disease; cancer or other medical condition likely to result in death within 2 years; diabetes; pregnancy or lactation; substance abuse; personal or family history of long QT syndrome; other IPF treatment; unable to take study medication; withdrawal from other IPF trials. Patients are orally dosed with either placebo or an amount of a compound of a preferred embodiment (1 mg/day-1000 mg/day). The primary outcome variable will be the absolute change in percent predicted FVC from Baseline to Week 52. Patients will receive blinded study treatment from the time of randomization until the last patient randomized has been treated for 52 weeks. Physical and clinical laboratory assessments will be performed at defined intervals during the treatment duration, for example at weeks 2, 4, 8, 13, 26, 39, and 52. Pulmonary function, exercise tolerance, and shortness-of-breath will be assessed at defined intervals during the treatment duration, for example at weeks 13, 26, 39, and 52. A Data Monitoring Committee (DMC) will periodically review safety and efficacy data to ensure patient safety.

### Example Trial in SSc

The efficacy of treatment with a compound of a preferred embodiment compared with placebo in patients with systemic sclerosis (SSc) and the safety of treatment with a compound of a preferred embodiment compared with placebo in patients with SSc is assessed. The primary outcome variable is the absolute change in Modified Rodnan Skin Score (mRSS) from baseline to Week 48. Other possible end-points would include, but are not limited to: mortality, percentage of patients with treatment-emergent adverse events (AEs) and serious adverse events (SAEs), composite measurement of disease progression, and change in biomarkers (molecular and cellular markers of disease activity, such as C-reactive protein). Secondary outcome measures include, but are not limited to: Scleroderma Health Assessment Questionnaire (SHAQ) score; the Health Assessment Questionnaire Disability Index (HAQ-DI); Functional Assessment of Chronic Illness Therapy-Fatigue (FACIT) score; severity of pruritus as measured by a standardized scale, such as the 5-D Itch Scale; St. George's Respiratory Questionnaire (SGRQ) score; Tender Joint Count 28 (TCJ28); lung function parameters; standard vital signs (including blood pressure, heart rate, and temperature); electrocardiogram measurements (ECGs); laboratory tests (clinical chemistry, hematology, and urinalysis); pharmacokinetics (PK) measurements. Included in these measurements and in addition, clinical and biomarker samples, such as skin biopsies and blood (or serum and/or plasma), will also be collected prior to initiation of treatment. Additionally, patients eligible for this study include, but are not limited to, those patients that satisfy the following criteria: Patients at least 18 years of age; diagnosis of SSc according to the American College of Rheumatology (ACR) and European League Against Rheumatism (EULAR) Criteria, meeting criteria for active disease and with a total disease duration of less than or equal to 60 months; 10=mRSS ≤35. Patients are excluded from this study if they satisfy any of the following criteria: major surgery within 8 weeks prior to screening; scleroderma limited to area distal to the elbows or knees; rheumatic autoimmune disease other than SSc; use of any investigational, biologic, or immunosuppressive therapies, including intra-articular or parenteral corticosteroids within 4 weeks of screening. Patients are orally dosed with either placebo or an amount of a compound of a preferred embodiment (1 mg/day-1000 mg/day). The primary outcome variable will be the absolute change in mRSS from Baseline to Week 48. Patients will receive blinded study treatment from the time of randomization until the last patient randomized has been treated for 48 weeks. Physical and clinical laboratory assessments will be performed at defined intervals during the treatment duration, such as Weeks 2, 4, 8, 12, 24, 36, and 48. Clinical and biomarker samples will also be collected at Week 48. A Data Monitoring Committee (DMC) will periodically review safety and efficacy data to ensure patient safety.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

Although the invention has been described with reference to embodiments and examples, it should be understood that numerous and various modifications can be made without departing from the spirit of the invention. Accordingly, the invention is limited only by the following claims.

### REFERENCES CITED

1. U.S. Pat. No. 5,145,684
2. Goll et al. (2003). “The calpain system.” Physiol Rev 83(3):731-801.
3. Schad et al. (2002). “A novel human small subunit of calpains.” Biochem J 362(Pt 2):383-8.
4. Ravulapalli et al. (2009). “Distinguishing between calpain heterodimerization and homodimerization.” FEBS J 276(4):973-82.
5. Dourdin et al. (2001). “Reduced cell migration and disruption of the actin cytoskeleton in calpain-deficient embryonic fibroblasts.” J Biol Chem 276(51):48382-8.
6. Leloup et al. (2006). “Involvement of calpains in growth factor-mediated migration.” Int J Biochem Cell Biol 38(12):2049-63.
7. Janossy et al. (2004). “Calpain as a multi-site regulator of cell cycle.” Biochem Pharmacol 67(8):1513-21.
8. Santos et al. (2012). “Distinct regulatory functions of calpain 1 and 2 during neural stem cell self-renewal and differentiation.” PLoS One 7(3):e33468.
9. Miettinen et al. (1994). “TGF-beta induced transdifferentiation of mammary epithelial cells to mesenchymal cells: involvement of type I receptors.” J Cell Biol 127(6 Pt 2):2021-36.
10. Lamouille et al. (2014). “Molecular mechanisms of epithelial-mesenchymal transition.” Nat Rev Mol Cell Biol 15(3):178-96.
11. Pegorier et al. (2010). “Bone Morphogenetic Protein (BMP)-4 and BMP-7 regulate differentially Transforming Growth Factor (TGF)-B1 in normal human lung fibroblasts (NHLF)” Respir Res 11:85.

What is claimed is:

1. A compound having the structure of the formula I: 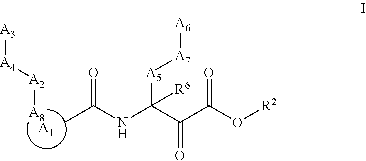
or a pharmaceutically acceptable salt thereof, wherein:
A₁ is selected from the group consisting of optionally substituted 5-10 membered heterocyclyl;
optionally substituted 5-, 8-, or 9-membered heteroaryl; and optionally substituted C₃₋₁₀ carbocyclyl;
A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;
A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)₆—, —(CR₂)ₙ—NR—(CR₂)₆—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)₆—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond,
when A₂ and A₄ are single bond, A₃ is directly attached to A₈;
A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, As is directly attached to the carbon to which R⁶ is attached;
A₈ is a ring member of A and is selected from the group consisting of C and N;
R is independently selected from —H, halo, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R² is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl;
R⁶ is independently selected from —H and optionally substituted C₁₋₄ alkyl; and
each n is independently selected to be an integer from 0 to 3.

2. The compound of claim 1, wherein when A₁ is optionally substituted 5-10 membered heterocyclyl, the 5-10 membered heterocyclyl is not substituted with oxo.

3. The compound of claim 1, wherein:
A₁ is selected from the group consisting of optionally substituted 6-10 membered heterocyclyl; optionally substituted 5-, 8-, or 9-membered heteroaryl; and optionally substituted C₃₋₁₀ carbocyclyl;
A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted —O—C₁₋₆ alkyl, optionally substituted —O—C₂₋₆ alkenyl, and any natural or non-natural amino acid side chain;
R is independently selected from —H, halo, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and
R² is independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, and optionally substituted C₆₋₁₀ aryl(C₁-C₆)alkyl.

4. The compound of claim 3, wherein when A₁ is optionally substituted 6-10 membered heterocyclyl, the 6-10-membered heterocyclyl is not substituted with oxo.

5. The compound of any one of claims 1-4 having the structure of formula I-a: 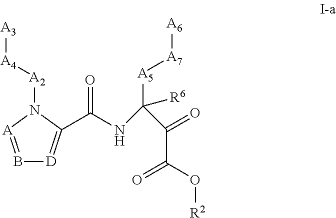
or a pharmaceutically acceptable salt thereof, wherein:
A, B, and D are each independently selected from the group consisting of C(R⁴) and N; and
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy.

6. The compound of claim 5, wherein A, B, and D are independently selected from the group consisting of CH and N.

7. The compound of claim 5, wherein A is N, B is CH, and D is CH.

8. The compound of claim 5, wherein A is CH, B is N, and D is CH.

9. The compound of claim 5, wherein A is N, B is N, and D is N.

10. The compound of any one of claims 1-4 having the structure of formula I-b: 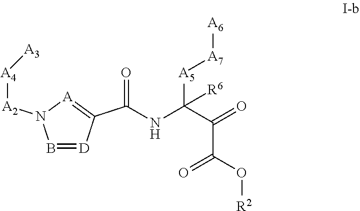
or a pharmaceutically acceptable salt thereof, wherein:
A, B, and D are each independently selected from the group consisting of C(R⁴) and N; and
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy.

11. The compound of claim 10, wherein A, B, and D are independently selected from the group consisting of CH and N.

12. The compound of any one of claims 1-4 having the structure of formula I-c: 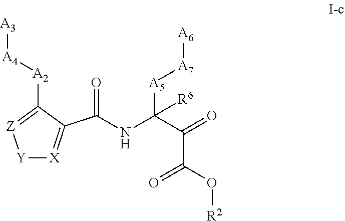
or a pharmaceutically acceptable salt thereof, wherein:
Y is selected from the group consisting of NR, O, S, and SO₂;
X and Z are each independently selected from the group consisting of C(R⁴) and N;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

13. The compound of claim 12, wherein Z is N, Y is NR⁵, and X is CH.

14. The compound of claim 13, wherein R⁵ is selected from the group consisting of —H, C₁₋₄ alkyl, C₁-C₄ haloalkyl, and cyclopropyl.

15. The compound of claim 12, wherein Z is N, Y is O, and X is C(R⁴).

16. The compound of claim 12, wherein Z is N, Y is S, and X is C(R⁴).

17. The compound of claim 12, wherein Z is C(R⁴), Y is S, and X is C(R⁴).

18. The compound of claim 12, wherein Z is C(R⁴), Y is O, and X is C(R⁴).

19. The compound of claim 12, wherein Z is C(R⁴), Y is S, and X is N.

20. The compound of claim 12, wherein Z is C(R⁴), Y is O, and X is N.

21. The compound of claim 12, wherein Z is N, Y is S, and X is N.

22. The compound of claim 12, wherein Z is N, Y is O, and X is N.

23. The compound of any one of claims 1-4 having the structure of formula I-d: 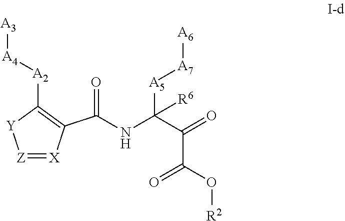
or a pharmaceutically acceptable salt thereof, wherein:
Y is selected from the group consisting of NR⁵, O, S, and SO₂;
X and Z are each independently selected from the group consisting of C(R⁴) and N;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

24. The compound of claim 23, wherein X and Z are independently selected from the group consisting of CH and N.

25. The compound of claim 23, wherein Y is NR⁵, Z is N, and X is CH.

26. The compound of claim 23, wherein Z is C(R⁴), Y is O, and X is N.

27. The compound of claim 23, wherein Z is C(R⁴), Y is S, and X is N.

28. The compound of any one of claims 1-4 having the structure of formula I-e: 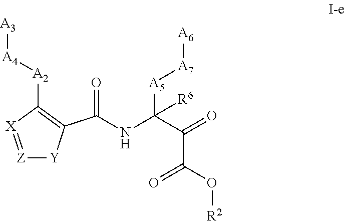
or a pharmaceutically acceptable salt thereof, wherein:
Y is selected from the group consisting of NR, O, S, and SO₂;
X and Z are each independently selected from the group consisting of C(R⁴) and N;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

29. The compound of claim 28, wherein X and Z are independently selected from the group consisting of CH and N.

30. The compound of claim 28, wherein X is CH, Z is N, and Y is NR³.

31. The compound of claim 28, wherein X is N, Z is C(R⁴), and Y is O.

32. The compound of claim 31, wherein R⁴ is selected from —H and C₁₋₄ alkyl.

33. The compound of claim 28, wherein X is N, Z is C(R⁴), and Y is S.

34. The compound of claim 28, wherein X is N, Z is N, and Y is S.

35. The compound of any one of claims 1-34, wherein at least one of the optionally substituted moieties of A₂, A₄, and A₃ is substituted with ¹⁸F.

36. The compound of any one of claims 1-35, wherein at least one of the optionally substituted moieties of A₂, A₄, and A₃ is substituted with C₁-C₆ alkyl containing one or more C.

37. The compound of any one of claims claim 1-36, wherein A₃ is selected from the group consisting of 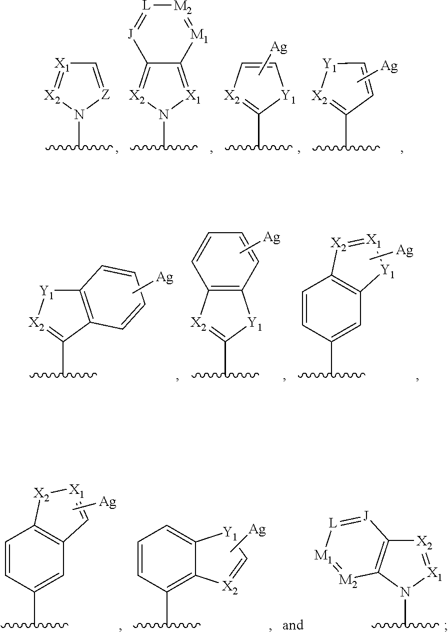
and
A₉ is selected from the group consisting of H, C₆₋₁₀ aryl, 5-10 membered heteroaryl, 3-10 membered heterocyclyl, and C₃₋₁₀ carbocyclyl, C₁₋₄ alkyl;
X₂, X₁, and Z are each independently selected from the group consisting of C(R⁴) and N;
Y₁ is selected from the group consisting of NR⁵, O, and S;
J, L, M₁ and M₂ are each independently selected from the group consisting of C(R⁴) and N;
R⁴ is selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy;
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

38. The compound of any one of the claims 1-36, wherein A₃ is optionally substituted C₆₋₁₀ aryl.

39. The compound of claim 38, wherein A₃ is phenyl.

40. The compound of claim 38, wherein A₃ is selected from the group consisting of

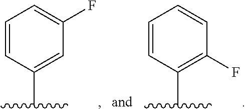 [SMILES: *c1cccc(F)c1 ; *c1ccccc1F]

41. The compound of any one of the claims 1-36, wherein A₃ is optionally substituted 5-10 membered heteroaryl.

42. The compound of any one of claims 1-40 wherein A₂ is single bond.

43. The compound of any one of the claims 1-40, wherein A₂ is —CH₂—.

44. The compound of any one of the claims 1-40, wherein A₂ is —CH═CH—.

45. The compound of any one of the claims 1-40, wherein A₂ is —O—.

46. The compound of any one of the claims 1-40, wherein A₂ is —S—.

47. The compound of any one of claims 1-40, wherein A₂ is phenyl.

48. The compound of any one of claims 1-40, wherein A₂ is selected from the group consisting of optionally substituted 3-10 membered heterocyclyl, optionally substituted C₆₋₁₀ aryl, optionally substituted 5- or 7-10 membered heteroaryl, optionally substituted C₃₋₁₀ carbocyclyl, —S—, —S(═O)—, —SO₂—, —C(═S)—, —C(═O)—, —NR—, —CH═CH—, —C≡C—, —OC(O)NH—, —NHC(O)NH—, —NHC(O)O—, —NHC(S)NH—, —NHC(S)O—, and —NHC(S)—.

49. The compound of any one of claims 1-40, wherein A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, and —C≡C—.

50. The compound of any one of claims 1-40, wherein A₂ is selected from the group consisting of optionally substituted 3-10 membered heterocyclyl, optionally substituted C₆₋₁₀ aryl, optionally substituted 5-10 membered heteroaryl, and optionally substituted C₃₋₁₀ carbocyclyl.

51. The compound of any one of claims 1-50, wherein A₄ is single bond.

52. A compound having the structure of the formula H: 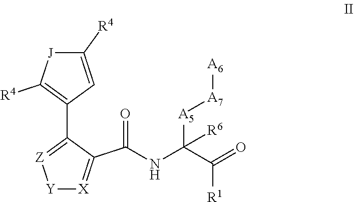
or a pharmaceutically acceptable salt thereof, wherein:
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁶ is attached;
Y is selected from the group consisting of NR⁵, and S;
X and Z are each independently selected from the group consisting of C(R⁴) and N;
J is selected from the group consisting of O and S;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);
R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃, 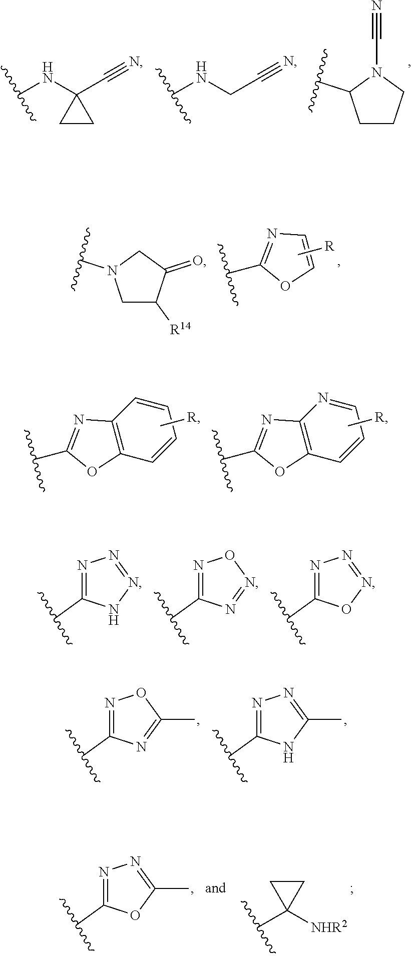
R¹⁴ is halo;
each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R⁶ is independently selected from —H and optionally substituted C₁₋₄ alkyl; and
each n is independently selected to be an integer from 0 to 3; and wherein the compound is not selected from the group consisting of

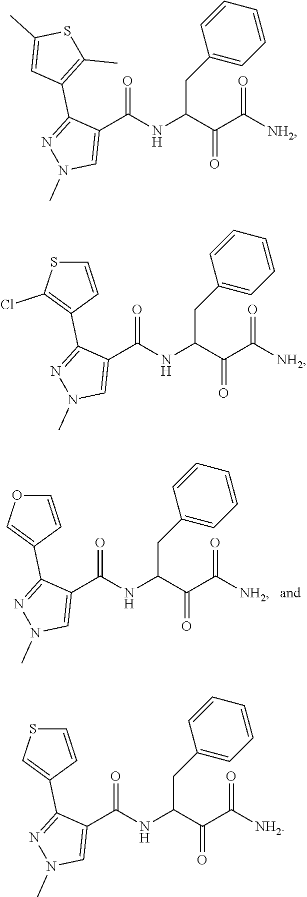 [SMILES: Cc1cc(-c2nn(C)cc2C(=O)NC(Cc2ccccc2)C(=O)C(N)=O)c(C)s1 ; Cn1cc(C(=O)NC(Cc2ccccc2)C(=O)C(N)=O)c(-c2ccsc2Cl)n1 ; Cn1cc(C(=O)NC(Cc2ccccc2)C(=O)C(N)=O)c(-c2ccoc2)n1 ; "and" ; Cn1cc(C(=O)NC(Cc2ccccc2)C(=O)C(N)=O)c(-c2ccsc2)n1]

53. The compound of claim 52, wherein Z is N, Y is NR, and X is CH.

54. The compound of claim 53, wherein R⁵ is selected from the group consisting of —H, C₁₋₄ alkyl, C₁-C₄ haloalkyl, and cyclopropyl.

55. The compound of claim 52, wherein Z is N, Y is S, and X is N.

56. The compound of claim 52, wherein R¹ is —CONR²R³.

57. The compound of claim 52, wherein R¹ is —CONH₂.

58. The compound of claim 56, wherein R² is —H and R³ is optionally substituted C₁₋₄ alkyl.

59. The compound of claim 56, wherein; R² is —H and R³ is selected from the group consisting of —H, C₁-C₄ alkyl optionally substituted with C-amido, and C₃-C₆ cycloalkyl.

60. The compound of claim 59, wherein R³ is selected from ethyl or cyclopropyl.

61. The compound of claim 59, wherein R³ is methyl substituted with C-amido.

62. The compound of claim 59, wherein R³ is —H.

63. The compound of claim 59, wherein R³ is optionally substituted C₁₋₄ alkyl.

64. The compound of claim 59, wherein R³ is benzyl.

65. The compound of claim 52, wherein when R¹ is —COOR².

66. The compound of claim 65, wherein; R² is selected from the group consisting of —H, C₁-C₄ alkyl optionally substituted with C-amido, and C₃-C₆ cycloalkyl.

67. A compound having the structure of the formula III: 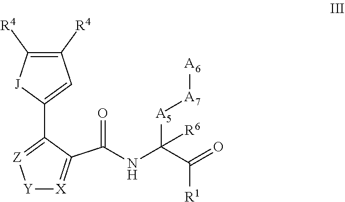
or a pharmaceutically acceptable salt thereof, wherein:
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OS₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁶ is attached;
Y is selected from the group consisting of NR⁵, and S;
X and Z are each independently selected from the group consisting of C(R⁴) and N;
J is selected from the group consisting of O and S;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);
R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃, 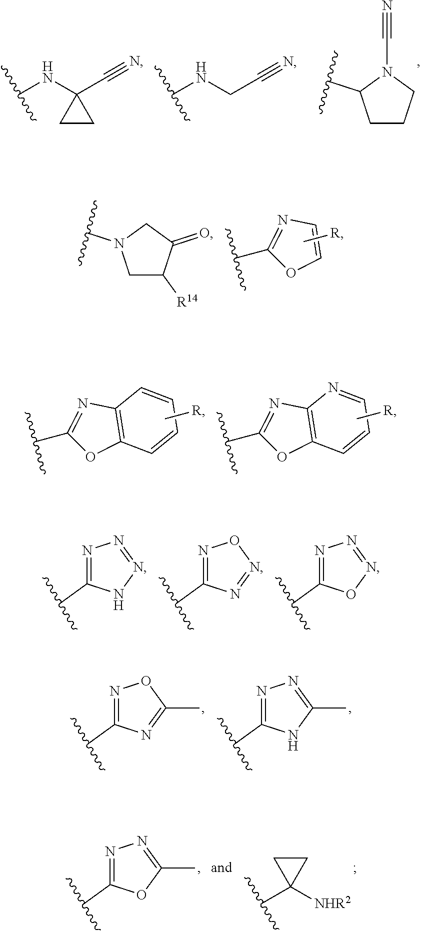
R¹⁴ is halo;
each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R⁶ is independently selected from —H and optionally substituted C₁₋₄ alkyl; and
each n is independently selected to be an integer from 0 to 3; and wherein the compound is not selected from the group consisting of

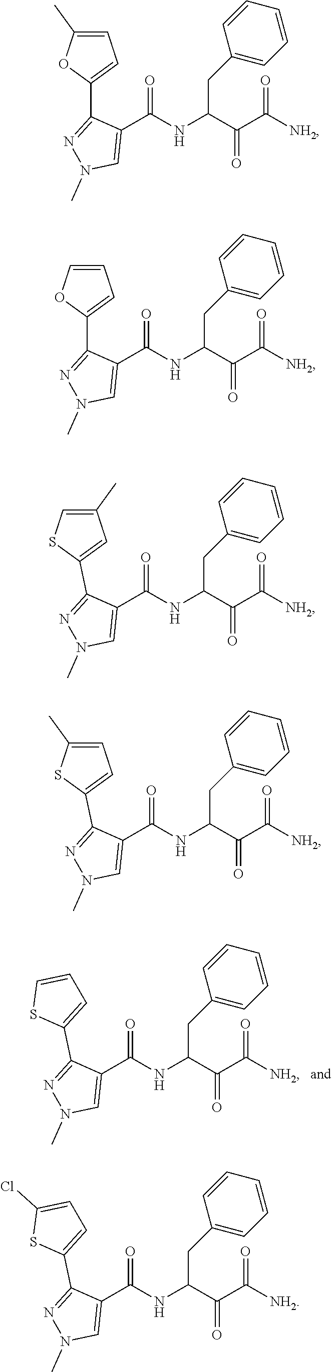

68. The compound of claim 67, wherein Z is N, Y is NR⁵, and X is CH.

69. The compound of claim 68, wherein R⁵ is selected from the group consisting of —H, C₁₋₄ alkyl, C₁-C₄ haloalkyl, and cyclopropyl.

70. The compound of claim 67, wherein Z is N, Y is S, and X is N.

71. The compound of claim 67, wherein R¹ is —CONR²R³.

72. The compound of claim 67, wherein R¹ is —CONH₂.

73. The compound of claim 71, wherein R² is —H and R³ is optionally substituted C₁₋₄ alkyl.

74. The compound of claim 71, wherein; R² is —H and R³ is selected from the group consisting of —H, C₁-C₄ alkyl optionally substituted with C-amido, and C₃-C₆ cycloalkyl.

75. The compound of claim 74, wherein R³ is selected from ethyl or cyclopropyl.

76. The compound of claim 74, wherein R³ is methyl substituted with C-amido.

77. The compound of claim 74, wherein R³ is —H.

78. The compound of claim 74, wherein R³ is optionally substituted C₁₋₄ alkyl.

79. The compound of claim 74, wherein R³ is benzyl.

80. The compound of claim 67, wherein when R¹ is —COOR².

81. The compound of claim 80, wherein; R² is selected from the group consisting of —H, C₁-C₄ alkyl optionally substituted with C-amido, and C₃-C₆ cycloalkyl.

82. A compound having the structure of the formula IV: 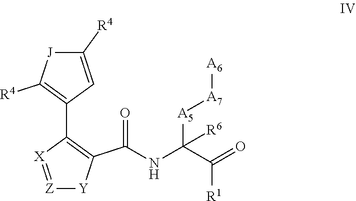
or a pharmaceutically acceptable salt thereof, wherein:
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₄ alkenyl, optionally substituted —O—C₁₋₆ alkyl, optionally substituted —O C₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁶ is attached;
Y is selected from the group consisting of NR⁵, O, S, and SO₂;
X and Z are each independently selected from the group consisting of C(R⁴) and N;
J is selected from the group consisting of O and S;
each R⁴ is independently selected from the group consisting of —H, C₁₋₄ alkyl, C₁₋₄ haloalkyl, C₃₋₇carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);
R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃, 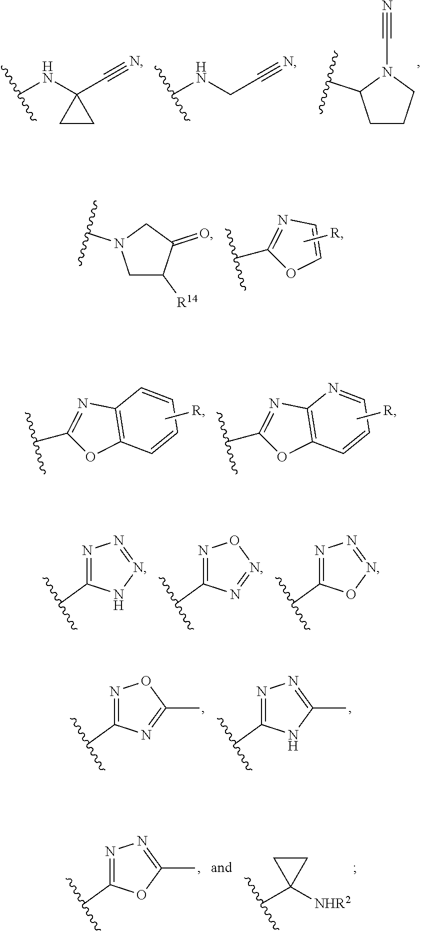
R¹⁴ is halo;
each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R⁶ is independently selected from —H and optionally substituted C₁₋₄ alkyl; and
each n is independently selected to be an integer from 0 to 3.

83. The compound of claim 82, wherein X and Z are independently selected from the group consisting of C(R⁴) and N.

84. The compound of claim 82, wherein X is N, Z is C(R⁴), and Y is O.

85. The compound of claim 84, wherein R⁴ is selected from —H and C₁₋₄ alkyl.

86. The compound of claim 82, wherein R¹ is —CONR²R³.

87. The compound of claim 82, wherein R¹ is —CONH₂.

88. The compound of claim 86, wherein R² is —H and R³ is optionally substituted C₁₋₄ alkyl.

89. The compound of claim 86, wherein; R² is —H and R³ is selected from the group consisting of —H, C₁-C₄ alkyl optionally substituted with C-amido, and C₃-C₆ cycloalkyl.

90. The compound of claim 89, wherein R³ is selected from ethyl or cyclopropyl.

91. The compound of claim 89, wherein R³ is methyl substituted with C-amido.

92. The compound of claim 89, wherein R³ is —H.

93. The compound of claim 89, wherein R³ is optionally substituted C₁₋₄ alkyl.

94. The compound of claim 89, wherein R³ is benzyl.

95. The compound of claim 82, wherein when RI is —COOR².

96. The compound of claim 95, wherein; R² is selected from the group consisting of —H, C₁-C₄ alkyl optionally substituted with C-amido, and C₃-C₆ cycloalkyl.

97. A compound having the structure of the formula V: 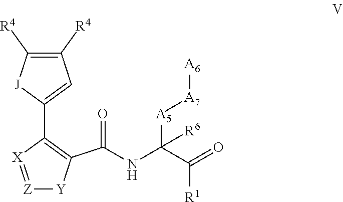
or a pharmaceutically acceptable salt thereof, wherein:
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, A₆ is directly attached to the carbon to which R⁶ is attached,
Y is selected from the group consisting of NR—, O, S, and SO₂;
X and Z are each independently selected from the group consisting of C(R⁴) and N;
J is selected from the group consisting of O and S;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy);
R¹ is selected from the group consisting of H, —OH, —COOR², C₁₋₄ haloalkyl, —COOH, —CH₂NO₂, —C(═O)NOR, —NH₂, —CONR²R³, —CH(CH₃)═CH₂, —CH(CF₃)NR²R³, —C(F)═CHCH₂CH₃, 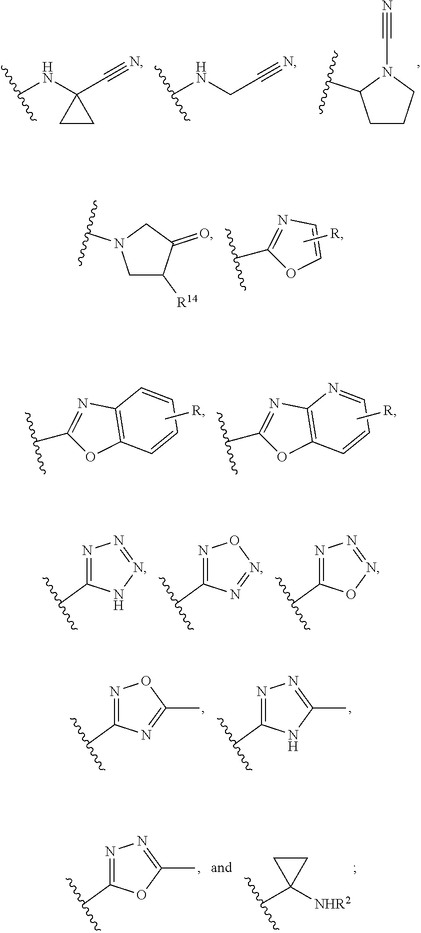
R¹⁴ is halo;
each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl;
R⁶ is independently selected from —H and optionally substituted C₁₋₄ alkyl; and
each n is independently selected to be an integer from 0 to 3.

98. The compound of claim 97, wherein X and Z are independently selected from the group consisting of C(R⁴) and N.

99. The compound of claim 97, wherein X is N, Z is C(R⁴), and Y is O.

100. The compound of claim 99, wherein R⁴ is selected from —H and C₁₋₄ alkyl.

101. The compound of claim 97, wherein RI is —CONR²R³.

102. The compound of claim 97, wherein R¹ is —CONH₂.

103. The compound of claim 101, wherein R² is —H and R³ is optionally substituted C₁₋₄ alkyl.

104. The compound of claim 101, wherein; R² is —H and R³ is selected from the group consisting of —H, C₁-C₄ alkyl optionally substituted with C-amido, and C₃-C₆ cycloalkyl.

105. The compound of claim 104, wherein R³ is selected from ethyl or cyclopropyl.

106. The compound of claim 104, wherein R³ is methyl substituted with C-amido.

107. The compound of claim 104, wherein R³ is —H.

108. The compound of claim 104, wherein R³ is optionally substituted C₁₋₄ alkyl.

109. The compound of claim 104, wherein R³ is benzyl.

110. The compound of claim 97, wherein when R¹ is —COOR².

111. The compound of claim 110, wherein; R² is selected from the group consisting of —H, C₁-C₄ alkyl optionally substituted with C-amido, and C₃-C₆ cycloalkyl.

112. A compound having the structure of the formula VI: 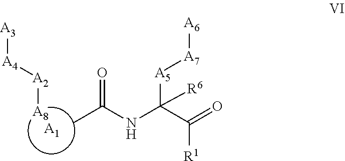
or a pharmaceutically acceptable salt thereof, wherein:
A₁ is selected from the group consisting of optionally substituted 5-10 membered heteroaryl; optionally substituted 5-10 membered heterocyclyl; and optionally substituted C₃₋₁₀ carbocyclyl;
A₂ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, —CR₂—, —S—, —S(═O)—, —SO₂—, —O—, —C(═S)—, —C(═O)—, —NR—, —CH═CH—, —C≡C—, —OC(O)NH—, —NHC(O)NH—, —NHC(O)O—, —NHC(O)—, —NHC(S)NH—, —NHC(S)O—, —NHC(S)—, and single bond;
A₄ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₄ alkyl, —(CR₂)ₙ—S—(CR₂)ₙ—, —(CR₂)ₙ—S(═O)—(CR₂)ₙ—, —(CR₂)ₙ—SO₂—(CR₂)ₙ—, —(CR₂)ₙ—O—(CR₂)ₙ—, —(CR₂)ₙ—C(═S)—(CR₂)ₙ—, —(CR₂)ₙ—C(═O)—(CR₂)ₙ—, —(CR₂)ₙ—NR—(CR₂)ₙ—, —(CR₂)ₙ—CH═CH—(CR₂)ₙ—, —(CR₂)ₙ—OC(O)NH—(CR₂)ₙ—, —(CR₂)ₙ—NHC(O)NH—(CR₂)ₙ—, —(CR₂)ₙ—NHC(O)O—(CR₂)ₙ—, —(CR₂)ₙ—NHC(O)—(CR₂)ₙ—, —(CR₂)ₙ—NHC(S)NH—(CR₂)ₙ—, —(CR₂)ₙ—NHC(S)O—(CR₂)ₙ—, —(CR₂)ₙ—NHC(S)—(CR₂)ₙ—, and single bond;
when A₂ and A₄ are single bond, A₃ is directly attached to A₈;
A₃ is selected from the group consisting of optionally substituted C₆₋₁₀ aryl, optionally substituted 5-10 membered heteroaryl, optionally substituted 3-10 membered heterocyclyl, and optionally substituted C₃₋₁₀ carbocyclyl, or if A₂ is selected from optionally substituted 3-10 membered heterocyclyl, optionally substituted C₆₋₁₀ aryl, optionally substituted 5-10 membered heteroaryl, and optionally substituted C₃₋₁₀ carbocyclyl, then A₃ is selected from the group consisting of hydrogen, optionally substituted C₆₋₁₀ aryl, optionally substituted 5-10 membered heteroaryl, optionally substituted 3-10 membered heterocyclyl, optionally substituted C₃₋₁₀ carbocyclyl, —C≡CH, and optionally substituted 2- to 5-membered polyethylene glycol;
A₈ is a ring member of A₁ and is selected from the group consisting of C and N;
A₅ is selected from the group consisting of optionally substituted 3-10 membered heterocyclyl, optionally substituted C₆₋₁₀ aryl, optionally substituted 5-10 membered heteroaryl, optionally substituted C₃₋₁₀ carbocyclyl, optionally substituted C₁₋₈ alkyl, —S—, —S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted C₂₋₈ alkenyl, optionally substituted —O—C₁₋₆ alkyl, optionally substituted —O C₂₋₆ alkenyl, —OSO₂CF₃, and any natural or non-natural amino acid side chain;
A₇ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, —S—, S(═O)—, —SO₂—, —O—, —C(═S)—, —C(═O)—, —NR—, —CH═CH—, —OC(O)NH—, —NHC(O)NH—, —NHC(O)O—, —NHC(O)—, —NHC(S)NH—, —NHC(S)O—, —NHC(S)—, and single bond;
when A₅ and A₇ are single bond, As is directly attached to the carbon to which R⁶ is attached;
R¹ is selected from the group consisting of —C(O)N(R²)O(R³), —C(O)N(R²)NR²R³, and —CR₂OR³;
each R, R², and R³ are independently selected from —H, optionally substituted C₁₋₄ alkyl, optionally substituted C₁₋₈ alkoxyalkyl, optionally substituted 2- to 5-membered polyethylene glycol, optionally substituted C₃₋₇ carbocyclyl, optionally substituted 5-10 membered heterocyclyl, optionally substituted C₆₋₁₀ aryl, optionally substituted C₆₋₁₀ aryl(C₁-C₆)alkyl, and optionally substituted 5-10 membered heteroaryl; and
R⁶ is independently selected from —H and optionally substituted C₁₋₄ alkyl; and each n is independently selected to be an integer from 0 to 3.

113. The compound of claim 112 having the structure of formula VI-a: 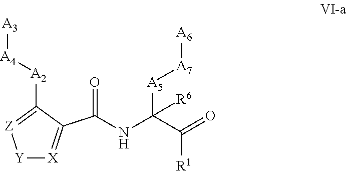
or a pharmaceutically acceptable salt thereof, wherein:
Y is selected from the group consisting of NR, O, S, and SO₂;
X and Z are each independently selected from the group consisting of C(R⁴) and N;
each R⁴ is independently selected from the group consisting of —H, C₁₋₄ alkyl, C₁₋₄ haloalkyl, C₃₋₇ carbocyclyl (optionally substituted with halo, C₁-C₆ alkyl, C₁-C₆ alkoxy, C₁-C₆ haloalkyl, and C₁-C₆ haloalkoxy), halo, hydroxy, and C₁-C₆ alkoxy; and
R⁵ is selected from the group consisting of —H, C₁₋₄ alkyl, C₁₋₄ haloalkyl, and C₃₋₇ carbocyclyl (optionally substituted with halo, C₁-C₆ alkyl, C₁-C₆ alkoxy, C₁-C₆ haloalkyl, and C₁-C₆ haloalkoxy).

114. The compound of claim 113, wherein Z is N, Y is NR⁵, and X is CH.

115. The compound of claim 114, wherein R⁵ is selected from the group consisting of —H, C₁₋₄ alkyl, C₁-C₄ haloalkyl, and cyclopropyl.

116. The compound of claim 113, wherein Z is N, Y is S, and X is N.

117. The compound of claim 113, wherein; R² is —H and R³ is selected from the group consisting of optionally substituted C₁₋₄ alkyl and C₃-C₆ cycloalkyl.

118. The compound of claim 117, wherein R² is —H and R³ is optionally substituted C₁₋₄ alkyl.

119. The compound of claim 118, wherein R³ is selected from methyl, ethyl, or cyclopropyl.

120. The compound of claim 118, wherein R² is —H.

121. The compound of claim 113, wherein R¹ is selected from the group consisting of —C(═O)NHOMe, —C(═O)NHN(Me)₂, and —CH₂OH.

122. The compound of any one of claims 1-121, wherein at least one of the optionally substituted moieties of A₅, A₇, and A₆ is substituted with ¹⁸F.

123. The compound of any one of claims 1-121, wherein at least one of the optionally substituted moieties of A₅, A₇, and A₆ is substituted with C₁-C₆ alkyl containing one or more ¹¹C.

124. The compound of anyone of the claims 1-121, wherein A₆ is phenyl.

125. The compound of anyone of claims 1-121, wherein A₆ is selected from the group consisting of optionally substituted C₆₋₁₀ aryl, optionally substituted 5-10 membered heteroaryl, optionally substituted 3-10 membered heterocyclyl, optionally substituted C₃₋₁₀ carbocyclyl, optionally substituted C₁₋₈ alkyl, optionally substituted —O—C₁₋₈ alkyl, and optionally substituted —O C₂₋₆ alkenyl.

126. The compound of any one of the claims 1-121, wherein A₇ is —CH₂—.

127. The compound of any one of the claims 1-121, wherein A₇ is O.

128. The compound of any one of the claims 1-121, wherein A₇ is —CH═CH—.

129. The compound of any one of the claims 1-121, wherein A₇ is S.

130. The compound of any one of the claims 1-121, wherein A₇ is single bond.

131. The compound of any one of the claims 1-121, wherein A₇ is optionally substituted C₆₋₁₀ aryl.

132. The compound of claim 131, wherein A₇ is phenyl.

133. The compound of anyone of claims 1-132, wherein A₅ is —CH₂—.

134. The compound of anyone of claims 1-121, wherein A₅ is —CH₂— or —CH₂CH₂—; A₇ is a single bond; and A₆ is selected from the group consisting of C₁-C₄ alkyl, optionally substituted phenyl, optionally substituted 5-10 membered heteroaryl.

135. The compound of claim 134, wherein A₆ is optionally substituted phenyl.

136. The compound of claim 134, wherein A₆ is unsubstituted phenyl.

137. The compound of claim 134, wherein A₆ is phenyl optionally substituted with one or more C₁₋₄ alkyl, C₃₋₇ carbocyclyl, halo, hydroxy, and C₁-C₆ alkoxy.

138. The compound of anyone of claims 1-121, wherein A₅ is a single bond, A₇ is a single bond; and A₆ is C₁-C₅ alkyl.

139. The compound of any one of the claims 1-138, wherein R² is —H and optionally substituted C₁₋₄ alkyl.

140. The compound of claim 139, wherein R² is selected from the group consisting of C₁-C₄ alkyl optionally substituted with C-amido, and C₃-C₆ cycloalkyl.

141. The compound of claim 139, wherein R² is selected from methyl or ethyl.

142. The compound of claim 139, wherein R² is benzyl.

143. The compound of any one of claims 1-142, wherein R⁶ is —H and optionally substituted C₁₋₄ alkyl.

144. The compound of claim 143, wherein R⁶ is optionally substituted C₁₋₄ alkyl.

145. The compound of claim 144, wherein R⁶ is methyl.

146. The compound of any one of claims 1-4, wherein A₁ is selected from the group consisting of optionally substituted 6-10 membered heterocyclyl; 5-membered heterocyclyl optionally substituted with one or more C₁₋₄ alkyl, C₃₋₇ carbocyclyl, halo, hydroxy, or C₁-C₆ alkoxy; optionally substituted 5-, 8-, or 9-membered heteroaryl; and optionally substituted C₃₋₁₀ carbocyclyl.

147. The compound of any one of claims 1-4, wherein A₁ is selected from the group consisting of 5-membered heterocyclyl optionally substituted with one or more C₁₋₄ alkyl, C₃₋₇ carbocyclyl, halo, hydroxy, or C₁-C₆ alkoxy and optionally substituted 5-membered heteroaryl.

148. The compound of any one of claims 1-4, wherein A₁ is optionally substituted 5-membered heteroaryl.

149. The compound of claim 1, having the structure selected from the group consisting of: 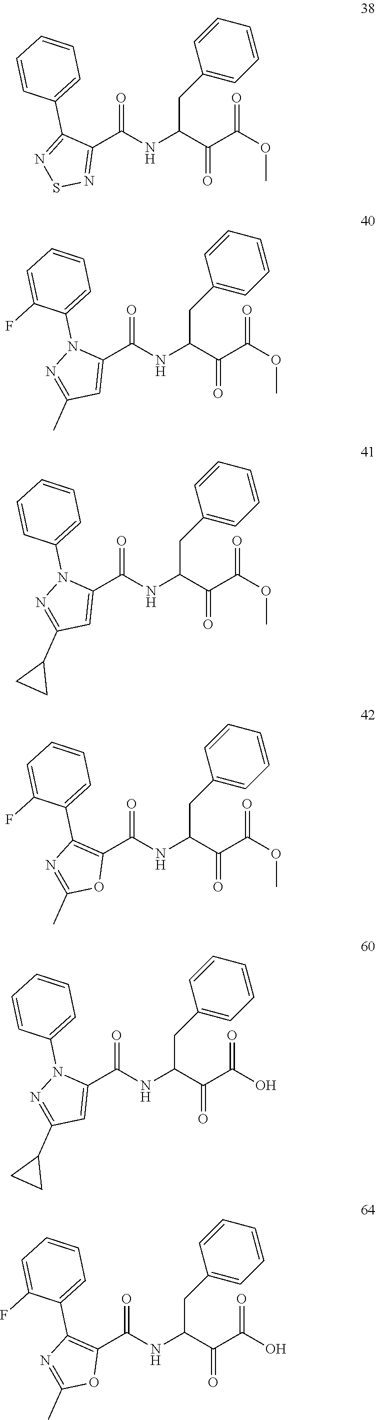 [SMILES: COC(=O)C(=O)C(Cc1ccccc1)NC(=O)c1nsnc1-c1ccccc1 ; COC(=O)C(=O)C(Cc1ccccc1)NC(=O)c1cc(C)nn1-c1ccccc1F ; COC(=O)C(=O)C(Cc1ccccc1)NC(=O)c1cc(C2CC2)nn1-c1ccccc1 ; COC(=O)C(=O)C(Cc1ccccc1)NC(=O)c1oc(C)nc1-c1ccccc1F ; O=C(O)C(=O)C(Cc1ccccc1)NC(=O)c1cc(C2CC2)nn1-c1ccccc1 ; Cc1nc(-c2ccccc2F)c(C(=O)NC(Cc2ccccc2)C(=O)C(=O)O)o1] 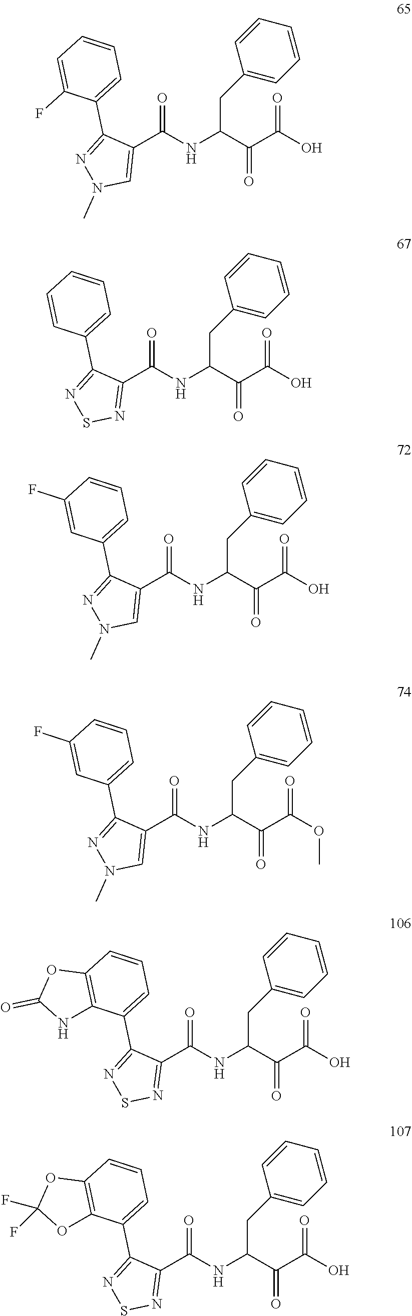 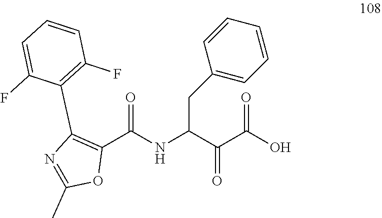 [SMILES: Cc1nc(-c2c(F)cccc2F)c(C(=O)NC(Cc2ccccc2)C(=O)C(=O)O)o1]
and pharmaceutically acceptable salts thereof.

150. The compound of claim 52, having the structure selected from the group consisting of: 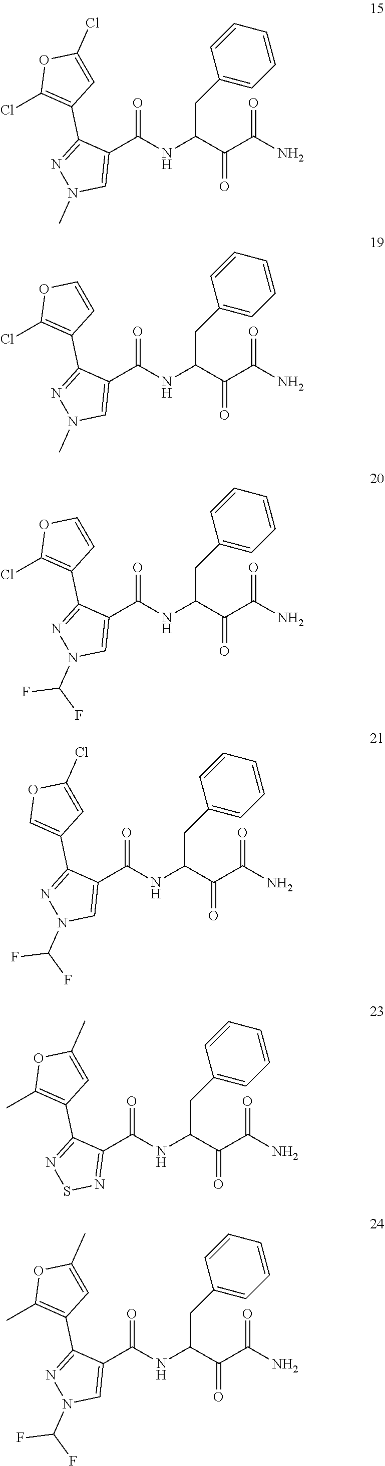 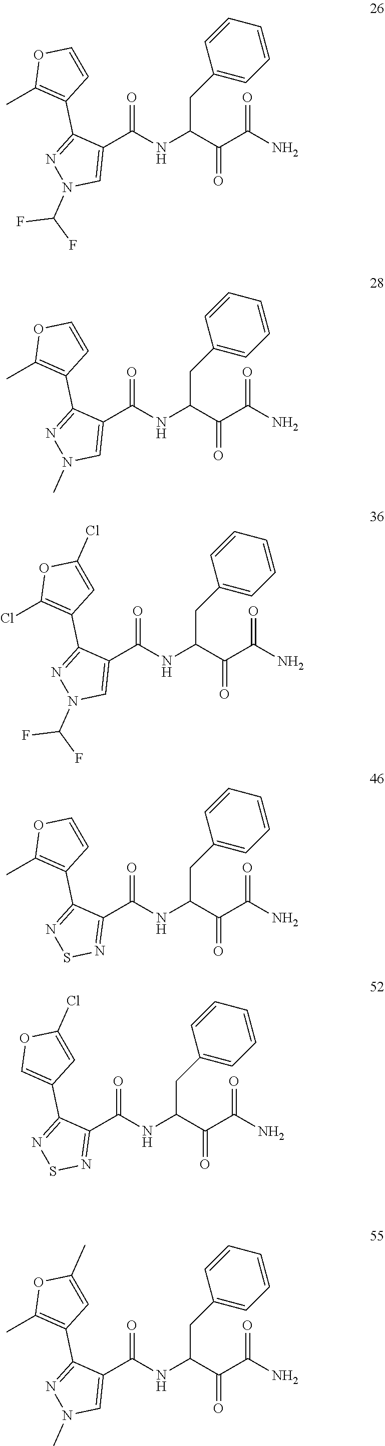 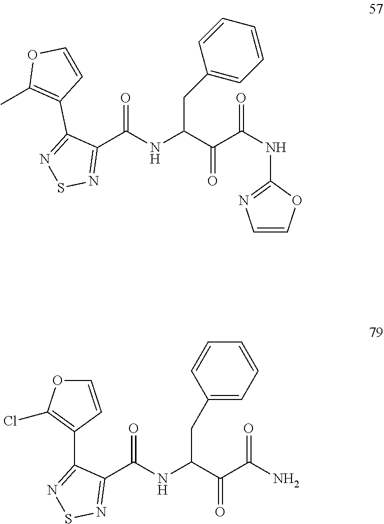 [SMILES: Cc1occc1-c1nsnc1C(=O)NC(Cc1ccccc1)C(=O)C(=O)Nc1ncco1 ; NC(=O)C(=O)C(Cc1ccccc1)NC(=O)c1nsnc1-c1ccoc1Cl]
and pharmaceutically acceptable salts thereof.

151. The compound of claim 82, having the structure selected from the group consisting of: 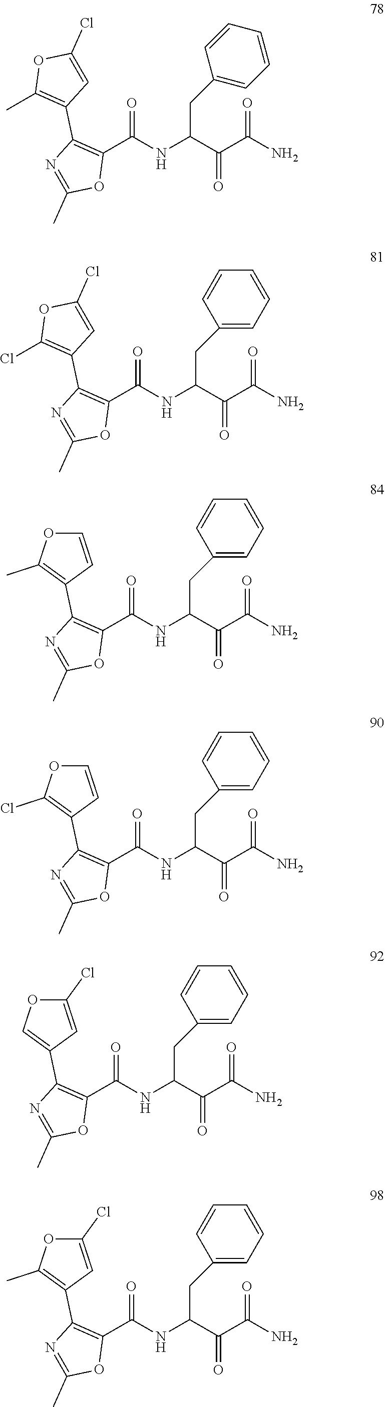 [SMILES: Cc1nc(-c2cc(Cl)oc2C)c(C(=O)NC(Cc2ccccc2)C(=O)C(N)=O)o1 ; Cc1nc(-c2cc(Cl)oc2Cl)c(C(=O)NC(Cc2ccccc2)C(=O)C(N)=O)o1 ; Cc1nc(-c2ccoc2C)c(C(=O)NC(Cc2ccccc2)C(=O)C(N)=O)o1 ; Cc1nc(-c2ccoc2Cl)c(C(=O)NC(Cc2ccccc2)C(=O)C(N)=O)o1 ; Cc1nc(-c2coc(Cl)c2)c(C(=O)NC(Cc2ccccc2)C(=O)C(N)=O)o1 ; Cc1nc(-c2cc(Cl)oc2C)c(C(=O)NC(Cc2ccccc2)C(=O)C(N)=O)o1]
and pharmaceutically acceptable salts thereof.

152. The compound of claim 112, having the structure selected from the group consisting of: 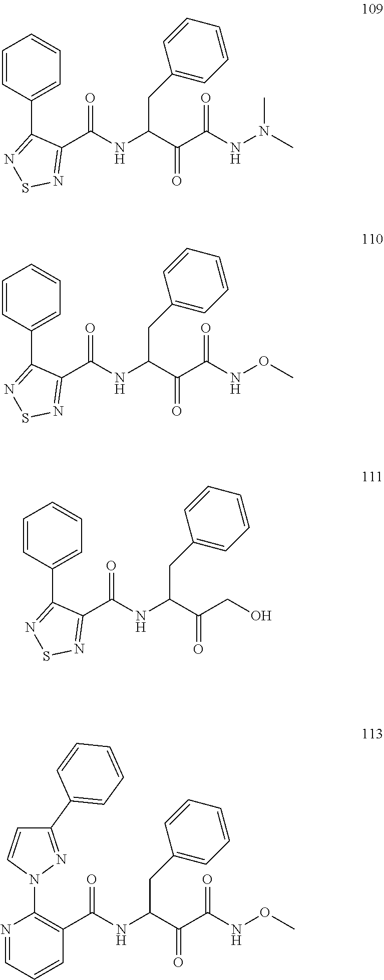 [SMILES: CN(C)NC(=O)C(=O)C(Cc1ccccc1)NC(=O)c1nsnc1-c1ccccc1 ; CONC(=O)C(=O)C(Cc1ccccc1)NC(=O)c1nsnc1-c1ccccc1 ; O=C(NC(Cc1ccccc1)C(=O)CO)c1nsnc1-c1ccccc1 ; CONC(=O)C(=O)C(Cc1ccccc1)NC(=O)c1cccnc1-n1ccc(-c2ccccc2)n1]
and pharmaceutically acceptable salts thereof.

153. A compound having the structure selected from the group consisting of: 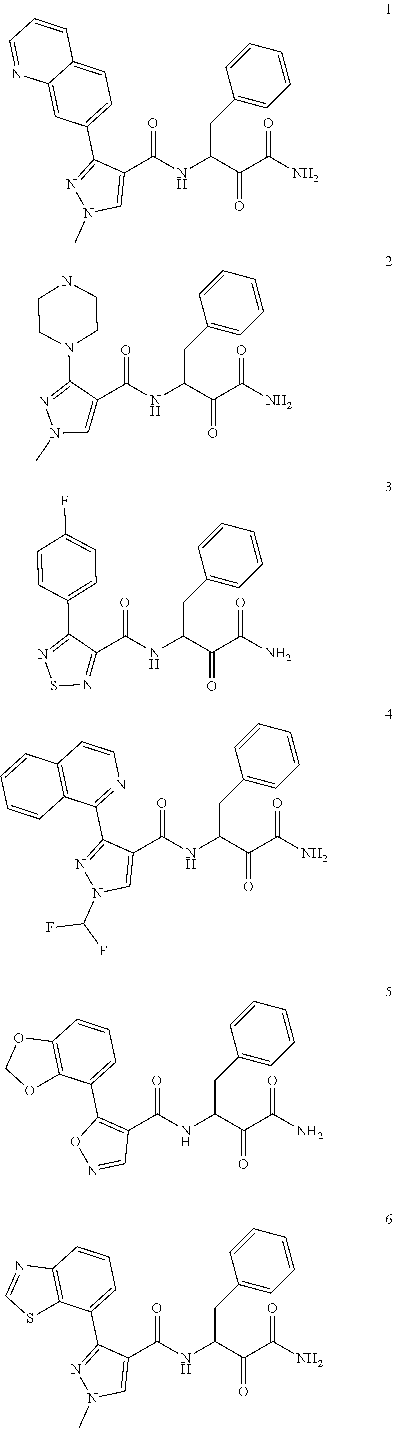 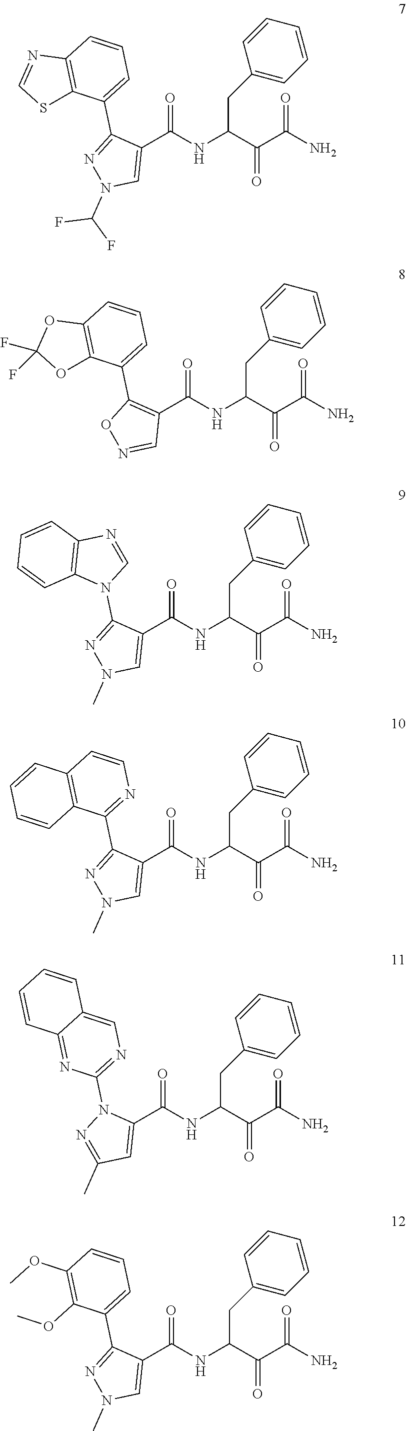 [SMILES: NC(=O)C(=O)C(Cc1ccccc1)NC(=O)c1cn(C(F)F)nc1-c1cccc2ncsc12 ; NC(=O)C(=O)C(Cc1ccccc1)NC(=O)c1cnoc1-c1cccc2c1OC(F)(F)O2 ; Cn1cc(C(=O)NC(Cc2ccccc2)C(=O)C(N)=O)c(-n2cnc3ccccc32)n1 ; Cn1cc(C(=O)NC(Cc2ccccc2)C(=O)C(N)=O)c(-c2nccc3ccccc23)n1 ; Cc1cc(C(=O)NC(Cc2ccccc2)C(=O)C(N)=O)n(-c2ncc3ccccc3n2)n1 ; COc1cccc(-c2nn(C)cc2C(=O)NC(Cc2ccccc2)C(=O)C(N)=O)c1OC] 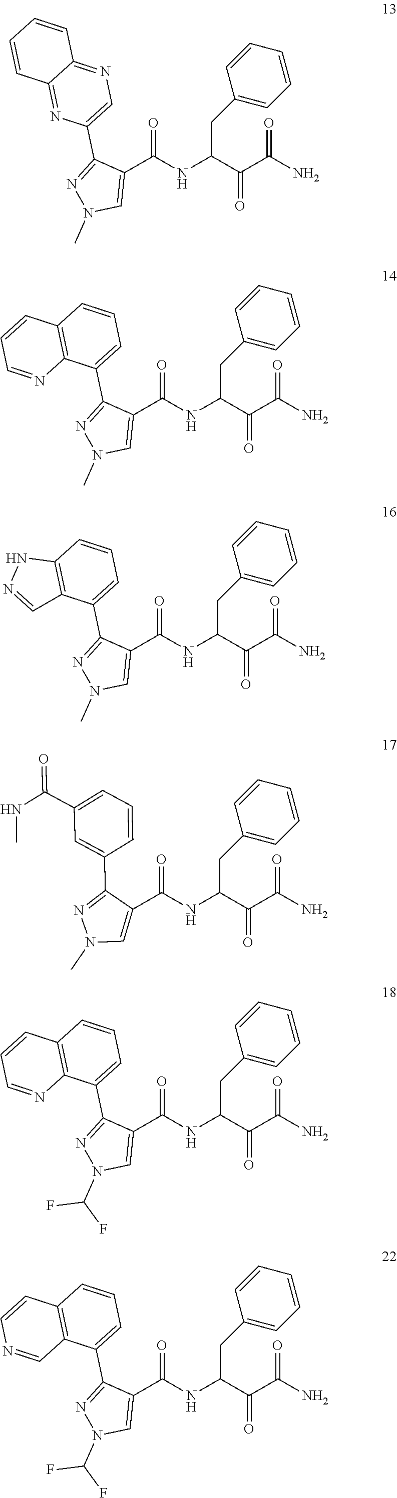 [SMILES: Cn1cc(C(=O)NC(Cc2ccccc2)C(=O)C(N)=O)c(-c2cnc3ccccc3n2)n1 ; Cn1cc(C(=O)NC(Cc2ccccc2)C(=O)C(N)=O)c(-c2cccc3cccnc23)n1 ; Cn1cc(C(=O)NC(Cc2ccccc2)C(=O)C(N)=O)c(-c2cccc3[nH]ncc23)n1 ; CNC(=O)c1cccc(-c2nn(C)cc2C(=O)NC(Cc2ccccc2)C(=O)C(N)=O)c1 ; NC(=O)C(=O)C(Cc1ccccc1)NC(=O)c1cn(C(F)F)nc1-c1cccc2cccnc12 ; NC(=O)C(=O)C(Cc1ccccc1)NC(=O)c1cn(C(F)F)nc1-c1cccc2ccncc12] 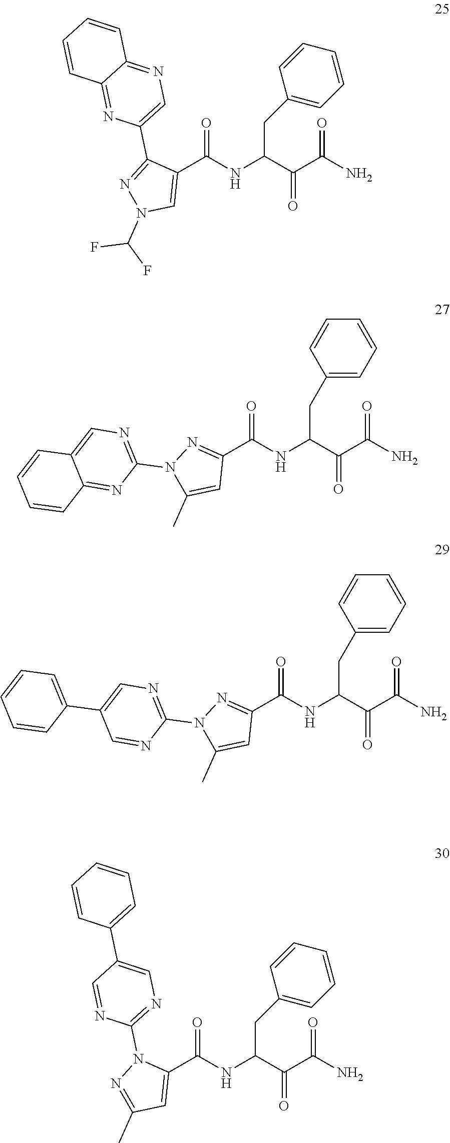 [SMILES: NC(=O)C(=O)C(Cc1ccccc1)NC(=O)c1cn(C(F)F)nc1-c1cnc2ccccc2n1 ; Cc1cc(C(=O)NC(Cc2ccccc2)C(=O)C(N)=O)nn1-c1ncc2ccccc2n1 ; Cc1cc(C(=O)NC(Cc2ccccc2)C(=O)C(N)=O)nn1-c1ncc(-c2ccccc2)cn1 ; Cc1cc(C(=O)NC(Cc2ccccc2)C(=O)C(N)=O)n(-c2ncc(-c3ccccc3)cn2)n1] 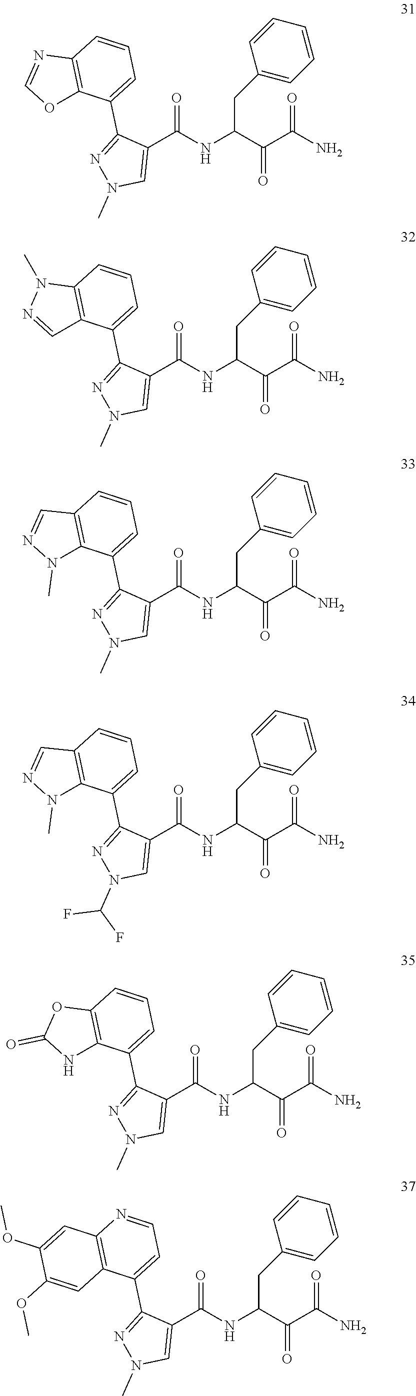 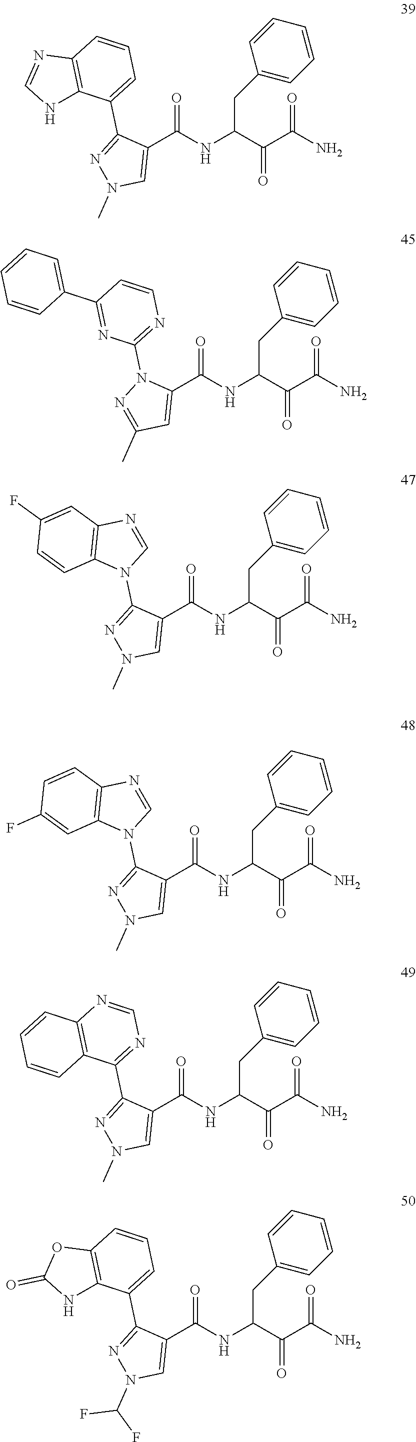 [SMILES: Cn1cc(C(=O)NC(Cc2ccccc2)C(=O)C(N)=O)c(-c2cccc3nc[nH]c23)n1 ; Cc1cc(C(=O)NC(Cc2ccccc2)C(=O)C(N)=O)n(-c2nccc(-c3ccccc3)n2)n1 ; Cn1cc(C(=O)NC(Cc2ccccc2)C(=O)C(N)=O)c(-n2cnc3cc(F)ccc32)n1 ; Cn1cc(C(=O)NC(Cc2ccccc2)C(=O)C(N)=O)c(-n2cnc3ccc(F)cc32)n1 ; Cn1cc(C(=O)NC(Cc2ccccc2)C(=O)C(N)=O)c(-c2ncnc3ccccc23)n1 ; NC(=O)C(=O)C(Cc1ccccc1)NC(=O)c1cn(C(F)F)nc1-c1cccc2oc(=O)[nH]c12] 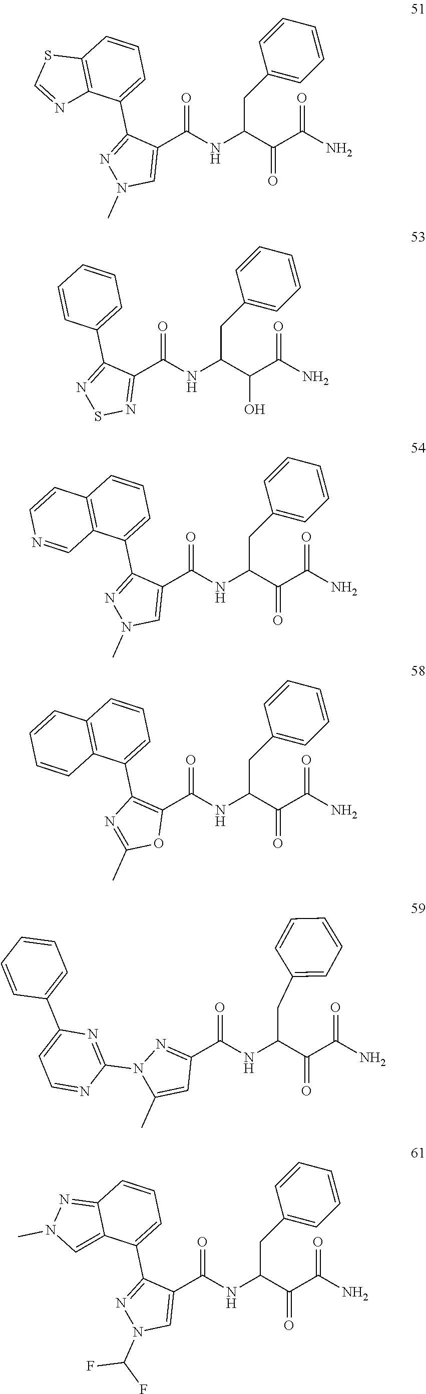 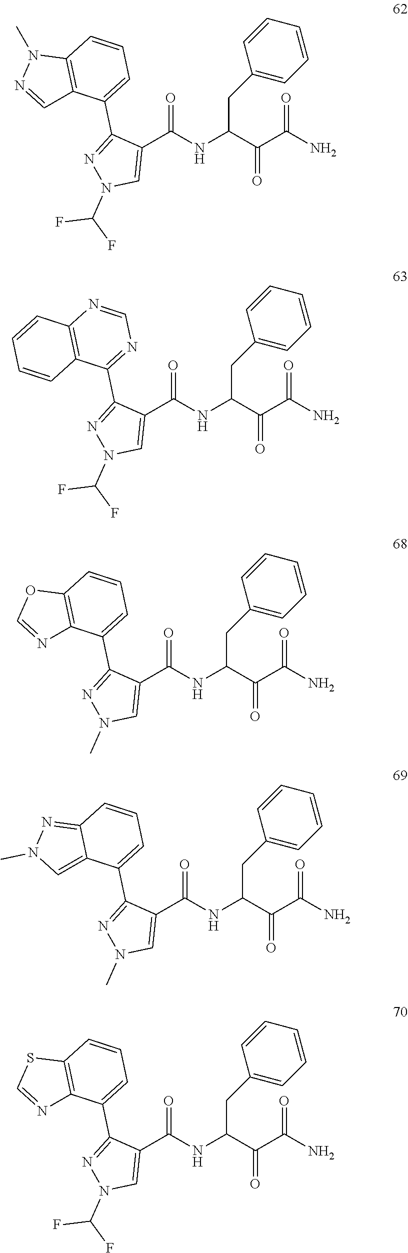 [SMILES: Cn1ncc2c(-c3nn(C(F)F)cc3C(=O)NC(Cc3ccccc3)C(=O)C(N)=O)cccc21 ; NC(=O)C(=O)C(Cc1ccccc1)NC(=O)c1cn(C(F)F)nc1-c1ncnc2ccccc12 ; Cn1cc(C(=O)NC(Cc2ccccc2)C(=O)C(N)=O)c(-c2cccc3ocnc23)n1 ; Cn1cc(C(=O)NC(Cc2ccccc2)C(=O)C(N)=O)c(-c2cccc3nn(C)cc23)n1 ; NC(=O)C(=O)C(Cc1ccccc1)NC(=O)c1cn(C(F)F)nc1-c1cccc2scnc12] 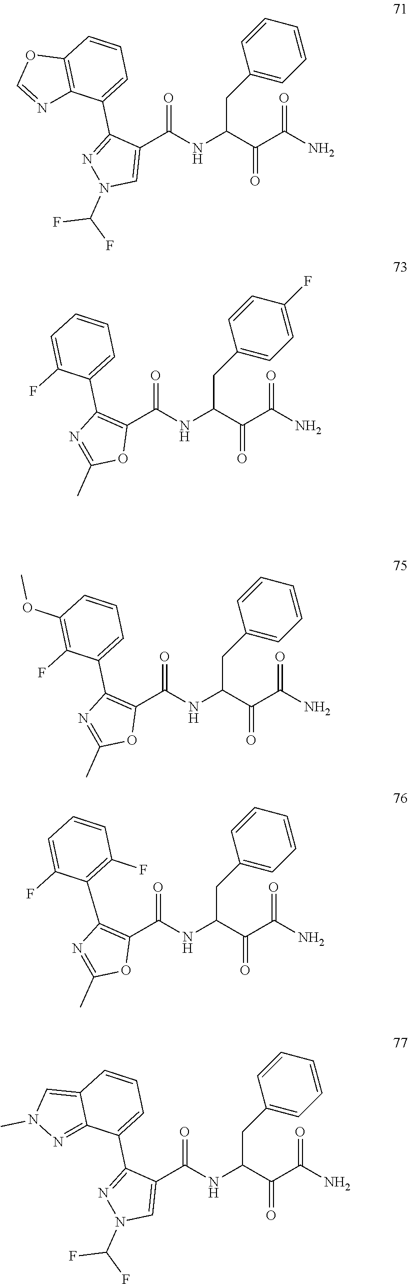 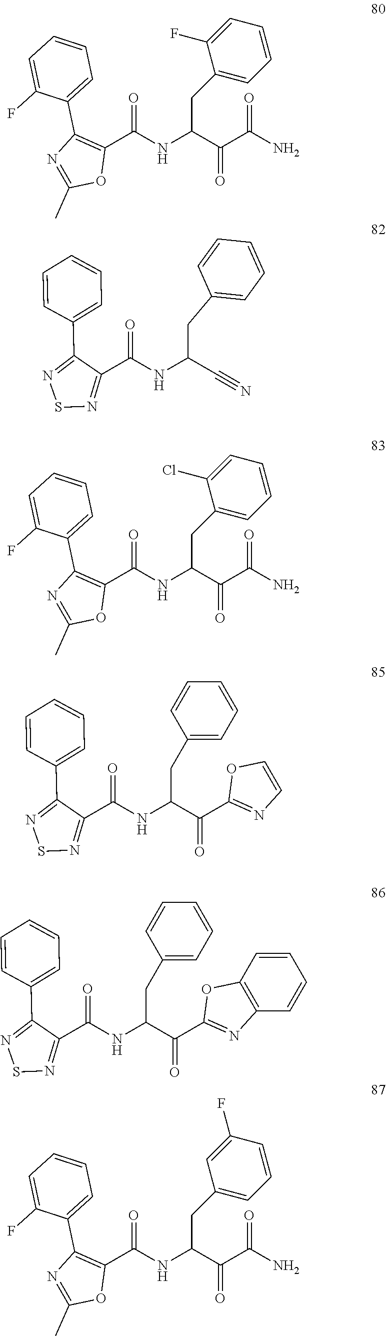 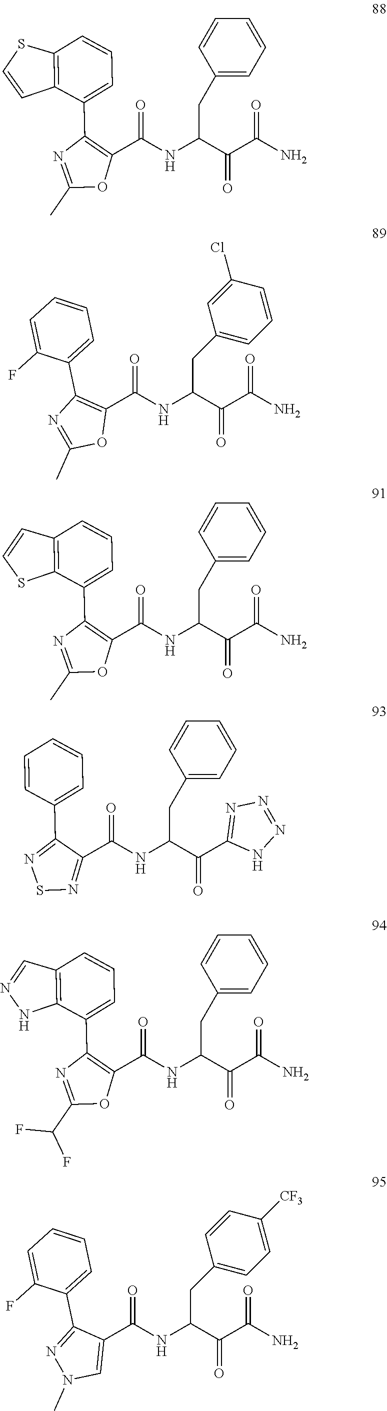 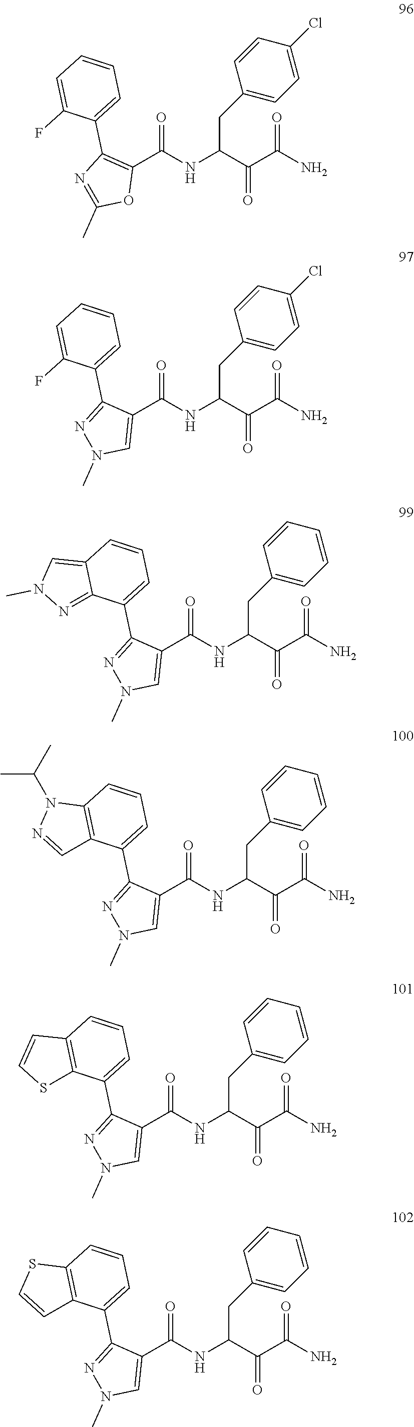 [SMILES: Cc1nc(-c2ccccc2F)c(C(=O)NC(Cc2ccc(Cl)cc2)C(=O)C(N)=O)o1 ; Cn1cc(C(=O)NC(Cc2ccc(Cl)cc2)C(=O)C(N)=O)c(-c2ccccc2F)n1 ; Cn1cc(C(=O)NC(Cc2ccccc2)C(=O)C(N)=O)c(-c2cccc3cn(C)nc23)n1 ; CC(C)n1ncc2c(-c3nn(C)cc3C(=O)NC(Cc3ccccc3)C(=O)C(N)=O)cccc21 ; Cn1cc(C(=O)NC(Cc2ccccc2)C(=O)C(N)=O)c(-c2cccc3ccsc23)n1 ; Cn1cc(C(=O)NC(Cc2ccccc2)C(=O)C(N)=O)c(-c2cccc3sccc23)n1] 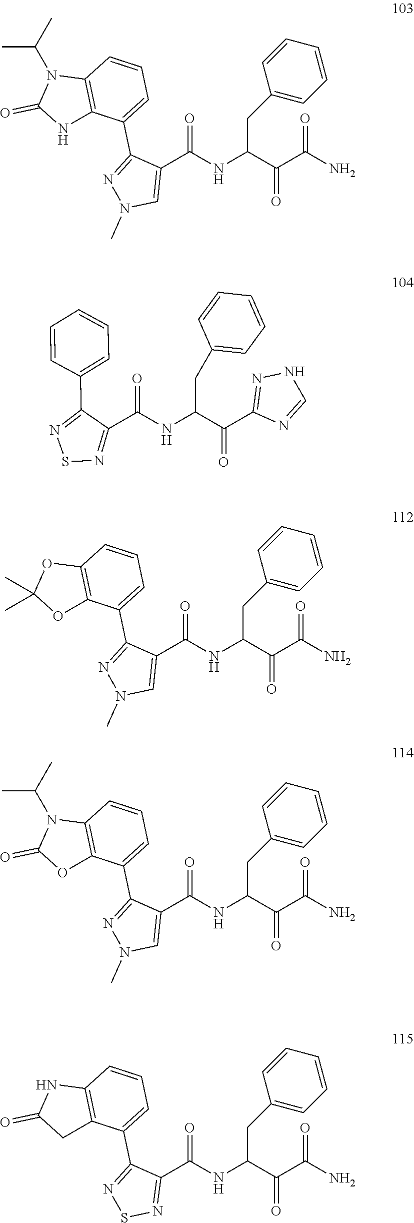 [SMILES: CC(C)n1c(=O)[nH]c2c(-c3nn(C)cc3C(=O)NC(Cc3ccccc3)C(=O)C(N)=O)cccc21 ; O=C(NC(Cc1ccccc1)C(=O)c1nc[nH]n1)c1nsnc1-c1ccccc1 ; Cn1cc(C(=O)NC(Cc2ccccc2)C(=O)C(N)=O)c(-c2cccc3c2OC(C)(C)O3)n1 ; CC(C)n1c(=O)oc2c(-c3nn(C)cc3C(=O)NC(Cc3ccccc3)C(=O)C(N)=O)cccc21 ; NC(=O)C(=O)C(Cc1ccccc1)NC(=O)c1nsnc1-c1cccc2c1CC(=O)N2]
and pharmaceutically acceptable salts thereof.

154. A pharmaceutical composition comprising a therapeutically effective amount of a compound of any one of claims 1-153 and a pharmaceutically acceptable excipient.

155. A method of treating fibrotic disease or a secondary disease state or condition thereof, comprising administering to a subject in need thereof, a compound according to any one of claims 1-153.

156. The method of claim 155, wherein the disease is selected from the group consisting of liver fibrosis, renal fibrosis, lung fibrosis, hypersensitivity pneumonitis, interstitial fibrosis, systemic scleroderma, macular degeneration, pancreatic fibrosis, fibrosis of the spleen, cardiac fibrosis, mediastinal fibrosis, mylofibrosis, endomyocardial fibrosis, retroperitoneal fibrosis, progressive massive fibrosis, nephrogenic systemic fibrosis, fibrotic complications of surgery, chronic allograft vasculopathy and/or chronic rejection in transplanted organs, ischemic-reperfusion injury associated fibrosis, injection fibrosis, cirrhosis, diffuse parenchymal lung disease, post-vasectomy pain syndrome, and rheumatoid arthritis.

157. The method of claim 155, wherein the treatment decreases the expression level and/or activity of a calpain.

158. The method of claim 157, wherein the calpain is CAPN1, CAPN2, or CAPN9.

159. The method of claim 155, wherein the treatment inhibits myofibroblast differentiation or treats a disease associated with myofibroblast differentiation.

160. The method of claim 155, wherein the treatment inhibits Fibroblast-to-Myofibroblast Transition (FMT).

161. The method of claim 155, wherein the treatment inhibits Epithelial to Mesenchymal Transition or Endothelial to Mesenchymal Transition.

162. The method of claim 161 wherein the myofibroblast differentiation is a TGFβ-mediated myofibroblast differentiation.

163. The method of claim 155, wherein the fibrotic disease is a cancer.

164. The method of claim 163, wherein the cancer is a cancer of epithelial origin.

165. The method of claim 164, wherein the cancer of epithelial origin is selected from the group consisting of breast cancer, basal cell carcinoma, adenocarcinoma, gastrointestinal cancer, lip cancer, mouth cancer, esophageal cancer, small bowel cancer, stomach cancer, colon cancer, liver cancer, brain, bladder cancer, pancreas cancer, ovary cancer, cervical cancer, lung cancer, skin cancer, prostate cancer, and renal cell carcinoma.

166. The method of claim 155, wherein the fibrotic disease is stiff skin syndrome (SKS).

167. The method of claim 155, wherein the compound is of Formula I.

168. The method of claim 155, wherein the subject is a mammal.

169. The method of claim 155, wherein the subject is a human.

170. The method of claim 155, wherein the route of administration is selected from the group consisting of: enteral, intravenous, oral, intraarticular, intramuscular, subcutaneous, intraperitoneal, epidural, transdermal, and transmucosal.

171. The method of claim 155, wherein the administration is intravenous.

172. A method of inhibiting myofibroblast differentiation comprising contacting a cell with a compound of anyone of claims 1-153.

173. The method of claim 172, wherein the cell is in a fibrotic tissue.

174. The method of claim 1721, wherein the cell is in a cancerous tissue.

175. The method of claim 172, wherein the cell is in a tissue with high TGFβ signaling.

176. A method for inhibiting calpain, the method comprising contacting a compound of any one of claims 1-153 with a CAPN1, CAPN2, and/or CAPN9 enzyme residing inside a subject.

177. A method of competitive binding with calpastatin (CAST), the method comprising contacting a compound of anyone of claims 1-153 with CAPN1, CAPN2, and/or CAPN9 enzymes residing inside a subject.
